(19) United States
(12) Patent Application Publication
(10) Pub. No.: US 2024/0069035 A1
(43) Pub. Date: Feb. 29, 2024

(54) METHODS AND SYSTEMS FOR DETECTION AND DISCOVERY OF BIOMARKERS

(71) Applicant: Biological Dynamics, Inc., San Diego, CA (US)

(72) Inventors: Rajaram KRISHNAN, San Diego, CA (US); Robert TURNER, San Diego, CA (US); Juan Pablo HINESTROSA SALAZAR, San Diego, CA (US); Jean LEWIS, San Diego, CA (US); Iryna CLARK, Del Mar, CA (US); David SEARSON, San Diego, CA (US)

(21) Appl. No.: 18/261,090

(22) PCT Filed: Jan. 12, 2022
(86) PCT No.: PCT/US2022/012149

§ 371 Date: Jul. 11, 2023

Related U.S. Application Data
(60) Provisional application No. 63/136,572, filed on Jan. 12, 2021.
(60) Provisional application No. 63/190,719, filed on May 19, 2021.
(60) Provisional application No. 63/191,886, filed on May 21, 2021.

(51) Int. Cl. G01N 33/68; G01N 1/34

(52) U.S. Cl. CPC G01N 33/6848; G01N 1/34; G01N 33/6893

(57) ABSTRACT

Provided herein are methods and systems for discovering biomarkers associated with risk of disease and methods using identified biomarkers for detecting disease prognosis and progression.

### CROSS REFERENCE

This application claims the benefit of U.S. Provisional Application No. 63/136,572, filed Jan. 12, 2021, U.S. Provisional Application No. 63/190,719, filed May 19, 2021, and U.S. Provisional Application No. 63/191,886, filed May 21, 2021, each of which is incorporated herein by reference in its entirety.

### BACKGROUND

The discovery and use of biomarkers for detecting, monitoring, and treating disease states shows promise in providing improved outcomes for patients. As diseases often have complex etiologies, selecting a biomarker for detecting, monitoring, and treating a disease is challenging. For example, early-stage, localized tumors are often cured by surgical resection. However, some lethal cancers produce few symptoms, causing delayed diagnosis. Detection of early-stage cancers could transform the field by simplifying treatment while increasing survival.

### SUMMARY

In an aspect, there are provided, methods for identifying a biomarker as associated with a disease state. In some cases the method comprises: (a) isolating a first plurality of analytes in a first biological sample of an individual known to have the disease state using an electrode array configured to generate an AC dielectrophoretic field; (b) isolating a second plurality of analytes in a second biological sample of healthy individual using an electrode array configured to generate an AC dielectrophoretic field; and (c) identifying a subset of the first plurality of analytes, wherein the subset is quantitatively different in the first biological sample compared with the second biological sample, wherein the subset is identified as associated with the disease state. In some cases, isolating comprises using electrodes configured to generate a dielectrophoretic low field region and a dielectrophoretic high field region. In some cases, isolating comprises capturing the first plurality of analytes or the second plurality of analytes on one or more electrode. In some cases, the subset comprises mass spectrometry analysis of the first plurality of analytes and the second plurality of analytes. In some cases, identifying the subset comprises quantifying each of the first plurality of analytes and the second plurality of analytes. In some cases, the analyte comprises a protein or a polypeptide. In some cases, the analyte comprises a nucleic acid. In some cases, the analyte comprises an exosome. In some cases, the disease state is a cancer, a neurological disease, an infection, or an inflammatory disease. In some cases, the cancer is a pancreatic cancer, an ovarian cancer, a bladder cancer, a colorectal cancer, a lung cancer, a brain cancer, a prostate cancer, a breast cancer, a skin cancer, a lymphoma, a tongue cancer, a mouth cancer, a pharynx cancer, an oral cavity cancer, an esophagus cancer, a stomach cancer, a small intestine cancer, a colon cancer, a rectum cancer, an anal cancer, an anorectum cancer, a liver cancer, an intrahepatic bile duct cancer, a gallbladder cancer, a biliary cancer, a digestive organ cancer, a larynx cancer, a bronchus cancer, a respiratory organ cancer, a bone cancer, a joint cancer, a soft tissue cancer, a heart cancer, a melanoma, a nonepithelial skin cancer, a uterine cancer, a cervical cancer, a vulva cancer, a vagina cancer, a penis cancer, a genital cancer, a testis cancer, a kidney cancer, a renal pelvis cancer, a ureter cancer, a urinary organ cancer, an eye cancer, an orbit cancer, a nervous system cancer, an endocrine cancer, a thyroid cancer, a Hodgkin lymphoma, a non-Hodgkin lymphoma, a myeloma, an acute lymphocytic leukemia, a chronic lymphocytic leukemia, an acute myeloid leukemia, a chronic myeloid leukemia, or a leukemia.

In another aspect, there are provided methods of analysis comprising (a) measuring an amount of an analyte in a biological sample from an individual; and (b) identifying the individual as being at risk of developing a disease when the amount of the analyte is greater than or less than the amount observed in a control sample, wherein the analyte comprises one or more biomarker identified in any of the method provided herein. In some cases, measuring comprises isolating the analytes in the biological sample using an electrode array configured to generate an AC dielectrophoretic field. In some cases, isolating comprises using electrodes configured to generate a dielectrophoretic low field region and a dielectrophoretic high field region. In some cases, isolating comprises capturing the first plurality of analytes or the second plurality of analytes on one or more electrode. In some cases, measuring comprises mass spectrometry analysis of the analyte. In some cases, the analyte comprises a protein or a polypeptide. In some cases, the analyte comprises a nucleic acid. In some cases, the analyte comprises an exosome. In some cases, the disease is a cancer, a neurological disease, an infection, or an inflammatory disease. In some cases, the cancer is a pancreatic cancer, an ovarian cancer, a bladder cancer, a colorectal cancer, a lung cancer, a brain cancer, a prostate cancer, a breast cancer, a skin cancer, a lymphoma, or a leukemia.

A further aspect, there are provided methods of identifying a therapeutic target, the method comprising: (a) isolating a first plurality of analytes in a first biological sample of an individual known to have the disease state using an electrode array configured to generate an AC dielectrophoretic field; (b) isolating a second plurality of analytes in a second biological sample of healthy individual using an electrode configured to generate an AC dielectrophoretic field; and (c) identifying a subset of the first plurality of analytes, wherein the subset is quantitatively different in the first biological sample compared with the second biological sample, wherein the subset is identified as the therapeutic target for drug discovery or drug development.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The patent or application file contains at least one drawing executed in color. Copies of this patent or patent application publication with color drawings(s) will be provided by the Office upon request and payment of the necessary fee.

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows (left) a tilted top view of an assay cartridge; (center) a visualization of blood cells separated from the sample away from the electrodes; and (left) a visualization of DNA and extracellular vesicles on the electrode.

FIG. 2 shows a workflow for biomarker analysis: (left) biomarkers isolated on an electrode, (center) various biomarkers to be analyzed; and (right) modalities for downstream analysis either off chip or on chip.

FIG. 3 shows an example of a method for isolating nucleic acids from cells.

FIG. 4 shows an example of a method for isolating extra-cellular nucleic acids from a fluid comprising cells.

FIG. 5 shows a flow diagram for biomarker discovery.

FIG. 6 shows (left) a cluster diagram of exosomal proteins isolated from pancreatic cancer patients (right) a heat map of biomarkers over expressed and under expressed in pancreatic cancer compared with healthy control.

FIG. 7 shows the subjects used in a multi-cancer test using samples from known cancer patients and healthy controls.

FIG. 8 shows results of a multicancer test using samples from known cancer patients and healthy controls.

FIGS. 9A-9C show an experimental outline. FIG. 9A shows a workflow diagram. FIG. 9B shows a statistical approach to develop and evaluate performance of the EXPLORE test: 100 iterations of randomly selected subjects were used for development (training set, 67% of subjects) and performance evaluation (test set, 33% of subjects). FIG. 9C shows relative concentration of 13 exoproteins used in the EXPLORE test by subject ID. The concentration levels were normalized to the highest concentration observed for each biomarker, with lowest expression depicted in white and highest expression in green.

FIGS. 10A-10C show performance of the EXPLORE test. FIG. 10A shows a ROC curve, cancer cohort to healthy controls: the black line represents the average curve of 100 iterations (gray lines). The red diamond denotes 99% specificity. FIG. 10B shows proportion of correctly classified cancer patients (sensitivity) by stage at >99% specificity. FIG. 10C shows proportion of detected cancer patients (sensitivity) based on cancer type at >99% specificity. Error bars indicate 95% confidence intervals.

FIGS. 11A-11C show EXPLORE Test Proportion Detected at >99% (Top), 97% (Middle) and 95% (Bottom) for Cancer Subtypes. FIG. 11A shows pancreatic ductal adenocarcinoma proportion detected for 21 stage I (96%, 97%, 98%) and 23 stage II (95%, 96%, 97%). FIG. 11B shows ovarian cancer proportion detected for 37 Stage I (65%, 69%, 76%) and 25 Stage IA patients (66%, 69%, 75%), as well as 22 Stage I & II serous adenocarcinoma patients (69%, 73%, 80%). FIG. 11C shows bladder cancer proportion detected for 27 Stage I (56%, 61%, 67%), 15 Low Grade (52%, 58%, 68%), and 33 High Grade (50%, 54%, 62%) in both stages I and II. Each diamond represents the mean, and the error bars represent the 95% confidence interval from the 100 test intervals.

FIGS. 12A-12B show size distribution and particle concentrations of exosomes isolated from healthy controls and cancer patients. FIG. 12A shows characterization of exosome samples by NTA analysis for particle size distribution. FIG. 12B shows characterization of exosome samples by NTA analysis for particle concentration. Median values and ranges were calculated for healthy controls (green) and cancer patients (red).

FIGS. 13A-13B show heatmaps of normalized concentration values for analyzed proteins for cancer and healthy cohorts. FIG. 13A shows a heatmap for exoproteins. FIG. 13B shows a heatmap for free proteins. Normalization is across the entire cohort for each marker. Each column represents a subject in the study.

FIG. 14 shows waterfall plots of the protein biomarkers used in the assay. values are sorted from high (left) to low (right). each column represents an individual patient sample (red, cancer patient; green, healthy control).

FIG. 15 shows EXPLORE test performance using exoproteins and free proteins. ROC curves were generated using the protein concentrations derived from the exo-proteins (black) or free-proteins in plasma (orange). AUROC is shown on the graph for each cohort with 95% confidence intervals.

FIGS. 16A-16B show correlation of protein levels. FIG. 16A shows Pearson correlation coefficients for exosomal proteins. FIG. 16B shows Pearson correlation coefficients for free proteins.

FIG. 17 shows a schematic of EV isolation workflows using AC electrokinetics (ACE) or ultracentrifugation methods. (Top) Workflow using the Verita™ Isolation platform. As plasma samples are flowed onto the energized AC Electrokinetics (ACE) microelectrode array, EVs are collected onto the electrodes. Unbound materials are removed with a buffer wash, the electric field turned off, and EVs are eluted into the buffer. (Bottom) Workflow for differential ultracentrifugation. Plasma samples are diluted, and large debris pelleted by low-speed centrifugation. Supernatants are removed and subjected to 2 additional cycles of low-speed centrifugation. EVs in the cleared supernatants are then ultracentrifuged two times and finally the pellet is resuspended in buffer.

FIGS. 18A-18C show characterization of EVs isolated by ACE or differential ultracentrifugation. FIG. 18A shows distribution of particle sizes as determined by nanoparticle tracking analysis. Verita-isolated EVs, shown in blue line; ultracentrifugation-isolated EVs, shown in grey line. FIG. 18B show levels of residual contaminating total proteins based on Qubit™ protein assay. FIG. 18C shows differentiation between controls (left boxes) and cancer cases (right boxes) shown for biomarkers CA 19-9 and CA 125. Top, EVs isolated using the Verita™ system; bottom, EVs isolated by differential ultracentrifugation.

FIG. 19 shows development of a classification algorithm for multi-cancer early detection. Biomarker selection is performed via recursive feature elimination (RFE) with cross validation. After the biomarkers are selected, the dataset is split into training and test sets. The training set is used for determination of the coefficients in the logistic regression for each biomarker and the test set is used to evaluate the performance of the logistic regression fit from the training set in a “hold-out” test set. Finally, the process of splitting the dataset into training and test sets is randomly repeated 100 times for performance confirmation.

FIGS. 20A-20C show overall performance for detecting the presence of early cancer using an EV protein-based logistic classifier. FIG. 20A shows ROC curves from comparison of the cancer cases to the controls on the hold-out test sets: the black line represents the average curve of 100 independently resampled hold-out test sets (grey lines). FIG. 20B shows sensitivity by stage at >99% specificity. Left bar represents combined sensitivity for detecting stage I pancreatic, ovarian, and bladder cancers; right bar represents combined sensitivity for detecting stage II for these cancers. FIG. 20C shows sensitivity by cancer type at >99% specificity. Left bar represents sensitivity for detecting stages I and II pancreatic cancer; center bar, stages I and II ovarian cancer, and right bar, stages I and II bladder cancer. The error bars represent the two-sided 95% Wilson confidence intervals.

FIGS. 21A-21C show sensitivity at >99% specificity for detecting three cancer types using EV protein biomarkers. FIG. 21A shows sensitivity for detecting either stage I or stage II pancreatic cancer. FIG. 21B shows sensitivity for detecting either stage I or stage II ovarian cancer. FIG. 21C shows sensitivity for detecting either stage I or stage II bladder cancer. Error bars represent the two-sided 95% Wilson confidence intervals.

FIGS. 22A-22C show comparison of NTA results from control and cancer cases. FIG. 22A shows particle concentration for Verita-purified EVs. Left box, EVs from control samples; right box, EVs from cancer cases. FIG. 22B shows Verita-purified EV particles, particle median size. Left box, EVs from control samples; right box, EVs from cancer cases FIG. 22C shows overall particle size distribution for cancers and controls. Top line, EVs from control samples; lower line, EVs from cancer cases.

FIGS. 23A-23B shows a comparison between EVs isolated using Verita™ or Differential Ultracentrifugation. FIG. 23A shows particle size distribution shown for controls and for ovarian, bladder, and pancreatic cancer samples. Blue lines, Verita-isolated EVs; grey lines, ultracentrifugation-isolated EVs. FIG. 23B shows protein bioanalyzer electropherograms for selected samples. The blue dashed lines show the protein size range for Albumin (50 to 60 kDa), the green dashed lines show the same range for Fibrinogen (70-85 kDa) and the cyan dash lines show the range for IgG (140-180 kDa).

FIGS. 24A-24B shows a heatmap of normalized concentration values for analyzed proteins for cancer and control cases. FIG. 24A shows Exo-Proteins. FIG. 24B shows Free Proteins. Normalization is across the entire cohort for each marker; each column represents a subject in the study.

FIG. 25 shows a comparison of EV protein concentrations for control and cancer cases across all biomarkers selected in the model. Controls, left boxes; cancer cases, right boxes.

FIG. 26 shows a Pearson Correlation Coefficients for Biomarkers Selected in the Logistic Classifier Model.

FIG. 27 shows ROC Comparison between EV Proteins and Free Proteins. ROC curves were generated using the protein concentrations derived from the exo-proteins (black line) or free proteins (orange line) using the biomarkers selected in the logistic classifier model.

FIGS. 28A-28B show performance of assay using EVs spiked into K2EDTA plasma at known particle concentrations. (A) The concentration of CA 19-9 measured in H1975 EVs at three different particle concentrations shows a linear response with input. The K2EDTA plasma with no EV spike showed negligible concentration of the marker. (B) Quantitative detection of expected proteins based on the EV type spiked into K2EDTA plasma. H1975 cell EVs, red markers; HeLa cell EVs, blue markers.

FIGS. 29A-29B show Pearson correlation of protein levels. FIG. 29A shows EV proteins. FIG. 29B shows free proteins.

### DETAILED DESCRIPTION

Metastatic cancer is deadly, for example pancreatic cancer is one of the deadliest with a dismal 5-year survival rate of ˜3%.3 Indeed, pancreatic ductal adenocarcinoma (PDAC) will soon become the second leading cause of all cancer-related deaths in the United States. In contrast, for the few patients (11%) diagnosed with localized disease, the 5-year survival rate is ˜40%. This large discrepancy in survival between early- and advanced-stage disease is not unique to pancreatic cancer. The 5-year survival rate for metastatic ovarian carcinoma is <31%, versus a remarkable 93% for the −15% of women with localized disease. Even with surgical management and adjuvant therapy, 80% of women with advanced disease develop recurrence, after which curing the malignancy is no longer an expectation. Similarly, in bladder cancer, detection of the disease that has not spread beyond the inner layer of bladder's wall results in a 5-year survival rate of 96%. Importantly, early detection limits the impact on quality of life, since surgical intervention may entail only a trans-urethral bladder tumor resection, whereas more invasive cancer can require radical removal of the entire bladder.

As with many other malignancies, there are no approved screening modalities for these three cancers. Several emerging blood-based multi-cancer detection assays attempt to address the early detection of these cancers by combining machine learning with DNA mutation/methylation and/or protein biomarkers. However, at the specificity (>99%) needed for implementation of widespread screening, many of these tests demonstrated sensitivities as low as 0% for stage I-II cancers (Liu, M. C., et al. Sensitive and specific multi-cancer detection and localization using methylation signatures in cell-free DNA. Annals of Oncology 31, 745-759 (2020); Cohen, J. D., et al. Detection and localization of surgically resectable cancers with a multi-analyte blood test. Science 359, 926-930 (2018)). Recently, proteins bound to exosomes (extracellular vesicles that mediate cell-to-cell communication) were shown to be promising biomarkers for identification of lung and pancreatic cancers (Hoshino, A., et al. Extracellular Vesicle and Particle Biomarkers Define Multiple Human Cancers. Cell 182, 1044-1061.e1018 (2020)). However, the exosome isolation required a one-day cumbersome ultracentrifugation process. Methods provided herein comprise use of exosomes isolated using an alternating current electrokinetic-based platform— Verita™ (Hinestrosa, J. P., et al. Simultaneous Isolation of Circulating Nucleic Acids and EV-Associated Protein Biomarkers From Unprocessed Plasma Using an AC Electrokinetics-Based Platform. Frontiers in Bioengineering and Biotechnology 8(2020)) and probed exosome-borne proteins (exo-proteins), enabling detection of pancreatic and brain cancers with a<2 hr workflow.

Provided herein are systems and methods that utilize circulating markers, such as, proteins associated with plasma exosomes, for use in a multi-cancer detection test for identification of stage I and II cancers. In some cases, methods herein are useful for detection of pancreatic, ovarian, and bladder cancers. In some cases, methods herein are useful in detecting cancers where early detection would provide high clinical value. Methods herein show a reliable detection of early-stage disease, in some cases, with an area under the curve (AUC) of 0.95 (95% Confidence Interval (CI)=0.94-0.97). In some cases, at 99% specificity, the proportions of detected stage I disease reached 97% in pancreatic, 65% in ovarian (66% in Stage IA) and 56% in bladder cancers.

Provided herein are systems and methods for discovery of biomarkers associated with a disease state.

Also provided herein are a plurality of biomarkers useful for identifying an individual at risk of disease or a prognosis or progression of the disease in the individual.

In some aspects, the method, device or system includes the isolation and/or identification of biomarkers from a biological complex, for example vesicles such as extracellular vesicles, exosomes, microvesicles, enveloped-particles, and other complex particles or biological parcels that include a combination of biological components, including DNA, RNA, proteins, lipids and other biological molecules. In some aspects, the method, device or systems described herein isolate biomarkers (e.g., DNA, RNA, nucleosomes, proteins or cell membrane fragments) from exosomes isolated from a biological sample.

In some embodiments, the method, device, or system further includes one or more of the following steps: concentrating exosomes in a first dielectrophoretic field region (e.g., a high field DEP region), and isolating a biomarker (e.g., DNA, RNA, nucleosomes, proteins, or cell membrane fragments) from exosomes. In other embodiments, the method, device, or system includes one or more of the following steps: concentrating larger particulates (e.g., cells) in a first dielectrophoretic field region (e.g., a low field DEP region), concentrating exosomes in a second dielectrophoretic field region (e.g., a high field DEP region), washing away the cells and residual material, and isolating biomarkers from the exosomes. The method also optionally includes devices and/or systems capable of performing one or more of the following steps: washing or otherwise removing residual (e.g., cellular) material from the exosomes (e.g., rinsing the array with water or buffer while the exosomes are concentrated and maintained within a high field DEP region of the array), optionally degrading residual proteins (e.g., residual proteins from lysed cells and/or other sources, such degradation occurring according to any suitable mechanism, such as with heat, a protease, or a chemical), flushing degraded proteins from the nucleic acid, and collecting the exosomes. In some embodiments, the result of the methods, operation of the devices, and operation of the systems described herein is an isolated particulate (e.g., exosomes comprising DNA, RNA, nucleosomes, proteins, cell membrane fragments), optionally of suitable quantity and purity for further analysis (e.g., mass spectroscopy, DNA sequencing).

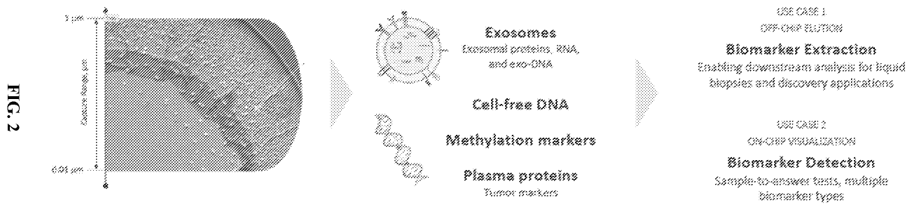

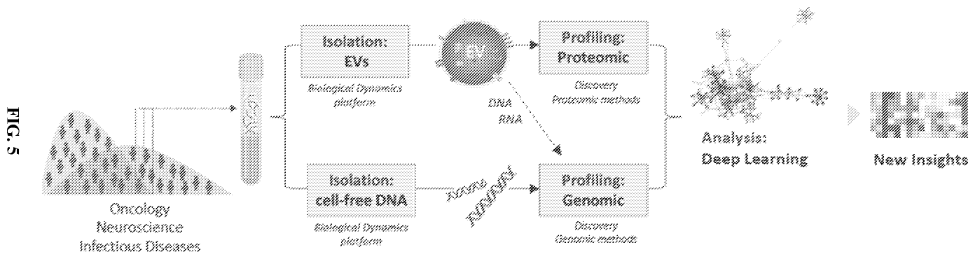

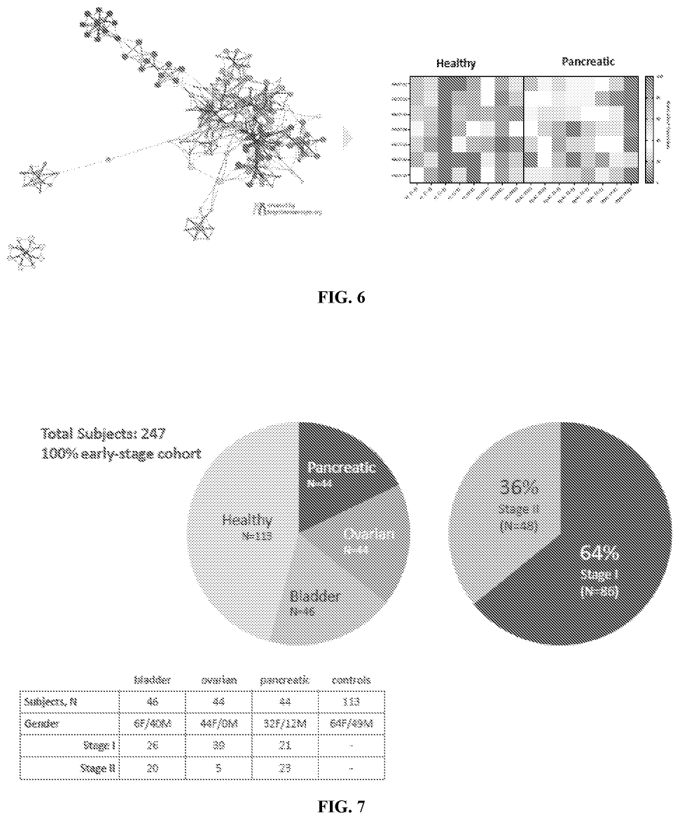

An example workflow is shown in FIG. 2. Biomarkers of a predetermined size range are isolated using dielectrophoresis, analytes captured include but are not limited to exosomes, (including exosomal proteins, RNA and exosomal DNA), cell-free DNA, methylation markers, and/or plasma proteins. Isolated biomarkers are either analyzed after being eluted from the chip or being detected on the chip. A more detailed workflow is shown in FIG. 5. Samples from patients of various disease states, including but not limited to, cancer, neurological disease, or infectious disease, are analyzed by isolating extracellular vesicles (or exosomes) and cell free DNA. Nucleic acids from the extracellular vesicles and the cell-free DNA are analyzed by genomic profiling. Proteins from the extracellular vesicles are analyzed using proteomic methods. Analysis of these analytes and analytes from healthy controls will be analyzed, in some cases using machine learning or deep learning algorithms to discover new biomarkers. Analysis of biomarkers may involve functional clustering and expression comparison (e.g., heat maps) as shown in FIG. 6.

In some instances, it is advantageous that the methods described herein are performed in a short amount of time, the devices are operated in a short amount of time, and the systems are operated in a short amount of time. In some embodiments, the period of time is short with reference to the “procedure time” measured from the time between adding the fluid to the device and obtaining isolated nucleic acid. In some embodiments, the procedure time is less than 3 hours, less than 2 hours, less than 1 hour, less than 30 minutes, less than 20 minutes, less than 10 minutes, or less than 5 minutes.

In another aspect, the period of time is short with reference to the “hands-on time” measured as the cumulative amount of time that a person must attend to the procedure from the time between adding the fluid to the device and obtaining isolated exosomes. In some embodiments, the hands-on time is less than 40 minutes, less than 20 minutes, less than 10 minutes, less than 5 minutes, less than 1 minute, or less than 30 seconds.

In some instances, it is advantageous that the devices described herein comprise a single vessel, the systems described herein comprise a device comprising a single vessel and the methods described herein can be performed in a single vessel, e.g., in a dielectrophoretic device as described herein. In some aspects, such a single-vessel embodiment minimizes the number of fluid handling steps and/or is performed in a short amount of time. In some instances, the present methods, devices and systems are contrasted with methods, devices and systems that use one or more centrifugation steps and/or medium exchanges. In some instances, centrifugation increases the amount of hands-on time required to isolate an analyte or biomarker from exosomes including but not limited to DNA, RNA, nucleosomes, proteins, and/or cell membrane fragments. In another aspect, the single-vessel procedure or device isolates analytes or biomarkers from exosomes (e.g. DNA, RNA, nucleosomes, proteins, and/or cell membrane fragments) using a minimal amount of consumable reagents.

### Devices and Systems

In some embodiments, described herein are devices for collecting exosome derived biomarkers from a fluid. In one aspect, described herein are devices for collecting a biomarker from a fluid comprising cells, from a cell-free portion of a fluid, or other particulate material.

In some embodiments, disclosed herein is a device for isolating cellular material, the device comprising: a. a housing; b. a heater or thermal source and/or a reservoir comprising a protein degradation agent; and c. a plurality of alternating current (AC) electrodes within the housing, the AC electrodes configured to be selectively energized to establish AC electrokinetic high field and AC electrokinetic low field regions, whereby AC electrokinetic effects provide for concentration of cells in low field regions of the device. In some embodiments, the plurality of electrodes is configured to be selectively energized to establish a dielectrophoretic high field and dielectrophoretic low field regions. In some embodiments, the protein degradation agent is a protease. In some embodiments, the protein degradation agent is Proteinase K. In some embodiments, the device further comprises a second reservoir comprising an eluant.

In some embodiments, disclosed herein is a device comprising: a. a plurality of alternating current (AC) electrodes, the AC electrodes configured to be selectively energized to establish AC electrokinetic high field and AC electrokinetic low field regions; and b. a module capable of thermocycling and performing PCR or other enzymatic reactions.

In some embodiments, disclosed herein is a device comprising: a. a plurality of alternating current (AC) electrodes, the AC electrodes configured to be selectively energized to establish AC electrokinetic high field and AC electrokinetic low field regions; and b. a module capable of imaging the material captured or isolated by the AC electrodes. Some embodiments also include chambers and fluidics for adding reagents and removing that allow for the visualization of the captured materials.

In some embodiments, the plurality of electrodes is configured to be selectively energized to establish a dielectrophoretic high field and dielectrophoretic low field regions. In some embodiments, the device is capable of isolating DNA, including cell-free DNA and DNA fragments, RNA, nucleosomes, exosomes, extracellular vesicles, proteins, cell membrane fragments, mitochondria and cellular vesicles from a biological sample comprising fluid. In some embodiments, the device is capable of isolating these materials from cells in the biological sample. In some embodiments, the device is capable of performing PCR amplification or other enzymatic reactions. In some embodiments, DNA is isolated and PCR or other enzymatic reaction is performed in a single chamber. In some embodiments, DNA is isolated and PCR or other enzymatic reaction is performed in multiple regions of a single chamber. In some embodiments, DNA is isolated and PCR or other enzymatic reaction is performed in multiple chambers. In some embodiments, a biomarker is eluted from the device for further analysis (e.g., mass spectroscopy).

In some embodiments, the device further comprises at least one of an elution tube, a chamber and a reservoir to perform PCR amplification or other enzymatic reaction. In some embodiments, PCR amplification or other enzymatic reaction is performed in a serpentine microchannel comprising a plurality of temperature zones. In some embodiments, PCR amplification or other enzymatic reaction is performed in aqueous droplets entrapped in immiscible fluids (i.e., digital PCR). In some embodiments, the thermocycling comprises convection. In some embodiments, the device comprises a surface contacting or proximal to the electrodes, wherein the surface is functionalized with biological ligands that are capable of selectively capturing biomolecules.

In some embodiments, disclosed herein is a system for isolating a cellular material from a biological sample, the system comprising: a. a device comprising a plurality of alternating current (AC) electrodes, the AC electrodes configured to be selectively energized to establish AC electrokinetic high field and AC electrokinetic low field regions, whereby AC electrokinetic effects provide for concentration of cells in high field regions of the device; and b. a sequencer, thermocycler or other device for performing enzymatic reactions on isolated or collected nucleic acid. In some embodiments, the plurality of electrodes is configured to be selectively energized to establish a dielectrophoretic high field and dielectrophoretic low field regions.

In various embodiments, DEP fields are created or capable of being created by selectively energizing an array of electrodes as described herein. The electrodes are optionally made of any suitable material resistant to corrosion, including metals, such as noble metals (e.g. platinum, platinum iridium alloy, palladium, gold, and the like). In various embodiments, electrodes are of any suitable size, of any suitable orientation, of any suitable spacing, energized or capable of being energized in any suitable manner, and the like such that suitable DEP and/or other electrokinetic fields are produced.

In some embodiments described herein are methods, devices and systems in which the electrodes are placed into separate chambers and positive DEP regions and negative DEP regions are created within an inner chamber by passage of the AC DEP field through pore or hole structures. Various geometries are used to form the desired positive DEP (high field) regions and DEP negative (low field) regions for carrying cellular, microparticle, nanoparticle, and nucleic acid separations. In some embodiments, pore or hole structures contain (or are filled with) porous material (hydrogels) or are covered with porous membrane structures. In some embodiments, by segregating the electrodes into separate chambers, such pore/hole structure DEP devices reduce electrochemistry effects, heating, or chaotic fluidic movement from occurring in the inner separation chamber during the DEP process.

In one aspect, described herein is a device comprising electrodes, wherein the electrodes are placed into separate chambers and DEP fields are created within an inner chamber by passage through pore structures. The exemplary device includes a plurality of electrodes and electrode-containing chambers within a housing. A controller of the device independently controls the electrodes, as described further in PCT patent publication WO 2009/146143 A2, which is incorporated herein for such disclosure.

In some embodiments, chambered devices are created with a variety of pore and/or hole structures (nanoscale, microscale and even macroscale) and contain membranes, gels or filtering materials which control, confine or prevent cells, nanoparticles or other entities from diffusing or being transported into the inner chambers while the AC/DC electric fields, solute molecules, buffer and other small molecules can pass through the chambers.

In various embodiments, a variety of configurations for the devices are possible. For example, a device comprising a larger array of electrodes, for example in a square or rectangular pattern configured to create a repeating non-uniform electric field to enable AC electrokinetics. For illustrative purposes only, a suitable electrode array may include, but is not limited to, a 10×10 electrode configuration, a 50×50 electrode configuration, a 10×100 electrode configuration, a 20×100 electrode configuration, or a 20×80 electrode configuration.

Such devices include, but are not limited to, multiplexed electrode and chambered devices, devices that allow reconfigurable electric field patterns to be created, devices that combine DC electrophoretic and fluidic processes; sample preparation devices, sample preparation, enzymatic manipulation of isolated nucleic acid molecules and diagnostic devices that include subsequent detection and analysis, lab-on-chip devices, point-of-care and other clinical diagnostic systems or versions.

In some embodiments, a planar platinum electrode array device comprises a housing through which a sample fluid flows. In some embodiments, fluid flows from an inlet end to an outlet end, optionally comprising a lateral analyte outlet. The exemplary device includes multiple AC electrodes. In some embodiments, the sample consists of a combination of micron-sized entities or cells, larger nanoparticulates and smaller nanoparticulates or biomolecules. In some instances, the larger nanoparticulates are cellular debris dispersed in the sample. In some embodiments, the smaller nanoparticulates are proteins, smaller DNA, RNA and cellular fragments. In some embodiments, the planar electrode array device is a 60×20 electrode array that is optionally sectioned into three 20×20 arrays that can be separately controlled but operated simultaneously. The optional auxiliary DC electrodes can be switched on to positive charge, while the optional DC electrodes are switched on to negative charge for electrophoretic purposes. In some instances, each of the controlled AC and DC systems is used in both a continuous and/or pulsed manner (e.g., each can be pulsed on and off at relatively short time intervals) in various embodiments. The optional planar electrode arrays along the sides of the sample flow, when over-layered with nanoporous material (e.g., a hydrogel of synthetic polymer), are optionally used to generate DC electrophoretic forces as well as AC DEP. Additionally, microelectrophoretic separation processes is optionally carried out within the nanopore layers using planar electrodes in the array and/or auxiliary electrodes in the x-y-z dimensions.

In various embodiments these methods, devices and systems are operated in the AC frequency range of from 1,000 Hz to 100 MHz, at voltages which could range from approximately 1 volt to 2000 volts pk-pk; at DC voltages from 1 volt to 1000 volts, at flow rates of from 10 microliters per minute to 10 milliliter per minute, and in temperature ranges from 1° C. to 120° C. In some embodiments, the methods, devices and systems are operated in AC frequency ranges of from about 3 to about 15 kHz. In some embodiments, the methods, devices, and systems are operated at voltages of from 5-25 volts pk-pk. In some embodiments, the methods, devices and systems are operated at voltages of from about 1 to about 50 volts/cm. In some embodiments, the methods, devices and systems are operated at DC voltages of from about 1 to about 5 volts. In some embodiments, the methods, devices and systems are operated at a flow rate of from about 10 microliters to about 500 microliters per minute. In some embodiments, the methods, devices and systems are operated in temperature ranges of from about 20° C. to about 60° C. In some embodiments, the methods, devices and systems are operated in AC frequency ranges of from 1,000 Hz to 10 MHz. In some embodiments, the methods, devices and systems are operated in AC frequency ranges of from 1,000 Hz to 1 MHz. In some embodiments, the methods, devices and systems are operated in AC frequency ranges of from 1,000 Hz to 100 kHz. In some embodiments, the methods, devices and systems are operated in AC frequency ranges of from 1,000 Hz to 10 kHz. In some embodiments, the methods, devices and systems are operated in AC frequency ranges of from 10 kHz to 100 kHz. In some embodiments, the methods, devices and systems are operated in AC frequency ranges of from 100 kHz to 1 MHz. In some embodiments, the methods, devices and systems are operated at voltages from approximately 1 volt to 1500 volts pk-pk. In some embodiments, the methods, devices and systems are operated at voltages from approximately 1 volt to 1500 volts pk-pk. In some embodiments, the methods, devices and systems are operated at voltages from approximately 1 volt to 1000 volts pk-pk. In some embodiments, the methods, devices and systems are operated at voltages from approximately 1 volt to 500 volts pk-pk. In some embodiments, the methods, devices and systems are operated at voltages from approximately 1 volt to 250 volts pk-pk. In some embodiments, the methods, devices and systems are operated at voltages from approximately 1 volt to 100 volts pk-pk. In some embodiments, the methods, devices and systems are operated at voltages from approximately 1 volt to 50 volts pk-pk. In some embodiments, the methods, devices and systems are operated at DC voltages from 1 volt to 1000 volts. In some embodiments, the methods, devices and systems are operated at DC voltages from 1 volt to 500 volts. In some embodiments, the methods, devices and systems are operated at DC voltages from 1 volt to 250 volts. In some embodiments, the methods, devices and systems are operated at DC voltages from 1 volt to 100 volts. In some embodiments, the methods, devices and systems are operated at DC voltages from 1 volt to 50 volts. In some embodiments, the methods, devices, and systems are operated at flow rates of from 10 microliters per minute to 1 ml per minute. In some embodiments, the methods, devices, and systems are operated at flow rates of from 0.1 microliters per minute to 500 microliters per minute. In some embodiments, the methods, devices, and systems are operated at flow rates of from 0.1 microliters per minute to 250 microliters per minute. In some embodiments, the methods, devices, and systems are operated at flow rates of from 0.1 microliters per minute to 100 microliters per minute. In some embodiments, the methods, devices, and systems are operated in temperature ranges from 1° C. to 100° C. In some embodiments, the methods, devices, and systems are operated in temperature ranges from 20° C. to 95° C. In some embodiments, the methods, devices, and systems are operated in temperature ranges from 25° C. to 100° C. In some embodiments, the methods, devices, and systems are operated at room temperature.

In some embodiments, the controller independently controls each of the electrodes. In some embodiments, the controller is externally connected to the device such as by a socket and plug connection, or is integrated with the device housing.

Also described herein are scaled sectioned (x-y dimensional) arrays of robust electrodes and strategically placed (x-y-z dimensional) arrangements of auxiliary electrodes that combine DEP, electrophoretic, and fluidic forces, and use thereof. In some embodiments, clinically relevant volumes of blood, serum, plasma, or other samples are more directly analyzed under higher ionic strength and/or conductance conditions. Described herein is the overlaying of robust electrode structures (e.g. platinum, palladium, gold, etc.) with one or more porous layers of materials (natural or synthetic porous hydrogels, membranes, controlled nanopore materials, and thin dielectric layered materials) to reduce the effects of any electrochemistry (electrolysis) reactions, heating, and chaotic fluid movement that may occur on or near the electrodes, and still allow the effective separation of cells, bacteria, virus, nanoparticles, exosomes, DNA, RNA, nucleosomes, extracellular vesicles, proteins, cell membrane fragments, mitochondria and cellular vesicles, and other biomolecules to be carried out. In some embodiments, in addition to using AC frequency cross-over points to achieve higher resolution separations, on-device (on-array) DC microelectrophoresis is used for secondary separations. For example, the separation of DNA nanoparticulates (20-50 kb), high molecular weight DNA (5-20 kb), intermediate molecular weight DNA (1-5 kb), and lower molecular weight DNA (0.1-1 kb) fragments may be accomplished through DC microelectrophoresis on the array. In some embodiments, the device is sub-sectioned, optionally for purposes of concurrent separations of different blood cells, bacteria and virus, and DNA carried out simultaneously on such a device.

In some embodiments, the device comprises a housing and a heater or thermal source and/or a reservoir comprising a protein degradation agent. In some embodiments, the heater or thermal source is capable of increasing the temperature of the fluid to a desired temperature (e.g., to a temperature suitable for degrading proteins, about 30° C., 40° C., 50° C., 60° C., 70° C., or the like). In some embodiments, the heater or thermal source is suitable for operation as a PCR thermocycler. IN other embodiments, the heater or thermal source is used to maintain a constant temperature (isothermal conditions). In some embodiments, the protein degradation agent is a protease. In other embodiments, the protein degradation agent is Proteinase K and the heater or thermal source is used to inactivate the protein degradation agent.

In some embodiments, the device also comprises a plurality of alternating current (AC) electrodes within the housing, the AC electrodes capable of being configured to be selectively energized to establish dielectrophoretic (DEP) high field and dielectrophoretic (DEP) low field regions, whereby AC electrokinetic effects provide for concentration of cells in low field regions of the device. In some embodiments, the electrodes are selectively energized to provide the first AC electrokinetic field region and subsequently or continuously selectively energized to provide the second AC electrokinetic field region. For example, further description of the electrodes and the concentration of cells in DEP fields is found in PCT patent publication WO 2009/146143 A2, which is incorporated herein for such disclosure.

In some embodiments, the device comprises a second reservoir comprising an eluant. The eluant is any fluid suitable for eluting the isolated cellular material from the device. In some instances the eluant is water or a buffer. In some instances, the eluant comprises reagents required for a DNA sequencing method. In some cases, the eluant comprises reagents required for a mass spectroscopy method.

In some embodiments, the device comprises a plurality of reservoirs, each reservoir containing a reagents useful in the staining and washing of the isolated cellular material in the device. Examples include antibodies, oligonucleotides, probes, and dyes, buffers, washes, water, detergents, and solvents.

Also provided herein are systems and devices comprising a plurality of alternating current (AC) electrodes, the AC electrodes configured to be selectively energized to establish dielectrophoretic (DEP) high field and dielectrophoretic (DEP) low field regions. In some instances, AC electrokinetic effects provide for concentration of cells in low field regions and/or concentration (or collection or isolation) of molecules (e.g., macromolecules, such as nucleic acid) in high field regions of the DEP field.

Also provided herein are systems and devices comprising a pluarilty of direct current (DC) electrodes. In some embodiments, the plurality of DC electrodes comprises at least two rectangular electrodes, spread throughout the array. In some embodiments, the electrodes are located at the edges of the array. In some embodiments, DC electrodes are interspersed between AC electrodes.

In some embodiments, a system or device described herein comprises a means for manipulating nucleic acid. In some embodiments, a system or device described herein includes a means of performing enzymatic reactions. In other embodiments, a system or device described herein includes a means of performing polymerase chain reaction, isothermal amplification, ligation reactions, restriction analysis, nucleic acid cloning, transcription or translation assays, or other enzymatic-based molecular biology assay.

In some embodiments, a system or device described herein comprises a nucleic acid sequencer. The sequencer is optionally any suitable DNA sequencing device including but not limited to a Sanger sequencer, pyro-sequencer, ion semiconductor sequencer, polony sequencer, sequencing by ligation device, DNA nanoball sequencing device, or single molecule sequencing device.

In some embodiments, a system or device described herein is capable of maintaining a constant temperature. In some embodiments, a system or device described herein is capable of cooling the array or chamber. In some embodiments, a system or device described herein is capable of heating the array or chamber. In some embodiments, a system or device described herein comprises a thermocycler. In some embodiments, the devices disclosed herein comprises a localized temperature control element. In some embodiments, the devices disclosed herein are capable of both sensing and controlling temperature.

In some embodiments, the devices further comprise heating or thermal elements. In some embodiments, a heating or thermal element is localized underneath an electrode. In some embodiments, the heating or thermal elements comprise a metal. In some embodiments, the heating or thermal elements comprise tantalum, aluminum, tungsten, or a combination thereof. Generally, the temperature achieved by a heating or thermal element is proportional to the current running through it. In some embodiments, the devices disclosed herein comprise localized cooling elements. In some embodiments, heat resistant elements are placed directly under the exposed electrode array. In some embodiments, the devices disclosed herein are capable of achieving and maintaining a temperature between about 20° C. and about 120° C. In some embodiments, the devices disclosed herein are capable of achieving and maintaining a temperature between about 30° C. and about 100° C. In other embodiments, the devices disclosed herein are capable of achieving and maintaining a temperature between about 20° C. and about 95° C. In some embodiments, the devices disclosed herein are capable of achieving and maintaining a temperature between about 25° C. and about 90° C., between about 25° C. and about 85° C., between about 25° C. and about 75° C., between about 25° C. and about 65° C. or between about 25° C. and about 55° C. In some embodiments, the devices disclosed herein are capable of achieving and maintaining a temperature of about 20° C., about 30° C., about 40° C., about 50° C., about 60° C., about 70° C., about 80° C., about 90° C., about 100° C., about 110° C. or about 120° C.

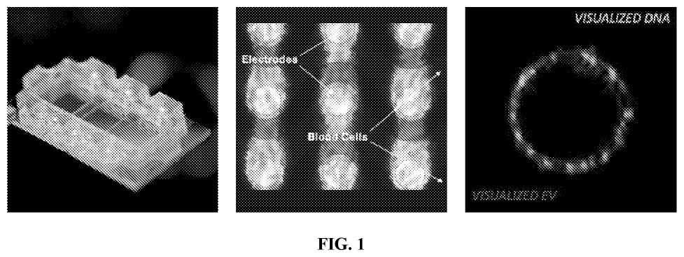

An example device is shown in FIG. 1 with the cartridge having the DEP electrodes in the left panel. A visualization of the electrodes after separation shows blood cells clustering away from the electrodes (center panel). The DNA and extracellular vesicles are seen in the right panel accumulating on the electrode.

Electrodes

The plurality of alternating current electrodes are optionally configured in any manner suitable for the separation processes described herein. For example, further description of the system or device including electrodes and/or concentration of cells in DEP fields is found in PCT patent publication WO 2009/146143, which is incorporated herein for such disclosure.

In some embodiments, the electrodes disclosed herein can comprise any suitable metal. In some embodiments, the electrodes can include but are not limited to: aluminum, copper, carbon, iron, silver, gold, palladium, platinum, iridium, platinum iridium alloy, ruthenium, rhodium, osmium, tantalum, titanium, tungsten, polysilicon, and indium tin oxide, or combinations thereof, as well as silicide materials such as platinum silicide, titanium silicide, gold silicide, or tungsten silicide. In some embodiments, the electrodes can comprise a conductive ink capable of being screen-printed.

In some embodiments, the edge to edge (E2E) to diameter ratio of an electrode is about 0.5 mm to about 5 mm. In some embodiments, the E2E to diameter ratio is about 1 mm to about 4 mm. In some embodiments, the E2E to diameter ratio is about 1 mm to about 3 mm. In some embodiments, the E2E to diameter ratio is about 1 mm to about 2 mm. In some embodiments, the E2E to diameter ratio is about 2 mm to about 5 mm. In some embodiments, the E2E to diameter ratio is about 1 mm. In some embodiments, the E2E to diameter ratio is about 2 mm. In some embodiments, the E2E to diameter ratio is about 3 mm. In some embodiments, the E2E to diameter ratio is about 4 mm. In some embodiments, the E2E to diameter ratio is about 5 mm.

In some embodiments, the electrodes disclosed herein are dry-etched. In some embodiments, the electrodes are wet etched. In some embodiments, the electrodes undergo a combination of dry etching and wet etching.

In some embodiments, each electrode is individually site-controlled.

In some embodiments, an array of electrodes is controlled as a unit.

In some embodiments, a passivation layer is employed. In some embodiments, a passivation layer can be formed from any suitable material known in the art. In some embodiments, the passivation layer comprises silicon nitride. In some embodiments, the passivation layer comprises silicon dioxide. In some embodiments, the passivation layer has a relative electrical permittivity of from about 2.0 to about 8.0. In some embodiments, the passivation layer has a relative electrical permittivity of from about 3.0 to about 8.0, about 4.0 to about 8.0 or about 5.0 to about 8.0. In some embodiments, the passivation layer has a relative electrical permittivity of about 2.0 to about 4.0. In some embodiments, the passivation layer has a relative electrical permittivity of from about 2.0 to about 3.0. In some embodiments, the passivation layer has a relative electrical permittivity of about 2.0, about 2.5, about 3.0, about 3.5 or about 4.0.

In some embodiments, the passivation layer is between about 0.1 microns and about 10 microns in thickness. In some embodiments, the passivation layer is between about 0.5 microns and 8 microns in thickness. In some embodiments, the passivation layer is between about 1.0 micron and 5 microns in thickness. In some embodiments, the passivation layer is between about 1.0 micron and 4 microns in thickness. In some embodiments, the passivation layer is between about 1.0 micron and 3 microns in thickness. In some embodiments, the passivation layer is between about 0.25 microns and 2 microns in thickness. In some embodiments, the passivation layer is between about 0.25 microns and 1 micron in thickness.

In some embodiments, the passivation layer is comprised of any suitable insulative low k dielectric material, including but not limited to silicon nitride or silicon dioxide. In some embodiments, the passivation layer is chosen from the group consisting of polyamids, carbon, doped silicon nitride, carbon doped silicon dioxide, fluorine doped silicon nitride, fluorine doped silicon dioxide, porous silicon dioxide, or any combinations thereof. In some embodiments, the passivation layer can comprise a dielectric ink capable of being screen-printed.

### Electrode Geometry

In some embodiments, the electrodes disclosed herein can be arranged in any manner suitable for practicing the methods disclosed herein.

In some embodiments, the electrodes are in a dot configuration, e.g. the electrodes comprises a generally circular or round configuration. In some embodiments, the angle of orientation between dots is from about 25° to about 60°. In some embodiments, the angle of orientation between dots is from about 30° to about 55°. In some embodiments, the angle of orientation between dots is from about 30° to about 50°. In some embodiments, the angle of orientation between dots is from about 35° to about 45°. In some embodiments, the angle of orientation between dots is about 25°. In some embodiments, the angle of orientation between dots is about 30°. In some embodiments, the angle of orientation between dots is about 35°. In some embodiments, the angle of orientation between dots is about 40°. In some embodiments, the angle of orientation between dots is about 45°. In some embodiments, the angle of orientation between dots is about 50°. In some embodiments, the angle of orientation between dots is about 55°. In some embodiments, the angle of orientation between dots is about 60°.

In some embodiments, the electrodes are in a substantially elongated configuration.

In some embodiments, the electrodes are in a configuration resembling wavy or nonlinear lines. In some embodiments, the array of electrodes is in a wavy or nonlinear line configuration, wherein the configuration comprises a repeating unit comprising the shape of a pair of dots connected by a linker, wherein the dots and linker define the boundaries of the electrode, wherein the linker tapers inward towards or at the midpoint between the pair of dots, wherein the diameters of the dots are the widest points along the length of the repeating unit, wherein the edge to edge distance between a parallel set of repeating units is equidistant, or roughly equidistant. In some embodiments, the electrodes are strips resembling wavy lines, as depicted in FIG. 8. In some embodiments, the edge to edge distance between the electrodes is equidistant, or roughly equidistant throughout the wavy line configuration. In some embodiments, the use of wavy line electrodes, as disclosed herein, lead to an enhanced DEP field gradient.

In some embodiments, the electrodes disclosed herein are in a planar configuration. In some embodiments, the electrodes disclosed herein are in a non-planar configuration.

In some embodiments, the devices disclosed herein surface selectively captures biomolecules on its surface. For example, the devices disclosed herein may capture biomolecules, such as nucleic acids, by, for example, a. nucleic acid hybridization; b. antibody—antigen interactions; c. biotin—avidin interactions; d. ionic or electrostatic interactions; or e. any combination thereof. The devices disclosed herein, therefore, may incorporate a functionalized surface which includes capture molecules, such as complementary nucleic acid probes, antibodies or other protein captures capable of capturing biomolecules (such as nucleic acids), biotin or other anchoring captures capable of capturing complementary target molecules such as avidin, capture molecules capable of capturing biomolecules (such as nucleic acids) by ionic or electrostatic interactions, or any combination thereof.

In some embodiments, the surface is functionalized to minimize and/or inhibit nonspecific binding interactions by: a. polymers (e.g., polyethylene glycol PEG); b. ionic or electrostatic interactions; c. surfactants; or d. any combination thereof. In some embodiments, the methods disclosed herein include use of additives which reduce non-specific binding interactions by interfering in such interactions, such as Tween 20 and the like, bovine serum albumin, nonspecific immunoglobulins, etc.

In some embodiments, the device comprises a plurality of microelectrode devices oriented (a) flat side by side, (b) facing vertically, or (c) facing horizontally. In other embodiments, the electrodes are in a sandwiched configuration, e.g. stacked on top of each other in a vertical format.

### Hydrogels

Overlaying electrode structures with one or more layers of materials can reduce the deleterious electrochemistry effects, including but not limited to electrolysis reactions, heating, and chaotic fluid movement that may occur on or near the electrodes, and still allow the effective separation of cells, bacteria, virus, nanoparticles, DNA, and other biomolecules to be carried out. In some embodiments, the materials layered over the electrode structures may be one or more porous layers. In other embodiments, the one or more porous layers is a polymer layer. In other embodiments, the one or more porous layers is a hydrogel.

In general, the hydrogel should have sufficient mechanical strength and be relatively chemically inert such that it will be able to endure the electrochemical effects at the electrode surface without disconfiguration or decomposition. In general, the hydrogel is sufficiently permeable to small aqueous ions, but keeps biomolecules away from the electrode surface.

In some embodiments, the hydrogel is a single layer, or coating.

In some embodiments, the hydrogel comprises a gradient of porosity, wherein the bottom of the hydrogel layer has greater porosity than the top of the hydrogel layer.

In some embodiments, the hydrogel comprises multiple layers or coatings. In some embodiments, the hydrogel comprises two coats. In some embodiments, the hydrogel comprises three coats. In some embodiments, the bottom (first) coating has greater porosity than subsequent coatings. In some embodiments, the top coat is has less porosity than the first coating. In some embodiments, the top coat has a mean pore diameter that functions as a size cut-off for particles of greater than 100 picometers in diameter.

In some embodiments, the hydrogel has a conductivity from about 0.001 S/m to about 10 S/m. In some embodiments, the hydrogel has a conductivity from about 0.01 S/m to about 10 S/m. In some embodiments, the hydrogel has a conductivity from about 0.1 S/m to about 10 S/m. In some embodiments, the hydrogel has a conductivity from about 1.0 S/m to about 10 S/m. In some embodiments, the hydrogel has a conductivity from about 0.01 S/m to about 5 S/m. In some embodiments, the hydrogel has a conductivity from about 0.01 S/m to about 4 S/m. In some embodiments, the hydrogel has a conductivity from about 0.01 S/m to about 3 S/m. In some embodiments, the hydrogel has a conductivity from about 0.01 S/m to about 2 S/m. In some embodiments, the hydrogel has a conductivity from about 0.1 S/m to about 5 S/m. In some embodiments, the hydrogel has a conductivity from about 0.1 S/m to about 4 S/m. In some embodiments, the hydrogel has a conductivity from about 0.1 S/m to about 3 S/m. In some embodiments, the hydrogel has a conductivity from about 0.1 S/m to about 2 S/m. In some embodiments, the hydrogel has a conductivity from about 0.1 S/m to about 1.5 S/m. In some embodiments, the hydrogel has a conductivity from about 0.1 S/m to about 1.0 S/m.

In some embodiments, the hydrogel has a conductivity of about 0.1 S/m. In some embodiments, the hydrogel has a conductivity of about 0.2 S/m. In some embodiments, the hydrogel has a conductivity of about 0.3 S/m. In some embodiments, the hydrogel has a conductivity of about 0.4 S/m. In some embodiments, the hydrogel has a conductivity of about 0.5 S/m. In some embodiments, the hydrogel has a conductivity of about 0.6 S/m. In some embodiments, the hydrogel has a conductivity of about 0.7 S/m. In some embodiments, the hydrogel has a conductivity of about 0.8 S/m. In some embodiments, the hydrogel has a conductivity of about 0.9 S/m. In some embodiments, the hydrogel has a conductivity of about 1.0 S/m.

In some embodiments, the hydrogel has a thickness from about 0.1 microns to about 10 microns. In some embodiments, the hydrogel has a thickness from about 0.1 microns to about 5 microns. In some embodiments, the hydrogel has a thickness from about 0.1 microns to about 4 microns. In some embodiments, the hydrogel has a thickness from about 0.1 microns to about 3 microns. In some embodiments, the hydrogel has a thickness from about 0.1 microns to about 2 microns. In some embodiments, the hydrogel has a thickness from about 1 micron to about 5 microns. In some embodiments, the hydrogel has a thickness from about 1 micron to about 4 microns. In some embodiments, the hydrogel has a thickness from about 1 micron to about 3 microns. In some embodiments, the hydrogel has a thickness from about 1 micron to about 2 microns. In some embodiments, the hydrogel has a thickness from about 0.5 microns to about 1 micron.

In some embodiments, the viscosity of a hydrogel solution prior to spin-coating ranges from about 0.5 cP to about 5 cP. In some embodiments, a single coating of hydrogel solution has a viscosity of between about 0.75 cP and 5 cP prior to spin-coating. In some embodiments, in a multi-coat hydrogel, the first hydrogel solution has a viscosity from about 0.5 cP to about 1.5 cP prior to spin coating. In some embodiments, the second hydrogel solution has a viscosity from about 1 cP to about 3 cP. The viscosity of the hydrogel solution is based on the polymers concentration (0.1%-10%) and polymers molecular weight (10,000 to 300,000) in the solvent and the starting viscosity of the solvent.

In some embodiments, the first hydrogel coating has a thickness between about 0.5 microns and 1 micron. In some embodiments, the first hydrogel coating has a thickness between about 0.5 microns and 0.75 microns. In some embodiments, the first hydrogel coating has a thickness between about 0.75 and 1 micron. In some embodiments, the second hydrogel coating has a thickness between about 0.2 microns and 0.5 microns. In some embodiments, the second hydrogel coating has a thickness between about 0.2 and 0.4 microns. In some embodiments, the second hydrogel coating has a thickness between about 0.2 and 0.3 microns. In some embodiments, the second hydrogel coating has a thickness between about 0.3 and 0.4 microns.

In some embodiments, the hydrogel comprises any suitable synthetic polymer forming a hydrogel. In general, any sufficiently hydrophilic and polymerizable molecule may be utilized in the production of a synthetic polymer hydrogel for use as disclosed herein. Polymerizable moieties in the monomers may include alkenyl moieties including but not limited to substituted or unsubstituted a,f3,unsaturated carbonyls wherein the double bond is directly attached to a carbon which is double bonded to an oxygen and single bonded to another oxygen, nitrogen, sulfur, halogen, or carbon; vinyl, wherein the double bond is singly bonded to an oxygen, nitrogen, halogen, phosphorus or sulfur; allyl, wherein the double bond is singly bonded to a carbon which is bonded to an oxygen, nitrogen, halogen, phosphorus or sulfur; homoallyl, wherein the double bond is singly bonded to a carbon which is singly bonded to another carbon which is then singly bonded to an oxygen, nitrogen, halogen, phosphorus or sulfur; alkynyl moieties wherein a triple bond exists between two carbon atoms. In some embodiments, acryloyl or acrylamido monomers such as acrylates, methacrylates, acrylamides, methacrylamides, etc., are useful for formation of hydrogels as disclosed herein. More preferred acrylamido monomers include acrylamides, N-substituted acrylamides, N-substituted methacrylamides, and methacrylamide. In some embodiments, a hydrogel comprises polymers such as epoxide-based polymers, vinyl-based polymers, allyl-based polymers, homoallyl-based polymers, cyclic anhydride-based polymers, ester-based polymers, ether-based polymers, alkylene-glycol based polymers (e.g., polypropylene glycol), and the like.

In some embodiments, the hydrogel comprises polyhydroxyethylmethacrylate (pHEMA), cellulose acetate, cellulose acetate phthalate, cellulose acetate butyrate, or any appropriate acrylamide or vinyl-based polymer, or a derivative thereof.

In some embodiments, the hydrogel is applied by vapor deposition.

In some embodiments, the hydrogel is polymerized via atom-transfer radical-polymerization via (ATRP).

In some embodiments, the hydrogel is polymerized via reversible addition—fragmentation chain-transfer (RAFT) polymerization.

In some embodiments, additives are added to a hydrogel to increase conductivity of the gel. In some embodiments, hydrogel additives are conductive polymers (e.g., PEDOT: PSS), salts (e.g., copper chloride), metals (e.g., gold), plasticizers (e.g., PEG200, PEG 400, or PEG 600), or co-solvents.

In some embodiments, the hydrogel also comprises compounds or materials which help maintain the stability of the DNA hybrids, including, but not limited to histidine, histidine peptides, polyhistidine, lysine, lysine peptides, and other cationic compounds or substances.

### Dielectrophoretic Fields

In some embodiments, the methods, devices and systems described herein provide a mechanism to collect, separate, and/or isolate cells, particles, and/or molecules (such as exosomes, DNA, RNA, nucleosomes, extracellular vesicles, proteins, cell membrane fragments, mitochondria and cellular vesicles) from a fluid material (which optionally contains other materials, such as contaminants, residual cellular material, or the like).

In some embodiments, an AC electrokinetic field is generated to collect, separate or isolate biomolecules, such as exosomes, DNA, RNA, nucleosomes, extracellular vesicles, proteins, cell membrane fragments, mitochondria and cellular vesicles. In some embodiments, the AC electrokinetic field is a dielectrophoretic field. Accordingly, in some embodiments dielectrophoresis (DEP) is utilized in various steps of the methods described herein.

In some embodiments, the devices and systems described herein are capable of generating DEP fields, and the like. In specific embodiments, DEP is used to concentrate cells and/or nucleic acids (e.g., concurrently or at different times). In certain embodiments, methods described herein further comprise energizing the array of electrodes so as to produce the first, second, and any further optional DEP fields. In some embodiments, the devices and systems described herein are capable of being energized so as to produce the first, second, and any further optional DEP fields.

DEP is a phenomenon in which a force is exerted on a dielectric particle when it is subjected to a non-uniform electric field. Depending on the step of the methods described herein, aspects of the devices and systems described herein, and the like, the dielectric particle in various embodiments herein is a biological cell and/or a molecule, such as a nucleic acid molecule. Different steps of the methods described herein or aspects of the devices or systems described herein may be utilized to isolate and separate different components, such as intact cells or other particular material; further, different field regions of the DEP field may be used in different steps of the methods or aspects of the devices and systems described herein. This dielectrophoretic force does not require the particle to be charged. In some instances, the strength of the force depends on the medium and the specific particles' electrical properties, on the particles' shape and size, as well as on the frequency of the electric field. In some instances, fields of a particular frequency selectivity manipulate particles. In certain aspects described herein, these processes allow for the separation of cells and/or smaller particles (such as molecules, including nucleic acid molecules) from other components (e.g., in a fluid medium) or each other.

In various embodiments provided herein, a method or device described herein comprises producing a plurality of DEP field regions. For example, a method or device comprises a first DEP field region and a second DEP field region with the array. In various embodiments provided herein, a device or system described herein is capable of producing a first DEP field region and a second DEP field region with the array. In some instances, the first and second field regions are part of a single field (e.g., the first and second regions are present at the same time, but are found at different locations within the device and/or upon the array). In some embodiments, the first and second field regions are different fields (e.g. the first region is created by energizing the electrodes at a first time, and the second region is created by energizing the electrodes a second time). In specific aspects, the first DEP field region is suitable for concentrating or isolating cells (e.g., into a low field DEP region). In some embodiments, the second DEP field region is suitable for concentrating smaller particles, such as molecules (e.g., nucleic acid, including cell-free nucleic acid), for example into a high field DEP region. In some instances, a method described herein optionally excludes use of either the first or second DEP field region.

As is described below, in some instances, the first DEP field is suitable for concentrating or isolating nucleic acids, including cell-free nucleic acids, above a size, below a size, or within a range of sizes. In some instances, the second DEP field is suitable for concentrating or isolating nucleic acids, including cell-free nucleic acids, above a size, below a size, or within a range of sizes. The first and second DEP fields can be configured to concentrate or isolate the same or different size nucleic acids. As such, the methods and devices disclosed herein can be used to assess nucleic acids of a variety of different sizes.

Also described herein are embodiments comprising three or more DEP field regions, wherein each of the field regions can be configured to operate in the same or different many as at least one other field regions. Thus, the embodiments can concentrate or isolate a variety of materials in the biological samples based upon a variety of properties. For example, a first DEP field region can be configured to isolate cells, a second DEP field region can be configured to isolate or concentrate cell-free DNA above 500 bp, a third DEP field region can be configured to isolate or concentrate cell-free DNA between 300 bp and 500 bp, and a fourth DEP field region can be configured to isolate or concentrate cell-free DNA below 300 bp. Some of such embodiments can include quantitating the amount of DNA isolated or concentrated within each field region.

In some embodiments, the first DEP field region is in the same chamber of a device as disclosed herein as the second DEP field region. In some embodiments, the first DEP field region and the second DEP field region occupy the same area of the array of electrodes.

In some embodiments, the first DEP field region is in a separate chamber of a device as disclosed herein, or a separate device entirely, from the second DEP field region.

### First DEP Field Region

In some aspects, e.g., high conductance buffers (>100 mS/m), the method described herein comprises applying a fluid comprising cells or other particulate material to a device comprising an array of electrodes, and, thereby, concentrating the cells in a first DEP field region. In some aspects, the devices and systems described herein are capable of applying a fluid comprising cells or other particulate material to the device comprising an array of electrodes, and, thereby, concentrating the cells in a first DEP field region. Subsequent or concurrent second, or optional third and fourth DEP regions, may collect or isolate other fluid components, including biomolecules, such as nucleic acids.

The first DEP field region may be any field region suitable for concentrating cells from a fluid. For this application, the cells are generally concentrated near the array of electrodes. In some embodiments, the first DEP field region is a dielectrophoretic low field region. In some embodiments, the first DEP field region is a dielectrophoretic high field region. In some aspects, e.g. low conductance buffers (<100 mS/m), the method described herein comprises applying a fluid comprising cells to a device comprising an array of electrodes, and, thereby, concentrating the cells or other particulate material in a first DEP field region.

In some aspects, the devices and systems described herein are capable of applying a fluid comprising cells or other particulate material to the device comprising an array of electrodes, and concentrating the cells in a first DEP field region. In various embodiments, the first DEP field region may be any field region suitable for concentrating cells from a fluid. In some embodiments, the cells are concentrated on the array of electrodes. In some embodiments, the cells are captured in a dielectrophoretic high field region. In some embodiments, the cells are captured in a dielectrophoretic low-field region. High versus low field capture is generally dependent on the conductivity of the fluid, wherein generally, the crossover point is between about 300-500 mS/m. In some embodiments, the first DEP field region is a dielectrophoretic low field region performed in fluid conductivity of greater than about 300 mS/m. In some embodiments, the first DEP field region is a dielectrophoretic low field region performed in fluid conductivity of less than about 300 mS/m. In some embodiments, the first DEP field region is a dielectrophoretic high field region performed in fluid conductivity of greater than about 300 mS/m. In some embodiments, the first DEP field region is a dielectrophoretic high field region performed in fluid conductivity of less than about 300 mS/m. In some embodiments, the first DEP field region is a dielectrophoretic low field region performed in fluid conductivity of greater than about 500 mS/m. In some embodiments, the first DEP field region is a dielectrophoretic low field region performed in fluid conductivity of less than about 500 mS/m. In some embodiments, the first DEP field region is a dielectrophoretic high field region performed in fluid conductivity of greater than about 500 mS/m. In some embodiments, the first DEP field region is a dielectrophoretic high field region performed in fluid conductivity of less than about 500 mS/m.

In some embodiments, the first dielectrophoretic field region is produced by an alternating current. The alternating current has any amperage, voltage, frequency, and the like suitable for concentrating cells. In some embodiments, the first dielectrophoretic field region is produced using an alternating current having an amperage of 0.1 micro Amperes −10 Amperes; a voltage of 1-50 Volts peak to peak; and/or a frequency of 1-10,000,000 Hz. In some embodiments, the first DEP field region is produced using an alternating current having a voltage of 5-25 volts peak to peak. In some embodiments, the first DEP field region is produced using an alternating current having a frequency of from 3-15 kHz. In some embodiments, the first DEP field region is produced using an alternating current having an amperage of 1 milliamp to 1 amp. In some embodiments, the first DEP field region is produced using an alternating current having an amperage of 0.1 micro Amperes −1 Ampere. In some embodiments, the first DEP field region is produced using an alternating current having an amperage of 1 micro Amperes −1 Ampere. In some embodiments, the first DEP field region is produced using an alternating current having an amperage of 100 micro Amperes −1 Ampere. In some embodiments, the first DEP field region is produced using an alternating current having an amperage of 500 micro Amperes −500 milli Amperes. In some embodiments, the first DEP field region is produced using an alternating current having a voltage of 1-25 Volts peak to peak. In some embodiments, the first DEP field region is produced using an alternating current having a voltage of 1-10 Volts peak to peak. In some embodiments, the first DEP field region is produced using an alternating current having a voltage of 25-50 Volts peak to peak. In some embodiments, the first DEP field region is produced using a frequency of from 10-1,000,000 Hz. In some embodiments, the first DEP field region is produced using a frequency of from 100-100,000 Hz. In some embodiments, the first DEP field region is produced using a frequency of from 100-10,000 Hz. In some embodiments, the first DEP field region is produced using a frequency of from 10,000-100,000 Hz. In some embodiments, the first DEP field region is produced using a frequency of from 100,000-1,000,000 Hz.

In some embodiments, the first dielectrophoretic field region is produced by a direct current. The direct current has any amperage, voltage, frequency, and the like suitable for concentrating cells. In some embodiments, the first dielectrophoretic field region is produced using a direct current having an amperage of 0.1micro Amperes −1 Amperes; a voltage of 10 milli Volts −10 Volts; and/or a pulse width of 1 milliseconds −1000 seconds and a pulse frequency of 0.001-1000 Hz. In some embodiments, the first DEP field region is produced using a direct current having an amperage of 1 micro Amperes −1 Amperes. In some embodiments, the first DEP field region is produced using a direct current having an amperage of 100 micro Amperes −500 milli Amperes. In some embodiments, the first DEP field region is produced using a direct current having an amperage of 1 milli Amperes—1 Amperes. In some embodiments, the first DEP field region is produced using a direct current having an amperage of 1 micro Amperes—1 milli Amperes. In some embodiments, the first DEP field region is produced using a direct current having a pulse width of 500 milliseconds-500 seconds. In some embodiments, the first DEP field region is produced using a direct current having a pulse width of 500 milliseconds-100 seconds. In some embodiments, the first DEP field region is produced using a direct current having a pulse width of 1 second −1000 seconds. In some embodiments, the first DEP field region is produced using a direct current having a pulse width of 500 milliseconds-1 second. In some embodiments, the first DEP field region is produced using a pulse frequency of 0.01-1000 Hz. In some embodiments, the first DEP field region is produced using a pulse frequency of 0.1-100 Hz. In some embodiments, the first DEP field region is produced using a pulse frequency of 1-100 Hz. In some embodiments, the first DEP field region is produced using a pulse frequency of 100-1000 Hz.

In some embodiments, the fluid comprises a mixture of cell types. For example, blood comprises red blood cells and white blood cells. Environmental samples comprise many types of cells and other particulate material over a wide range of concentrations. In some embodiments, one cell type (or any number of cell types less than the total number of cell types comprising the sample) is preferentially concentrated in the first DEP field. Without limitation, this embodiment is beneficial for focusing the nucleic acid isolation procedure on a particular environmental contaminant, such as a fecal coliform bacterium, whereby DNA sequencing may be used to identify the source of the contaminant. In another non-limiting example, the first DEP field is operated in a manner that specifically concentrates viruses and not cells (e.g., in a fluid with conductivity of greater than 300 mS/m, viruses concentrate in a DEP high field region, while larger cells will concentrate in a DEP low field region).

In some embodiments, a method, device or system described herein is suitable for isolating or separating specific cell types. In some embodiments, the DEP field of the method, device or system is specifically tuned to allow for the separation or concentration of a specific type of cell into a field region of the DEP field. In some embodiments, a method, device or system described herein provides more than one field region wherein more than one type of cell is isolated or concentrated. In some embodiments, a method, device, or system described herein is tunable so as to allow isolation or concentration of different types of cells within the DEP field regions thereof. In some embodiments, a method provided herein further comprises tuning the DEP field. In some embodiments, a device or system provided herein is capable of having the DEP field tuned. In some instances, such tuning may be in providing a DEP particularly suited for the desired purpose. For example, modifications in the array, the energy, or another parameter are optionally utilized to tune the DEP field. Tuning parameters for finer resolution include electrode diameter, edge to edge distance between electrodes, voltage, frequency, fluid conductivity and hydrogel composition.

In some embodiments, the first DEP field region comprises the entirety of an array of electrodes. In some embodiments, the first DEP field region comprises a portion of an array of electrodes. In some embodiments, the first DEP field region comprises about 90%, about 80%, about 70%, about 60%, about 50%, about 40%, about 30%, about 25%, about 20%, or about 10% of an array of electrodes. In some embodiments, the first DEP field region comprises about a third of an array of electrodes.

### Second DEP Field Region

The second DEP field region can be configured to be the same or different than the first DEP field region. As described above, the second DEP field region can be configured to isolate or concentrate the same or different macromolecules and cellular components as the first DEP field region. These include macromolecules and cellular components include exosomes, DNA, RNA, nucleosomes, extracellular vesicles, proteins, cell membrane fragments, mitochondria and cellular vesicles.

In some aspects, the first DEP field region and second DEP field region can be configured to isolate or concentrate different subsets of the same type of macromolecule or cellular component. For example, in some embodiments, the first DEP field region can be configured to isolate or concentrate a first macromolecule or first cellular component of a first size or first range of sizes and the second DEP field region can be configured to isolate or concentrate the first macromolecule or first cellular component of a second size or second range of sizes. In one example, the first DEP field region can be configured to isolate or concentrate cell-free DNA between 300-500 bp and the second DEP field region can be configured to isolate or concentrate cell-free DNA smaller than 300 bp. Thus, the plurality of field regions can be used to discriminate between subsets of the same type of macromolecule or cellular components. In an exemplary advantage, use of a plurality of field regions can also allow for the quantification of one or more subsets of the same type of macromolecule or cellular component.

In one aspect, following lysis of the cells (as provided below), the methods described herein involve concentrating the nucleic acid in a second DEP field region. In another aspect, the devices and systems described herein are capable of concentrating the nucleic acid in a second DEP field region. In some embodiments, the second DEP field region is any field region suitable for concentrating nucleic acids. In some embodiments, the nucleic acids are concentrated on the array of electrodes. In some embodiments, the second DEP field region is a dielectrophoretic high field region. The second DEP field region is, optionally, the same as the first DEP field region.

In some embodiments, the second dielectrophoretic field region is produced by an alternating current. In some embodiments, the alternating current has any amperage, voltage, frequency, and the like suitable for concentrating nucleic acids. In some embodiments, the second dielectrophoretic field region is produced using an alternating current having an amperage of 0.1 micro Amperes −10 Amperes; a voltage of 1-50 Volts peak to peak; and/or a frequency of 1-10,000,000 Hz. In some embodiments, the second DEP field region is produced using an alternating current having an amperage of 0.1 micro Amperes −1 Ampere. In some embodiments, the second DEP field region is produced using an alternating current having an amperage of 1 micro Amperes −1 Ampere. In some embodiments, the second DEP field region is produced using an alternating current having an amperage of 100 micro Amperes −1 Ampere. In some embodiments, the second DEP field region is produced using an alternating current having an amperage of 500 micro Amperes −500 milli Amperes. In some embodiments, the second DEP field region is produced using an alternating current having a voltage of 1-25 Volts peak to peak. In some embodiments, the second DEP field region is produced using an alternating current having a voltage of 1-10 Volts peak to peak. In some embodiments, the second DEP field region is produced using an alternating current having a voltage of 25-50 Volts peak to peak. In some embodiments, the second DEP field region is produced using a frequency of from 10-1,000,000 Hz. In some embodiments, the second DEP field region is produced using a frequency of from 100-100,000 Hz. In some embodiments, the second DEP field region is produced using a frequency of from 100-10,000 Hz. In some embodiments, the second DEP field region is produced using a frequency of from 10,000-100,000 Hz. In some embodiments, the second DEP field region is produced using a frequency of from 100,000-1,000,000 Hz.

In some embodiments, the second dielectrophoretic field region is produced by a direct current. In some embodiments, the direct current has any amperage, voltage, frequency, and the like suitable for concentrating nucleic acids. In some embodiments, the second dielectrophoretic field region is produced using a direct current having an amperage of 0.1micro Amperes −1 Amperes; a voltage of 10 milli Volts—10 Volts; and/or a pulse width of 1 milliseconds −1000 seconds and a pulse frequency of 0.001-1000 Hz. In some embodiments, the second DEP field region is produced using an alternating current having a voltage of 5-25 volts peak to peak. In some embodiments, the second DEP field region is produced using an alternating current having a frequency of from 3-15 kHz. In some embodiments, the second DEP field region is produced using an alternating current having an amperage of 1 milliamp to 1 amp. In some embodiments, the second DEP field region is produced using a direct current having an amperage of 1 micro Amperes-1 Amperes. In some embodiments, the second DEP field region is produced using a direct current having an amperage of 100 micro Amperes −500 milli Amperes. In some embodiments, the second DEP field region is produced using a direct current having an amperage of 1 milli Amperes—1 Amperes. In some embodiments, the second DEP field region is produced using a direct current having an amperage of 1 micro Amperes—1 milli Amperes. In some embodiments, the second DEP field region is produced using a direct current having a pulse width of 500 milliseconds-500 seconds. In some embodiments, the second DEP field region is produced using a direct current having a pulse width of 500 milliseconds-100 seconds. In some embodiments, the second DEP field region is produced using a direct current having a pulse width of 1 second −1000 seconds. In some embodiments, the second DEP field region is produced using a direct current having a pulse width of 500 milliseconds-1 second. In some embodiments, the second DEP field region is produced using a pulse frequency of 0.01-1000 Hz. In some embodiments, the second DEP field region is produced using a pulse frequency of 0.1-100 Hz. In some embodiments, the second DEP field region is produced using a pulse frequency of 1-100 Hz. In some embodiments, the second DEP field region is produced using a pulse frequency of 100-1000 Hz.

In some embodiments, the second DEP field region comprises the entirety of an array of electrodes. In some embodiments, the second DEP field region comprises a portion of an array of electrodes. In some embodiments, the second DEP field region comprises about 90%, about 80%, about 70%, about 60%, about 50%, about 40%, about 30%, about 25%, about 20%, or about 10% of an array of electrodes. In some embodiments, the second DEP field region comprises about a third of an array of electrodes.

### Isolating Biomarkers

In some aspects, described herein are methods, devices and systems for isolating a biomarker from a biological complex, for example vesicles such as extracellular vesicles, exosomes, microvesicles, enveloped-particles, and other complex particles or biological parcels that include a combination of biological components, including DNA, RNA, proteins, lipids and other biological molecules.

In one aspect, described herein is a method for isolating a biomarker from an exosome (e.g., DNA, RNA, nucleosomes, proteins, and/or cell membrane fragments) from a fluid. In some embodiments, the biomarkers are cell-free nucleic acids. In some embodiments, the method comprises: applying a fluid to a device, the device comprising an array of electrodes; concentrating a plurality of exosomes in a first AC electrokinetic (e.g., dielectrophoretic) field region; and eluting the exosomes from the device for further analysis (e.g., sequencing, mass spectroscopy, etc).

In some embodiments, disclosed herein is method for isolating a cell-free nucleic acid from a fluid, the method comprising: a. applying the fluid to a device, the device comprising an array of electrodes; b. concentrating a plurality of cellular materials in a first AC electrokinetic (e.g., dielectrophoretic) field region; c. isolating nucleic acid in a second AC electrokinetic (e.g., dielectrophoretic) field region; and d. flushing the cellular materials away. In some instances, residual cellular material is concentrated near the low field region. In some embodiments, the residual material is washed from the device and/or washed from the nucleic acids. In some embodiments, the nucleic acid is concentrated in the second AC electrokinetic field region.

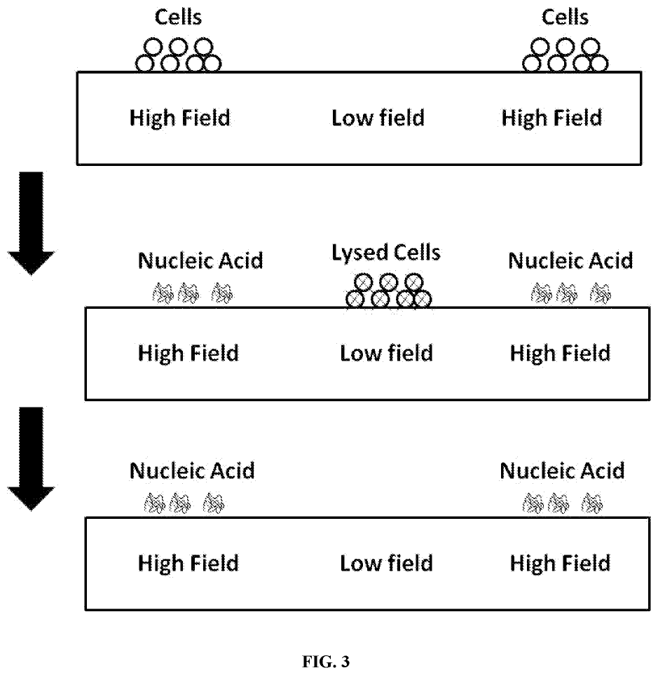

In some embodiments, the biomarker nucleic acids are initially inside the cells. As seen in FIG. 3, the method comprises concentrating the cells near a high field region in some instances. In some embodiments, disclosed herein is method for isolating a nucleic acid from a fluid comprising cells, the method comprising: a. applying the fluid to a device, the device comprising an array of electrodes; b. concentrating a plurality of cells in a first AC electrokinetic (e.g., dielectrophoretic) field region; c. isolating nucleic acid in a second AC electrokinetic (e.g., dielectrophoretic) field region; and d. flushing cells away. In some instances, the cells are lysed in the high field region. Following lysis, the nucleic acids remain in the high field region and/or are concentrated in the high field region. In some instances, residual cellular material is concentrated near the low field region. In some embodiments, the residual material is washed from the device and/or washed from the nucleic acids. In some embodiments, the nucleic acid is concentrated in the second AC electrokinetic field region.

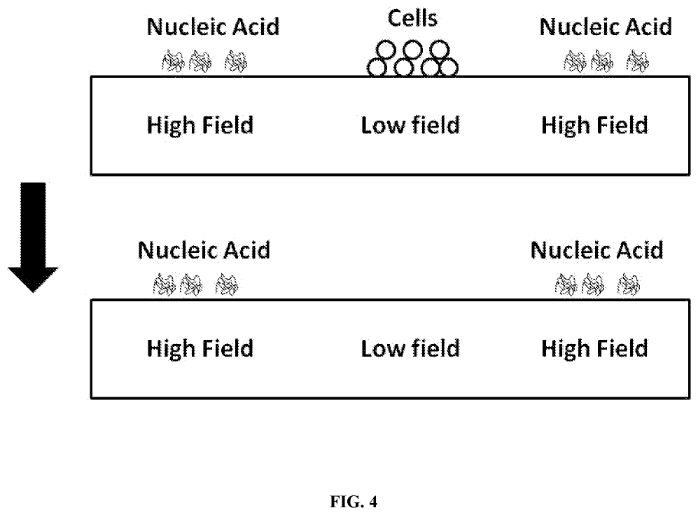

In one aspect, described herein is a method for isolating a biomarker from a fluid comprising cells or other particulate material. In some embodiments, the biomarkers are not inside the cells (e.g., cell-free DNA in fluid). In some embodiments, disclosed herein is a method for isolating a biomarker from a fluid comprising cells or other particulate material, the method comprising: a. applying the fluid to a device, the device comprising an array of electrodes; b. concentrating a plurality of cells in a first AC electrokinetic (e.g., dielectrophoretic) field region; c. isolating biomarkers (e.g., exosomes, DNA, RNA, nucleosomes, extracellular vesicles, proteins, cell membrane fragments, mitochondria and cellular vesicles) in a second AC electrokinetic (e.g., dielectrophoretic) field region; and d. flushing cells away. In some embodiments, the method further comprises degrading residual proteins after flushing cells away. FIG. 4 shows an exemplary method for isolating extra-cellular nucleic acids from a fluid comprising cells. A similar method is used to isolate other small particulates from cells, such as vesicles such as extracellular vesicles, exosomes, microvesicles, enveloped-particles, and other complex particles or biological parcels that include a combination of biological components, including DNA, RNA, proteins, lipids and other biological molecules. In some embodiments, cells are concentrated on or near a low field region and nucleic acids (or other small particulates) are concentrated on or near a high field region. In some instances, the cells are washed from the device and/or washed from the nucleic acids (or other small particulates).

In one aspect, the methods, systems and devices described herein isolate nucleic acid from a fluid comprising cells or other particulate material. In one aspect, dielectrophoresis is used to concentrate cells. In some embodiments, the fluid is a liquid, optionally water or an aqueous solution or dispersion. In some embodiments, the fluid is any suitable fluid including a bodily fluid. Exemplary bodily fluids include blood, serum, plasma, bile, milk, cerebrospinal fluid, gastric juice, ejaculate, mucus, peritoneal fluid, saliva, sweat, tears, urine, and the like. In some embodiments, nucleic acids are isolated from bodily fluids using the methods, systems or devices described herein as part of a medical therapeutic or diagnostic procedure, device or system. In some embodiments, the fluid is tissues and/or cells solubilized and/or dispersed in a fluid. For example, the tissue can be a cancerous tumor from which nucleic acid can be isolated using the methods, devices or systems described herein.

In some embodiments, the fluid may also comprise other particulate material. Such particulate material may be, for example, inclusion bodies (e.g., ceroids or Mallory bodies), cellular casts (e.g., granular casts, hyaline casts, cellular casts, waxy casts and pseudo casts), Pick's bodies, Lewy bodies, fibrillary tangles, fibril formations, cellular debris and other particulate material. In some embodiments, particulate material is an aggregated protein (e.g., beta-amyloid).

The fluid can have any conductivity including a high or low conductivity. In some embodiments, the conductivity is between about 1 μS/m to about 10 mS/m. In some embodiments, the conductivity is between about 10 μS/m to about 10 mS/m. In other embodiments, the conductivity is between about 50 μSim to about 10 mS/m. In yet other embodiments, the conductivity is between about 100 μSim to about 10 mS/m, between about 100 μSim to about 8 mS/m, between about 100 μS/m to about 6 mS/m, between about 100 μS/m to about 5 mS/m, between about 100 μSim to about 4 mS/m, between about 100 μSim to about 3 mS/m, between about 100 μSim to about 2 mS/m, or between about 100 μSim to about 1 mS/m.

In some embodiments, the conductivity is about 1 μS/m. In some embodiments, the conductivity is about 10 μS/m. In some embodiments, the conductivity is about 100 μS/m. In some embodiments, the conductivity is about 1 mS/m. In other embodiments, the conductivity is about 2 mS/m. In some embodiments, the conductivity is about 3 mS/m. In yet other embodiments, the conductivity is about 4 mS/m. In some embodiments, the conductivity is about 5 mS/m. In some embodiments, the conductivity is about 10 mS/m. In still other embodiments, the conductivity is about 100 mS/m. In some embodiments, the conductivity is about 1 S/m. In other embodiments, the conductivity is about 10 S/m.

In some embodiments, the conductivity is at least 1 μS/m. In yet other embodiments, the conductivity is at least 10 μS/m. In some embodiments, the conductivity is at least 100 μS/m. In some embodiments, the conductivity is at least 1 mS/m. In additional embodiments, the conductivity is at least 10 mS/m. In yet other embodiments, the conductivity is at least 100 mS/m. In some embodiments, the conductivity is at least 1 S/m. In some embodiments, the conductivity is at least 10 S/m. In some embodiments, the conductivity is at most 1 μS/m. In some embodiments, the conductivity is at most 10 μS/m. In other embodiments, the conductivity is at most 100 μS/m. In some embodiments, the conductivity is at most 1 mS/m. In some embodiments, the conductivity is at most 10 mS/m. In some embodiments, the conductivity is at most 100 mS/m. In yet other embodiments, the conductivity is at most 1 S/m. In some embodiments, the conductivity is at most 10 S/m.

In some embodiments, the fluid is a small volume of liquid including less than 10 ml. In some embodiments, the fluid is less than 8 ml. In some embodiments, the fluid is less than 5 ml. In some embodiments, the fluid is less than 2 ml. In some embodiments, the fluid is less than 1 ml. In some embodiments, the fluid is less than 500 μl. In some embodiments, the fluid is less than 200 μl. In some embodiments, the fluid is less than 100 μl. In some embodiments, the fluid is less than 50 μl. In some embodiments, the fluid is less than 1011.1. In some embodiments, the fluid is less than 5 μl. In some embodiments, the fluid is less than 111.1.

In some embodiments, the quantity of fluid applied to the device or used in the method comprises less than about 100,000,000 cells. In some embodiments, the fluid comprises less than about 10,000,000 cells. In some embodiments, the fluid comprises less than about 1,000,000 cells. In some embodiments, the fluid comprises less than about 100,000 cells. In some embodiments, the fluid comprises less than about 10,000 cells. In some embodiments, the fluid comprises less than about 1,000 cells. In some embodiments, the fluid is cell-free.

In some embodiments, isolation of nucleic acid from a fluid comprising cells or other particulate material with the devices, systems and methods described herein takes less than about 30 minutes, less than about 20 minutes, less than about 15 minutes, less than about 10 minutes, less than about 5 minutes or less than about 1 minute. In other embodiments, isolation of nucleic acid from a fluid comprising cells or other particulate material with the devices, systems and methods described herein takes not more than 30 minutes, not more than about 20 minutes, not more than about 15 minutes, not more than about 10 minutes, not more than about 5 minutes, not more than about 2 minutes or not more than about 1 minute. In additional embodiments, isolation of nucleic acid from a fluid comprising cells or other particulate material with the devices, systems and methods described herein takes less than about 15 minutes, preferably less than about 10 minutes or less than about 5 minutes.

In some instances, exosomes, extra-cellular DNA, cell-free DNA fragments, or other nucleic acids (outside cells) are isolated from a fluid comprising cells of other particulate material. In some embodiments, the fluid comprises cells. In some embodiments, the fluid does not comprise cells.

### Cell Lysis

In one aspect, following concentrating the cells in a first dielectrophoretic field region, the method involves freeing nucleic acids from the cells. In another aspect, the devices and systems described herein are capable of freeing nucleic acids from the cells. In some embodiments, the nucleic acids are freed from the cells in the first DEP field region.

In some embodiments, the methods described herein free nucleic acids from a plurality of cells by lysing the cells. In some embodiments, the devices and systems described herein are capable of freeing nucleic acids from a plurality of cells by lysing the cells. One method of cell lysis involves applying a direct current to the cells after isolation of the cells on the array. The direct current has any suitable amperage, voltage, and the like suitable for lysing cells. In some embodiments, the current has a voltage of about 1 Volt to about 500 Volts. In some embodiments, the current has a voltage of about 10 Volts to about 500 Volts. In other embodiments, the current has a voltage of about 10 Volts to about 250 Volts. In still other embodiments, the current has a voltage of about 50 Volts to about 150 Volts. Voltage is generally the driver of cell lysis, as high electric fields result in failed membrane integrity.

In some embodiments, the direct current used for lysis comprises one or more pulses having any duration, frequency, and the like suitable for lysing cells. In some embodiments, a voltage of about 100 volts is applied for about 1 millisecond to lyse cells. In some embodiments, the voltage of about 100 volts is applied 2 or 3 times over the source of a second.

In some embodiments, the frequency of the direct current depends on volts/cm, pulse width, and the fluid conductivity. In some embodiments, the pulse has a frequency of about 0.001 to about 1000 Hz. In some embodiments, the pulse has a frequency from about 10 to about 200 Hz. In other embodiments, the pulse has a frequency of about 0.01 Hz-1000 Hz. In still other embodiments, the pulse has a frequency of about 0.1 Hz-1000 Hz, about 1 Hz-1000 Hz, about 1 Hz-500 Hz, about 1 Hz-400 Hz, about 1 Hz-300 Hz, or about 1 Hz—about 250 Hz. In some embodiments, the pulse has a frequency of about 0.1 Hz. In other embodiments, the pulse has a frequency of about 1 Hz. In still other embodiments, the pulse has a frequency of about 5 Hz, about 10 Hz, about 50 Hz, about 100 Hz, about 200 Hz, about 300 Hz, about 400 Hz, about 500 Hz, about 600 Hz, about 700 Hz, about 800 Hz, about 900 Hz or about 1000 Hz.

In other embodiments, the pulse has a duration of about 1 millisecond (ms) −1000 seconds (s). In some embodiments, the pulse has a duration of about 10 ms −1000 s. In still other embodiments, the pulse has a duration of about 100 ms −1000 s, about 1 s-1000 s, about 1 s −500 s, about 1 s-250 s or about 1 s-150 s. In some embodiments, the pulse has a duration of about 1 ms, about 10 ms, about 100 ms, about 1 s, about 2 s, about 3 s, about 4 s, about 5 s, about 6 s, about 7 s, about 8 s, about 9 s, about 10 s, about 20 s, about 50 s, about 100 s, about 200 s, about 300 s, about 500 s or about 1000 s. In some embodiments, the pulse has a frequency of 0.2 to 200 Hz with duty cycles from 10-50%.

In some embodiments, the direct current is applied once, or as multiple pulses. Any suitable number of pulses may be applied including about 1-20 pulses. There is any suitable amount of time between pulses including about 1 millisecond −1000 seconds. In some embodiments, the pulse duration is 0.01 to 10 seconds.

In some embodiments, the cells are lysed using other methods in combination with a direct current applied to the isolated cells. In yet other embodiments, the cells are lysed without use of direct current. In various aspects, the devices and systems are capable of lysing cells with direct current in combination with other means, or may be capable of lysing cells without the use of direct current. Any method of cell lysis known to those skilled in the art may be suitable including, but not limited to application of a chemical lysing agent (e.g., an acid), an enzymatic lysing agent, heat, pressure, shear force, sonic energy, osmotic shock, or combinations thereof. Lysozyme is an example of an enzymatic-lysing agent.

### Removal of Residual Material

In some embodiments, following concentration of the targeted cellular material in the second DEP field region, the method includes optionally flushing residual material from the targeted cellular material. In some embodiments, the devices or systems described herein are capable of optionally comprising a reservoir comprising a fluid suitable for flushing residual material from the targeted cellular material. In some embodiments, the targeted cellular material is held near the array of electrodes, such as in the second DEP field region, by continuing to energize the electrodes. “Residual material” is anything originally present in the fluid, originally present in the cells, added during the procedure, created through any step of the process including but not limited to lysis of the cells (i.e. residual cellular material), and the like. For example, residual material includes non-lysed cells, cell wall fragments, proteins, lipids, carbohydrates, minerals, salts, buffers, plasma, and undesired nucleic acids. In some embodiments, the lysed cellular material comprises residual protein freed from the plurality of cells upon lysis. It is possible that not all of the targeted cellular material will be concentrated in the second DEP field. In some embodiments, a certain amount of targeted cellular material is flushed with the residual material.

In some embodiments, the residual material is flushed in any suitable fluid, for example in water, TBE buffer, or the like. In some embodiments, the residual material is flushed with any suitable volume of fluid, flushed for any suitable period of time, flushed with more than one fluid, or any other variation. In some embodiments, the method of flushing residual material is related to the desired level of isolation of the targeted cellular material with higher purity targeted cellular material requiring more stringent flushing and/or washing. In other embodiments, the method of flushing residual material is related to the particular starting material and its composition. In some instances, a starting material that is high in lipid requires a flushing procedure that involves a hydrophobic fluid suitable for solubilizing lipids.

In some embodiments, the method includes degrading residual material including residual protein. In some embodiments, the devices or systems are capable of degrading residual material including residual protein. For example, proteins are degraded by one or more of chemical degradation (e.g. acid hydrolysis) and enzymatic degradation. In some embodiments, the enzymatic degradation agent is a protease. In other embodiments, the protein degradation agent is Proteinase K. The optional step of degradation of residual material is performed for any suitable time, temperature, and the like. In some embodiments, the degraded residual material (including degraded proteins) is flushed from the nucleic acid.

In some embodiments, the agent used to degrade the residual material is inactivated or degraded. In some embodiments, the devices or systems are capable of degrading or inactivating the agent used to degrade the residual material. In some embodiments, an enzyme used to degrade the residual material is inactivated by heat (e.g., 50 to 95° C. for 5-15 minutes). For example, enzymes including proteases, (for example, Proteinase K) are degraded and/or inactivated using heat (typically, 15 minutes, 70° C.). In some embodiments wherein the residual proteins are degraded by an enzyme, the method further comprises inactivating the degrading enzyme (e.g., Proteinase K) following degradation of the proteins. In some embodiments, heat is provided by a heating module in the device (temperature range, e.g., from 30 to 95° C.).

The order and/or combination of certain steps of the method can be varied. In some embodiments, the devices or methods are capable of performing certain steps in any order or combination. For example, in some embodiments, the residual material and the degraded proteins are flushed in separate or concurrent steps. That is, the residual material is flushed, followed by degradation of residual proteins, followed by flushing degraded proteins from the nucleic acid. In some embodiments, one first degrades the residual proteins, and then flush both the residual material and degraded proteins from the nucleic acid in a combined step.

In some embodiments, the targeted cellular materials are retained in the device and optionally used in further procedures such as PCR or other procedures manipulating or amplifying nucleic acid. In some embodiments, the devices and systems are capable of performing PCR or other optional procedures. In other embodiments, the targeted cellular materials are collected and/or eluted from the device. In some embodiments, the devices and systems are capable of allowing collection and/or elution of targeted cellular material from the device or system. In some embodiments, the isolated cellular material is collected by (i) turning off the second dielectrophoretic field region; and (ii) eluting the material from the array in an eluant. Exemplary eluants include water, TE, TBE and L-Histidine buffer.

### Biological Molecules

In some embodiments, the method, device, or system described herein is optionally utilized to obtain, isolate, or separate any desired biological material that may be obtained from such a method, device or system, such as extracellular vesicles, exosomes, microvesicles, enveloped-particles, and other complex particles or biological parcels that include a combination of biological components, including DNA, RNA, proteins, lipids and other biological molecules. Nucleic acids isolated by the methods, devices and systems described herein include DNA (deoxyribonucleic acid), RNA (ribonucleic acid), and combinations thereof. DNA can include cell-free DNA and DNA fragments. In some embodiments, the nucleic acid is isolated in a form suitable for sequencing or further manipulation of the nucleic acid, including amplification, ligation or cloning. Proteins isolated by the methods devices and systems described herein include protein complexes, full length proteins, processed proteins, and protein fragments. In some embodiments, the protein is isolated in a form suitable for mass spectroscopy or antibody-based analysis (e.g., ELISA, Western blot, immunofluorescence).

In some embodiments, the isolated, separated, or captured nucleic acid comprises DNA fragments that are selectively or preferentially isolated, separated, or captured based on their sizes. In some embodiments, the DNA fragments that are selectively or preferentially isolated, separated, or captured are between 250-600 bp, 250-275 bp, 275-300 bp, 300-325 bp, 325-350 bp, 350-375 bp, 375-400 bp, 400-425 bp, 425-450 bp, 450-475 bp, 475-500 bp, 500-525 bp, 525-550 bp, 550-575 bp, 575-600 bp, 300-400 bp, 400-500 bp, and/or 300-500 bp in length. In some embodiments, the DNA fragments that are selectively or preferentially isolated, separated, or captured are between 600-700 bp, 700-800 bp, 800-900 bp, 900-1000 bp, 1-2 kbp, 2-3 kbp, 3-4 kbp, 4-5 kbp, 5-6 kbp, 6-7 kbp, 7-8 kbp, 8-9 kbp, or 9-10 kbp. In some embodiments, the DNA fragments that are selectively or preferentially isolated, separated, or captured are greater than 300, 400, 500, 600, 700, 800, 900, or 1000 bp in size.

In some embodiments, the DNA fragments are cell-free DNA fragments.

In various embodiments, an isolated or separated nucleic acid is a composition comprising nucleic acid that is free from at least 99% by mass of other materials, free from at least 99% by mass of residual cellular material (e.g., from lysed cells from which the nucleic acid is obtained), free from at least 98% by mass of other materials, free from at least 98% by mass of residual cellular material, free from at least 95% by mass of other materials, free from at least 95% by mass of residual cellular material, free from at least 90% by mass of other materials, free from at least 90% by mass of residual cellular material, free from at least 80% by mass of other materials, free from at least 80% by mass of residual cellular material, free from at least 70% by mass of other materials, free from at least 70% by mass of residual cellular material, free from at least 60% by mass of other materials, free from at least 60% by mass of residual cellular material, free from at least 50% by mass of other materials, free from at least 50% by mass of residual cellular material, free from at least 30% by mass of other materials, free from at least 30% by mass of residual cellular material, free from at least 10% by mass of other materials, free from at least 10% by mass of residual cellular material, free from at least 5% by mass of other materials, or free from at least 5% by mass of residual cellular material.

In various embodiments, the nucleic acid has any suitable purity. For example, if a DNA sequencing procedure can work with nucleic acid samples having about 20% residual cellular material, then isolation of the nucleic acid to 80% is suitable. In some embodiments, the isolated nucleic acid comprises less than about 80%, less than about 70%, less than about 60%, less than about 50%, less than about 40%, less than about 30%, less than about 20%, less than about 10%, less than about 5%, or less than about 2% non-nucleic acid cellular material and/or protein by mass. In some embodiments, the isolated nucleic acid comprises greater than about 99%, greater than about 98%, greater than about 95%, greater than about 90%, greater than about 80%, greater than about 70%, greater than about 60%, greater than about 50%, greater than about 40%, greater than about 30%, greater than about 20%, or greater than about 10% nucleic acid by mass.

The nucleic acids are isolated in any suitable form including unmodified, derivatized, fragmented, non-fragmented, and the like. In some embodiments, the nucleic acid is collected in a form suitable for sequencing. In some embodiments, the nucleic acid is collected in a fragmented form suitable for shotgun-sequencing, amplification or other manipulation. The nucleic acid may be collected from the device in a solution comprising reagents used in, for example, a DNA sequencing procedure, such as nucleotides as used in sequencing by synthesis methods.

In some embodiments, the methods described herein result in an isolated nucleic acid sample that is approximately representative of the nucleic acid of the starting sample. In some embodiments, the devices and systems described herein are capable of isolating nucleic acid from a sample that is approximately representative of the nucleic acid of the starting sample. That is, the population of nucleic acids collected by the method, or capable of being collected by the device or system, are substantially in proportion to the population of nucleic acids present in the cells in the fluid. In some embodiments, this aspect is advantageous in applications in which the fluid is a complex mixture of many cell types and the practitioner desires a nucleic acid-based procedure for determining the relative populations of the various cell types.

In some embodiments, the nucleic acid isolated using the methods described herein or capable of being isolated by the devices described herein is high-quality and/or suitable for using directly in downstream procedures such as DNA sequencing, nucleic acid amplification, such as PCR, or other nucleic acid manipulation, such as ligation, cloning or further translation or transformation assays. In some embodiments, the collected nucleic acid comprises at most 0.01% protein. In some embodiments, the collected nucleic acid comprises at most 0.5% protein. In some embodiments, the collected nucleic acid comprises at most 0.1% protein. In some embodiments, the collected nucleic acid comprises at most 1% protein. In some embodiments, the collected nucleic acid comprises at most 2% protein. In some embodiments, the collected nucleic acid comprises at most 3% protein. In some embodiments, the collected nucleic acid comprises at most 4% protein. In some embodiments, the collected nucleic acid comprises at most 5% protein.

In some embodiments, the nucleic acid isolated by the methods described herein or capable of being isolated by the devices described herein has a concentration of at least 0.5 ng/mL. In some embodiments, the nucleic acid isolated by the methods described herein or capable of being isolated by the devices described herein has a concentration of at least 1 ng/mL. In some embodiments, the nucleic acid isolated by the methods described herein or capable of being isolated by the devices described herein has a concentration of at least 5 ng/mL. In some embodiments, the nucleic acid isolated by the methods described herein or capable of being isolated by the devices described herein has a concentration of at least 10 ng/ml.

In some embodiments, about 50 pico-grams of nucleic acid is isolated from about 5,000 cells using the methods, systems or devices described herein. In some embodiments, the methods, systems or devices described herein yield at least 10 pico-grams of nucleic acid from about 5,000 cells. In some embodiments, the methods, systems or devices described herein yield at least 20 pico-grams of nucleic acid from about 5,000 cells. In some embodiments, the methods, systems or devices described herein yield at least 50 pico-grams of nucleic acid from about 5,000 cells. In some embodiments, the methods, systems or devices described herein yield at least 75 pico-grams of nucleic acid from about 5,000 cells. In some embodiments, the methods, systems or devices described herein yield at least 100 pico-grams of nucleic acid from about 5,000 cells. In some embodiments, the methods, systems or devices described herein yield at least 200 pico-grams of nucleic acid from about 5,000 cells. In some embodiments, the methods, systems or devices described herein yield at least 300 pico-grams of nucleic acid from about 5,000 cells. In some embodiments, the methods, systems or devices described herein yield at least 400 pico-grams of nucleic acid from about 5,000 cells. In some embodiments, the methods, systems or devices described herein yield at least 500 pico-grams of nucleic acid from about 5,000 cells. In some embodiments, the methods, systems or devices described herein yield at least 1,000 pico-grams of nucleic acid from about 5,000 cells. In some embodiments, the methods, systems or devices described herein yield at least 10,000 pico-grams of nucleic acid from about 5,000 cells.

### Assays and Applications

In some embodiments, the methods described herein further comprise optionally amplifying the isolated nucleic acid by polymerase chain reaction (PCR). In some embodiments, the PCR reaction is performed on or near the array of electrodes or in the device. In some embodiments, the device or system comprise a heater and/or temperature control mechanisms suitable for thermocycling.

PCR is optionally done using traditional thermocycling by placing the reaction chemistry analytes in between two efficient thermoconductive elements (e.g., aluminum or silver) and regulating the reaction temperatures using TECs. Additional designs optionally use infrared heating through optically transparent material like glass or thermo polymers. In some instances, designs use smart polymers or smart glass that comprise conductive wiring networked through the substrate. This conductive wiring enables rapid thermal conductivity of the materials and (by applying appropriate DC voltage) provides the required temperature changes and gradients to sustain efficient PCR reactions. In certain instances, heating is applied using resistive chip heaters and other resistive elements that will change temperature rapidly and proportionally to the amount of current passing through them.

In some embodiments, used in conjunction with traditional fluorometry (ccd, pmt, other optical detector, and optical filters), fold amplification is monitored in real-time or on a timed interval. In certain instances, quantification of final fold amplification is reported via optical detection converted to AFU (arbitrary fluorescence units correlated to analyze doubling) or translated to electrical signal via impedance measurement or other electrochemical sensing.

Given the small size of the micro electrode array, these elements are optionally added around the micro electrode array and the PCR reaction will be performed in the main sample processing chamber (over the DEP array) or the analytes to be amplified are optionally transported via fluidics to another chamber within the fluidic cartridge to enable on-cartridge Lab-On-Chip

### Processing

In some instances, light delivery schemes are utilized to provide the optical excitation and/or emission and/or detection of fold amplification. In certain embodiments, this includes using the flow cell materials (thermal polymers like acrylic (PMMA) cyclic olefin polymer (COP), cyclic olefin co-polymer, (COC), etc.) as optical wave guides to remove the need to use external components. In addition, in some instances light sources—light emitting diodes—LEDs, vertical-cavity surface-emitting lasers—VCSELs, and other lighting schemes are integrated directly inside the flow cell or built directly onto the micro electrode array surface to have internally controlled and powered light sources. Miniature PMTs, CCDs, or CMOS detectors can also be built into the flow cell. This minimization and miniaturization enables compact devices capable of rapid signal delivery and detection while reducing the footprint of similar traditional devices (i.e. a standard bench top PCR/QPCR/Fluorometer).

### Amplification on Chip

In some instances, silicon microelectrode arrays can withstand thermal cycling necessary for PCR. In some applications, on-chip PCR is advantageous because small amounts of target nucleic acids can be lost during transfer steps. In certain embodiments of devices, systems or processes described herein, any one or more of multiple PCR techniques are optionally used, such techniques optionally including any one or more of the following: thermal cycling in the flow cell directly; moving the material through microchannels with different temperature zones; and moving volume into a PCR tube that can be amplified on system or transferred to a PCR machine. In some instances, droplet PCR is performed if the outlet contains a T-junction that contains an immiscible fluid and interfacial stabilizers (surfactants, etc.). In certain embodiments, droplets are thermal cycled in by any suitable method.

In some embodiments, amplification is performed using an isothermal reaction, for example, transcription mediated amplification, nucleic acid sequence-based amplification, signal mediated amplification of RNA technology, strand displacement amplification, rolling circle amplification, loop-mediated isothermal amplification of DNA, isothermal multiple displacement amplification, helicase-dependent amplification, single primer isothermal amplification or circular helicase-dependent amplification.

In various embodiments, amplification is performed in homogenous solution or as heterogeneous system with anchored primer(s). In some embodiments of the latter, the resulting amplicons are directly linked to the surface for higher degree of multiplex. In some embodiments, the amplicon is denatured to render single stranded products on or near the electrodes. Hybridization reactions are then optionally performed to interrogate the genetic information, such as single nucleotide polymorphisms (SNPs), Short Tandem Repeats (STRs), mutations, insertions/deletions, methylation, etc. Methylation is optionally determined by parallel analysis where one DNA sample is bisulfite treated and one is not. Bisulfite depurinates unmodified C becoming a U. Methylated C is unaffected in some instances. In some embodiments, allele specific base extension is used to report the base of interest.

Rather than specific interactions, the surface is optionally modified with nonspecific moieties for capture. For example, surface could be modified with polycations, i.e., polylysine, to capture DNA molecules which can be released by reverse bias (−V). In some embodiments, modifications to the surface are uniform over the surface or patterned specifically for functionalizing the electrodes or non electrode regions. In certain embodiments, this is accomplished with photolithography, electrochemical activation, spotting, and the like.

In some applications, a chip may include multiple regions, each region configured to capture DNA fragments of a specific or different size. Chip regions can sometimes vary with respect to voltage, amperage, frequency, pitch, electrode diameter, the depth of the well, or other factors to selectively capture fragments of different sizes in different regions. In some embodiments, each region comprises an array of multiple electrodes.

In various embodiments, devices or regions are run sequentially or in parallel. In some embodiments, multiple chip designs are used to narrow the size range of material collected creating a band pass filter. In some instances, current chip geometry (e.g., 80 um diameter electrodes on 200 um center-center pitch ( 80/200) acts as 500 bp cutoff filter (e.g., using voltage and frequency conditions around 10 Vpp and 10 kHz). In such instances, a nucleic acid of greater than 500 bp is captured, and a nucleic acid of less than 500 bp is not. Alternate electrode diameter and pitch geometries have different cutoff sizes such that a combination of chips should provide a desired fragment size. In some instances, a 40 um diameter electrode on 100 um center-center pitch ( 40/100) has a lower cutoff threshold, whereas a 160 um diameter electrode on 400 um center-center pitch (160/400) has a higher cutoff threshold relative to the 80/200 geometry, under similar conditions. In various embodiments, geometries on a single chip or multiple chips are combined to select for a specific sized fragments or particles. For example a 600 bp cutoff chip would leave a nucleic acid of less than 600 bp in solution, then that material is optionally recaptured with a 500 bp cutoff chip (which is opposing the 600 bp chip). This leaves a nucleic acid population comprising 500-600 bp in solution. This population is then optionally amplified in the same chamber, a side chamber, or any other configuration. In some embodiments, size selection is accomplished using a single electrode geometry, wherein nucleic acid of >500 bp is isolated on the electrodes, followed by washing, followed by reduction of the ACEK high field strength (change voltage, frequency, conductivity) in order to release nucleic acids of <600 bp, resulting in a supernatant nucleic acid population between 500-600 bp. In some embodiments, the device is configured to selectively capture nucleic acid fragments between 250-600 bp, 250-275 bp, 275-300 bp, 300-325 bp, 325-350 bp, 350-375 bp, 375-400 bp, 400-425 bp, 425-450 bp, 450-475 bp, 475-500 bp, 500-525 bp, 525-550 bp, 550-575 bp, 575-600 bp, 300-400 bp, 400-500 bp, and/or 300-500 bp in length.

In some embodiments, the chip device is oriented vertically with a heater at the bottom edge which creates a temperature gradient column. In certain instances, the bottom is at denaturing temperature, the middle at annealing temperature, the top at extension temperature. In some instances, convection continually drives the process. In some embodiments, provided herein are methods or systems comprising an electrode design that specifically provides for electrothermal flows and acceleration of the process. In some embodiments, such design is optionally on the same device or on a separate device positioned appropriately. In some instances, active or passive cooling at the top, via fins or fans, or the like, provides a steep temperature gradient. In some instances, the device or system described herein comprises, or a method described herein uses, temperature sensors on the device or in the reaction chamber monitor temperature and such sensors are optionally used to adjust temperature on a feedback basis. In some instances, such sensors are coupled with materials possessing different thermal transfer properties to create continuous and/or discontinuous gradient profiles.

In some embodiments, the amplification proceeds at a constant temperature (i.e, isothermal amplification).

In some embodiments, the methods disclosed herein further comprise sequencing the nucleic acid isolated as disclosed herein. In some embodiments, the nucleic acid is sequenced by Sanger sequencing or next generation sequencing (NGS). In some embodiments, the next generation sequencing methods include, but are not limited to, pyrosequencing, ion semiconductor sequencing, polony sequencing, sequencing by ligation, DNA nanoball sequencing, sequencing by ligation, or single molecule sequencing.

In some embodiments, the isolated nucleic acids disclosed herein are used in Sanger sequencing. In some embodiments, Sanger sequencing is performed within the same device as the nucleic acid isolation (Lab-on-Chip). Lab-on-Chip workflow for sample prep and Sanger sequencing results would incorporate the following steps: a) sample extraction using ACE chips; b) performing amplification of target sequences on chip; c) capture PCR products by ACE; d) perform cycle sequencing to enrich target strand; e) capture enriched target strands; f) perform Sanger chain termination reactions; perform electrophoretic separation of target sequences by capillary electrophoresis with on chip multi-color fluorescence detection. Washing nucleic acids, adding reagent, and turning off voltage is performed as necessary. Reactions can be performed on a single chip with plurality of capture zones or on separate chips and/or reaction chambers.

In some embodiments, the method disclosed herein further comprise performing a reaction on the nucleic acids (e.g., fragmentation, restriction digestion, ligation of DNA or RNA). In some embodiments, the reaction occurs on or near the array or in a device, as disclosed herein.

### Other Assays

The isolated nucleic acids disclosed herein may be further utilized in a variety of assay formats. For instance, devices which are addressed with nucleic acid probes or amplicons may be utilized in dot blot or reverse dot blot analyses, base-stacking single nucleotide polymorphism (SNP) analysis, SNP analysis with electronic stringency, or in STR analysis. In addition, such devices disclosed herein may be utilized in formats for enzymatic nucleic acid modification, or protein-nucleic acid interaction, such as, e.g., gene expression analysis with enzymatic reporting, anchored nucleic acid amplification, or other nucleic acid modifications suitable for solid-phase formats including restriction endonuclease cleavage, endo- or exo-nuclease cleavage, minor groove binding protein assays, terminal transferase reactions, polynucleotide kinase or phosphatase reactions, ligase reactions, topoisomerase reactions, and other nucleic acid binding or modifying protein reactions.

In addition, the devices disclosed herein can be useful in immunoassays. For instance, in some embodiments, locations of the devices can be linked with antigens (e.g., peptides, proteins, carbohydrates, lipids, proteoglycans, glycoproteins, etc.) in order to assay for antibodies in a bodily fluid sample by sandwich assay, competitive assay, or other formats. Alternatively, the locations of the device may be addressed with antibodies, in order to detect antigens in a sample by sandwich assay, competitive assay, or other assay formats. As the isoelectric point of antibodies and proteins can be determined fairly easily by experimentation or pH/charge computations, the electronic addressing and electronic concentration advantages of the devices may be utilized by simply adjusting the pH of the buffer so that the addressed or analyte species will be charged.

In additional aspects, the devices disclosed herein are useful in analysis of biomarkers via mass spectroscopy.

In some embodiments, the isolated nucleic acids are useful for use in immunoassay-type arrays or nucleic acid arrays.

### Definitions and Abbreviations

The articles “a”, “an” and “the” are non-limiting. For example, “the method” includes the broadest definition of the meaning of the phrase, which can be more than one method.
“Vpp” is the peak-to-peak voltage.
“DEP” is an abbreviation for dielectrophoresis.

### EXAMPLES

### Example 1: Chip Construction

A 45×20 custom 80 μm diameter circular platinum microelectrode array on 200 um center-center pitch was fabricated based upon previous results (see references 1-3, below). All 900 microelectrodes are activated together and AC biased to form a checkerboard field geometry. The positive DEP regions occur directly over microelectrodes, and negative low field regions occur between microelectrodes. The array is over-coated with a 200 nm-500 nm thick porous poly-Hema hydrogel layer (Procedure: 12% pHema in ethanol stock solution, purchased from PolySciences Inc., that is diluted to 5% using ethanol. 70 uL of the 5% solution is spun on the above mentioned chip at a 6K RPM spin speed using a spin coater. The chip+hydrogel layer is then put in a 60° C. oven for 45 minutes) and enclosed in a microfluidic cartridge, forming a 50 μL sample chamber covered with an acrylic window. Electrical connections to microelectrodes are accessed from Molex connectors from the PCB board in the flow cell. A function generator (HP 3245A) provided sinusoidal electrical signal at 10 KHz and 10-14V peak-peak, depending on solution conductivity. Images were captured with a fluorescent microscope (Leica) and an EGFP cube (485 nm emission and 525 nm excitation bandpass filters). The excitation source was a PhotoFluor II 200 W Hg arc lamp.
[1] R. Krishnan, B. D. Sullivan, R. L. Mifflin, S. C. Esener, and M. J. Heller, “Alternating current electrokinetic separation and detection of DNA nanoparticles in high-conductance solutions.” Electrophoresis, vol. 29, pages 1765-1774, 2008.
[2] R. Krishnan and M. J. Heller, “An AC electrokinetic method for enhanced detection of DNA nanoparticles.” J. Biophotonics, vol. 2, pages 253-261, 2009.
[3] R. Krishnan, D. A. Dehlinger, G. J. Gemmen, R. L. Mifflin, S. C. Esener, and M. J. Heller, “Interaction of nanoparticles at the DEP microelectrode interface under high conductance conditions” Electrochem. Comm., vol. 11, pages 1661-1666, 2009.

### Example 2: Biomarker Discovery Methods

A plasma sample was obtained from individuals having pancreatic cancer. Extracellular vesicles were isolated from a portion of the plasma samples and cell-free nucleic acids were obtained from the plasma sample using AC dielectrophoretic methods. Nucleic acids from the extracellular vesicles and the cell-free nucleic acids were subject to genomic profiling via next-generation sequencing. In parallel, proteins from the extracellular vesicles were subject to proteomic analysis via mass spectroscopy. Combined analysis when compared to plasma samples from healthy individuals lead to the discovery of biomarkers that were either overexpressed or under expressed in the sample were identified as biomarkers for pancreatic cancer. A flow diagram of the method is shown in FIG. 5. A cluster diagram and heat map of expression of various biomarkers is shown in FIG. 6.

### Example 3: Multi-Cancer Test

A multi-cancer test was developed to determine whether an individual has one of four different cancers with a single test. To validate this approach 247 early stage cancer patients and healthy controls were tested for various biomarkers. The breakdown of experimental subjects is shown in FIG. 7. The results are shown in FIG. 8 where 97% specificity and 87% sensitivity was shown overall with specificity for each cancer type and stage was established. This example shows that multiple cancers can be tested for in a single assay.

### Example 4: Detection of Cancer

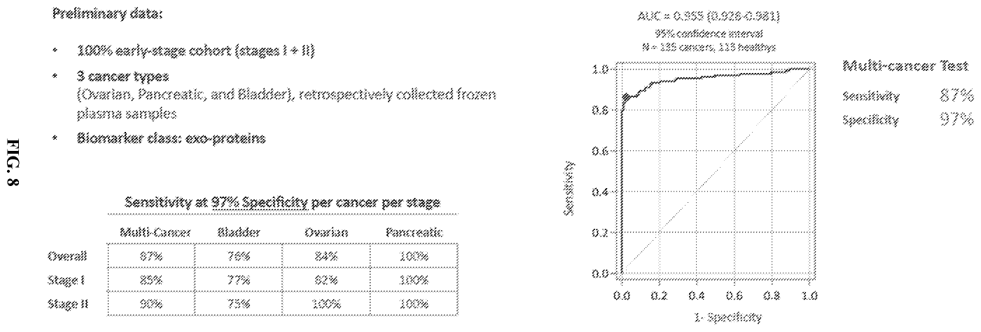

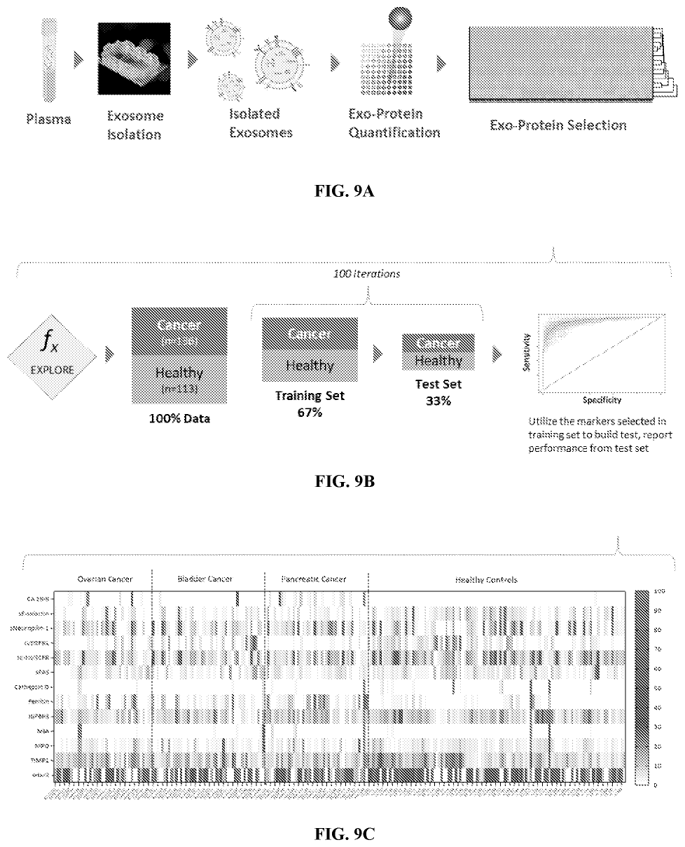

Exosomes were isolated from blood plasma (FIG. 9A) of 134 treatment-naïve cancer patients (42-ovarian, 44-pancreatic, 48-bladder) and 110 healthy individuals (see Methods for details). All cancer patients were histopathologically confirmed per American Joint Commission on Cancer (AJCC) as stage I or stage II, with a median age of 59 years (Tables 1-2). Notably, 63% of the overall cancer (48%-pancreatic, 88%-ovarian and 56%-bladder) patients were stage I; the remaining 37%, stage II. There were also 25 stage IAs (60% of ovarian) in the ovarian cohort. The healthy individuals had no known history of cancer or autoimmune disease, with a median age of 53 years.

TABLE 1
Study Cohorts Overview

| | | | | | Stage | Stage | | | | | | Smokers |
|---|---|---|---|---|---|---|---|---|---|---|---|---|
| | | Histological | | | I | II | Total, | | | Age | BMI | (n, |
| Group | Cohort | subtype | n | % | (A/B/C) | (A/B) | n | Gender | M:F | (Median) | (Median) | %) |
| Cancer | Pancreatic | adenocarcinoma | 44 | 100% | 21 | 23 | 44 | 12M, | 1:2.7 | 49-74 | 14.5-36.5 | 4 |
| | | | | | (4/17/—) | (7/16) | | 32F | | (62) | (24.7) | (9.1%) |
| | Ovarian | All | | | 37 | 5 | 42 | 0M, | 0:1 | 21-76 | 17.7-48.1 | 2 |
| | Cancer | Ovarian | | | (25/8/4) | (3/2) | | 42F | | (51) | (29.2) | (4.8%) |
| | | Cancers | | | | | | | | | | |
| | | serous | 22 | 52% | 19 | 3 | | | | | | |
| | | adenocarcinoma | | | (13/4/2) | (1/2) | | | | | | |
| | | endometrioid | 15 | 36% | 13 | 2 | | | | | | |
| | | adenocarcinoma | | | (7/4/2) | (2/—) | | | | | | |
| | | mucinous | 3 | 7% | 3 | — | | | | | | |
| | | adenocarcinoma | | | (3/—/—) | | | | | | | |
| | | clear cell | 2 | 5% | 2 | — | | | | | | |
| | | adenocarcinoma | | | (2/—/—) | | | | | | | |
| | Bladder | All | | | 27 | 21 | 48 | 42M, | 7:1 | 40-76 | 18-47.8 | 19 |
| | Cancer | Bladder | | | | | | 6F | | (62) | (27.2) | (39.6%) |
| | | Cancers | | | | | | | | | | |
| | | urothelial | 15 | 31% | 14 | 1 | | | | | | |
| | | carcinoma - | | | | | | | | | | |
| | | low grade | | | | | | | | | | |
| | | urothelial | 33 | 69% | 13 | 20 | | | | | | |
| | | carcinoma - | | | | | | | | | | |
| | | high grade | | | | | | | | | | |
| | All | | | | 85 | 49 | 134 | 54M, | 1:1.5 | 21-76 | 14.5-48.1 | 25 |
| | Cancer | | | | (63%) | (37%) | | 80F | | (59) | (27.0) | (18.7%) |
| Healthy | Healthy | n/a | n/a | n/a | n/a | n/a | 110 | 46M, | 1:1.4 | 40-71 | 21-37.8 | 12 |
| | Controls | | | | | | | 64F | | (53) | (26.3) | (10.9%) |
| All | All | n/a | n/a | n/a | 85 | 49 | 244 | 110M, | 1:1.3 | 21-76 | 14.5-48.1 | 37 |
| | | | | | | | | 144F | | (57) | (26.7) | (15.2%) |

TABLE 2
Donor Histopathology and EXPLORE Performance

| Subject | | | | | | | EXPLORE | At >99% | At 97% | At 95% |
|---|---|---|---|---|---|---|---|---|---|---|
| Cohort | | | | | AJCC | | Logistic | Speci- | Speci- | Speci- |
| ID | Age | Sex | Cohort | Histopathology | Stage | A/B/C | Regression* | ficity** | ficity** | ficity** |
| OVAR | 52 | F | ovarian | serous | I | A | 0.9270 | Neg | Pos | Pos |
| 001 | | | cancer | adenocarcinoma | | | | | | |
| OVAR | 59 | F | ovarian | serous | I | A | 1.0000 | Pos | Pos | Pos |
| 002 | | | cancer | papillary | | | | | | |
| | | | | cystadenocarcinoma | | | | | | |
| OVAR | 40 | F | ovarian | serous | I | B | 0.8777 | Neg | Neg | Neg |
| 003 | | | cancer | papillary | | | | | | |
| | | | | cystadenocarcinoma | | | | | | |
| OVAR | 34 | F | ovarian | serous | I | A | 0.9799 | Neg | Pos | Pos |
| 004 | | | cancer | adenocarcinoma | | | | | | |
| OVAR | 63 | F | ovarian | serous | I | C | 0.9883 | Pos | Pos | Pos |
| 005 | | | cancer | papillary | | | | | | |
| | | | | adenocarcinoma | | | | | | |
| OVAR | 23 | F | ovarian | serous | I | A | 0.0810 | Neg | Neg | Neg |
| 006 | | | cancer | papillary | | | | | | |
| | | | | cystadenocarcinoma | | | | | | |
| OVAR | 26 | F | ovarian | serous | I | A | 1.0000 | Pos | Pos | Pos |
| 007 | | | cancer | papillary | | | | | | |
| | | | | cystadenocarcinoma | | | | | | |
| OVAR | 59 | F | ovarian | serous | II | B | 1.0000 | Pos | Pos | Pos |
| 008 | | | cancer | cystadenocarcinoma | | | | | | |
| OVAR | 44 | F | ovarian | serous | II | B | 1.0000 | Pos | Pos | Pos |
| 009 | | | cancer | papillary | | | | | | |
| | | | | adenocarcinoma | | | | | | |
| OVAR | 53 | F | ovarian | serous | I | A | 0.1335 | Neg | Neg | Neg |
| 010 | | | cancer | papillary | | | | | | |
| | | | | cystadenocarcinoma | | | | | | |
| OVAR | 68 | F | ovarian | serous | I | A | 0.9981 | Pos | Pos | Pos |
| 011 | | | cancer | papillary | | | | | | |
| | | | | cystadenocarcinoma | | | | | | |
| OVAR | 58 | F | ovarian | serous | I | B | 1.0000 | Pos | Pos | Pos |
| 012 | | | cancer | papillary | | | | | | |
| | | | | cystadenocarcinoma | | | | | | |
| OVAR | 58 | F | ovarian | clear cell | I | A | 0.1594 | Neg | Neg | Neg |
| 013 | | | cancer | adenocarcinoma | | | | | | |
| OVAR | 44 | F | ovarian | endometrioid | I | B | 0.9999 | Pos | Pos | Pos |
| 014 | | | cancer | adenocarcinoma | | | | | | |
| OVAR | 67 | F | ovarian | endometrioid | I | B | 1.0000 | Pos | Pos | Pos |
| 015 | | | cancer | adenocarcinoma | | | | | | |
| OVAR | 61 | F | ovarian | endometrioid | I | A | 0.9998 | Pos | Pos | Pos |
| 016 | | | cancer | adenocarcinoma | | | | | | |
| OVAR | 47 | F | ovarian | endometrioid | I | A | 0.9979 | Pos | Pos | Pos |
| 017 | | | cancer | adenocarcinoma | | | | | | |
| OVAR | 48 | F | ovarian | endometrioid | I | A | 0.9617 | Neg | Pos | Pos |
| 018 | | | cancer | adenocarcinoma | | | | | | |
| OVAR | 64 | F | ovarian | serous | I | C | 1.0000 | Pos | Pos | Pos |
| 019 | | | cancer | adenocarcinoma | | | | | | |
| OVAR | 43 | F | ovarian | endometrioid | I | B | 1.0000 | Pos | Pos | Pos |
| 020 | | | cancer | adenocarcinoma | | | | | | |
| OVAR | 54 | F | ovarian | serous | I | A | 0.9993 | Pos | Pos | Pos |
| 021 | | | cancer | cystadenocarcinoma | | | | | | |
| OVAR | 61 | F | ovarian | Endometrioid | I | A | 1.0000 | Pos | Pos | Pos |
| 022 | | | cancer | adenocarcinoma | | | | | | |
| OVAR | 46 | F | ovarian | Endometrioid | II | A | 0.9958 | Pos | Pos | Pos |
| 023 | | | cancer | adenocarcinoma | | | | | | |
| OVAR | 74 | F | ovarian | Endometrioid | I | A | 1.0000 | Pos | Pos | Pos |
| 024 | | | cancer | adenocarcinoma | | | | | | |
| OVAR | 76 | F | ovarian | Endometrioid | I | C | 1.0000 | Pos | Pos | Pos |
| 025 | | | cancer | adenocarcinoma | | | | | | |
| OVAR | 48 | F | ovarian | endometrioid | I | C | 0.5986 | Neg | Neg | Neg |
| 026 | | | cancer | adenocarcinoma | | | | | | |
| OVAR | 40 | F | ovarian | serous | I | A | 0.9767 | Neg | Pos | Pos |
| 027 | | | cancer | adenocarcinoma | | | | | | |
| OVAR | 44 | F | ovarian | serous | I | B | 0.9854 | Pos | Pos | Pos |
| 028 | | | cancer | papillary | | | | | | |
| | | | | adenocarcinoma | | | | | | |
| OVAR | 64 | F | ovarian | endometrioid | I | A | 1.0000 | Pos | Pos | Pos |
| 029 | | | cancer | adenocarcinoma | | | | | | |
| OVAR | 54 | F | ovarian | serous | I | B | 0.9792 | Neg | Pos | Pos |
| 030 | | | cancer | adenocarcinoma | | | | | | |
| OVAR | 68 | F | ovarian | mucinous | I | A | 0.9988 | Pos | Pos | Pos |
| 032 | | | cancer | adenocarcinoma | | | | | | |
| OVAR | 41 | F | ovarian | clear cell | I | A | 0.9222 | Neg | Pos | Pos |
| 033 | | | cancer | adenocarcinoma | | | | | | |
| OVAR | 59 | F | ovarian | serous | I | A | 1.0000 | Pos | Pos | Pos |
| 034 | | | cancer | adenocarcinoma | | | | | | |
| OVAR | 58 | F | ovarian | Mucinous | I | A | 1.0000 | Pos | Pos | Pos |
| 035 | | | cancer | adenocarcinoma | | | | | | |
| OVAR | 37 | F | ovarian | Mucinous | I | A | 0.7666 | Neg | Neg | Neg |
| 036 | | | cancer | cystadenocarcinoma | | | | | | |
| OVAR | 62 | F | ovarian | serous | II | A | 0.9662 | Neg | Pos | Pos |
| 037 | | | cancer | papillary | | | | | | |
| | | | | cystadenocarcinoma | | | | | | |
| OVAR | 33 | F | ovarian | Serous and | I | A | 0.9658 | Neg | Pos | Pos |
| 038 | | | cancer | mucinous | | | | | | |
| | | | | adenocarcinoma | | | | | | |
| OVAR | 67 | F | ovarian | serous | I | A | 1.0000 | Pos | Pos | Pos |
| 039 | | | cancer | adenocarcinoma | | | | | | |
| OVAR | 40 | F | ovarian | endometrioid | I | A | 0.5037 | Neg | Neg | Neg |
| 040 | | | cancer | adenocarcinoma | | | | | | |
| OVAR | 21 | F | ovarian | endometrioid | I | B | 0.6192 | Neg | Neg | Neg |
| 041 | | | cancer | adenocarcinoma | | | | | | |
| OVAR | 52 | F | ovarian | serous | I | A | 0.9842 | Pos | Pos | Pos |
| 042 | | | cancer | cystadenocarcinoma | | | | | | |
| OVAR | 52 | F | ovarian | endometrioid | II | A | 1.0000 | Pos | Pos | Pos |
| 043 | | | cancer | adenocarcinoma | | | | | | |
| BLDR | 65 | M | bladder | urothelial | II | | 1.0000 | Pos | Pos | Pos |
| 001 | | | cancer | carcinoma | | | | | | |
| BLDR | 64 | M | bladder | urothelial | I | | 0.9996 | Pos | Pos | Pos |
| 002 | | | cancer | carcinoma | | | | | | |
| BLDR | 70 | M | bladder | urothelial | I | | 0.9995 | Pos | Pos | Pos |
| 003 | | | cancer | carcinoma | | | | | | |
| BLDR | 45 | F | bladder | urothelial | I | | 0.9910 | Pos | Pos | Pos |
| 004 | | | cancer | carcinoma | | | | | | |
| BLDR | 57 | M | bladder | urothelial | II | | 0.8134 | Neg | Neg | Neg |
| 005 | | | cancer | carcinoma | | | | | | |
| BLDR | 60 | M | bladder | urothelial | I | | 0.7595 | Neg | Neg | Neg |
| 006 | | | cancer | carcinoma | | | | | | |
| BLDR | 72 | F | bladder | urothelial | I | | 1.0000 | Pos | Pos | Pos |
| 007 | | | cancer | carcinoma | | | | | | |
| BLDR | 67 | M | bladder | urothelial | II | | 0.5886 | Neg | Neg | Neg |
| 008 | | | cancer | carcinoma | | | | | | |
| BLDR | 76 | M | bladder | urothelial | I | | 0.9632 | Neg | Pos | Pos |
| 009 | | | cancer | carcinoma | | | | | | |
| BLDR | 75 | M | bladder | urothelial | II | | 0.8390 | Neg | Neg | Neg |
| 010 | | | cancer | carcinoma | | | | | | |
| BLDR | 63 | M | bladder | urothelial | II | | 0.8272 | Neg | Neg | Neg |
| 011 | | | cancer | carcinoma | | | | | | |
| BLDR | 73 | M | bladder | urothelial | II | | 0.1915 | Neg | Neg | Neg |
| 012 | | | cancer | carcinoma | | | | | | |
| BLDR | 57 | M | bladder | urothelial | I | | 0.1970 | Neg | Neg | Neg |
| 013 | | | cancer | carcinoma | | | | | | |
| BLDR | 59 | M | bladder | urothelial | I | | 0.9479 | Neg | Pos | Pos |
| 014 | | | cancer | carcinoma | | | | | | |
| BLDR | 70 | M | bladder | urothelial | I | | 0.0090 | Neg | Neg | Neg |
| 015 | | | cancer | carcinoma | | | | | | |
| BLDR | 70 | F | bladder | urothelial | I | | 0.7337 | Neg | Neg | Neg |
| 016 | | | cancer | carcinoma | | | | | | |
| BLDR | 48 | M | bladder | urothelial | II | | 0.8991 | Neg | Neg | Pos |
| 017 | | | cancer | carcinoma | | | | | | |
| BLDR | 74 | M | bladder | urothelial | I | | 0.9918 | Pos | Pos | Pos |
| 018 | | | cancer | carcinoma | | | | | | |
| BLDR | 62 | M | bladder | urothelial | II | | 0.9995 | Pos | Pos | Pos |
| 019 | | | cancer | carcinoma | | | | | | |
| BLDR | 68 | M | bladder | urothelial | II | | 0.9788 | Neg | Pos | Pos |
| 020 | | | cancer | carcinoma | | | | | | |
| BLDR | 62 | M | bladder | urothelial | I | | 0.9999 | Pos | Pos | Pos |
| 021 | | | cancer | carcinoma | | | | | | |
| BLDR | 61 | M | bladder | urothelial | I | | 0.0264 | Neg | Neg | Neg |
| 022 | | | cancer | carcinoma | | | | | | |
| BLDR | 63 | M | bladder | urothelial | I | | 0.7452 | Neg | Neg | Neg |
| 023 | | | cancer | carcinoma | | | | | | |
| BLDR | 67 | M | bladder | urothelial | I | | 0.9758 | Neg | Pos | Pos |
| 024 | | | cancer | carcinoma | | | | | | |
| BLDR | 60 | F | bladder | urothelial | I | | 0.9689 | Neg | Pos | Pos |
| 025 | | | cancer | carcinoma | | | | | | |
| BLDR | 72 | M | bladder | urothelial | II | | 1.0000 | Pos | Pos | Pos |
| 026 | | | cancer | carcinoma | | | | | | |
| BLDR | 66 | M | bladder | urothelial | I | | 0.9996 | Pos | Pos | Pos |
| 027 | | | cancer | carcinoma | | | | | | |
| BLDR | 57 | M | bladder | urothelial | I | | 0.2839 | Neg | Neg | Neg |
| 028 | | | cancer | carcinoma | | | | | | |
| BLDR | 54 | M | bladder | urothelial | II | | 0.4066 | Neg | Neg | Neg |
| 029 | | | cancer | carcinoma | | | | | | |
| BLDR | 55 | M | bladder | urothelial | II | | 0.8279 | Neg | Neg | Neg |
| 030 | | | cancer | carcinoma | | | | | | |
| BLDR | 53 | M | bladder | urothelial | II | | 0.9331 | Neg | Pos | Pos |
| 031 | | | cancer | carcinoma | | | | | | |
| BLDR | 53 | M | bladder | urothelial | II | | 0.2417 | Neg | Neg | Neg |
| 032 | | | cancer | carcinoma | | | | | | |
| BLDR | 72 | M | bladder | urothelial | I | | 1.0000 | Pos | Pos | Pos |
| 033 | | | cancer | carcinoma | | | | | | |
| BLDR | 68 | M | bladder | urothelial | II | | 0.9993 | Pos | Pos | Pos |
| 034 | | | cancer | carcinoma | | | | | | |
| BLDR | 63 | M | bladder | urothelial | II | | 1.0000 | Pos | Pos | Pos |
| 035 | | | cancer | carcinoma | | | | | | |
| BLDR | 70 | M | bladder | urothelial | I | | 1.0000 | Pos | Pos | Pos |
| 036 | | | cancer | carcinoma | | | | | | |
| BLDR | 58 | M | bladder | urothelial | II | | 1.0000 | Pos | Pos | Pos |
| 037 | | | cancer | carcinoma | | | | | | |
| BLDR | 69 | M | bladder | urothelial | II | | 1.0000 | Pos | Pos | Pos |
| 038 | | | cancer | carcinoma | | | | | | |
| BLDR | 73 | M | bladder | urothelial | I | | 1.0000 | Pos | Pos | Pos |
| 039 | | | cancer | carcinoma | | | | | | |
| BLDR | 58 | M | bladder | urothelial | I | | 0.9998 | Pos | Pos | Pos |
| 040 | | | cancer | carcinoma | | | | | | |
| BLDR | 40 | F | bladder | urothelial | II | | 0.9927 | Pos | Pos | Pos |
| 041 | | | cancer | carcinoma | | | | | | |
| BLDR | 61 | M | bladder | urothelial | II | | 0.2326 | Neg | Neg | Neg |
| 042 | | | cancer | carcinoma | | | | | | |
| BLDR | 63 | M | bladder | urothelial | I | | 0.2862 | Neg | Neg | Neg |
| 043 | | | cancer | carcinoma | | | | | | |
| BLDR | 58 | M | bladder | urothelial | I | | 0.9640 | Neg | Pos | Pos |
| 044 | | | cancer | carcinoma | | | | | | |
| BLDR | 63 | M | bladder | urothelial | II | | 0.0154 | Neg | Neg | Neg |
| 045 | | | cancer | carcinoma | | | | | | |
| BLDR | 41 | F | bladder | urothelial | I | | 0.3225 | Neg | Neg | Neg |
| 046 | | | cancer | carcinoma | | | | | | |
| BLDR | 68 | M | bladder | urothelial | I | | 0.9998 | Pos | Pos | Pos |
| 047 | | | cancer | carcinoma | | | | | | |
| BLDR | 56 | M | bladder | urothelial | I | | 0.9982 | Pos | Pos | Pos |
| 048 | | | cancer | carcinoma | | | | | | |
| PDAC | 60 | F | pancreatic | adenocarcinoma | I | A | 0.9999 | Pos | Pos | Pos |
| 001 | | | cancer | | | | | | | |
| PDAC | 49 | F | pancreatic | adenocarcinoma | II | B | 0.9962 | Pos | Pos | Pos |
| 002 | | | cancer | | | | | | | |
| PDAC | 65 | F | pancreatic | adenocarcinoma | II | A | 1.0000 | Pos | Pos | Pos |
| 003 | | | cancer | | | | | | | |
| PDAC | 60 | F | pancreatic | adenocarcinoma | II | A | 1.0000 | Pos | Pos | Pos |
| 004 | | | cancer | | | | | | | |
| PDAC | 58 | F | pancreatic | adenocarcinoma | II | A | 0.8765 | Neg | Neg | Neg |
| 005 | | | cancer | | | | | | | |
| PDAC | 56 | F | pancreatic | adenocarcinoma | II | B | 0.9998 | Pos | Pos | Pos |
| 006 | | | cancer | | | | | | | |
| PDAC | 66 | F | pancreatic | adenocarcinoma | II | A | 1.0000 | Pos | Pos | Pos |
| 007 | | | cancer | | | | | | | |
| PDAC | 63 | F | pancreatic | adenocarcinoma | II | B | 1.0000 | Pos | Pos | Pos |
| 008 | | | cancer | | | | | | | |
| PDAC | 59 | F | pancreatic | adenocarcinoma | II | B | 1.0000 | Pos | Pos | Pos |
| 009 | | | cancer | | | | | | | |
| PDAC | 65 | F | pancreatic | adenocarcinoma | I | B | 1.0000 | Pos | Pos | Pos |
| 010 | | | cancer | | | | | | | |
| PDAC | 57 | M | pancreatic | adenocarcinoma | II | B | 1.0000 | Pos | Pos | Pos |
| 011 | | | cancer | | | | | | | |
| PDAC | 64 | F | pancreatic | adenocarcinoma | I | B | 0.9991 | Pos | Pos | Pos |
| 012 | | | cancer | | | | | | | |
| PDAC | 55 | F | pancreatic | adenocarcinoma | I | B | 1.0000 | Pos | Pos | Pos |
| 013 | | | cancer | | | | | | | |
| PDAC | 66 | F | pancreatic | adenocarcinoma | II | A | 1.0000 | Pos | Pos | Pos |
| 014 | | | cancer | | | | | | | |
| PDAC | 68 | F | pancreatic | adenocarcinoma | II | B | 0.9944 | Pos | Pos | Pos |
| 015 | | | cancer | | | | | | | |
| PDAC | 68 | M | pancreatic | adenocarcinoma | I | B | 1.0000 | Pos | Pos | Pos |
| 016 | | | cancer | | | | | | | |
| PDAC | 59 | F | pancreatic | adenocarcinoma | II | B | 0.9489 | Neg | Pos | Pos |
| 017 | | | cancer | | | | | | | |
| PDAC | 66 | M | pancreatic | adenocarcinoma | II | B | 1.0000 | Pos | Pos | Pos |
| 018 | | | cancer | | | | | | | |
| PDAC | 68 | F | pancreatic | adenocarcinoma | II | B | 1.0000 | Pos | Pos | Pos |
| 019 | | | cancer | | | | | | | |
| PDAC | 52 | M | pancreatic | adenocarcinoma | I | A | 1.0000 | Pos | Pos | Pos |
| 020 | | | cancer | | | | | | | |
| PDAC | 64 | F | pancreatic | adenocarcinoma | I | B | 1.0000 | Pos | Pos | Pos |
| 021 | | | cancer | | | | | | | |
| PDAC | 56 | F | pancreatic | adenocarcinoma | II | B | 1.0000 | Pos | Pos | Pos |
| 022 | | | cancer | | | | | | | |
| PDAC | 50 | F | pancreatic | adenocarcinoma | II | B | 0.9998 | Pos | Pos | Pos |
| 023 | | | cancer | | | | | | | |
| PDAC | 49 | M | pancreatic | adenocarcinoma | II | B | 1.0000 | Pos | Pos | Pos |
| 024 | | | cancer | | | | | | | |
| PDAC | 63 | F | pancreatic | adenocarcinoma | II | B | 1.0000 | Pos | Pos | Pos |
| 025 | | | cancer | | | | | | | |
| PDAC | 70 | F | pancreatic | adenocarcinoma | II | B | 0.9983 | Pos | Pos | Pos |
| 026 | | | cancer | | | | | | | |
| PDAC | 57 | F | pancreatic | adenocarcinoma | II | B | 0.9946 | Pos | Pos | Pos |
| 027 | | | cancer | | | | | | | |
| PDAC | 61 | F | pancreatic | adenocarcinoma | II | B | 1.0000 | Pos | Pos | Pos |
| 028 | | | cancer | | | | | | | |
| PDAC | 71 | F | pancreatic | adenocarcinoma | I | B | 1.0000 | Pos | Pos | Pos |
| 029 | | | cancer | | | | | | | |
| PDAC | 59 | M | pancreatic | adenocarcinoma | I | B | 1.0000 | Pos | Pos | Pos |
| 030 | | | cancer | | | | | | | |
| PDAC | 61 | F | pancreatic | adenocarcinoma | I | B | 0.9734 | Neg | Pos | Pos |
| 031 | | | cancer | | | | | | | |
| PDAC | 60 | F | pancreatic | adenocarcinoma | I | A | 0.9998 | Pos | Pos | Pos |
| 032 | | | cancer | | | | | | | |
| PDAC | 72 | M | pancreatic | adenocarcinoma | I | B | 1.0000 | Pos | Pos | Pos |
| 033 | | | cancer | | | | | | | |
| PDAC | 64 | M | pancreatic | adenocarcinoma | I | B | 1.0000 | Pos | Pos | Pos |
| 034 | | | cancer | | | | | | | |
| PDAC | 70 | F | pancreatic | adenocarcinoma | I | B | 1.0000 | Pos | Pos | Pos |
| 035 | | | cancer | | | | | | | |
| PDAC | 72 | F | pancreatic | adenocarcinoma | I | B | 1.0000 | Pos | Pos | Pos |
| 036 | | | cancer | | | | | | | |
| PDAC | 58 | M | pancreatic | adenocarcinoma | I | B | 1.0000 | Pos | Pos | Pos |
| 037 | | | cancer | | | | | | | |
| PDAC | 64 | F | pancreatic | adenocarcinoma | I | B | 0.9963 | Pos | Pos | Pos |
| 038 | | | cancer | | | | | | | |
| PDAC | 74 | M | pancreatic | adenocarcinoma | I | A | 1.0000 | Pos | Pos | Pos |
| 039 | | | cancer | | | | | | | |
| PDAC | 66 | F | pancreatic | adenocarcinoma | I | B | 0.9988 | Pos | Pos | Pos |
| 040 | | | cancer | | | | | | | |
| PDAC | 69 | M | pancreatic | adenocarcinoma | I | B | 0.9919 | Pos | Pos | Pos |
| 041 | | | cancer | | | | | | | |
| PDAC | 59 | F | pancreatic | adenocarcinoma | I | B | 0.9972 | Pos | Pos | Pos |
| 042 | | | cancer | | | | | | | |
| PDAC | 74 | F | pancreatic | adenocarcinoma | II | A | 1.0000 | Pos | Pos | Pos |
| 043 | | | cancer | | | | | | | |
| PDAC | 59 | M | pancreatic | adenocarcinoma | II | A | 1.0000 | Pos | Pos | Pos |
| 044 | | | cancer | | | | | | | |
| HC 001 | 61 | F | healthy | | | | 0.9854 | Pos | Pos | Pos |
| | | | control | | | | | | | |
| HC 002 | 64 | F | healthy | | | | 0.0133 | Neg | Neg | Neg |
| | | | control | | | | | | | |
| HC 003 | 71 | F | healthy | | | | 0.0001 | Neg | Neg | Neg |
| | | | control | | | | | | | |
| HC 004 | 69 | F | healthy | | | | 0.8215 | Neg | Neg | Neg |
| | | | control | | | | | | | |
| HC 005 | 70 | F | healthy | | | | 0.1141 | Neg | Neg | Neg |
| | | | control | | | | | | | |
| HC 006 | 54 | F | healthy | | | | 0.0000 | Neg | Neg | Neg |
| | | | control | | | | | | | |
| HC 007 | 50 | F | healthy | | | | 0.0544 | Neg | Neg | Neg |
| | | | control | | | | | | | |
| HC 008 | 53 | F | healthy | | | | 0.0018 | Neg | Neg | Neg |
| | | | control | | | | | | | |
| HC 009 | 62 | M | healthy | | | | 0.0049 | Neg | Neg | Neg |
| | | | control | | | | | | | |
| HC 010 | 63 | F | healthy | | | | 0.0823 | Neg | Neg | Neg |
| | | | control | | | | | | | |
| HC 011 | 60 | M | healthy | | | | 0.0108 | Neg | Neg | Neg |
| | | | control | | | | | | | |
| HC 012 | 55 | F | healthy | | | | 0.0933 | Neg | Neg | Neg |
| | | | control | | | | | | | |
| HC 013 | 65 | F | healthy | | | | 0.7494 | Neg | Neg | Neg |
| | | | control | | | | | | | |
| HC 014 | 67 | F | healthy | | | | 0.0034 | Neg | Neg | Neg |
| | | | control | | | | | | | |
| HC 015 | 63 | F | healthy | | | | 0.9028 | Neg | Neg | Neg |
| | | | control | | | | | | | |
| HC 016 | 60 | F | healthy | | | | 0.0123 | Neg | Neg | Neg |
| | | | control | | | | | | | |
| HC 017 | 65 | M | healthy | | | | 0.0061 | Neg | Neg | Neg |
| | | | control | | | | | | | |
| HC 018 | 57 | F | healthy | | | | 0.0365 | Neg | Neg | Neg |
| | | | control | | | | | | | |
| HC 019 | 55 | F | healthy | | | | 0.1680 | Neg | Neg | Neg |
| | | | control | | | | | | | |
| HC 020 | 68 | F | healthy | | | | 0.0059 | Neg | Neg | Neg |
| | | | control | | | | | | | |
| HC 021 | 65 | F | healthy | | | | 0.0672 | Neg | Neg | Neg |
| | | | control | | | | | | | |
| HC 022 | 67 | F | healthy | | | | 0.0312 | Neg | Neg | Neg |
| | | | control | | | | | | | |
| HC 023 | 70 | F | healthy | | | | 0.7480 | Neg | Neg | Neg |
| | | | control | | | | | | | |
| HC 024 | 71 | F | healthy | | | | 0.0819 | Neg | Neg | Neg |
| | | | control | | | | | | | |
| HC 025 | 60 | M | healthy | | | | 0.0006 | Neg | Neg | Neg |
| | | | control | | | | | | | |
| HC 026 | 63 | F | healthy | | | | 0.1964 | Neg | Neg | Neg |
| | | | control | | | | | | | |
| HC 028 | 65 | M | healthy | | | | 0.0001 | Neg | Neg | Neg |
| | | | control | | | | | | | |
| HC 029 | 65 | M | healthy | | | | 0.8717 | Neg | Neg | Neg |
| | | | control | | | | | | | |
| HC 030 | 61 | F | healthy | | | | 0.0025 | Neg | Neg | Neg |
| | | | control | | | | | | | |
| HC 031 | 70 | M | healthy | | | | 0.3978 | Neg | Neg | Neg |
| | | | control | | | | | | | |
| HC 032 | 60 | M | healthy | | | | 0.8952 | Neg | Neg | Neg |
| | | | control | | | | | | | |
| HC 033 | 62 | F | healthy | | | | 0.0000 | Neg | Neg | Neg |
| | | | control | | | | | | | |
| HC 034 | 65 | F | healthy | | | | 0.0074 | Neg | Neg | Neg |
| | | | control | | | | | | | |
| HC 035 | 61 | M | healthy | | | | 0.0189 | Neg | Neg | Neg |
| | | | control | | | | | | | |
| HC 036 | 67 | F | healthy | | | | 0.0000 | Neg | Neg | Neg |
| | | | control | | | | | | | |
| HC 037 | 69 | F | healthy | | | | 0.0076 | Neg | Neg | Neg |
| | | | control | | | | | | | |
| HC 038 | 70 | M | healthy | | | | 0.0000 | Neg | Neg | Neg |
| | | | control | | | | | | | |
| HC 039 | 65 | F | healthy | | | | 0.0000 | Neg | Neg | Neg |
| | | | control | | | | | | | |
| HC 040 | 71 | F | healthy | | | | 0.9821 | Neg | Pos | Neg |
| | | | control | | | | | | | |
| HC 041 | 60 | M | healthy | | | | 0.0000 | Neg | Neg | Neg |
| | | | control | | | | | | | |
| HC 042 | 63 | M | healthy | | | | 0.7679 | Neg | Neg | Neg |
| | | | control | | | | | | | |
| HC 043 | 55 | F | healthy | | | | 0.0307 | Neg | Neg | Neg |
| | | | control | | | | | | | |
| HC 044 | 60 | M | healthy | | | | 0.0002 | Neg | Neg | Neg |
| | | | control | | | | | | | |
| HC 045 | 60 | M | healthy | | | | 0.0134 | Neg | Neg | Neg |
| | | | control | | | | | | | |
| HC 046 | 54 | F | healthy | | | | 0.3174 | Neg | Neg | Neg |
| | | | control | | | | | | | |
| HC 047 | 60 | M | healthy | | | | 0.4969 | Neg | Neg | Neg |
| | | | control | | | | | | | |
| HC 048 | 59 | F | healthy | | | | 0.5460 | Neg | Neg | Neg |
| | | | control | | | | | | | |
| HC 049 | 50 | F | healthy | | | | 0.0002 | Neg | Neg | Neg |
| | | | control | | | | | | | |
| HC 050 | 50 | M | healthy | | | | 0.1443 | Neg | Neg | Neg |
| | | | control | | | | | | | |
| HC 051 | 54 | M | healthy | | | | 0.0253 | Neg | Neg | Neg |
| | | | control | | | | | | | |
| HC 052 | 54 | F | healthy | | | | 0.4239 | Neg | Neg | Neg |
| | | | control | | | | | | | |
| HC 053 | 48 | M | healthy | | | | 0.1588 | Neg | Neg | Neg |
| | | | control | | | | | | | |
| HC 054 | 44 | M | healthy | | | | 0.0006 | Neg | Neg | Neg |
| | | | control | | | | | | | |
| HC 055 | 42 | M | healthy | | | | 0.0522 | Neg | Neg | Neg |
| | | | control | | | | | | | |
| HC 056 | 46 | M | healthy | | | | 0.2665 | Neg | Neg | Neg |
| | | | control | | | | | | | |
| HC 057 | 49 | F | healthy | | | | 0.0045 | Neg | Neg | Neg |
| | | | control | | | | | | | |
| HC 058 | 42 | F | healthy | | | | 0.0000 | Neg | Neg | Neg |
| | | | control | | | | | | | |
| HC 059 | 50 | F | healthy | | | | 0.0001 | Neg | Neg | Neg |
| | | | control | | | | | | | |
| HC 060 | 49 | F | healthy | | | | 0.2130 | Neg | Neg | Neg |
| | | | control | | | | | | | |
| HC 061 | 48 | F | healthy | | | | 0.2776 | Neg | Neg | Neg |
| | | | control | | | | | | | |
| HC 062 | 48 | M | healthy | | | | 0.7137 | Neg | Neg | Neg |
| | | | control | | | | | | | |
| HC 063 | 43 | M | healthy | | | | 0.9562 | Neg | Pos | Neg |
| | | | control | | | | | | | |
| HC 064 | 48 | M | healthy | | | | 0.5076 | Neg | Neg | Neg |
| | | | control | | | | | | | |
| HC 065 | 45 | M | healthy | | | | 0.0319 | Neg | Neg | Neg |
| | | | control | | | | | | | |
| HC 066 | 44 | F | healthy | | | | 0.5744 | Neg | Neg | Neg |
| | | | control | | | | | | | |
| HC 067 | 46 | M | healthy | | | | 0.0348 | Neg | Neg | Neg |
| | | | control | | | | | | | |
| HC 068 | 42 | M | healthy | | | | 0.0000 | Neg | Neg | Neg |
| | | | control | | | | | | | |
| HC 069 | 46 | M | healthy | | | | 0.0315 | Neg | Neg | Neg |
| | | | control | | | | | | | |
| HC 070 | 47 | M | healthy | | | | 0.1190 | Neg | Neg | Neg |
| | | | control | | | | | | | |
| HC 071 | 41 | F | healthy | | | | 0.0013 | Neg | Neg | Neg |
| | | | control | | | | | | | |
| HC 072 | 49 | M | healthy | | | | 0.8005 | Neg | Neg | Neg |
| | | | control | | | | | | | |
| HC 073 | 45 | F | healthy | | | | 0.0004 | Neg | Neg | Neg |
| | | | control | | | | | | | |
| HC 074 | 46 | F | healthy | | | | 0.0057 | Neg | Neg | Neg |
| | | | control | | | | | | | |
| HC 075 | 47 | F | healthy | | | | 0.0836 | Neg | Neg | Neg |
| | | | control | | | | | | | |
| HC 076 | 50 | M | healthy | | | | 0.0002 | Neg | Neg | Neg |
| | | | control | | | | | | | |
| HC 077 | 48 | F | healthy | | | | 0.0002 | Neg | Neg | Neg |
| | | | control | | | | | | | |
| HC 078 | 48 | F | healthy | | | | 0.0019 | Neg | Neg | Neg |
| | | | control | | | | | | | |
| HC 079 | 49 | M | healthy | | | | 0.0003 | Neg | Neg | Neg |
| | | | control | | | | | | | |
| HC 080 | 47 | F | healthy | | | | 0.0008 | Neg | Neg | Neg |
| | | | control | | | | | | | |
| HC 082 | 48 | F | healthy | | | | 0.0006 | Neg | Neg | Neg |
| | | | control | | | | | | | |
| HC 084 | 43 | M | healthy | | | | 0.8909 | Neg | Neg | Neg |
| | | | control | | | | | | | |
| HC 085 | 47 | F | healthy | | | | 0.0253 | Neg | Neg | Neg |
| | | | control | | | | | | | |
| HC 086 | 49 | M | healthy | | | | 0.0041 | Neg | Neg | Neg |
| | | | control | | | | | | | |
| HC 087 | 45 | F | healthy | | | | 0.0042 | Neg | Neg | Neg |
| | | | control | | | | | | | |
| HC 088 | 45 | F | healthy | | | | 0.0552 | Neg | Neg | Neg |
| | | | control | | | | | | | |
| HC 089 | 45 | M | healthy | | | | 0.1830 | Neg | Neg | Neg |
| | | | control | | | | | | | |
| HC 090 | 43 | M | healthy | | | | 0.1206 | Neg | Neg | Neg |
| | | | control | | | | | | | |
| HC 091 | 44 | F | healthy | | | | 0.0003 | Neg | Neg | Neg |
| | | | control | | | | | | | |
| HC 092 | 40 | M | healthy | | | | 0.4735 | Neg | Neg | Neg |
| | | | control | | | | | | | |
| HC 093 | 47 | F | healthy | | | | 0.0078 | Neg | Neg | Neg |
| | | | control | | | | | | | |
| HC 094 | 48 | M | healthy | | | | 0.0668 | Neg | Neg | Neg |
| | | | control | | | | | | | |
| HC 095 | 41 | M | healthy | | | | 0.0075 | Neg | Neg | Neg |
| | | | control | | | | | | | |
| HC 096 | 48 | M | healthy | | | | 0.0000 | Neg | Neg | Neg |
| | | | control | | | | | | | |
| HC 097 | 45 | M | healthy | | | | 0.0000 | Neg | Neg | Neg |
| | | | control | | | | | | | |
| HC 098 | 45 | F | healthy | | | | 0.0075 | Neg | Neg | Neg |
| | | | control | | | | | | | |
| HC 099 | 67 | M | healthy | | | | 0.0000 | Neg | Neg | Neg |
| | | | control | | | | | | | |
| HC 100 | 60 | F | healthy | | | | 0.0006 | Neg | Neg | Neg |
| | | | control | | | | | | | |
| HC 101 | 41 | F | healthy | | | | 0.4586 | Neg | Neg | Neg |
| | | | control | | | | | | | |
| HC 102 | 45 | F | healthy | | | | 0.0065 | Neg | Neg | Neg |
| | | | control | | | | | | | |
| HC 103 | 40 | F | healthy | | | | 0.1371 | Neg | Neg | Neg |
| | | | control | | | | | | | |
| HC 104 | 45 | F | healthy | | | | 0.3566 | Neg | Neg | Neg |
| | | | control | | | | | | | |
| HC 105 | 41 | M | healthy | | | | 0.0001 | Neg | Neg | Neg |
| | | | control | | | | | | | |
| HC 106 | 44 | M | healthy | | | | 0.0000 | Neg | Neg | Neg |
| | | | control | | | | | | | |
| HC 107 | 44 | M | healthy | | | | 0.0662 | Neg | Neg | Neg |
| | | | control | | | | | | | |
| HC 108 | 41 | F | healthy | | | | 0.0001 | Neg | Neg | Neg |
| | | | control | | | | | | | |
| HC 109 | 43 | F | healthy | | | | 0.3816 | Neg | Neg | Neg |
| | | | control | | | | | | | |
| HC 110 | 42 | F | healthy | | | | 0.6466 | Neg | Neg | Neg |
| | | | control | | | | | | | |
| HC 111 | 49 | F | healthy | | | | 0.4949 | Neg | Neg | Neg |
| | | | control | | | | | | | |
| HC 112 | 40 | F | healthy | | | | 0.0169 | Neg | Neg | Neg |
| | | | control | | | | | | | |
| HC 113 | 60 | M | healthy | | | | 0.2657 | Neg | Neg | Neg |
| | | | control | | | | | | | |

*Probability from representative logistic regression instance
**Pos/Neg Test results within confidence interval of Average ROC curve shown in FIG. 2A

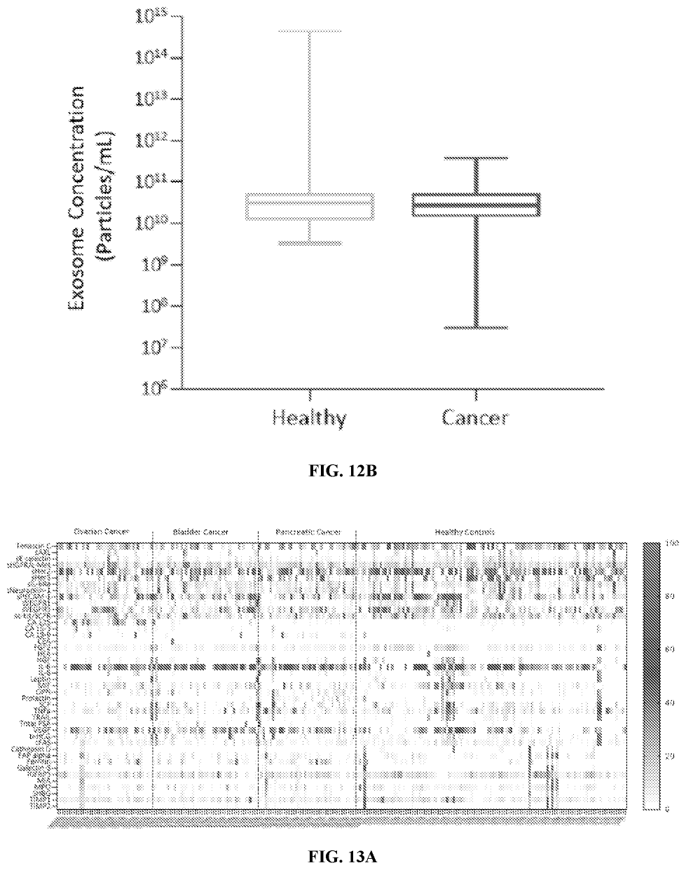

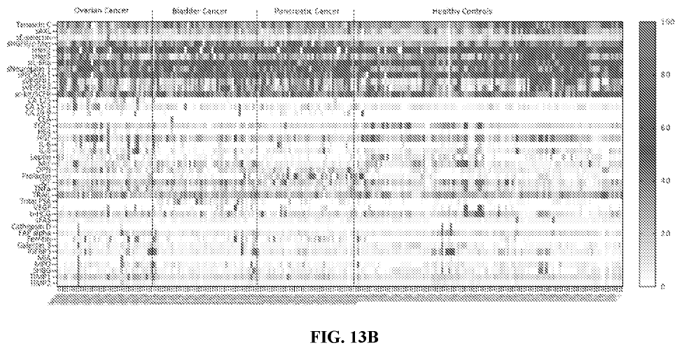

Using existing literature on cancer-related proteins, 42 protein biomarkers were selected and 2 other factors (age and sex) for evaluation (Table 4). It was found that these proteins could be reproducibly evaluated through an immunoassay platform, and exo-protein levels of the 42 markers were measured in plasma of all subjects (Table 5). Particle size distribution and concentration confirmed equivalent exosome isolation in both cohorts (Table 3; FIG. 12). Protein abundance heatmaps for exo-proteins and free-proteins (total circulating plasma proteins) are shown in FIG. 13. All biomarkers in Table 4 were evaluated for inclusion into a logistic regression model developed to detect cancer at early stages—the EXPLORE test.

TABLE 3
Donor Histopathology and Exosome Characterization

| | | | | | | | | Median |
|---|---|---|---|---|---|---|---|---|
| | | | | | | | | size of |
| Subject | | | | | | | EV particle | isolated |
| Cohort | | | | | AJCC | | concentration | EVs |
| ID | Age | Sex | Cohort | Histopathology | Stage | A/B/C | (particles/mL) | (nm) |
| OVAR | 52 | F | ovarian | serous | I | A | 4.31E+10 | 133.65 |
| 001 | | | cancer | adenocarcinoma | | | | |
| OVAR | 59 | F | ovarian | serous papillary | I | A | 2.73E+10 | 128.03 |
| 002 | | | cancer | cystadenocarcinoma | | | | |
| OVAR | 40 | F | ovarian | serous papillary | I | B | 2.65E+10 | 132.33 |
| 003 | | | cancer | cystadenocarcinoma | | | | |
| OVAR | 34 | F | ovarian | serous | I | A | 2.07E+10 | 134.67 |
| 004 | | | cancer | adenocarcinoma | | | | |
| OVAR | 63 | F | ovarian | serous papillary | I | C | 2.57E+10 | 149.88 |
| 005 | | | cancer | adenocarcinoma | | | | |
| OVAR | 23 | F | ovarian | serous papillary | I | A | 1.25E+10 | 130.35 |
| 006 | | | cancer | cystadenocarcinoma | | | | |
| OVAR | 26 | F | ovarian | serous papillary | I | A | 3.05E+10 | 125.13 |
| 007 | | | cancer | cystadenocarcinoma | | | | |
| OVAR | 59 | F | ovarian | serous | II | B | 3.10E+10 | 124.13 |
| 008 | | | cancer | cystadenocarcinoma | | | | |
| OVAR | 44 | F | ovarian | serous papillary | II | B | 2.80E+10 | 118.60 |
| 009 | | | cancer | adenocarcinoma | | | | |
| OVAR | 53 | F | ovarian | serous papillary | I | A | 1.21E+11 | 142.75 |
| 010 | | | cancer | cystadenocarcinoma | | | | |
| OVAR | 68 | F | ovarian | serous papillary | I | A | 3.30E+10 | 126.15 |
| 011 | | | cancer | cystadenocarcinoma | | | | |
| OVAR | 58 | F | ovarian | serous papillary | I | B | 2.14E+10 | 134.33 |
| 012 | | | cancer | cystadenocarcinoma | | | | |
| OVAR | 58 | F | ovarian | clear cell | I | A | 4.69E+10 | 111.95 |
| 013 | | | cancer | adenocarcinoma | | | | |
| OVAR | 44 | F | ovarian | endometrioid | I | B | 8.20E+10 | 102.55 |
| 014 | | | cancer | adenocarcinoma | | | | |
| OVAR | 67 | F | ovarian | endometrioid | I | B | 8.30E+10 | 106.05 |
| 015 | | | cancer | adenocarcinoma | | | | |
| OVAR | 61 | F | ovarian | endometrioid | I | A | 1.80E+11 | 121.58 |
| 016 | | | cancer | adenocarcinoma | | | | |
| OVAR | 47 | F | ovarian | endometrioid | I | A | 3.17E+11 | 113.33 |
| 017 | | | cancer | adenocarcinoma | | | | |
| OVAR | 48 | F | ovarian | endometrioid | I | A | 7.42E+10 | 119.73 |
| 018 | | | cancer | adenocarcinoma | | | | |
| OVAR | 64 | F | ovarian | serous | I | C | 2.00E+10 | 125.50 |
| 019 | | | cancer | adenocarcinoma | | | | |
| OVAR | 43 | F | ovarian | endometrioid | I | B | 2.35E+10 | 117.35 |
| 020 | | | cancer | adenocarcinoma | | | | |
| OVAR | 54 | F | ovarian | serous | I | A | 1.50E+10 | 122.75 |
| 021 | | | cancer | cystadenocarcinoma | | | | |
| OVAR | 61 | F | ovarian | Endometrioid | I | A | 6.10E+10 | 96.90 |
| 022 | | | cancer | adenocarcinoma | | | | |
| OVAR | 46 | F | ovarian | Endometrioid | II | A | 5.95E+10 | 109.95 |
| 023 | | | cancer | adenocarcinoma | | | | |
| OVAR | 74 | F | ovarian | Endometrioid | I | A | 5.90E+10 | 108.85 |
| 024 | | | cancer | adenocarcinoma | | | | |
| OVAR | 76 | F | ovarian | Endometrioid | I | C | 4.30E+10 | 110.20 |
| 025 | | | cancer | adenocarcinoma | | | | |
| OVAR | 48 | F | ovarian | endometrioid | I | C | 4.10E+10 | 111.05 |
| 026 | | | cancer | adenocarcinoma | | | | |
| OVAR | 40 | F | ovarian | serous | I | A | 5.00E+10 | 102.37 |
| 027 | | | cancer | adenocarcinoma | | | | |
| OVAR | 44 | F | ovarian | serous papillary | I | B | 1.45E+10 | 124.80 |
| 028 | | | cancer | adenocarcinoma | | | | |
| OVAR | 64 | F | ovarian | endometrioid | I | A | 2.31E+10 | 117.45 |
| 029 | | | cancer | adenocarcinoma | | | | |
| OVAR | 54 | F | ovarian | serous | I | B | 3.50E+10 | 119.85 |
| 030 | | | cancer | adenocarcinoma | | | | |
| OVAR | 68 | F | ovarian | mucinous | I | A | 5.18E+09 | 141.18 |
| 032 | | | cancer | adenocarcinoma | | | | |
| OVAR | 41 | F | ovarian | clear cell | I | A | 6.95E+09 | 130.85 |
| 033 | | | cancer | adenocarcinoma | | | | |
| OVAR | 59 | F | ovarian | serous | I | A | 7.55E+09 | 145.70 |
| 034 | | | cancer | adenocarcinoma | | | | |
| OVAR | 58 | F | ovarian | Mucinous | I | A | 7.70E+10 | 104.50 |
| 035 | | | cancer | adenocarcinoma | | | | |
| OVAR | 37 | F | ovarian | Mucinous | I | A | 4.30E+10 | 90.70 |
| 036 | | | cancer | cystadenocarcinoma | | | | |
| OVAR | 62 | F | ovarian | serous papillary | II | A | 1.05E+10 | 122.85 |
| 037 | | | cancer | cystadenocarcinoma | | | | |
| OVAR | 33 | F | ovarian | Serous and | I | A | #N/A | #N/A |
| 038 | | | cancer | mucinous | | | | |
| | | | | adenocarcinoma | | | | |
| OVAR | 67 | F | ovarian | serous | I | A | 4.90E+09 | 161.70 |
| 039 | | | cancer | adenocarcinoma | | | | |
| OVAR | 40 | F | ovarian | endometrioid | I | A | 3.29E+10 | 137.23 |
| 040 | | | cancer | adenocarcinoma | | | | |
| OVAR | 21 | F | ovarian | endometrioid | I | B | 2.67E+10 | 164.53 |
| 041 | | | cancer | adenocarcinoma | | | | |
| OVAR | 52 | F | ovarian | serous | I | A | 6.13E+09 | 142.30 |
| 042 | | | cancer | cystadenocarcinoma | | | | |
| OVAR | 52 | F | ovarian | endometrioid | II | A | 7.83E+09 | 140.07 |
| 043 | | | cancer | adenocarcinoma | | | | |
| BLDR | 65 | M | bladder | urothelial | II | | 1.69E+11 | 124.18 |
| 001 | | | cancer | carcinoma | | | | |
| BLDR | 64 | M | bladder | urothelial | I | | 1.37E+10 | 132.06 |
| 002 | | | cancer | carcinoma | | | | |
| BLDR | 70 | M | bladder | urothelial | I | | 1.14E+11 | 112.30 |
| 003 | | | cancer | carcinoma | | | | |
| BLDR | 45 | F | bladder | urothelial | I | | 1.35E+10 | 159.63 |
| 004 | | | cancer | carcinoma | | | | |
| BLDR | 57 | M | bladder | urothelial | II | | 3.81E+11 | 114.20 |
| 005 | | | cancer | carcinoma | | | | |
| BLDR | 60 | M | bladder | urothelial | I | | 1.98E+11 | 109.10 |
| 006 | | | cancer | carcinoma | | | | |
| BLDR | 72 | F | bladder | urothelial | I | | 2.10E+10 | 112.70 |
| 007 | | | cancer | carcinoma | | | | |
| BLDR | 67 | M | bladder | urothelial | II | | 2.16E+10 | 128.47 |
| 008 | | | cancer | carcinoma | | | | |
| BLDR | 76 | M | bladder | urothelial | I | | 2.74E+10 | 129.80 |
| 009 | | | cancer | carcinoma | | | | |
| BLDR | 75 | M | bladder | urothelial | II | | 5.05E+10 | 115.93 |
| 010 | | | cancer | carcinoma | | | | |
| BLDR | 63 | M | bladder | urothelial | II | | 3.43E+10 | 136.43 |
| 011 | | | cancer | carcinoma | | | | |
| BLDR | 73 | M | bladder | urothelial | II | | 7.60E+10 | 101.90 |
| 012 | | | cancer | carcinoma | | | | |
| BLDR | 57 | M | bladder | urothelial | I | | 8.70E+10 | 122.40 |
| 013 | | | cancer | carcinoma | | | | |
| BLDR | 59 | M | bladder | urothelial | I | | 5.88E+10 | 113.65 |
| 014 | | | cancer | carcinoma | | | | |
| BLDR | 70 | M | bladder | urothelial | I | | 1.05E+11 | 132.67 |
| 015 | | | cancer | carcinoma | | | | |
| BLDR | 70 | F | bladder | urothelial | I | | 2.00E+10 | 119.10 |
| 016 | | | cancer | carcinoma | | | | |
| BLDR | 48 | M | bladder | urothelial | II | | 2.23E+10 | 135.93 |
| 017 | | | cancer | carcinoma | | | | |
| BLDR | 74 | M | bladder | urothelial | I | | 1.30E+10 | 126.50 |
| 018 | | | cancer | carcinoma | | | | |
| BLDR | 62 | M | bladder | urothelial | II | | 2.03E+10 | 123.97 |
| 019 | | | cancer | carcinoma | | | | |
| BLDR | 68 | M | bladder | urothelial | II | | 1.90E+10 | 131.20 |
| 020 | | | cancer | carcinoma | | | | |
| BLDR | 62 | M | bladder | urothelial | I | | 2.70E+11 | 129.33 |
| 021 | | | cancer | carcinoma | | | | |
| BLDR | 61 | M | bladder | urothelial | I | | 1.96E+10 | 119.85 |
| 022 | | | cancer | carcinoma | | | | |
| BLDR | 63 | M | bladder | urothelial | I | | 5.43E+09 | 134.17 |
| 023 | | | cancer | carcinoma | | | | |
| BLDR | 67 | M | bladder | urothelial | I | | 7.78E+09 | 135.78 |
| 024 | | | cancer | carcinoma | | | | |
| BLDR | 60 | F | bladder | urothelial | I | | 2.90E+10 | 117.03 |
| 025 | | | cancer | carcinoma | | | | |
| BLDR | 72 | M | bladder | urothelial | II | | 9.60E+09 | 133.97 |
| 026 | | | cancer | carcinoma | | | | |
| BLDR | 66 | M | bladder | urothelial | I | | 3.00E+07 | 109.90 |
| 027 | | | cancer | carcinoma | | | | |
| BLDR | 57 | M | bladder | urothelial | I | | 2.35E+09 | 135.55 |
| 028 | | | cancer | carcinoma | | | | |
| BLDR | 54 | M | bladder | urothelial | II | | 1.31E+10 | 130.50 |
| 029 | | | cancer | carcinoma | | | | |
| BLDR | 55 | M | bladder | urothelial | II | | 1.31E+10 | 134.52 |
| 030 | | | cancer | carcinoma | | | | |
| BLDR | 53 | M | bladder | urothelial | II | | 3.62E+10 | 113.85 |
| 031 | | | cancer | carcinoma | | | | |
| BLDR | 53 | M | bladder | urothelial | II | | 4.50E+10 | 99.10 |
| 032 | | | cancer | carcinoma | | | | |
| BLDR | 72 | M | bladder | urothelial | I | | 3.90E+10 | 122.55 |
| 033 | | | cancer | carcinoma | | | | |
| BLDR | 68 | M | bladder | urothelial | II | | 1.43E+10 | 128.37 |
| 034 | | | cancer | carcinoma | | | | |
| BLDR | 63 | M | bladder | urothelial | II | | 3.86E+10 | 115.72 |
| 035 | | | cancer | carcinoma | | | | |
| BLDR | 70 | M | bladder | urothelial | I | | 5.00E+10 | 103.80 |
| 036 | | | cancer | carcinoma | | | | |
| BLDR | 58 | M | bladder | urothelial | II | | 2.32E+10 | 118.30 |
| 037 | | | cancer | carcinoma | | | | |
| BLDR | 69 | M | bladder | urothelial | II | | 2.83E+10 | 120.53 |
| 038 | | | cancer | carcinoma | | | | |
| BLDR | 73 | M | bladder | urothelial | I | | 4.38E+10 | 102.60 |
| 039 | | | cancer | carcinoma | | | | |
| BLDR | 58 | M | bladder | urothelial | I | | 1.66E+10 | 119.18 |
| 040 | | | cancer | carcinoma | | | | |
| BLDR | 40 | F | bladder | urothelial | II | | 2.70E+10 | 118.70 |
| 041 | | | cancer | carcinoma | | | | |
| BLDR | 61 | M | bladder | urothelial | II | | 8.27E+09 | 131.43 |
| 042 | | | cancer | carcinoma | | | | |
| BLDR | 63 | M | bladder | urothelial | I | | 1.14E+10 | 124.03 |
| 043 | | | cancer | carcinoma | | | | |
| BLDR | 58 | M | bladder | urothelial | I | | 4.70E+10 | 101.47 |
| 044 | | | cancer | carcinoma | | | | |
| BLDR | 63 | M | bladder | urothelial | II | | 5.47E+09 | 156.10 |
| 045 | | | cancer | carcinoma | | | | |
| BLDR | 41 | F | bladder | urothelial | I | | 1.70E+10 | 154.80 |
| 046 | | | cancer | carcinoma | | | | |
| BLDR | 68 | M | bladder | urothelial | I | | 9.98E+09 | 129.48 |
| 047 | | | cancer | carcinoma | | | | |
| BLDR | 56 | M | bladder | urothelial | I | | 6.05E+10 | 139.90 |
| 048 | | | cancer | carcinoma | | | | |
| PDAC | 60 | F | pancreatic | adenocarcinoma | I | A | 6.50E+10 | 114.75 |
| 001 | | | cancer | | | | | |
| PDAC | 49 | F | pancreatic | adenocarcinoma | II | B | 1.25E+10 | 127.14 |
| 002 | | | cancer | | | | | |
| PDAC | 65 | F | pancreatic | adenocarcinoma | II | A | 1.97E+10 | 126.15 |
| 003 | | | cancer | | | | | |
| PDAC | 60 | F | pancreatic | adenocarcinoma | II | A | 3.40E+10 | 163.53 |
| 004 | | | cancer | | | | | |
| PDAC | 58 | F | pancreatic | adenocarcinoma | II | A | 5.46E+10 | 124.40 |
| 005 | | | cancer | | | | | |
| PDAC | 56 | F | pancreatic | adenocarcinoma | II | B | 9.03E+09 | 139.20 |
| 006 | | | cancer | | | | | |
| PDAC | 66 | F | pancreatic | adenocarcinoma | II | A | 7.76E+09 | 139.22 |
| 007 | | | cancer | | | | | |
| PDAC | 63 | F | pancreatic | adenocarcinoma | II | B | 1.00E+11 | 121.38 |
| 008 | | | cancer | | | | | |
| PDAC | 59 | F | pancreatic | adenocarcinoma | II | B | 1.38E+10 | 146.10 |
| 009 | | | cancer | | | | | |
| PDAC | 65 | F | pancreatic | adenocarcinoma | I | B | 9.73E+09 | 153.43 |
| 010 | | | cancer | | | | | |
| PDAC | 57 | M | pancreatic | adenocarcinoma | II | B | 3.23E+10 | 135.57 |
| 011 | | | cancer | | | | | |
| PDAC | 64 | F | pancreatic | adenocarcinoma | I | B | 2.30E+10 | 151.73 |
| 012 | | | cancer | | | | | |
| PDAC | 55 | F | pancreatic | adenocarcinoma | I | B | 2.00E+10 | 152.45 |
| 013 | | | cancer | | | | | |
| PDAC | 66 | F | pancreatic | adenocarcinoma | II | A | 7.40E+10 | 146.58 |
| 014 | | | cancer | | | | | |
| PDAC | 68 | F | pancreatic | adenocarcinoma | II | B | 5.40E+10 | 138.70 |
| 015 | | | cancer | | | | | |
| PDAC | 68 | M | pancreatic | adenocarcinoma | I | B | 2.21E+10 | 155.20 |
| 016 | | | cancer | | | | | |
| PDAC | 59 | F | pancreatic | adenocarcinoma | II | B | 6.58E+09 | 127.98 |
| 017 | | | cancer | | | | | |
| PDAC | 66 | M | pancreatic | adenocarcinoma | II | B | 2.63E+10 | 140.54 |
| 018 | | | cancer | | | | | |
| PDAC | 68 | F | pancreatic | adenocarcinoma | II | B | 2.03E+10 | 123.80 |
| 019 | | | cancer | | | | | |
| PDAC | 52 | M | pancreatic | adenocarcinoma | I | A | 5.41E+10 | 137.23 |
| 020 | | | cancer | | | | | |
| PDAC | 64 | F | pancreatic | adenocarcinoma | I | B | 7.13E+09 | 141.80 |
| 021 | | | cancer | | | | | |
| PDAC | 56 | F | pancreatic | adenocarcinoma | II | B | 1.81E+11 | 114.98 |
| 022 | | | cancer | | | | | |
| PDAC | 50 | F | pancreatic | adenocarcinoma | II | B | 3.70E+10 | 126.30 |
| 023 | | | cancer | | | | | |
| PDAC | 49 | M | pancreatic | adenocarcinoma | II | B | 1.60E+10 | 147.70 |
| 024 | | | cancer | | | | | |
| PDAC | 63 | F | pancreatic | adenocarcinoma | II | B | 1.00E+10 | 137.50 |
| 025 | | | cancer | | | | | |
| PDAC | 70 | F | pancreatic | adenocarcinoma | II | B | 7.75E+10 | 95.30 |
| 026 | | | cancer | | | | | |
| PDAC | 57 | F | pancreatic | adenocarcinoma | II | B | 1.01E+11 | 113.65 |
| 027 | | | cancer | | | | | |
| PDAC | 61 | F | pancreatic | adenocarcinoma | II | B | 7.53E+10 | 78.37 |
| 028 | | | cancer | | | | | |
| PDAC | 71 | F | pancreatic | adenocarcinoma | I | B | 2.24E+10 | 139.80 |
| 029 | | | cancer | | | | | |
| PDAC | 59 | M | pancreatic | adenocarcinoma | I | B | 2.86E+10 | 108.80 |
| 030 | | | cancer | | | | | |
| PDAC | 61 | F | pancreatic | adenocarcinoma | I | B | 3.48E+10 | 161.60 |
| 031 | | | cancer | | | | | |
| PDAC | 60 | F | pancreatic | adenocarcinoma | I | A | 1.95E+11 | 138.95 |
| 032 | | | cancer | | | | | |
| PDAC | 72 | M | pancreatic | adenocarcinoma | I | B | 2.28E+10 | 127.05 |
| 033 | | | cancer | | | | | |
| PDAC | 64 | M | pancreatic | adenocarcinoma | I | B | 2.57E+10 | 123.48 |
| 034 | | | cancer | | | | | |
| PDAC | 70 | F | pancreatic | adenocarcinoma | I | B | 7.37E+09 | 149.93 |
| 035 | | | cancer | | | | | |
| PDAC | 72 | F | pancreatic | adenocarcinoma | I | B | 3.53E+09 | 139.13 |
| 036 | | | cancer | | | | | |
| PDAC | 58 | M | pancreatic | adenocarcinoma | I | B | 3.70E+09 | 145.00 |
| 037 | | | cancer | | | | | |
| PDAC | 64 | F | pancreatic | adenocarcinoma | I | B | 1.03E+11 | 133.33 |
| 038 | | | cancer | | | | | |
| PDAC | 74 | M | pancreatic | adenocarcinoma | I | A | 2.63E+10 | 146.00 |
| 039 | | | cancer | | | | | |
| PDAC | 66 | F | pancreatic | adenocarcinoma | I | B | 2.33E+10 | 165.83 |
| 040 | | | cancer | | | | | |
| PDAC | 69 | M | pancreatic | adenocarcinoma | I | B | 5.13E+10 | 155.60 |
| 041 | | | cancer | | | | | |
| PDAC | 59 | F | pancreatic | adenocarcinoma | I | B | 3.17E+10 | 140.20 |
| 042 | | | cancer | | | | | |
| PDAC | 74 | F | pancreatic | adenocarcinoma | II | A | 5.36E+10 | 120.38 |
| 043 | | | cancer | | | | | |
| PDAC | 59 | M | pancreatic | adenocarcinoma | II | A | 4.45E+10 | 116.85 |
| 044 | | | cancer | | | | | |
| HC | 61 | F | healthy | | | | 3.45E+10 | 102.10 |
| 001 | | | control | | | | | |
| HC | 64 | F | healthy | | | | 7.50E+09 | 147.55 |
| 002 | | | control | | | | | |
| HC | 71 | F | healthy | | | | 6.72E+11 | 129.75 |
| 003 | | | control | | | | | |
| HC | 69 | F | healthy | | | | 4.85E+10 | 116.45 |
| 004 | | | control | | | | | |
| HC | 70 | F | healthy | | | | 5.25E+10 | 125.55 |
| 005 | | | control | | | | | |
| HC | 54 | F | healthy | | | | 2.90E+10 | 115.10 |
| 006 | | | control | | | | | |
| HC | 50 | F | healthy | | | | 1.30E+10 | 141.05 |
| 007 | | | control | | | | | |
| HC | 53 | F | healthy | | | | 5.30E+10 | 120.75 |
| 008 | | | control | | | | | |
| HC | 62 | M | healthy | | | | 6.90E+09 | 136.75 |
| 009 | | | control | | | | | |
| HC | 63 | F | healthy | | | | 4.40E+10 | 122.75 |
| 010 | | | control | | | | | |
| HC | 60 | M | healthy | | | | 1.80E+10 | 123.10 |
| 011 | | | control | | | | | |
| HC | 55 | F | healthy | | | | #N/A | #N/A |
| 012 | | | control | | | | | |
| HC | 65 | F | healthy | | | | 3.40E+10 | 110.00 |
| 013 | | | control | | | | | |
| HC | 67 | F | healthy | | | | #N/A | #N/A |
| 014 | | | control | | | | | |
| HC | 63 | F | healthy | | | | 1.90E+10 | 115.20 |
| 015 | | | control | | | | | |
| HC | 60 | F | healthy | | | | 1.26E+10 | 141.50 |
| 016 | | | control | | | | | |
| HC | 65 | M | healthy | | | | 2.25E+10 | 130.55 |
| 017 | | | control | | | | | |
| HC | 57 | F | healthy | | | | 1.95E+10 | 135.10 |
| 018 | | | control | | | | | |
| HC | 55 | F | healthy | | | | 1.70E+10 | 152.90 |
| 019 | | | control | | | | | |
| HC | 68 | F | healthy | | | | 5.40E+10 | 119.70 |
| 020 | | | control | | | | | |
| HC | 65 | F | healthy | | | | 1.51E+11 | 116.33 |
| 021 | | | control | | | | | |
| HC | 67 | F | healthy | | | | 1.13E+11 | 109.00 |
| 022 | | | control | | | | | |
| HC | 70 | F | healthy | | | | 6.62E+11 | 105.55 |
| 023 | | | control | | | | | |
| HC | 71 | F | healthy | | | | 1.74E+11 | 106.73 |
| 024 | | | control | | | | | |
| HC | 60 | M | healthy | | | | 6.30E+10 | 113.05 |
| 025 | | | control | | | | | |
| HC | 63 | F | healthy | | | | 9.63E+09 | 133.60 |
| 026 | | | control | | | | | |
| HC | 65 | M | healthy | | | | 1.00E+11 | 114.20 |
| 028 | | | control | | | | | |
| HC | 65 | M | healthy | | | | 7.60E+10 | 125.38 |
| 029 | | | control | | | | | |
| HC | 61 | F | healthy | | | | 7.10E+10 | 90.10 |
| 030 | | | control | | | | | |
| HC | 70 | M | healthy | | | | 8.07E+10 | 86.67 |
| 031 | | | control | | | | | |
| HC | 60 | M | healthy | | | | 1.46E+10 | 121.23 |
| 032 | | | control | | | | | |
| HC | 62 | F | healthy | | | | 7.30E+10 | 102.07 |
| 033 | | | control | | | | | |
| HC | 65 | F | healthy | | | | 3.37E+10 | 105.73 |
| 034 | | | control | | | | | |
| HC | 61 | M | healthy | | | | 3.70E+10 | 114.57 |
| 035 | | | control | | | | | |
| HC | 67 | F | healthy | | | | 8.15E+10 | 100.28 |
| 036 | | | control | | | | | |
| HC | 69 | F | healthy | | | | 5.33E+10 | 101.97 |
| 037 | | | control | | | | | |
| HC | 70 | M | healthy | | | | 5.90E+10 | 111.33 |
| 038 | | | control | | | | | |
| HC | 65 | F | healthy | | | | 9.10E+10 | 94.47 |
| 039 | | | control | | | | | |
| HC | 71 | F | healthy | | | | 1.77E+14 | 116.43 |
| 040 | | | control | | | | | |
| HC | 60 | M | healthy | | | | 5.27E+10 | 102.90 |
| 041 | | | control | | | | | |
| HC | 63 | M | healthy | | | | 4.40E+14 | 102.90 |
| 042 | | | control | | | | | |
| HC | 55 | F | healthy | | | | 1.14E+10 | 132.65 |
| 043 | | | control | | | | | |
| HC | 60 | M | healthy | | | | 1.80E+10 | 132.30 |
| 044 | | | control | | | | | |
| HC | 60 | M | healthy | | | | 2.15E+10 | 116.80 |
| 045 | | | control | | | | | |
| HC | 54 | F | healthy | | | | 2.15E+10 | 125.20 |
| 046 | | | control | | | | | |
| HC | 60 | M | healthy | | | | 2.20E+10 | 131.75 |
| 047 | | | control | | | | | |
| HC | 59 | F | healthy | | | | 1.90E+10 | 114.25 |
| 048 | | | control | | | | | |
| HC | 50 | F | healthy | | | | 2.90E+10 | 125.65 |
| 049 | | | control | | | | | |
| HC | 50 | M | healthy | | | | #N/A | #N/A |
| 050 | | | control | | | | | |
| HC | 54 | M | healthy | | | | 3.52E+10 | 112.75 |
| 051 | | | control | | | | | |
| HC | 54 | F | healthy | | | | #N/A | #N/A |
| 052 | | | control | | | | | |
| HC | 48 | M | healthy | | | | 1.30E+10 | 126.80 |
| 053 | | | control | | | | | |
| HC | 44 | M | healthy | | | | 1.11E+10 | 132.58 |
| 054 | | | control | | | | | |
| HC | 42 | M | healthy | | | | 3.78E+10 | 105.40 |
| 055 | | | control | | | | | |
| HC | 46 | M | healthy | | | | 2.03E+14 | 103.63 |
| 056 | | | control | | | | | |
| HC | 49 | F | healthy | | | | 3.32E+10 | 118.60 |
| 057 | | | control | | | | | |
| HC | 42 | F | healthy | | | | 4.68E+10 | 118.43 |
| 058 | | | control | | | | | |
| HC | 50 | F | healthy | | | | 2.45E+10 | 122.05 |
| 059 | | | control | | | | | |
| HC | 49 | F | healthy | | | | 2.85E+11 | 113.80 |
| 060 | | | control | | | | | |
| HC | 48 | F | healthy | | | | 3.25E+09 | 145.55 |
| 061 | | | control | | | | | |
| HC | 48 | M | healthy | | | | 5.30E+09 | 142.70 |
| 062 | | | control | | | | | |
| HC | 43 | M | healthy | | | | 4.70E+09 | 149.50 |
| 063 | | | control | | | | | |
| HC | 48 | M | healthy | | | | 4.80E+09 | 140.00 |
| 064 | | | control | | | | | |
| HC | 45 | M | healthy | | | | 4.30E+09 | 144.80 |
| 065 | | | control | | | | | |
| HC | 44 | F | healthy | | | | 3.80E+09 | 158.90 |
| 066 | | | control | | | | | |
| HC | 46 | M | healthy | | | | 3.60E+10 | 109.80 |
| 067 | | | control | | | | | |
| HC | 42 | M | healthy | | | | 3.05E+10 | 132.80 |
| 068 | | | control | | | | | |
| HC | 46 | M | healthy | | | | 6.68E+09 | 137.45 |
| 069 | | | control | | | | | |
| HC | 47 | M | healthy | | | | 2.80E+10 | 132.23 |
| 070 | | | control | | | | | |
| HC | 41 | F | healthy | | | | 9.50E+09 | 121.00 |
| 071 | | | control | | | | | |
| HC | 49 | M | healthy | | | | 8.90E+10 | 119.90 |
| 072 | | | control | | | | | |
| HC | 45 | F | healthy | | | | 8.50E+09 | 128.70 |
| 073 | | | control | | | | | |
| HC | 46 | F | healthy | | | | 1.20E+10 | 119.90 |
| 074 | | | control | | | | | |
| HC | 47 | F | healthy | | | | 4.12E+10 | 123.30 |
| 075 | | | control | | | | | |
| HC | 50 | M | healthy | | | | 7.67E+09 | 149.73 |
| 076 | | | control | | | | | |
| HC | 48 | F | healthy | | | | 4.60E+10 | 116.65 |
| 077 | | | control | | | | | |
| HC | 48 | F | healthy | | | | 4.70E+10 | 113.80 |
| 078 | | | control | | | | | |
| HC | 49 | M | healthy | | | | 3.52E+10 | 125.95 |
| 079 | | | control | | | | | |
| HC | 47 | F | healthy | | | | 3.65E+10 | 124.30 |
| 080 | | | control | | | | | |
| HC | 48 | F | healthy | | | | 3.50E+10 | 104.10 |
| 082 | | | control | | | | | |
| HC | 43 | M | healthy | | | | 1.59E+10 | 139.38 |
| 084 | | | control | | | | | |
| HC | 47 | F | healthy | | | | 8.40E+09 | 135.60 |
| 085 | | | control | | | | | |
| HC | 49 | M | healthy | | | | 5.05E+10 | 122.10 |
| 086 | | | control | | | | | |
| HC | 45 | F | healthy | | | | 3.40E+10 | 111.50 |
| 087 | | | control | | | | | |
| HC | 45 | F | healthy | | | | 5.30E+09 | 147.85 |
| 088 | | | control | | | | | |
| HC | 45 | M | healthy | | | | 1.58E+10 | 143.98 |
| 089 | | | control | | | | | |
| HC | 43 | M | healthy | | | | 2.67E+10 | 134.60 |
| 090 | | | control | | | | | |
| HC | 44 | F | healthy | | | | 4.00E+10 | 108.90 |
| 091 | | | control | | | | | |
| HC | 40 | M | healthy | | | | 3.70E+10 | 135.30 |
| 092 | | | control | | | | | |
| HC | 47 | F | healthy | | | | 9.13E+09 | 130.30 |
| 093 | | | control | | | | | |
| HC | 48 | M | healthy | | | | 5.48E+10 | 119.35 |
| 094 | | | control | | | | | |
| HC | 41 | M | healthy | | | | 3.67E+14 | 107.87 |
| 095 | | | control | | | | | |
| HC | 48 | M | healthy | | | | 3.41E+11 | 107.90 |
| 096 | | | control | | | | | |
| HC | 45 | M | healthy | | | | 3.90E+11 | 107.80 |
| 097 | | | control | | | | | |
| HC | 45 | F | healthy | | | | 5.30E+09 | 136.90 |
| 098 | | | control | | | | | |
| HC | 67 | M | healthy | | | | 2.98E+10 | 126.70 |
| 099 | | | control | | | | | |
| HC | 60 | F | healthy | | | | 1.41E+10 | 141.43 |
| 100 | | | control | | | | | |
| HC | 41 | F | healthy | | | | #N/A | #N/A |
| 101 | | | control | | | | | |
| HC | 45 | F | healthy | | | | 3.40E+10 | 98.30 |
| 102 | | | control | | | | | |
| HC | 40 | F | healthy | | | | 1.72E+10 | 137.33 |
| 103 | | | control | | | | | |
| HC | 45 | F | healthy | | | | 3.70E+09 | 146.70 |
| 104 | | | control | | | | | |
| HC | 41 | M | healthy | | | | 1.15E+10 | 120.35 |
| 105 | | | control | | | | | |
| HC | 44 | M | healthy | | | | 7.88E+10 | 112.58 |
| 106 | | | control | | | | | |
| HC | 44 | M | healthy | | | | 3.77E+09 | 128.93 |
| 107 | | | control | | | | | |
| HC | 41 | F | healthy | | | | 5.67E+09 | 133.00 |
| 108 | | | control | | | | | |
| HC | 43 | F | healthy | | | | 5.03E+09 | 138.23 |
| 109 | | | control | | | | | |
| HC | 42 | F | healthy | | | | 2.25E+10 | 125.25 |
| 110 | | | control | | | | | |
| HC | 49 | F | healthy | | | | 7.30E+09 | 128.90 |
| 111 | | | control | | | | | |
| HC | 40 | F | healthy | | | | 8.70E+09 | 139.30 |
| 112 | | | control | | | | | |
| HC | 60 | M | healthy | | | | 6.67E+13 | 134.93 |
| 113 | | | control | | | | | |

TABLE 4
Proteins Measured in Immunoassay

| | Evaluated in | | | Evaluated in | |
|---|---|---|---|---|---|
| | cancer and | | | cancer and | |
| Protein | healthy | Included in | Patient-related | healthy | Included in |
| biomarker | individuals | EXPLORE test | biomarker | individuals | EXPLORE test |
| Tenascin C | Yes | No | Age at time of | Yes | Yes |
| | | | collection | | |
| sAXL | Yes | No | Sex of | Yes | No |
| | | | individual | | |
| sE-selectin | Yes | Yes | | | |
| sHGFR/c-Met | Yes | No | | | |
| sHer2 | Yes | Yes | | | |
| sHer3 | Yes | No | | | |
| sIL-6Ra | Yes | No | | | |
| sNeuropilin-1 | Yes | Yes | | | |
| sPECAM-1 | Yes | No | | | |
| sVEGFR1 | Yes | Yes | | | |
| sVEGFR3 | Yes | No | | | |
| sc-kit/SCFR | Yes | Yes | | | |
| CA 125 | Yes | No | | | |
| CA 15-3 | Yes | No | | | |
| CA 19-9 | Yes | Yes | | | |
| CEA | Yes | No | | | |
| FGF2 | Yes | No | | | |
| HE4 | Yes | No | | | |
| HGF | Yes | No | | | |
| IL-6 | Yes | No | | | |
| IL-8 | Yes | No | | | |
| Leptin | Yes | No | | | |
| MIF | Yes | No | | | |
| OPN | Yes | No | | | |
| Prolactin | Yes | No | | | |
| SCF | Yes | No | | | |
| TNFa | Yes | No | | | |
| TRAIL | Yes | No | | | |
| Total PSA | Yes | No | | | |
| VEGF | Yes | No | | | |
| b-HCG | Yes | No | | | |
| sFAS | Yes | Yes | | | |
| Cathepsin D | Yes | Yes | | | |
| FAP alpha | Yes | No | | | |
| Ferritin | Yes | Yes | | | |
| Galectin-3 | Yes | No | | | |
| IGFBP3 | Yes | Yes | | | |
| MIA | Yes | Yes | | | |
| MPO | Yes | Yes | | | |
| SHBG | Yes | No | | | |
| TIMP1 | Yes | Yes | | | |
| TIMP2 | Yes | No | | | |

TABLE 5
Protein concentrations in plasma samples for cancer donors and healthy controls

| Subject | | | | | | | |
|---|---|---|---|---|---|---|---|
| Cohort | Sample | Tumor | AJCC | | | | |
| ID | Type | type | Stage | A/B/C | Tenascin C | sAXL | sE-selectin |
| OVAR | Exosomal | Ovarian | I | A | 570.84 | 5.60 | 529.35 |
| 001 | Protein | | | | | | |
| OVAR | Exosomal | Ovarian | I | A | 909.63 | 5.60 | 277.22 |
| 002 | Protein | | | | | | |
| OVAR | Exosomal | Ovarian | I | B | 871.12 | 5.60 | 366.33 |
| 003 | Protein | | | | | | |
| OVAR | Exosomal | Ovarian | I | A | 20.20 | 5.60 | 247.80 |
| 004 | Protein | | | | | | |
| OVAR | Exosomal | Ovarian | I | C | 755.39 | 5.60 | 751.42 |
| 005 | Protein | | | | | | |
| OVAR | Exosomal | Ovarian | I | A | 1493.60 | 5.60 | 291.26 |
| 006 | Protein | | | | | | |
| OVAR | Exosomal | Ovarian | I | A | 762.23 | 5.60 | 291.26 |
| 007 | Protein | | | | | | |
| OVAR | Exosomal | Ovarian | II | B | 20.20 | 5.60 | 247.80 |
| 008 | Protein | | | | | | |
| OVAR | Exosomal | Ovarian | II | B | 1200.95 | 5.60 | 247.80 |
| 009 | Protein | | | | | | |
| OVAR | Exosomal | Ovarian | I | A | 20.20 | 5.60 | 247.80 |
| 010 | Protein | | | | | | |
| OVAR | Exosomal | Ovarian | I | A | 91.82 | 5.60 | 247.80 |
| 011 | Protein | | | | | | |
| OVAR | Exosomal | Ovarian | I | B | 803.31 | 5.60 | 247.80 |
| 012 | Protein | | | | | | |
| OVAR | Exosomal | Ovarian | I | A | 20.20 | 8.72 | 1537.83 |
| 013 | Protein | | | | | | |
| OVAR | Exosomal | Ovarian | I | B | 1235.54 | 5.60 | 1074.19 |
| 014 | Protein | | | | | | |
| OVAR | Exosomal | Ovarian | I | B | 20.20 | 5.60 | 410.09 |
| 015 | Protein | | | | | | |
| OVAR | Exosomal | Ovarian | I | A | 20.20 | 5.60 | 247.80 |
| 016 | Protein | | | | | | |
| OVAR | Exosomal | Ovarian | I | A | 20.20 | 5.60 | 615.92 |
| 017 | Protein | | | | | | |
| OVAR | Exosomal | Ovarian | I | A | 515.16 | 5.60 | 256.02 |
| 018 | Protein | | | | | | |
| OVAR | Exosomal | Ovarian | I | C | 685.70 | 5.60 | 247.80 |
| 019 | Protein | | | | | | |
| OVAR | Exosomal | Ovarian | I | B | 404.70 | 5.60 | 247.80 |
| 020 | Protein | | | | | | |
| OVAR | Exosomal | Ovarian | I | A | 253.39 | 8.62 | 496.83 |
| 021 | Protein | | | | | | |
| OVAR | Exosomal | Ovarian | I | A | 213.09 | 5.60 | 496.83 |
| 022 | Protein | | | | | | |
| OVAR | Exosomal | Ovarian | II | A | 388.52 | 15.83 | 12692.00 |
| 023 | Protein | | | | | | |
| OVAR | Exosomal | Ovarian | I | A | 631.36 | 9.93 | 586.58 |
| 024 | Protein | | | | | | |
| OVAR | Exosomal | Ovarian | I | C | 731.02 | 5.60 | 435.29 |
| 025 | Protein | | | | | | |
| OVAR | Exosomal | Ovarian | I | C | 301.26 | 14.51 | 273.13 |
| 026 | Protein | | | | | | |
| OVAR | Exosomal | Ovarian | I | A | 20.20 | 6.97 | 2333.89 |
| 027 | Protein | | | | | | |
| OVAR | Exosomal | Ovarian | I | B | 484.35 | 5.60 | 391.24 |
| 028 | Protein | | | | | | |
| OVAR | Exosomal | Ovarian | I | A | 531.91 | 5.60 | 247.80 |
| 029 | Protein | | | | | | |
| OVAR | Exosomal | Ovarian | I | B | 754.37 | 5.60 | 977.96 |
| 030 | Protein | | | | | | |
| OVAR | Exosomal | Ovarian | I | A | 2513.03 | 5.60 | 1220.41 |
| 031 | Protein | | | | | | |
| OVAR | Exosomal | Ovarian | I | A | 692.66 | 5.60 | 247.80 |
| 032 | Protein | | | | | | |
| OVAR | Exosomal | Ovarian | I | A | 252.77 | 5.60 | 247.80 |
| 033 | Protein | | | | | | |
| OVAR | Exosomal | Ovarian | I | A | 914.14 | 5.60 | 559.24 |
| 034 | Protein | | | | | | |
| OVAR | Exosomal | Ovarian | I | A | 20.20 | 12.35 | 1220.41 |
| 035 | Protein | | | | | | |
| OVAR | Exosomal | Ovarian | I | A | 2797.86 | 6.10 | 1220.41 |
| 036 | Protein | | | | | | |
| OVAR | Exosomal | Ovarian | II | A | 20.20 | 5.60 | 247.80 |
| 037 | Protein | | | | | | |
| OVAR | Exosomal | Ovarian | I | A | 336.88 | 5.60 | 247.80 |
| 038 | Protein | | | | | | |
| OVAR | Exosomal | Ovarian | I | A | 20.20 | 5.60 | 247.80 |
| 039 | Protein | | | | | | |
| OVAR | Exosomal | Ovarian | I | A | 20.20 | 8.11 | 483.75 |
| 040 | Protein | | | | | | |
| OVAR | Exosomal | Ovarian | I | B | 258.96 | 5.60 | 259.94 |
| 041 | Protein | | | | | | |
| OVAR | Exosomal | Ovarian | I | A | 953.99 | 6.24 | 410.09 |
| 042 | Protein | | | | | | |
| OVAR | Exosomal | Ovarian | II | A | 20.20 | 5.60 | 410.09 |
| 043 | Protein | | | | | | |
| OVAR | Exosomal | Ovarian | I | C2 | 376.44 | 5.60 | 638.62 |
| 044 | Protein | | | | | | |
| BLDR | Exosomal | Bladder | II | | 20.20 | 7.60 | 371.26 |
| 001 | Protein | | | | | | |
| BLDR | Exosomal | Bladder | I | | 760.85 | 5.60 | 277.22 |
| 002 | Protein | | | | | | |
| BLDR | Exosomal | Bladder | I | | 623.45 | 5.60 | 247.80 |
| 003 | Protein | | | | | | |
| BLDR | Exosomal | Bladder | I | | 20.20 | 5.60 | 559.24 |
| 004 | Protein | | | | | | |
| BLDR | Exosomal | Bladder | II | | 20.20 | 7.85 | 1445.50 |
| 005 | Protein | | | | | | |
| BLDR | Exosomal | Bladder | I | | 20.20 | 5.60 | 1553.06 |
| 006 | Protein | | | | | | |
| BLDR | Exosomal | Bladder | I | | 819.94 | 5.60 | 421.41 |
| 007 | Protein | | | | | | |
| BLDR | Exosomal | Bladder | II | | 20.20 | 5.60 | 398.87 |
| 008 | Protein | | | | | | |
| BLDR | Exosomal | Bladder | I | | 760.85 | 5.60 | 247.80 |
| 009 | Protein | | | | | | |
| BLDR | Exosomal | Bladder | II | | 20.20 | 5.60 | 247.80 |
| 010 | Protein | | | | | | |
| BLDR | Exosomal | Bladder | II | | 663.94 | 5.60 | 528.29 |
| 011 | Protein | | | | | | |
| BLDR | Exosomal | Bladder | II | | 20.20 | 57.54 | 3091.22 |
| 012 | Protein | | | | | | |
| BLDR | Exosomal | Bladder | I | | 547.88 | 14.30 | 1012.37 |
| 013 | Protein | | | | | | |
| BLDR | Exosomal | Bladder | I | | 20.20 | 5.60 | 435.29 |
| 014 | Protein | | | | | | |
| BLDR | Exosomal | Bladder | I | | 1034.60 | 5.60 | 709.25 |
| 015 | Protein | | | | | | |
| BLDR | Exosomal | Bladder | I | | 585.84 | 5.60 | 270.54 |
| 016 | Protein | | | | | | |
| BLDR | Exosomal | Bladder | II | | 1029.49 | 5.60 | 788.30 |
| 017 | Protein | | | | | | |
| BLDR | Exosomal | Bladder | I | | 1958.90 | 5.60 | 1334.78 |
| 018 | Protein | | | | | | |
| BLDR | Exosomal | Bladder | II | | 20.20 | 5.60 | 371.26 |
| 019 | Protein | | | | | | |
| BLDR | Exosomal | Bladder | II | | 20.20 | 5.60 | 247.80 |
| 020 | Protein | | | | | | |
| BLDR | Exosomal | Bladder | I | | 412.15 | 5.60 | 391.24 |
| 021 | Protein | | | | | | |
| BLDR | Exosomal | Bladder | I | | 20.20 | 5.60 | 450.82 |
| 022 | Protein | | | | | | |
| BLDR | Exosomal | Bladder | I | | 698.46 | 5.60 | 615.92 |
| 023 | Protein | | | | | | |
| BLDR | Exosomal | Bladder | I | | 1016.96 | 5.60 | 247.80 |
| 024 | Protein | | | | | | |
| BLDR | Exosomal | Bladder | I | | 20.20 | 5.60 | 247.80 |
| 025 | Protein | | | | | | |
| BLDR | Exosomal | Bladder | II | | 20.20 | 6.24 | 311.32 |
| 026 | Protein | | | | | | |
| BLDR | Exosomal | Bladder | I | | 20.20 | 5.60 | 455.35 |
| 027 | Protein | | | | | | |
| BLDR | Exosomal | Bladder | I | | 20.20 | 7.02 | 754.26 |
| 028 | Protein | | | | | | |
| BLDR | Exosomal | Bladder | II | | 305.85 | 5.60 | 247.80 |
| 029 | Protein | | | | | | |
| BLDR | Exosomal | Bladder | II | | 20.20 | 5.60 | 366.33 |
| 030 | Protein | | | | | | |
| BLDR | Exosomal | Bladder | II | | 20.20 | 5.60 | 490.03 |
| 031 | Protein | | | | | | |
| BLDR | Exosomal | Bladder | II | | 216.25 | 14.14 | 642.34 |
| 032 | Protein | | | | | | |
| BLDR | Exosomal | Bladder | I | | 20.20 | 5.60 | 247.80 |
| 033 | Protein | | | | | | |
| BLDR | Exosomal | Bladder | II | | 20.20 | 5.60 | 247.80 |
| 034 | Protein | | | | | | |
| BLDR | Exosomal | Bladder | II | | 20.20 | 5.63 | 1412.22 |
| 035 | Protein | | | | | | |
| BLDR | Exosomal | Bladder | I | | 835.18 | 5.99 | 273.13 |
| 036 | Protein | | | | | | |
| BLDR | Exosomal | Bladder | II | | 20.20 | 5.60 | 564.22 |
| 037 | Protein | | | | | | |
| BLDR | Exosomal | Bladder | II | | 385.67 | 5.60 | 289.62 |
| 038 | Protein | | | | | | |
| BLDR | Exosomal | Bladder | I | | 20.20 | 5.60 | 371.26 |
| 039 | Protein | | | | | | |
| BLDR | Exosomal | Bladder | I | | 20.20 | 5.60 | 247.80 |
| 040 | Protein | | | | | | |
| BLDR | Exosomal | Bladder | II | | 20.20 | 6.24 | 1140.86 |
| 041 | Protein | | | | | | |
| BLDR | Exosomal | Bladder | II | | 497.48 | 5.60 | 531.84 |
| 042 | Protein | | | | | | |
| BLDR | Exosomal | Bladder | I | | 20.20 | 9.95 | 1140.86 |
| 043 | Protein | | | | | | |
| BLDR | Exosomal | Bladder | I | | 20.20 | 11.18 | 638.62 |
| 044 | Protein | | | | | | |
| BLDR | Exosomal | Bladder | II | | 1359.62 | 5.62 | 638.62 |
| 045 | Protein | | | | | | |
| BLDR | Exosomal | Bladder | I | | 20.20 | 5.60 | 1101.76 |
| 046 | Protein | | | | | | |
| BLDR | Exosomal | Bladder | I | | 20.20 | 5.60 | 247.80 |
| 047 | Protein | | | | | | |
| BLDR | Exosomal | Bladder | I | | 1015.29 | 5.60 | 247.80 |
| 048 | Protein | | | | | | |
| PDAC | Exosomal | Pancreatic | I | A | 903.37 | 5.60 | 559.24 |
| 001 | Protein | | | | | | |
| PDAC | Exosomal | Pancreatic | II | B | 20.20 | 7.85 | 1958.37 |
| 002 | Protein | | | | | | |
| PDAC | Exosomal | Pancreatic | II | A | 20.20 | 5.60 | 391.24 |
| 003 | Protein | | | | | | |
| PDAC | Exosomal | pancreatic | II | A | 20.20 | 5.60 | 247.80 |
| 004 | Protein | | | | | | |
| PDAC | Exosomal | Pancreatic | II | A | 20.20 | 5.60 | 977.96 |
| 005 | Protein | | | | | | |
| PDAC | Exosomal | Pancreatic | II | B | 748.51 | 5.60 | 339.72 |
| 006 | Protein | | | | | | |
| PDAC | Exosomal | Pancreatic | II | A | 1289.36 | 5.60 | 339.72 |
| 007 | Protein | | | | | | |
| PDAC | Exosomal | Pancreatic | II | B | 684.32 | 5.60 | 483.75 |
| 008 | Protein | | | | | | |
| PDAC | Exosomal | Pancreatic | II | B | 20.20 | 5.60 | 247.80 |
| 009 | Protein | | | | | | |
| PDAC | Exosomal | pancreatic | I | B | 826.24 | 5.60 | 365.27 |
| 010 | Protein | | | | | | |
| PDAC | Exosomal | Pancreatic | II | B | 1254.43 | 5.60 | 692.21 |
| 011 | Protein | | | | | | |
| PDAC | Exosomal | pancreatic | I | B | 20.20 | 5.60 | 247.80 |
| 012 | Protein | | | | | | |
| PDAC | Exosomal | pancreatic | I | B | 20.20 | 5.60 | 1101.76 |
| 013 | Protein | | | | | | |
| PDAC | Exosomal | pancreatic | II | A | 20.20 | 5.60 | 247.80 |
| 014 | Protein | | | | | | |
| PDAC | Exosomal | Pancreatic | II | B | 944.44 | 9.01 | 1386.39 |
| 015 | Protein | | | | | | |
| PDAC | Exosomal | pancreatic | I | B | 424.84 | 5.60 | 366.33 |
| 016 | Protein | | | | | | |
| PDAC | Exosomal | Pancreatic | II | B | 297.57 | 5.60 | 247.80 |
| 017 | Protein | | | | | | |
| PDAC | Exosomal | Pancreatic | II | B | 20.20 | 5.60 | 247.80 |
| 018 | Protein | | | | | | |
| PDAC | Exosomal | Pancreatic | II | B | 20.20 | 5.60 | 339.72 |
| 019 | Protein | | | | | | |
| PDAC | Exosomal | pancreatic | I | A | 1122.65 | 5.60 | 977.96 |
| 020 | Protein | | | | | | |
| PDAC | Exosomal | pancreatic | I | B | 887.02 | 5.60 | 247.80 |
| 021 | Protein | | | | | | |
| PDAC | Exosomal | Pancreatic | II | B | 20.20 | 21.08 | 964.96 |
| 022 | Protein | | | | | | |
| PDAC | Exosomal | Pancreatic | II | B | 20.20 | 5.60 | 306.76 |
| 023 | Protein | | | | | | |
| PDAC | Exosomal | Pancreatic | II | B | 808.67 | 5.60 | 641.67 |
| 024 | Protein | | | | | | |
| PDAC | Exosomal | Pancreatic | II | B | 20.20 | 5.60 | 559.24 |
| 025 | Protein | | | | | | |
| PDAC | Exosomal | Pancreatic | II | B | 20.20 | 5.60 | 740.89 |
| 026 | Protein | | | | | | |
| PDAC | Exosomal | Pancreatic | II | B | 20.20 | 6.97 | 709.25 |
| 027 | Protein | | | | | | |
| PDAC | Exosomal | Pancreatic | II | B | 854.89 | 5.60 | 895.77 |
| 028 | Protein | | | | | | |
| PDAC | Exosomal | pancreatic | I | B | 779.49 | 5.60 | 331.51 |
| 029 | Protein | | | | | | |
| PDAC | Exosomal | pancreatic | I | B | 20.20 | 5.60 | 250.40 |
| 030 | Protein | | | | | | |
| PDAC | Exosomal | pancreatic | I | B | 214.01 | 5.60 | 247.80 |
| 031 | Protein | | | | | | |
| PDAC | Exosomal | pancreatic | I | A | 428.69 | 5.60 | 601.91 |
| 032 | Protein | | | | | | |
| PDAC | Exosomal | pancreatic | I | B | 339.94 | 5.60 | 247.80 |
| 033 | Protein | | | | | | |
| PDAC | Exosomal | pancreatic | I | B | 1051.29 | 8.74 | 1038.77 |
| 034 | Protein | | | | | | |
| PDAC | Exosomal | pancreatic | I | B | 185.93 | 5.60 | 371.26 |
| 035 | Protein | | | | | | |
| PDAC | Exosomal | pancreatic | I | B | 286.63 | 5.60 | 247.80 |
| 036 | Protein | | | | | | |
| PDAC | Exosomal | pancreatic | I | B | 592.37 | 5.60 | 247.80 |
| 037 | Protein | | | | | | |
| PDAC | Exosomal | pancreatic | I | B | 1298.18 | 5.94 | 1388.07 |
| 038 | Protein | | | | | | |
| PDAC | Exosomal | pancreatic | I | A | 1292.75 | 5.60 | 247.80 |
| 039 | Protein | | | | | | |
| PDAC | Exosomal | pancreatic | I | B | 673.97 | 5.60 | 247.80 |
| 040 | Protein | | | | | | |
| PDAC | Exosomal | Pancreatic | I | B | 20.20 | 5.60 | 825.64 |
| 041 | Protein | | | | | | |
| PDAC | Exosomal | pancreatic | I | B | 1185.51 | 5.60 | 319.28 |
| 042 | Protein | | | | | | |
| PDAC | Exosomal | pancreatic | II | A | 1279.57 | 5.60 | 247.80 |
| 043 | Protein | | | | | | |
| PDAC | Exosomal | Pancreatic | II | A | 20.20 | 5.60 | 247.80 |
| 044 | Protein | | | | | | |
| HC 001 | Exosomal | | | | 20.20 | 5.60 | 366.33 |
| | Protein | | | | | | |
| HC 002 | Exosomal | | | | 412.88 | 5.60 | 247.80 |
| | Protein | | | | | | |
| HC 003 | Exosomal | | | | 757.43 | 12.71 | 1777.33 |
| | Protein | | | | | | |
| HC 004 | Exosomal | | | | 552.75 | 6.65 | 814.86 |
| | Protein | | | | | | |
| HC 005 | Exosomal | | | | 743.10 | 5.60 | 366.33 |
| | Protein | | | | | | |
| HC 006 | Exosomal | | | | 20.20 | 5.60 | 247.80 |
| | Protein | | | | | | |
| HC 007 | Exosomal | | | | 20.20 | 5.60 | 322.61 |
| | Protein | | | | | | |
| HC 008 | Exosomal | | | | 20.20 | 5.60 | 605.74 |
| | Protein | | | | | | |
| HC 009 | Exosomal | | | | 1833.91 | 5.60 | 709.25 |
| | Protein | | | | | | |
| HC 010 | Exosomal | | | | 1475.91 | 5.60 | 567.89 |
| | Protein | | | | | | |
| HC 011 | Exosomal | | | | 20.20 | 6.54 | 1101.76 |
| | Protein | | | | | | |
| HC 012 | Exosomal | | | | 1216.68 | 10.93 | 715.77 |
| | Protein | | | | | | |
| HC 013 | Exosomal | | | | 1040.83 | 12.00 | 889.90 |
| | Protein | | | | | | |
| HC 014 | Exosomal | | | | 1027.67 | 8.44 | 661.37 |
| | Protein | | | | | | |
| HC 015 | Exosomal | | | | 711.08 | 5.60 | 247.80 |
| | Protein | | | | | | |
| HC 016 | Exosomal | | | | 1626.82 | 5.60 | 529.35 |
| | Protein | | | | | | |
| HC 017 | Exosomal | | | | 20.20 | 8.74 | 2513.21 |
| | Protein | | | | | | |
| HC 018 | Exosomal | | | | 1101.16 | 5.60 | 605.74 |
| | Protein | | | | | | |
| HC 019 | Exosomal | | | | 671.65 | 5.60 | 247.80 |
| | Protein | | | | | | |
| HC 020 | Exosomal | | | | 1035.98 | 24.88 | 3932.25 |
| | Protein | | | | | | |
| HC 021 | Exosomal | | | | 687.27 | 5.60 | 449.85 |
| | Protein | | | | | | |
| HC 022 | Exosomal | | | | 781.34 | 5.60 | 449.85 |
| | Protein | | | | | | |
| HC 023 | Exosomal | | | | 20.20 | 5.60 | 322.61 |
| | Protein | | | | | | |
| HC 024 | Exosomal | | | | 566.80 | 5.60 | 366.33 |
| | Protein | | | | | | |
| HC 025 | Exosomal | | | | 985.00 | 5.60 | 977.96 |
| | Protein | | | | | | |
| HC 026 | Exosomal | | | | 849.84 | 5.60 | 277.22 |
| | Protein | | | | | | |
| HC 027 | Exosomal | | | | 513.45 | 5.60 | 306.76 |
| | Protein | | | | | | |
| HC 028 | Exosomal | | | | 20.20 | 5.60 | 559.24 |
| | Protein | | | | | | |
| HC 029 | Exosomal | | | | 801.13 | 5.60 | 496.83 |
| | Protein | | | | | | |
| HC 030 | Exosomal | | | | 20.20 | 8.72 | 910.24 |
| | Protein | | | | | | |
| HC 031 | Exosomal | | | | 20.20 | 9.95 | 1029.26 |
| | Protein | | | | | | |
| HC 032 | Exosomal | | | | 20.20 | 5.60 | 638.62 |
| | Protein | | | | | | |
| HC 033 | Exosomal | | | | 638.15 | 5.60 | 867.94 |
| | Protein | | | | | | |
| HC 034 | Exosomal | | | | 897.62 | 5.60 | 735.65 |
| | Protein | | | | | | |
| HC 035 | Exosomal | | | | 947.22 | 13.65 | 1884.81 |
| | Protein | | | | | | |
| HC 036 | Exosomal | | | | 446.29 | 6.86 | 1061.08 |
| | Protein | | | | | | |
| HC 037 | Exosomal | | | | 680.39 | 5.60 | 910.24 |
| | Protein | | | | | | |
| HC 038 | Exosomal | | | | 20.20 | 18.57 | 2149.70 |
| | Protein | | | | | | |
| HC 039 | Exosomal | | | | 276.10 | 5.60 | 638.62 |
| | Protein | | | | | | |
| HC 040 | Exosomal | | | | 20.20 | 5.60 | 440.95 |
| | Protein | | | | | | |
| HC 041 | Exosomal | | | | 707.67 | 18.57 | 1829.48 |
| | Protein | | | | | | |
| HC 042 | Exosomal | | | | 20.20 | 5.60 | 311.32 |
| | Protein | | | | | | |
| HC 043 | Exosomal | | | | 20.20 | 5.60 | 709.25 |
| | Protein | | | | | | |
| HC 044 | Exosomal | | | | 1536.39 | 5.60 | 2054.64 |
| | Protein | | | | | | |
| HC 045 | Exosomal | | | | 1534.28 | 5.60 | 1553.06 |
| | Protein | | | | | | |
| HC 046 | Exosomal | | | | 2369.98 | 5.60 | 1220.41 |
| | Protein | | | | | | |
| HC 047 | Exosomal | | | | 1480.80 | 5.60 | 977.96 |
| | Protein | | | | | | |
| HC 048 | Exosomal | | | | 20.20 | 5.60 | 709.25 |
| | Protein | | | | | | |
| HC 049 | Exosomal | | | | 20.20 | 5.60 | 247.80 |
| | Protein | | | | | | |
| HC 050 | Exosomal | | | | 585.52 | 5.60 | 391.24 |
| | Protein | | | | | | |
| HC 051 | Exosomal | | | | 1965.45 | 12.35 | 2054.64 |
| | Protein | | | | | | |
| HC 052 | Exosomal | | | | 20.20 | 5.60 | 1101.76 |
| | Protein | | | | | | |
| HC 053 | Exosomal | | | | 1462.57 | 5.60 | 709.65 |
| | Protein | | | | | | |
| HC 054 | Exosomal | | | | 20.20 | 6.97 | 2601.08 |
| | Protein | | | | | | |
| HC 055 | Exosomal | | | | 1593.33 | 5.60 | 1334.78 |
| | Protein | | | | | | |
| HC 056 | Exosomal | | | | 20.20 | 7.31 | 1168.91 |
| | Protein | | | | | | |
| HC 057 | Exosomal | | | | 656.68 | 5.63 | 455.35 |
| | Protein | | | | | | |
| HC 058 | Exosomal | | | | 20.20 | 17.91 | 3025.14 |
| | Protein | | | | | | |
| HC 059 | Exosomal | | | | 20.20 | 5.60 | 977.96 |
| | Protein | | | | | | |
| HC 060 | Exosomal | | | | 1381.98 | 18.79 | 3461.91 |
| | Protein | | | | | | |
| HC 061 | Exosomal | | | | 20.20 | 5.60 | 847.76 |
| | Protein | | | | | | |
| HC 062 | Exosomal | | | | 20.20 | 5.60 | 709.25 |
| | Protein | | | | | | |
| HC 063 | Exosomal | | | | 20.20 | 15.11 | 1278.09 |
| | Protein | | | | | | |
| HC 064 | Exosomal | | | | 20.20 | 7.04 | 1094.81 |
| | Protein | | | | | | |
| HC 065 | Exosomal | | | | 700.07 | 6.10 | 977.96 |
| | Protein | | | | | | |
| HC 066 | Exosomal | | | | 870.19 | 5.60 | 709.25 |
| | Protein | | | | | | |
| HC 067 | Exosomal | | | | 644.77 | 8.79 | 972.99 |
| | Protein | | | | | | |
| HC 068 | Exosomal | | | | 1140.77 | 5.60 | 709.25 |
| | Protein | | | | | | |
| HC 069 | Exosomal | | | | 785.91 | 5.60 | 496.83 |
| | Protein | | | | | | |
| HC 070 | Exosomal | | | | 447.96 | 5.60 | 247.80 |
| | Protein | | | | | | |
| HC 071 | Exosomal | | | | 608.64 | 5.60 | 366.33 |
| | Protein | | | | | | |
| HC 072 | Exosomal | | | | 528.22 | 5.60 | 1038.77 |
| | Protein | | | | | | |
| HC 073 | Exosomal | | | | 20.20 | 5.60 | 247.80 |
| | Protein | | | | | | |
| HC 074 | Exosomal | | | | 692.71 | 5.60 | 408.67 |
| | Protein | | | | | | |
| HC 075 | Exosomal | | | | 711.08 | 7.73 | 567.89 |
| | Protein | | | | | | |
| HC 076 | Exosomal | | | | 854.89 | 5.60 | 410.56 |
| | Protein | | | | | | |
| HC 077 | Exosomal | | | | 297.46 | 5.60 | 247.80 |
| | Protein | | | | | | |
| HC 078 | Exosomal | | | | 20.20 | 5.60 | 247.80 |
| | Protein | | | | | | |
| HC 079 | Exosomal | | | | 378.75 | 5.60 | 488.03 |
| | Protein | | | | | | |
| HC 080 | Exosomal | | | | 499.61 | 5.60 | 277.22 |
| | Protein | | | | | | |
| HC 081 | Exosomal | | | | 20.20 | 5.60 | 247.80 |
| | Protein | | | | | | |
| HC 082 | Exosomal | | | | 1092.83 | 5.60 | 642.97 |
| | Protein | | | | | | |
| HC 083 | Exosomal | | | | 376.79 | 5.60 | 247.80 |
| | Protein | | | | | | |
| HC 084 | Exosomal | | | | 1628.59 | 6.48 | 1388.07 |
| | Protein | | | | | | |
| HC 085 | Exosomal | | | | 1027.67 | 5.60 | 605.74 |
| | Protein | | | | | | |
| HC 086 | Exosomal | | | | 881.96 | 5.60 | 371.26 |
| | Protein | | | | | | |
| HC 087 | Exosomal | | | | 347.46 | 5.60 | 256.02 |
| | Protein | | | | | | |
| HC 088 | Exosomal | | | | 578.26 | 5.60 | 247.80 |
| | Protein | | | | | | |
| HC 089 | Exosomal | | | | 20.20 | 5.60 | 250.40 |
| | Protein | | | | | | |
| HC 090 | Exosomal | | | | 20.20 | 5.94 | 805.20 |
| | Protein | | | | | | |
| HC 091 | Exosomal | | | | 1236.22 | 5.60 | 679.63 |
| | Protein | | | | | | |
| HC 092 | Exosomal | | | | 20.20 | 10.79 | 1593.23 |
| | Protein | | | | | | |
| HC 093 | Exosomal | | | | 20.20 | 5.60 | 247.80 |
| | Protein | | | | | | |
| HC 094 | Exosomal | | | | 20.20 | 5.60 | 247.80 |
| | Protein | | | | | | |
| HC 095 | Exosomal | | | | 817.10 | 5.60 | 355.73 |
| | Protein | | | | | | |
| HC 096 | Exosomal | | | | 1180.18 | 15.11 | 1860.27 |
| | Protein | | | | | | |
| HC 097 | Exosomal | | | | 1627.22 | 5.60 | 709.25 |
| | Protein | | | | | | |
| HC 098 | Exosomal | | | | 614.05 | 5.60 | 322.61 |
| | Protein | | | | | | |
| HC 099 | Exosomal | | | | 2291.18 | 8.74 | 1760.16 |
| | Protein | | | | | | |
| HC 100 | Exosomal | | | | 20.20 | 5.60 | 247.80 |
| | Protein | | | | | | |
| HC 101 | Exosomal | | | | 20.20 | 5.60 | 322.61 |
| | Protein | | | | | | |
| HC 102 | Exosomal | | | | 737.65 | 9.51 | 855.85 |
| | Protein | | | | | | |
| HC 103 | Exosomal | | | | 722.65 | 5.60 | 247.80 |
| | Protein | | | | | | |
| HC 104 | Exosomal | | | | 438.15 | 5.60 | 247.80 |
| | Protein | | | | | | |
| HC 105 | Exosomal | | | | 20.20 | 5.60 | 1101.76 |
| | Protein | | | | | | |
| HC 106 | Exosomal | | | | 2422.03 | 5.60 | 1101.76 |
| | Protein | | | | | | |
| HC 107 | Exosomal | | | | 3095.59 | 5.60 | 559.24 |
| | Protein | | | | | | |
| HC 108 | Exosomal | | | | 20.20 | 37.88 | 4576.75 |
| | Protein | | | | | | |
| HC 109 | Exosomal | | | | 939.94 | 5.60 | 277.22 |
| | Protein | | | | | | |
| HC 110 | Exosomal | | | | 355.95 | 5.60 | 642.97 |
| | Protein | | | | | | |
| HC 111 | Exosomal | | | | 998.62 | 5.60 | 322.61 |
| | Protein | | | | | | |
| HC 112 | Exosomal | | | | 531.81 | 5.60 | 490.03 |
| | Protein | | | | | | |
| HC 113 | Exosomal | | | | 20.20 | 6.24 | 788.30 |
| | Protein | | | | | | |
| OVAR | Free | Ovarian | I | A | 9510.12 | 733.82 | 78647.04 |
| 001 | Protein | | | | | | |
| OVAR | Free | Ovarian | I | A | 8205.87 | 2098.20 | 53569.75 |
| 002 | Protein | | | | | | |
| OVAR | Free | Ovarian | I | B | 14483.34 | 2044.44 | 107764.02 |
| 003 | Protein | | | | | | |
| OVAR | Free | Ovarian | I | A | 5461.09 | 1726.04 | 52041.59 |
| 004 | Protein | | | | | | |
| OVAR | Free | Ovarian | I | C | 16121.68 | 798.20 | 85453.25 |
| 005 | Protein | | | | | | |
| OVAR | Free | Ovarian | I | A | 9834.44 | 717.79 | 56600.25 |
| 006 | Protein | | | | | | |
| OVAR | Free | Ovarian | I | A | 9665.85 | 1487.26 | 72901.05 |
| 007 | Protein | | | | | | |
| OVAR | Free | Ovarian | II | B | 10759.53 | 1843.12 | 85048.69 |
| 008 | Protein | | | | | | |
| OVAR | Free | Ovarian | II | B | 15580.58 | 2068.31 | 35813.88 |
| 009 | Protein | | | | | | |
| OVAR | Free | Ovarian | I | A | 14307.91 | 1635.69 | 43889.09 |
| 010 | Protein | | | | | | |
| OVAR | Free | Ovarian | I | A | 10116.63 | 1966.18 | 40741.02 |
| 011 | Protein | | | | | | |
| OVAR | Free | Ovarian | I | B | 9721.85 | 957.67 | 112640.82 |
| 012 | Protein | | | | | | |
| OVAR | Free | Ovarian | I | A | 3480.07 | 1890.24 | 80787.55 |
| 013 | Protein | | | | | | |
| OVAR | Free | Ovarian | I | B | 11122.99 | 1459.25 | 149507.14 |
| 014 | Protein | | | | | | |
| OVAR | Free | Ovarian | I | B | 19717.57 | 1078.32 | 41617.73 |
| 015 | Protein | | | | | | |
| OVAR | Free | Ovarian | I | A | 20.20 | 1226.43 | 57091.94 |
| 016 | Protein | | | | | | |
| OVAR | Free | Ovarian | I | A | 20.20 | 5.60 | 247.80 |
| 017 | Protein | | | | | | |
| OVAR | Free | Ovarian | I | A | 5891.62 | 650.82 | 48730.97 |
| 018 | Protein | | | | | | |
| OVAR | Free | Ovarian | I | C | 12094.09 | 1768.85 | 54221.95 |
| 019 | Protein | | | | | | |
| OVAR | Free | Ovarian | I | B | 10817.10 | 1766.91 | 92803.52 |
| 020 | Protein | | | | | | |
| OVAR | Free | Ovarian | I | A | 4951.46 | 728.47 | 51219.12 |
| 021 | Protein | | | | | | |
| OVAR | Free | Ovarian | I | A | 8600.58 | 1328.02 | 62148.70 |
| 022 | Protein | | | | | | |
| OVAR | Free | Ovarian | II | A | 32187.40 | 2471.81 | 1000320.26 |
| 023 | Protein | | | | | | |
| OVAR | Free | Ovarian | I | A | 6143.91 | 1884.34 | 66409.64 |
| 024 | Protein | | | | | | |
| OVAR | Free | Ovarian | I | C | 1992.07 | 1041.51 | 62042.87 |
| 025 | Protein | | | | | | |
| OVAR | Free | Ovarian | I | C | 8572.13 | 1458.68 | 39823.54 |
| 026 | Protein | | | | | | |
| OVAR | Free | Ovarian | I | A | 10264.54 | 1494.90 | 134295.27 |
| 027 | Protein | | | | | | |
| OVAR | Free | Ovarian | I | B | 8812.50 | 374.04 | 47956.05 |
| 028 | Protein | | | | | | |
| OVAR | Free | Ovarian | I | A | 12267.91 | 1389.42 | 77048.30 |
| 029 | Protein | | | | | | |
| OVAR | Free | Ovarian | I | B | 12392.13 | 1809.38 | 110045.51 |
| 030 | Protein | | | | | | |
| OVAR | Free | Ovarian | I | #N/A | 9836.16 | 1028.24 | 61402.26 |
| 031 | Protein | | | | | | |
| OVAR | Free | Ovarian | I | A | 16071.09 | 757.43 | 37215.20 |
| 032 | Protein | | | | | | |
| OVAR | Free | Ovarian | I | A | 10584.29 | 1782.17 | 59534.18 |
| 033 | Protein | | | | | | |
| OVAR | Free | Ovarian | I | A | 13548.99 | 1836.60 | 39759.29 |
| 034 | Protein | | | | | | |
| OVAR | Free | Ovarian | I | A | 16573.79 | 2886.65 | 68878.89 |
| 035 | Protein | | | | | | |
| OVAR | Free | Ovarian | I | A | 34971.78 | 1362.03 | 62180.24 |
| 036 | Protein | | | | | | |
| OVAR | Free | Ovarian | II | A | 9580.06 | 1272.12 | 36684.56 |
| 037 | Protein | | | | | | |
| OVAR | Free | Ovarian | I | A | 9154.64 | 485.93 | 41271.97 |
| 038 | Protein | | | | | | |
| OVAR | Free | Ovarian | I | A | 10139.77 | 1149.55 | 36298.04 |
| 039 | Protein | | | | | | |
| OVAR | Free | Ovarian | I | A | 14099.43 | 2768.81 | 56976.80 |
| 040 | Protein | | | | | | |
| OVAR | Free | Ovarian | I | B | 6540.74 | 462.48 | 45793.43 |
| 041 | Protein | | | | | | |
| OVAR | Free | Ovarian | I | A | 7292.36 | 2022.93 | 75767.20 |
| 042 | Protein | | | | | | |
| OVAR | Free | Ovarian | II | A | 9156.31 | 1395.41 | 76195.13 |
| 043 | Protein | | | | | | |
| OVAR | Free | Ovarian | I | #N/A | 8041.98 | 1405.09 | 52232.79 |
| 044 | Protein | | | | | | |
| BLDR | Free | Bladder | II | | 12819.34 | 2359.31 | 70693.89 |
| 001 | Protein | | | | | | |
| BLDR | Free | Bladder | I | | 9670.57 | 857.72 | 102523.35 |
| 002 | Protein | | | | | | |
| BLDR | Free | Bladder | I | | 9892.45 | 1086.90 | 33430.88 |
| 003 | Protein | | | | | | |
| BLDR | Free | Bladder | I | | 18279.84 | 811.04 | 61356.47 |
| 004 | Protein | | | | | | |
| BLDR | Free | Bladder | II | | 16090.64 | 1134.78 | 54131.06 |
| 005 | Protein | | | | | | |
| BLDR | Free | Bladder | I | | 12112.23 | 638.77 | 90920.07 |
| 006 | Protein | | | | | | |
| BLDR | Free | Bladder | I | | 19747.75 | 1019.49 | 74497.44 |
| 007 | Protein | | | | | | |
| BLDR | Free | Bladder | II | | 7926.95 | 1327.18 | 60459.29 |
| 008 | Protein | | | | | | |
| BLDR | Free | Bladder | I | | 10099.04 | 523.34 | 45546.13 |
| 009 | Protein | | | | | | |
| BLDR | Free | Bladder | II | | 17064.98 | 2655.77 | 41095.46 |
| 010 | Protein | | | | | | |
| BLDR | Free | Bladder | II | | 10456.64 | 1108.97 | 52230.47 |
| 011 | Protein | | | | | | |
| BLDR | Free | Bladder | II | | 8016.51 | 1325.15 | 68344.30 |
| 012 | Protein | | | | | | |
| BLDR | Free | Bladder | I | | 12084.87 | 1855.78 | 57522.59 |
| 013 | Protein | | | | | | |
| BLDR | Free | Bladder | I | | 2538.87 | 1002.67 | 67606.57 |
| 014 | Protein | | | | | | |
| BLDR | Free | Bladder | I | | 10692.41 | 2541.45 | 98314.17 |
| 015 | Protein | | | | | | |
| BLDR | Free | Bladder | I | | 17063.28 | 1286.65 | 62360.27 |
| 016 | Protein | | | | | | |
| BLDR | Free | Bladder | II | | 6732.49 | 1302.80 | 99282.04 |
| 017 | Protein | | | | | | |
| BLDR | Free | Bladder | I | | 15219.45 | 1934.25 | 106373.75 |
| 018 | Protein | | | | | | |
| BLDR | Free | Bladder | II | | 11921.69 | 2299.04 | 76303.36 |
| 019 | Protein | | | | | | |
| BLDR | Free | Bladder | II | | 6842.19 | 1790.65 | 69961.56 |
| 020 | Protein | | | | | | |
| BLDR | Free | Bladder | I | | 13153.52 | 730.74 | 52150.72 |
| 021 | Protein | | | | | | |
| BLDR | Free | Bladder | I | | 7988.40 | 1099.93 | 106471.38 |
| 022 | Protein | | | | | | |
| BLDR | Free | Bladder | I | | 8566.60 | 1466.80 | 130602.70 |
| 023 | Protein | | | | | | |
| BLDR | Free | Bladder | I | | 15369.74 | 1512.20 | 104378.24 |
| 024 | Protein | | | | | | |
| BLDR | Free | Bladder | I | | 14007.30 | 1923.61 | 43151.73 |
| 025 | Protein | | | | | | |
| BLDR | Free | Bladder | II | | 9939.78 | 1449.48 | 33405.56 |
| 026 | Protein | | | | | | |
| BLDR | Free | Bladder | I | | 12113.41 | 1640.81 | 101897.04 |
| 027 | Protein | | | | | | |
| BLDR | Free | Bladder | I | | 13288.46 | 2325.33 | 83732.42 |
| 028 | Protein | | | | | | |
| BLDR | Free | Bladder | II | | 13805.56 | 1286.56 | 134992.84 |
| 029 | Protein | | | | | | |
| BLDR | Free | Bladder | II | | 8545.49 | 1144.48 | 105567.88 |
| 030 | Protein | | | | | | |
| BLDR | Free | Bladder | II | | 16529.10 | 2178.42 | 107374.38 |
| 031 | Protein | | | | | | |
| BLDR | Free | Bladder | II | | 12010.43 | 1949.35 | 67559.03 |
| 032 | Protein | | | | | | |
| BLDR | Free | Bladder | I | | 10896.77 | 1192.86 | 60896.40 |
| 033 | Protein | | | | | | |
| BLDR | Free | Bladder | II | | 8310.44 | 739.84 | 51342.30 |
| 034 | Protein | | | | | | |
| BLDR | Free | Bladder | II | | 17363.90 | 2453.42 | 174926.86 |
| 035 | Protein | | | | | | |
| BLDR | Free | Bladder | I | | 11729.96 | 2162.77 | 81306.18 |
| 036 | Protein | | | | | | |
| BLDR | Free | Bladder | II | | 11067.93 | 375.17 | 39927.05 |
| 037 | Protein | | | | | | |
| BLDR | Free | Bladder | II | | 9519.56 | 2612.65 | 76662.84 |
| 038 | Protein | | | | | | |
| BLDR | Free | Bladder | I | | 6369.52 | 1417.74 | 110554.90 |
| 039 | Protein | | | | | | |
| BLDR | Free | Bladder | I | | 10615.98 | 900.69 | 45687.95 |
| 040 | Protein | | | | | | |
| BLDR | Free | Bladder | II | | 11663.75 | 1716.98 | 73497.90 |
| 041 | Protein | | | | | | |
| BLDR | Free | Bladder | II | | 6928.13 | 2159.00 | 57813.52 |
| 042 | Protein | | | | | | |
| BLDR | Free | Bladder | I | | 11573.22 | 1979.94 | 59235.27 |
| 043 | Protein | | | | | | |
| BLDR | Free | Bladder | I | | 9255.10 | 2185.63 | 101173.81 |
| 044 | Protein | | | | | | |
| BLDR | Free | Bladder | II | | 10412.43 | 1489.16 | 70472.12 |
| 045 | Protein | | | | | | |
| BLDR | Free | Bladder | I | | 3902.82 | 981.61 | 69056.36 |
| 046 | Protein | | | | | | |
| BLDR | Free | Bladder | I | | 15309.59 | 368.01 | 82870.39 |
| 047 | Protein | | | | | | |
| BLDR | Free | Bladder | I | | 14314.18 | 1068.43 | 103568.72 |
| 048 | Protein | | | | | | |
| PDAC | Free | Pancreatic | I | A | 7912.80 | 581.80 | 68132.54 |
| 001 | Protein | | | | | | |
| PDAC | Free | Pancreatic | II | B | 9640.42 | 1264.15 | 116191.04 |
| 002 | Protein | | | | | | |
| PDAC | Free | Pancreatic | II | A | 12713.00 | 1148.58 | 73828.58 |
| 003 | Protein | | | | | | |
| PDAC | Free | pancreatic | II | A | 18951.54 | 2108.18 | 28356.13 |
| 004 | Protein | | | | | | |
| PDAC | Free | Pancreatic | II | A | 7944.29 | 848.56 | 107664.72 |
| 005 | Protein | | | | | | |
| PDAC | Free | Pancreatic | II | B | 10286.87 | 1675.61 | 70004.29 |
| 006 | Protein | | | | | | |
| PDAC | Free | Pancreatic | II | A | 11599.03 | 1594.56 | 39065.21 |
| 007 | Protein | | | | | | |
| PDAC | Free | Pancreatic | II | B | 10724.36 | 1784.46 | 59653.02 |
| 008 | Protein | | | | | | |
| PDAC | Free | Pancreatic | II | B | 7212.18 | 336.53 | 35634.38 |
| 009 | Protein | | | | | | |
| PDAC | Free | pancreatic | I | B | 17949.92 | 2727.49 | 113875.30 |
| 010 | Protein | | | | | | |
| PDAC | Free | Pancreatic | II | B | 19339.11 | 2525.03 | 132217.61 |
| 011 | Protein | | | | | | |
| PDAC | Free | pancreatic | I | B | 11438.55 | 1354.42 | 40462.42 |
| 012 | Protein | | | | | | |
| PDAC | Free | pancreatic | I | B | 8101.82 | 1407.39 | 58959.40 |
| 013 | Protein | | | | | | |
| PDAC | Free | pancreatic | II | A | 9684.93 | 1533.14 | 60876.61 |
| 014 | Protein | | | | | | |
| PDAC | Free | Pancreatic | II | B | 10849.08 | 1847.04 | 108061.89 |
| 015 | Protein | | | | | | |
| PDAC | Free | pancreatic | I | B | 8929.64 | 1714.39 | 89085.41 |
| 016 | Protein | | | | | | |
| PDAC | Free | Pancreatic | II | B | 8989.82 | 1731.87 | 51164.19 |
| 017 | Protein | | | | | | |
| PDAC | Free | Pancreatic | II | B | 12078.00 | 2459.55 | 40578.31 |
| 018 | Protein | | | | | | |
| PDAC | Free | Pancreatic | II | B | 15534.74 | 1215.54 | 70730.12 |
| 019 | Protein | | | | | | |
| PDAC | Free | pancreatic | I | A | 11512.35 | 2927.71 | 128508.39 |
| 020 | Protein | | | | | | |
| PDAC | Free | pancreatic | I | B | 13923.99 | 1782.51 | 73210.41 |
| 021 | Protein | | | | | | |
| PDAC | Free | Pancreatic | II | B | 11660.04 | 2246.56 | 74857.25 |
| 022 | Protein | | | | | | |
| PDAC | Free | Pancreatic | II | B | 11708.22 | 583.55 | 54330.50 |
| 023 | Protein | | | | | | |
| PDAC | Free | Pancreatic | II | B | 11663.25 | 2365.88 | 157913.53 |
| 024 | Protein | | | | | | |
| PDAC | Free | Pancreatic | II | B | 14066.93 | 1396.02 | 43336.35 |
| 025 | Protein | | | | | | |
| PDAC | Free | Pancreatic | II | B | 19235.45 | 2012.68 | 115695.92 |
| 026 | Protein | | | | | | |
| PDAC | Free | Pancreatic | II | B | 3814.48 | 1718.27 | 45264.89 |
| 027 | Protein | | | | | | |
| PDAC | Free | Pancreatic | II | B | 11727.50 | 1290.41 | 102195.82 |
| 028 | Protein | | | | | | |
| PDAC | Free | pancreatic | I | B | 10548.29 | 1251.81 | 38201.23 |
| 029 | Protein | | | | | | |
| PDAC | Free | pancreatic | I | B | 13735.06 | 2223.12 | 73096.07 |
| 030 | Protein | | | | | | |
| PDAC | Free | pancreatic | I | B | 8995.24 | 1073.98 | 32748.55 |
| 031 | Protein | | | | | | |
| PDAC | Free | pancreatic | I | A | 9412.70 | 1376.32 | 49551.17 |
| 032 | Protein | | | | | | |
| PDAC | Free | pancreatic | I | B | 13632.63 | 1425.28 | 53437.20 |
| 033 | Protein | | | | | | |
| PDAC | Free | pancreatic | I | B | 8193.01 | 1278.76 | 66630.84 |
| 034 | Protein | | | | | | |
| PDAC | Free | pancreatic | I | B | 4931.79 | 2030.21 | 69861.10 |
| 035 | Protein | | | | | | |
| PDAC | Free | pancreatic | I | B | 3311.89 | 1478.14 | 52473.14 |
| 036 | Protein | | | | | | |
| PDAC | Free | pancreatic | I | B | 20177.04 | 4564.67 | 92716.26 |
| 037 | Protein | | | | | | |
| PDAC | Free | pancreatic | I | B | 9922.17 | 1459.25 | 127061.61 |
| 038 | Protein | | | | | | |
| PDAC | Free | pancreatic | I | A | 9770.39 | 598.32 | 70563.78 |
| 039 | Protein | | | | | | |
| PDAC | Free | pancreatic | I | B | 8156.89 | 829.24 | 34485.30 |
| 040 | Protein | | | | | | |
| PDAC | Free | Pancreatic | I | B | 12165.21 | 2419.85 | 114683.92 |
| 041 | Protein | | | | | | |
| PDAC | Free | pancreatic | I | B | 17255.67 | 2049.07 | 83363.45 |
| 042 | Protein | | | | | | |
| PDAC | Free | pancreatic | II | A | 8946.90 | 346.51 | 49223.56 |
| 043 | Protein | | | | | | |
| PDAC | Free | Pancreatic | II | A | 10835.68 | 2609.45 | 56091.68 |
| 044 | Protein | | | | | | |
| HC 001 | Free | | | | 11374.06 | 1784.89 | 71265.12 |
| | Protein | | | | | | |
| HC 002 | Free | | | | 11982.08 | 1892.70 | 74455.38 |
| | Protein | | | | | | |
| HC 003 | Free | | | | 8995.24 | 911.71 | 97408.83 |
| | Protein | | | | | | |
| HC 004 | Free | | | | 11466.02 | 756.23 | 93378.41 |
| | Protein | | | | | | |
| HC 005 | Free | | | | 8202.04 | 1421.51 | 60896.40 |
| | Protein | | | | | | |
| HC 006 | Free | | | | 10719.69 | 2298.15 | 79884.73 |
| | Protein | | | | | | |
| HC 007 | Free | | | | 13125.58 | 1752.28 | 75149.98 |
| | Protein | | | | | | |
| HC 008 | Free | | | | 11165.82 | 656.26 | 94723.82 |
| | Protein | | | | | | |
| HC 009 | Free | | | | 13885.40 | 959.51 | 62443.67 |
| | Protein | | | | | | |
| HC 010 | Free | | | | 11208.67 | 659.89 | 92608.68 |
| | Protein | | | | | | |
| HC 011 | Free | | | | 14489.92 | 893.35 | 61413.15 |
| | Protein | | | | | | |
| HC 012 | Free | | | | 8971.07 | 1376.32 | 46134.26 |
| | Protein | | | | | | |
| HC 013 | Free | | | | 8235.15 | 1715.89 | 56881.72 |
| | Protein | | | | | | |
| HC 014 | Free | | | | 9467.22 | 1706.33 | 53009.31 |
| | Protein | | | | | | |
| HC 015 | Free | | | | 8313.45 | 1629.98 | 54821.68 |
| | Protein | | | | | | |
| HC 016 | Free | | | | 11880.56 | 847.54 | 97983.25 |
| | Protein | | | | | | |
| HC 017 | Free | | | | 10829.58 | 754.41 | 94051.37 |
| | Protein | | | | | | |
| HC 018 | Free | | | | 11760.68 | 919.05 | 102377.37 |
| | Protein | | | | | | |
| HC 019 | Free | | | | 12031.33 | 1161.21 | 99800.24 |
| | Protein | | | | | | |
| HC 020 | Free | | | | 11591.79 | 1096.22 | 105234.88 |
| | Protein | | | | | | |
| HC 021 | Free | | | | 7363.64 | 1077.69 | 57249.48 |
| | Protein | | | | | | |
| HC 022 | Free | | | | 7183.72 | 1245.10 | 56513.38 |
| | Protein | | | | | | |
| HC 023 | Free | | | | 8482.18 | 1455.47 | 67238.87 |
| | Protein | | | | | | |
| HC 024 | Free | | | | 8862.33 | 1263.79 | 58766.91 |
| | Protein | | | | | | |
| HC 025 | Free | | | | 9776.46 | 1198.45 | 83309.55 |
| | Protein | | | | | | |
| HC 026 | Free | | | | 13907.27 | 2037.98 | 92897.41 |
| | Protein | | | | | | |
| HC 027 | Free | | | #N/A | 12388.90 | 3034.95 | 94435.69 |
| | Protein | | | | | | |
| HC 028 | Free | | | | 12432.12 | 2973.96 | 95827.49 |
| | Protein | | | | | | |
| HC 029 | Free | | | | 9539.93 | 1756.11 | 101518.93 |
| | Protein | | | | | | |
| HC 030 | Free | | | | 8543.81 | 1851.40 | 77232.64 |
| | Protein | | | | | | |
| HC 031 | Free | | | | 10115.27 | 3897.47 | 112119.50 |
| | Protein | | | | | | |
| HC 032 | Free | | | | 9375.96 | 1515.09 | 87549.15 |
| | Protein | | | | | | |
| HC 033 | Free | | | | 8431.19 | 747.85 | 92666.18 |
| | Protein | | | | | | |
| HC 034 | Free | | | | 10810.02 | 1033.59 | 97699.51 |
| | Protein | | | | | | |
| HC 035 | Free | | | | 10088.45 | 1263.48 | 80996.76 |
| | Protein | | | | | | |
| HC 036 | Free | | | | 8188.90 | 721.35 | 90909.15 |
| | Protein | | | | | | |
| HC 037 | Free | | | | 1362.26 | 18.80 | 7062.58 |
| | Protein | | | | | | |
| HC 038 | Free | | | | 10331.01 | 1789.01 | 90531.41 |
| | Protein | | | | | | |
| HC 039 | Free | | | | 8287.25 | 862.65 | 93619.73 |
| | Protein | | | | | | |
| HC 040 | Free | | | | 13742.47 | 1745.55 | 120792.37 |
| | Protein | | | | | | |
| HC 041 | Free | | | | 8660.72 | 2433.30 | 110369.01 |
| | Protein | | | | | | |
| HC 042 | Free | | | | 11937.04 | 2857.68 | 127688.26 |
| | Protein | | | | | | |
| HC 043 | Free | | | | 11377.12 | 2018.55 | 78360.90 |
| | Protein | | | | | | |
| HC 044 | Free | | | | 7303.65 | 674.40 | 104235.41 |
| | Protein | | | | | | |
| HC 045 | Free | | | | 7264.67 | 721.64 | 106661.53 |
| | Protein | | | | | | |
| HC 046 | Free | | | | 9455.10 | 1077.69 | 83992.04 |
| | Protein | | | | | | |
| HC 047 | Free | | | | 13459.99 | 2005.55 | 109278.43 |
| | Protein | | | | | | |
| HC 048 | Free | | | | 13135.70 | 1988.19 | 110062.97 |
| | Protein | | | | | | |
| HC 049 | Free | | | | 14727.12 | 1489.55 | 126385.11 |
| | Protein | | | | | | |
| HC 050 | Free | | | | 20668.35 | 2534.90 | 49837.19 |
| | Protein | | | | | | |
| HC 051 | Free | | | | 21307.89 | 2439.71 | 50531.41 |
| | Protein | | | | | | |
| HC 052 | Free | | | | 14485.11 | 1286.52 | 116042.49 |
| | Protein | | | | | | |
| HC 053 | Free | | | | 19429.46 | 1096.76 | 162202.71 |
| | Protein | | | | | | |
| HC 054 | Free | | | | 29377.22 | 1450.58 | 217940.88 |
| | Protein | | | | | | |
| HC 055 | Free | | | | 8483.54 | 659.92 | 80974.18 |
| | Protein | | | | | | |
| HC 056 | Free | | | | 7994.89 | 433.38 | 63255.72 |
| | Protein | | | | | | |
| HC 057 | Free | | | | 13881.77 | 2638.21 | 77164.78 |
| | Protein | | | | | | |
| HC 058 | Free | | | | 16604.30 | 2207.24 | 83749.05 |
| | Protein | | | | | | |
| HC 059 | Free | | | | 17267.61 | 2748.30 | 105011.37 |
| | Protein | | | | | | |
| HC 060 | Free | | | | 11111.57 | 1008.50 | 113120.30 |
| | Protein | | | | | | |
| HC 061 | Free | | | | 15251.77 | 1304.58 | 145766.28 |
| | Protein | | | | | | |
| HC 062 | Free | | | | 23689.89 | 7807.86 | 127221.66 |
| | Protein | | | | | | |
| HC 063 | Free | | | | 23285.33 | 7739.19 | 128338.13 |
| | Protein | | | | | | |
| HC 064 | Free | | | | 6455.45 | 855.78 | 73519.59 |
| | Protein | | | | | | |
| HC 065 | Free | | | | 22717.06 | 5106.94 | 122728.07 |
| | Protein | | | | | | |
| HC 066 | Free | | | | 18627.27 | 3380.42 | 137113.59 |
| | Protein | | | | | | |
| HC 067 | Free | | | | 13058.73 | 1266.03 | 91197.40 |
| | Protein | | | | | | |
| HC 068 | Free | | | | 19933.93 | 4486.96 | 97839.49 |
| | Protein | | | | | | |
| HC 069 | Free | | | | 24318.99 | 1218.69 | 222561.25 |
| | Protein | | | | | | |
| HC 070 | Free | | | | 17450.85 | 4889.28 | 88807.09 |
| | Protein | | | | | | |
| HC 071 | Free | | | | 17869.96 | 2951.15 | 88768.43 |
| | Protein | | | | | | |
| HC 072 | Free | | | | 16378.61 | 4153.98 | 79049.04 |
| | Protein | | | | | | |
| HC 073 | Free | | | | 16766.86 | 2525.80 | 71398.59 |
| | Protein | | | | | | |
| HC 074 | Free | | | | 16192.78 | 2546.28 | 69939.22 |
| | Protein | | | | | | |
| HC 075 | Free | | | | 16719.27 | 2745.99 | 73280.97 |
| | Protein | | | | | | |
| HC 076 | Free | | | | 6620.24 | 4083.08 | 104503.41 |
| | Protein | | | | | | |
| HC 077 | Free | | | | 14781.83 | 2934.71 | 109396.08 |
| | Protein | | | | | | |
| HC 078 | Free | | | | 15699.80 | 3024.17 | 110180.69 |
| | Protein | | | | | | |
| HC 079 | Free | | | | 6912.64 | 4689.67 | 112183.78 |
| | Protein | | | | | | |
| HC 080 | Free | | | | 14064.67 | 2541.72 | 99084.12 |
| | Protein | | | | | | |
| HC 081 | Free | | | #N/A | 7152.65 | 4857.63 | 120112.28 |
| | Protein | | | | | | |
| HC 082 | Free | | | | 12704.77 | 1750.37 | 130974.66 |
| | Protein | | | | | | |
| HC 083 | Free | | | #N/A | 20100.02 | 2709.02 | 100174.06 |
| | Protein | | | | | | |
| HC 084 | Free | | | | 11855.72 | 1710.24 | 139733.40 |
| | Protein | | | | | | |
| HC 085 | Free | | | | 12000.56 | 1983.86 | 133818.69 |
| | Protein | | | | | | |
| HC 086 | Free | | | | 14891.33 | 2649.18 | 171061.29 |
| | Protein | | | | | | |
| HC 087 | Free | | | | 12336.77 | 1206.78 | 136872.12 |
| | Protein | | | | | | |
| HC 088 | Free | | | | 15126.28 | 2507.62 | 73473.10 |
| | Protein | | | | | | |
| HC 089 | Free | | | | 19489.15 | 2580.49 | 95896.86 |
| | Protein | | | | | | |
| HC 090 | Free | | | | 19537.61 | 2460.04 | 91128.30 |
| | Protein | | | | | | |
| HC 091 | Free | | | | 13166.54 | 1979.52 | 133337.43 |
| | Protein | | | | | | |
| HC 092 | Free | | | | 20014.93 | 2857.50 | 97139.85 |
| | Protein | | | | | | |
| HC 093 | Free | | | | 14711.49 | 3443.48 | 84366.15 |
| | Protein | | | | | | |
| HC 094 | Free | | | | 14165.76 | 4726.29 | 88072.66 |
| | Protein | | | | | | |
| HC 095 | Free | | | | 13181.52 | 2602.80 | 67391.33 |
| | Protein | | | | | | |
| HC 096 | Free | | | | 14200.77 | 4847.09 | 91941.45 |
| | Protein | | | | | | |
| HC 097 | Free | | | | 14867.86 | 4012.46 | 79510.94 |
| | Protein | | | | | | |
| HC 098 | Free | | | | 12345.80 | 1851.87 | 78046.77 |
| | Protein | | | | | | |
| HC 099 | Free | | | | 11497.99 | 1273.11 | 75315.24 |
| | Protein | | | | | | |
| HC 100 | Free | | | | 8576.36 | 1246.31 | 74765.28 |
| | Protein | | | | | | |
| HC 101 | Free | | | | 8933.18 | 1242.49 | 59740.78 |
| | Protein | | | | | | |
| HC 102 | Free | | | | 9089.91 | 1328.77 | 60991.62 |
| | Protein | | | | | | |
| HC 103 | Free | | | | 9611.32 | 1700.21 | 69310.72 |
| | Protein | | | | | | |
| HC 104 | Free | | | | 13088.27 | 1700.21 | 66783.66 |
| | Protein | | | | | | |
| HC 105 | Free | | | | 18643.14 | 1189.04 | 94702.96 |
| | Protein | | | | | | |
| HC 106 | Free | | | | 16587.23 | 1118.69 | 87318.14 |
| | Protein | | | | | | |
| HC 107 | Free | | | | 15845.94 | 887.18 | 69869.51 |
| | Protein | | | | | | |
| HC 108 | Free | | | | 16817.79 | 2085.94 | 119895.17 |
| | Protein | | | | | | |
| HC 109 | Free | | | | 9177.75 | 1296.12 | 79958.14 |
| | Protein | | | | | | |
| HC 110 | Free | | | | 16985.22 | 1925.86 | 119468.49 |
| | Protein | | | | | | |
| HC 111 | Free | | | | 9227.96 | 1255.88 | 76275.70 |
| | Protein | | | | | | |
| HC 112 | Free | | | | 17881.28 | 2037.77 | 123814.45 |
| | Protein | | | | | | |
| HC 113 | Free | | | | 18001.04 | 1280.47 | 133344.58 |
| | Protein | | | | | | |

| Subject | | | | | | | |
|---|---|---|---|---|---|---|---|
| Cohort | SHGFR/ | | | | sNeuropilin- | sPECAM- | |
| ID | c-Met | sHer2 | sHer3 | sIL-6Ra | 1 | 1 | sVEGFR1 |
| OVAR 001 | 426.44 | 247.02 | 43.30 | 297.27 | 3479.92 | 78.82 | 11.51 |
| OVAR 002 | 479.15 | 965.97 | 17.90 | 364.22 | 2017.59 | 1025.42 | 9.50 |
| OVAR 003 | 701.38 | 1588.43 | 37.94 | 139.27 | 1388.99 | 1636.11 | 5.10 |
| OVAR 004 | 88.26 | 168.41 | 17.90 | 58.87 | 151.00 | 15.50 | 5.10 |
| OVAR 005 | 507.81 | 900.51 | 105.46 | 343.20 | 6281.80 | 363.32 | 6.53 |
| OVAR 006 | 335.58 | 592.18 | 17.90 | 116.74 | 3267.60 | 725.84 | 5.10 |
| OVAR 007 | 329.90 | 1001.79 | 17.90 | 372.05 | 3727.17 | 1337.68 | 5.10 |
| OVAR 008 | 192.58 | 246.70 | 34.70 | 78.97 | 1131.09 | 228.35 | 5.95 |
| OVAR 009 | 329.90 | 11.90 | 17.90 | 133.78 | 5703.26 | 469.90 | 5.10 |
| OVAR 010 | 335.58 | 11.90 | 17.90 | 32.47 | 151.00 | 293.28 | 5.10 |
| OVAR 011 | 164.69 | 368.60 | 17.90 | 317.99 | 729.70 | 117.12 | 5.10 |
| OVAR 012 | 257.27 | 11.90 | 17.90 | 593.89 | 2761.19 | 142.25 | 5.10 |
| OVAR 013 | 1327.93 | 11.90 | 17.90 | 15.10 | 11111.24 | 15.50 | 5.97 |
| OVAR 014 | 398.90 | 651.28 | 17.90 | 411.22 | 5152.80 | 15.50 | 5.10 |
| OVAR 015 | 439.01 | 11.90 | 17.90 | 315.44 | 4611.10 | 526.17 | 9.51 |
| OVAR 016 | 336.27 | 875.66 | 17.90 | 262.41 | 1655.76 | 15.50 | 5.10 |
| OVAR 017 | 348.14 | 11.90 | 17.90 | 389.09 | 5290.46 | 15.50 | 9.30 |
| OVAR 018 | 218.59 | 347.81 | 17.90 | 111.99 | 1170.86 | 94.02 | 5.10 |
| OVAR 019 | 322.45 | 421.82 | 70.34 | 170.81 | 6930.85 | 94.02 | 5.10 |
| OVAR 020 | 175.97 | 903.67 | 17.90 | 57.53 | 151.00 | 364.47 | 5.10 |
| OVAR 021 | 815.09 | 11.90 | 45.34 | 517.93 | 8355.69 | 15.50 | 5.10 |
| OVAR 022 | 24.20 | 11.90 | 17.90 | 15.10 | 2895.12 | 15.50 | 5.10 |
| OVAR 023 | 1354.12 | 683.31 | 17.90 | 681.25 | 52622.19 | 15.50 | 9.30 |
| OVAR 024 | 788.37 | 661.27 | 17.90 | 304.13 | 4995.51 | 799.66 | 6.20 |
| OVAR 025 | 1068.01 | 936.45 | 36.89 | 281.47 | 3148.43 | 620.55 | 5.10 |
| OVAR 026 | 1250.56 | 601.54 | 17.90 | 851.33 | 14697.12 | 591.50 | 5.10 |
| OVAR 027 | 550.44 | 11.90 | 46.57 | 377.02 | 7746.54 | 355.89 | 17.23 |
| OVAR 028 | 136.40 | 515.42 | 161.77 | 50.92 | 151.00 | 121.50 | 7.64 |
| OVAR 029 | 115.22 | 220.54 | 17.90 | 84.52 | 463.00 | 94.85 | 5.10 |
| OVAR 030 | 273.98 | 470.62 | 136.65 | 183.25 | 299.42 | 225.73 | 17.23 |
| OVAR 031 | 836.09 | 394.15 | 39.95 | 348.61 | 7613.55 | 251.61 | 5.10 |
| OVAR 032 | 187.56 | 251.16 | 17.90 | 144.27 | 319.73 | 146.29 | 5.10 |
| OVAR 033 | 201.33 | 70.76 | 49.75 | 80.88 | 1104.03 | 43.93 | 5.10 |
| OVAR 034 | 242.71 | 11.90 | 38.31 | 264.29 | 2075.02 | 44.64 | 7.23 |
| OVAR 035 | 887.86 | 301.76 | 209.10 | 15.10 | 19178.63 | 101.97 | 25.61 |
| OVAR 036 | 510.30 | 11.90 | 77.02 | 407.86 | 9953.16 | 1231.25 | 9.32 |
| OVAR 037 | 226.42 | 11.90 | 17.90 | 115.74 | 3325.95 | 642.66 | 5.10 |
| OVAR 038 | 135.80 | 1022.69 | 17.90 | 43.60 | 151.00 | 1183.59 | 5.10 |
| OVAR 039 | 81.94 | 288.86 | 17.90 | 43.13 | 151.00 | 193.79 | 5.10 |
| OVAR 040 | 475.41 | 844.66 | 83.06 | 205.64 | 5627.66 | 745.12 | 5.10 |
| OVAR 041 | 87.57 | 483.66 | 44.29 | 78.45 | 151.00 | 634.12 | 5.10 |
| OVAR 042 | 632.35 | 1121.83 | 61.86 | 69.88 | 151.00 | 1209.57 | 9.51 |
| OVAR 043 | 94.53 | 11.90 | 17.90 | 52.76 | 151.00 | 962.02 | 13.04 |
| OVAR 044 | 244.65 | 601.53 | 17.90 | 136.72 | 322.68 | 776.47 | 14.81 |
| BLDR 001 | 758.88 | 423.79 | 93.52 | 883.72 | 29523.38 | 188.96 | 10.14 |
| BLDR 002 | 360.92 | 1106.44 | 17.90 | 123.30 | 896.45 | 572.58 | 5.55 |
| BLDR 003 | 275.66 | 165.54 | 17.90 | 175.04 | 1873.49 | 79.38 | 5.10 |
| BLDR 004 | 173.81 | 11.90 | 75.31 | 87.13 | 151.00 | 48.66 | 5.10 |
| BLDR 005 | 861.95 | 11.90 | 136.65 | 1189.32 | 26759.18 | 427.56 | 15.43 |
| BLDR 006 | 651.57 | 278.63 | 17.90 | 433.28 | 10330.17 | 123.68 | 10.17 |
| BLDR 007 | 226.77 | 1059.99 | 17.90 | 142.22 | 2484.91 | 1293.09 | 5.10 |
| BLDR 008 | 756.64 | 11.90 | 17.90 | 15.10 | 2157.50 | 15.50 | 26.02 |
| BLDR 009 | 252.74 | 416.37 | 17.90 | 15.10 | 151.00 | 50.93 | 5.10 |
| BLDR 010 | 268.29 | 203.33 | 17.90 | 112.77 | 151.00 | 115.67 | 5.10 |
| BLDR 011 | 302.70 | 529.39 | 78.74 | 234.29 | 2781.82 | 425.26 | 5.53 |
| BLDR 012 | 3487.33 | 11.90 | 17.90 | 3302.70 | 43680.78 | 15.50 | 10.85 |
| BLDR 013 | 1418.60 | 538.72 | 17.90 | 817.91 | 12606.79 | 276.74 | 5.10 |
| BLDR 014 | 1320.25 | 11.90 | 28.99 | 210.16 | 2304.12 | 692.59 | 5.10 |
| BLDR 015 | 211.67 | 822.01 | 124.20 | 261.06 | 1143.62 | 289.85 | 14.53 |
| BLDR 016 | 394.90 | 350.33 | 75.18 | 65.73 | 151.00 | 252.85 | 5.10 |
| BLDR 017 | 702.67 | 709.64 | 70.62 | 479.09 | 4085.62 | 197.79 | 11.65 |
| BLDR 018 | 465.37 | 575.15 | 129.53 | 389.62 | 5909.99 | 212.92 | 20.91 |
| BLDR 019 | 658.36 | 882.91 | 17.90 | 366.50 | 5599.96 | 304.04 | 7.77 |
| BLDR 020 | 168.14 | 11.90 | 17.90 | 330.02 | 1209.19 | 900.74 | 5.10 |
| BLDR 021 | 113.29 | 1061.47 | 84.78 | 98.70 | 151.00 | 575.32 | 11.89 |
| BLDR 022 | 479.67 | 400.66 | 17.90 | 386.53 | 1754.58 | 169.01 | 5.10 |
| BLDR 023 | 485.69 | 538.57 | 28.99 | 186.74 | 2719.24 | 78.12 | 5.10 |
| BLDR 024 | 730.97 | 819.85 | 31.60 | 129.28 | 1500.32 | 102.05 | 5.10 |
| BLDR 025 | 324.42 | 11.90 | 17.90 | 130.49 | 1796.91 | 853.00 | 5.10 |
| BLDR 026 | 455.71 | 320.16 | 17.90 | 390.09 | 5389.80 | 311.68 | 10.85 |
| BLDR 027 | 639.58 | 11.90 | 43.58 | 131.94 | 3375.82 | 621.58 | 5.34 |
| BLDR 028 | 566.85 | 815.66 | 65.15 | 244.45 | 9722.25 | 895.48 | 9.31 |
| BLDR 029 | 33.59 | 356.66 | 17.90 | 23.58 | 151.00 | 135.74 | 5.10 |
| BLDR 030 | 347.87 | 711.08 | 53.77 | 169.51 | 1756.66 | 884.17 | 8.50 |
| BLDR 031 | 428.63 | 259.81 | 17.90 | 344.68 | 2031.27 | 15.50 | 15.57 |
| BLDR 032 | 865.62 | 357.54 | 17.90 | 324.90 | 9645.58 | 123.98 | 6.18 |
| BLDR 033 | 171.60 | 11.90 | 17.90 | 167.19 | 153.68 | 233.40 | 5.10 |
| BLDR 034 | 199.64 | 11.90 | 17.90 | 99.79 | 1313.83 | 75.37 | 5.10 |
| BLDR 035 | 975.64 | 11.90 | 17.90 | 466.30 | 7422.35 | 760.66 | 19.55 |
| BLDR 036 | 387.81 | 1159.80 | 36.89 | 221.55 | 5105.60 | 2018.97 | 10.36 |
| BLDR 037 | 531.52 | 472.57 | 32.46 | 286.22 | 5381.79 | 241.86 | 5.10 |
| BLDR 038 | 193.63 | 427.37 | 37.63 | 200.31 | 3459.00 | 698.94 | 5.10 |
| BLDR 039 | 309.17 | 11.90 | 17.90 | 196.75 | 2557.74 | 15.50 | 8.94 |
| BLDR 040 | 234.17 | 850.82 | 17.90 | 87.49 | 1017.98 | 624.73 | 5.10 |
| BLDR 041 | 502.39 | 11.90 | 22.24 | 444.80 | 3992.76 | 1450.57 | 30.70 |
| BLDR 042 | 159.90 | 1102.94 | 22.24 | 85.12 | 151.00 | 907.18 | 8.92 |
| BLDR 043 | 678.99 | 1249.29 | 65.48 | 754.70 | 8249.27 | 1687.19 | 24.81 |
| BLDR 044 | 217.64 | 11.90 | 17.90 | 238.42 | 460.61 | 522.84 | 60.80 |
| BLDR 045 | 738.97 | 1004.79 | 64.88 | 127.15 | 151.00 | 930.86 | 29.52 |
| BLDR 046 | 490.30 | 340.81 | 136.65 | 392.23 | 5989.05 | 51.70 | 6.82 |
| BLDR 047 | 381.49 | 11.90 | 23.68 | 94.70 | 3948.67 | 53.37 | 5.10 |
| BLDR 048 | 483.44 | 11.90 | 17.90 | 29.57 | 151.00 | 55.89 | 5.10 |
| PDAC 001 | 129.44 | 242.76 | 97.81 | 118.11 | 641.06 | 19.31 | 5.10 |
| PDAC 002 | 555.47 | 244.92 | 129.53 | 503.54 | 15611.35 | 59.86 | 11.03 |
| PDAC 003 | 127.13 | 140.37 | 44.91 | 99.04 | 151.00 | 41.64 | 6.82 |
| PDAC 004 | 154.38 | 11.90 | 17.90 | 100.52 | 1298.09 | 245.27 | 5.10 |
| PDAC 005 | 337.11 | 341.93 | 17.90 | 301.12 | 3456.17 | 25.96 | 5.22 |
| PDAC 006 | 362.00 | 628.70 | 98.37 | 155.69 | 3211.78 | 480.40 | 5.69 |
| PDAC 007 | 312.59 | 1226.06 | 43.63 | 134.83 | 1655.76 | 439.98 | 5.10 |
| PDAC 008 | 387.17 | 801.20 | 88.60 | 227.64 | 3136.76 | 679.67 | 5.10 |
| PDAC 009 | 435.63 | 855.47 | 17.90 | 245.92 | 3577.98 | 139.23 | 5.10 |
| PDAC 010 | 591.24 | 310.40 | 67.43 | 317.42 | 6431.23 | 147.04 | 5.10 |
| PDAC 011 | 612.74 | 1007.25 | 42.82 | 146.74 | 3645.29 | 1232.43 | 5.10 |
| PDAC 012 | 580.19 | 11.90 | 17.90 | 155.25 | 4063.28 | 317.63 | 5.10 |
| PDAC 013 | 802.56 | 11.90 | 118.89 | 521.44 | 11499.19 | 145.78 | 7.23 |
| PDAC 014 | 134.04 | 149.89 | 17.90 | 34.57 | 485.21 | 72.71 | 5.10 |
| PDAC 015 | 1234.41 | 598.62 | 17.90 | 339.45 | 16308.57 | 307.69 | 5.10 |
| PDAC 016 | 244.15 | 924.59 | 17.90 | 96.12 | 603.86 | 1194.33 | 10.50 |
| PDAC 017 | 171.87 | 93.33 | 17.90 | 64.34 | 3431.76 | 29.96 | 5.10 |
| PDAC 018 | 242.12 | 508.50 | 75.15 | 96.52 | 1897.73 | 212.76 | 5.10 |
| PDAC 019 | 535.97 | 332.72 | 31.93 | 252.90 | 5094.95 | 91.56 | 5.10 |
| PDAC 020 | 356.68 | 456.59 | 70.17 | 157.96 | 2355.03 | 184.14 | 5.10 |
| PDAC 021 | 234.17 | 11.90 | 17.90 | 64.73 | 151.00 | 641.52 | 5.10 |
| PDAC 022 | 2294.11 | 1235.66 | 81.26 | 926.89 | 23634.93 | 219.68 | 8.26 |
| PDAC 023 | 540.00 | 1391.88 | 18.86 | 287.50 | 4195.29 | 1195.57 | 5.10 |
| PDAC 024 | 416.95 | 367.28 | 141.14 | 77.63 | 2030.28 | 108.50 | 5.10 |
| PDAC 025 | 400.94 | 575.15 | 136.65 | 401.15 | 6668.69 | 117.13 | 5.10 |
| PDAC 026 | 24.20 | 310.37 | 17.90 | 100.33 | 3243.49 | 15.50 | 5.10 |
| PDAC 027 | 510.30 | 11.90 | 49.90 | 530.99 | 4376.62 | 196.74 | 5.22 |
| PDAC 028 | 1110.61 | 1199.06 | 17.90 | 647.57 | 11919.98 | 1324.68 | 6.40 |
| PDAC 029 | 763.11 | 175.61 | 29.69 | 279.20 | 13173.13 | 125.87 | 6.85 |
| PDAC 030 | 463.59 | 11.90 | 17.90 | 258.28 | 7666.32 | 356.49 | 7.77 |
| PDAC 031 | 364.18 | 390.37 | 17.90 | 236.78 | 10871.61 | 246.98 | 5.10 |
| PDAC 032 | 396.96 | 413.15 | 32.46 | 650.75 | 1183.12 | 244.42 | 5.10 |
| PDAC 033 | 110.83 | 305.20 | 17.90 | 78.03 | 151.00 | 66.15 | 5.10 |
| PDAC 034 | 1168.62 | 365.39 | 29.14 | 879.71 | 42449.70 | 238.47 | 8.23 |
| PDAC 035 | 135.72 | 1075.42 | 17.90 | 123.72 | 331.86 | 343.41 | 5.10 |
| PDAC 036 | 178.56 | 806.28 | 17.90 | 126.22 | 2636.60 | 321.28 | 5.10 |
| PDAC 037 | 253.12 | 520.81 | 17.90 | 129.21 | 3293.16 | 390.87 | 13.72 |
| PDAC 038 | 580.19 | 804.47 | 39.20 | 556.45 | 11771.83 | 838.98 | 5.10 |
| PDAC 039 | 189.06 | 203.33 | 17.90 | 65.94 | 632.84 | 222.50 | 5.10 |
| PDAC 040 | 154.38 | 267.28 | 28.59 | 78.03 | 331.86 | 146.03 | 5.10 |
| PDAC 041 | 305.19 | 11.90 | 17.90 | 136.72 | 1973.70 | 573.07 | 10.69 |
| PDAC 042 | 339.34 | 294.30 | 44.68 | 177.77 | 1799.85 | 111.64 | 5.71 |
| PDAC 043 | 483.44 | 482.58 | 17.90 | 119.97 | 2278.75 | 215.03 | 5.10 |
| PDAC 044 | 141.05 | 11.90 | 17.90 | 42.43 | 151.00 | 18.37 | 5.10 |
| HC 001 | 378.35 | 619.98 | 86.76 | 310.48 | 2521.45 | 447.26 | 15.57 |
| HC 002 | 175.75 | 739.91 | 22.90 | 135.62 | 1014.83 | 526.47 | 5.10 |
| HC 003 | 1251.43 | 1746.40 | 43.30 | 926.82 | 15239.92 | 1739.30 | 12.52 |
| HC 004 | 588.50 | 1024.34 | 62.45 | 299.59 | 4015.21 | 542.51 | 6.20 |
| HC 005 | 209.87 | 902.23 | 34.14 | 116.05 | 151.00 | 1350.48 | 12.52 |
| HC 006 | 1367.77 | 11.90 | 26.99 | 715.89 | 15627.30 | 15.50 | 22.80 |
| HC 007 | 175.75 | 698.39 | 22.17 | 97.22 | 151.00 | 1530.46 | 8.50 |
| HC 008 | 439.59 | 877.33 | 17.90 | 184.32 | 1336.50 | 2242.56 | 11.51 |
| HC 009 | 500.29 | 11.90 | 199.94 | 128.58 | 1320.06 | 2235.69 | 15.43 |
| HC 010 | 625.37 | 1422.83 | 21.43 | 227.41 | 1746.32 | 1330.60 | 11.51 |
| HC 011 | 540.39 | 11.90 | 17.90 | 15.10 | 3845.23 | 15.50 | 17.23 |
| HC 012 | 634.29 | 1239.34 | 58.08 | 492.57 | 7849.26 | 1783.34 | 17.11 |
| HC 013 | 789.08 | 1059.48 | 55.73 | 552.80 | 9961.47 | 804.44 | 10.50 |
| HC 014 | 490.16 | 1862.04 | 68.34 | 344.38 | 5770.97 | 2482.95 | 16.59 |
| HC 015 | 133.34 | 1309.70 | 17.90 | 89.52 | 391.91 | 1623.54 | 6.53 |
| HC 016 | 419.86 | 943.54 | 25.13 | 170.08 | 1632.35 | 453.32 | 8.50 |
| HC 017 | 887.86 | 681.05 | 170.81 | 767.71 | 16068.20 | 251.61 | 17.23 |
| HC 018 | 448.37 | 802.90 | 17.90 | 217.60 | 2079.13 | 159.68 | 5.55 |
| HC 019 | 120.68 | 519.68 | 17.90 | 36.89 | 438.02 | 111.51 | 5.10 |
| HC 020 | 2280.42 | 564.69 | 52.60 | 1550.21 | 55227.09 | 377.49 | 7.51 |
| HC 021 | 363.10 | 1589.33 | 17.90 | 206.67 | 1479.66 | 1505.99 | 8.01 |
| HC 022 | 291.53 | 934.93 | 17.90 | 168.94 | 714.69 | 1324.77 | 12.52 |
| HC 023 | 214.14 | 553.00 | 17.90 | 128.33 | 197.42 | 1196.57 | 15.06 |
| HC 024 | 261.35 | 1095.99 | 18.86 | 142.36 | 457.11 | 2171.43 | 12.52 |
| HC 025 | 346.89 | 11.90 | 272.06 | 218.70 | 3069.10 | 822.88 | 10.17 |
| HC 026 | 248.45 | 865.33 | 211.00 | 167.81 | 1000.54 | 901.61 | 11.51 |
| HC 027 | 253.70 | 1224.46 | 17.90 | 86.08 | 275.70 | 603.07 | 5.19 |
| HC 028 | 136.40 | 11.90 | 35.04 | 82.53 | 151.00 | 397.14 | 5.10 |
| HC 029 | 397.75 | 471.04 | 76.51 | 190.43 | 3303.29 | 148.22 | 9.30 |
| HC 030 | 552.39 | 11.90 | 17.90 | 678.38 | 3966.90 | 15.50 | 25.99 |
| HC 031 | 237.90 | 11.90 | 17.90 | 161.62 | 151.00 | 1760.69 | 31.87 |
| HC 032 | 659.00 | 11.90 | 17.90 | 53.71 | 151.00 | 539.52 | 8.33 |
| HC 033 | 321.95 | 1552.19 | 17.90 | 280.81 | 399.20 | 2346.77 | 19.52 |
| HC 034 | 285.04 | 11.90 | 17.90 | 232.65 | 844.63 | 1501.93 | 5.97 |
| HC 035 | 572.38 | 11.90 | 83.56 | 283.65 | 5397.37 | 2395.88 | 97.64 |
| HC 036 | 465.69 | 1282.24 | 27.16 | 404.29 | 729.98 | 1812.66 | 19.52 |
| HC 037 | 452.36 | 1286.26 | 22.24 | 281.72 | 1702.07 | 1687.19 | 9.51 |
| HC 038 | 579.04 | 11.90 | 62.46 | 359.80 | 8458.15 | 1391.68 | 119.18 |
| HC 039 | 204.10 | 846.98 | 17.90 | 114.24 | 151.00 | 869.18 | 23.63 |
| HC 040 | 434.34 | 11.90 | 17.90 | 199.27 | 857.28 | 2418.59 | 5.10 |
| HC 041 | 432.34 | 656.33 | 168.55 | 320.26 | 1286.01 | 1464.90 | 68.50 |
| HC 042 | 24.20 | 11.90 | 17.90 | 15.10 | 455.87 | 15.50 | 5.10 |
| HC 043 | 470.35 | 11.90 | 30.17 | 157.27 | 545.71 | 181.86 | 5.10 |
| HC 044 | 550.44 | 1140.52 | 156.37 | 601.78 | 5082.10 | 308.01 | 12.33 |
| HC 045 | 415.75 | 555.94 | 133.09 | 477.36 | 4306.47 | 79.62 | 9.32 |
| HC 046 | 950.24 | 801.83 | 158.17 | 203.89 | 6559.74 | 61.92 | 18.60 |
| HC 047 | 746.02 | 11.90 | 106.56 | 133.33 | 3650.45 | 33.73 | 9.32 |
| HC 048 | 605.93 | 540.40 | 133.09 | 87.13 | 1884.21 | 49.67 | 14.09 |
| HC 049 | 124.03 | 234.62 | 17.90 | 37.96 | 151.00 | 22.61 | 5.10 |
| HC 050 | 104.12 | 83.57 | 75.31 | 62.40 | 151.00 | 22.14 | 5.61 |
| HC 051 | 717.86 | 251.40 | 17.90 | 684.02 | 21529.63 | 188.71 | 11.89 |
| HC 052 | 510.30 | 770.44 | 28.56 | 434.79 | 1483.01 | 150.25 | 11.89 |
| HC 053 | 679.50 | 696.67 | 213.31 | 135.96 | 2039.16 | 242.85 | 5.10 |
| HC 054 | 535.37 | 872.76 | 223.81 | 449.81 | 8670.65 | 304.37 | 12.77 |
| HC 055 | 435.56 | 684.75 | 176.25 | 294.59 | 6342.42 | 201.35 | 6.82 |
| HC 056 | 1114.99 | 903.40 | 17.90 | 597.93 | 12240.17 | 211.48 | 5.10 |
| HC 057 | 259.67 | 484.40 | 101.57 | 214.90 | 1261.64 | 404.84 | 7.03 |
| HC 058 | 1165.48 | 11.90 | 113.59 | 1860.89 | 34042.76 | 1695.07 | 11.03 |
| HC 059 | 298.17 | 11.90 | 17.90 | 385.54 | 3255.49 | 1395.54 | 10.17 |
| HC 060 | 2019.69 | 591.94 | 53.95 | 1295.94 | 35651.96 | 304.67 | 5.10 |
| HC 061 | 332.23 | 364.52 | 158.17 | 181.16 | 258.12 | 136.90 | 25.61 |
| HC 062 | 320.04 | 11.90 | 106.56 | 156.59 | 2191.03 | 286.24 | 18.15 |
| HC 063 | 475.33 | 564.33 | 54.92 | 420.55 | 10237.62 | 226.90 | 19.06 |
| HC 064 | 985.82 | 509.24 | 17.90 | 485.60 | 15066.22 | 804.86 | 5.10 |
| HC 065 | 245.11 | 567.94 | 75.31 | 231.45 | 6675.96 | 163.71 | 8.48 |
| HC 066 | 298.17 | 467.11 | 101.30 | 147.66 | 1707.28 | 121.50 | 5.22 |
| HC 067 | 820.16 | 570.18 | 30.24 | 359.68 | 18277.46 | 869.19 | 6.18 |
| HC 068 | 235.53 | 193.86 | 203.60 | 155.90 | 1170.76 | 61.92 | 10.17 |
| HC 069 | 417.67 | 622.66 | 17.90 | 156.66 | 1436.66 | 218.32 | 5.10 |
| HC 070 | 57.34 | 94.37 | 17.90 | 44.00 | 151.00 | 58.42 | 5.10 |
| HC 071 | 422.05 | 385.23 | 17.90 | 395.43 | 5259.06 | 174.57 | 5.10 |
| HC 072 | 352.70 | 11.90 | 17.90 | 335.77 | 4392.24 | 75.37 | 5.10 |
| HC 073 | 199.19 | 1030.84 | 22.17 | 138.99 | 986.24 | 162.64 | 6.53 |
| HC 074 | 417.68 | 1433.47 | 65.96 | 390.07 | 3701.99 | 658.93 | 6.53 |
| HC 075 | 701.38 | 1461.88 | 97.30 | 705.72 | 7713.70 | 1045.81 | 7.51 |
| HC 076 | 114.11 | 11.90 | 17.90 | 87.50 | 755.24 | 496.51 | 6.62 |
| HC 077 | 422.05 | 1408.65 | 17.90 | 88.98 | 151.00 | 455.92 | 9.50 |
| HC 078 | 479.15 | 1322.04 | 17.90 | 153.36 | 418.86 | 462.88 | 9.50 |
| HC 079 | 97.85 | 171.28 | 17.90 | 70.56 | 1695.68 | 64.85 | 5.10 |
| HC 080 | 532.11 | 907.39 | 17.90 | 157.04 | 1388.99 | 265.29 | 14.55 |
| HC 081 | 24.20 | 11.90 | 17.90 | 15.10 | 151.00 | 15.50 | 5.10 |
| HC 082 | 510.01 | 1035.18 | 17.90 | 289.66 | 1014.83 | 430.89 | 7.51 |
| HC 083 | 144.16 | 23.66 | 17.90 | 75.68 | 3007.03 | 15.50 | 5.10 |
| HC 084 | 882.74 | 1086.82 | 59.96 | 501.91 | 27528.77 | 969.14 | 5.10 |
| HC 085 | 380.53 | 1097.73 | 17.90 | 155.90 | 2761.79 | 359.99 | 5.10 |
| HC 086 | 168.14 | 11.90 | 17.90 | 152.93 | 151.00 | 182.56 | 5.10 |
| HC 087 | 330.35 | 489.52 | 17.90 | 82.77 | 151.00 | 89.10 | 5.10 |
| HC 088 | 141.80 | 146.25 | 30.36 | 125.53 | 532.80 | 57.86 | 5.10 |
| HC 089 | 463.59 | 11.90 | 41.46 | 85.36 | 1131.09 | 274.70 | 5.10 |
| HC 090 | 818.37 | 11.90 | 70.61 | 421.33 | 12486.35 | 414.05 | 5.95 |
| HC 091 | 470.34 | 1152.66 | 21.43 | 331.98 | 2140.60 | 446.39 | 10.00 |
| HC 092 | 1276.71 | 11.90 | 71.20 | 833.85 | 36025.01 | 15.50 | 6.85 |
| HC 093 | 137.57 | 850.76 | 29.61 | 97.77 | 151.00 | 313.26 | 5.55 |
| HC 094 | 185.55 | 11.90 | 44.31 | 90.37 | 872.98 | 133.26 | 5.10 |
| HC 095 | 466.39 | 1052.43 | 43.37 | 176.03 | 1819.60 | 256.61 | 9.23 |
| HC 096 | 1128.52 | 936.78 | 172.62 | 781.62 | 18379.33 | 677.44 | 12.77 |
| HC 097 | 269.16 | 355.46 | 194.45 | 232.16 | 1891.01 | 170.49 | 5.22 |
| HC 098 | 330.50 | 363.20 | 59.65 | 265.71 | 2927.30 | 107.93 | 5.10 |
| HC 099 | 784.54 | 11.90 | 233.04 | 645.30 | 12020.86 | 2352.89 | 16.33 |
| HC 100 | 280.74 | 576.39 | 17.90 | 189.18 | 674.23 | 139.06 | 9.50 |
| HC 101 | 330.50 | 317.73 | 17.90 | 243.61 | 457.11 | 74.61 | 9.00 |
| HC 102 | 746.29 | 537.99 | 17.90 | 781.08 | 4489.50 | 349.24 | 7.51 |
| HC 103 | 226.99 | 677.26 | 17.90 | 103.29 | 151.00 | 860.74 | 5.10 |
| HC 104 | 141.80 | 232.69 | 101.38 | 121.07 | 950.40 | 62.73 | 5.10 |
| HC 105 | 633.80 | 11.90 | 75.31 | 296.76 | 3110.48 | 91.26 | 6.82 |
| HC 106 | 893.04 | 11.90 | 123.31 | 238.56 | 1877.40 | 72.28 | 5.22 |
| HC 107 | 440.52 | 374.74 | 106.56 | 145.61 | 484.31 | 71.24 | 5.22 |
| HC 108 | 2490.10 | 515.52 | 30.36 | 1697.19 | 46735.14 | 462.88 | 8.50 |
| HC 109 | 554.25 | 928.04 | 17.90 | 164.40 | 1566.53 | 240.21 | 5.10 |
| HC 110 | 308.83 | 332.31 | 81.92 | 211.84 | 2673.86 | 201.73 | 5.10 |
| HC 111 | 505.60 | 667.97 | 17.90 | 164.69 | 1853.01 | 195.66 | 5.55 |
| HC 112 | 226.99 | 471.58 | 40.23 | 118.84 | 996.97 | 281.14 | 5.55 |
| HC 113 | 881.11 | 11.90 | 86.74 | 313.23 | 2665.75 | 232.19 | 8.41 |
| OVAR 001 | 27355.35 | 6039.27 | 3006.57 | 22056.04 | 590685.13 | 4494.08 | 328.82 |
| OVAR 002 | 30362.35 | 4824.40 | 2756.66 | 23947.85 | 407469.53 | 6292.20 | 1750.75 |
| OVAR 003 | 33703.40 | 5955.74 | 4302.60 | 24356.62 | 396344.59 | 9565.46 | 1130.59 |
| OVAR 004 | 23188.86 | 3015.63 | 1044.12 | 12350.16 | 490870.79 | 4967.45 | 1211.97 |
| OVAR 005 | 9792.32 | 4086.30 | 2154.52 | 23211.16 | 579291.70 | 4375.73 | 261.31 |
| OVAR 006 | 19437.96 | 3678.49 | 1130.87 | 14851.21 | 337200.75 | 3543.28 | 211.46 |
| OVAR 007 | 39115.65 | 5306.90 | 3824.87 | 33394.10 | 1098306.08 | 5840.31 | 773.27 |
| OVAR 008 | 58333.19 | 7275.79 | 9292.16 | 19361.11 | 215937.47 | 6976.28 | 1481.99 |
| OVAR 009 | 35585.49 | 4746.29 | 2661.69 | 17156.24 | 501697.91 | 6745.32 | 1321.86 |
| OVAR 010 | 25590.59 | 4779.44 | 2255.40 | 20297.17 | 923608.80 | 5935.90 | 272.34 |
| OVAR 011 | 36756.86 | 11.90 | 4774.00 | 44763.42 | 883538.17 | 5719.71 | 409.91 |
| OVAR 012 | 50225.42 | 6790.87 | 3381.31 | 36199.28 | 989591.44 | 10605.53 | 406.02 |
| OVAR 013 | 51622.80 | 7519.14 | 2674.91 | 23375.50 | 1112069.37 | 7367.81 | 1105.10 |
| OVAR 014 | 27734.57 | 10514.47 | 4847.61 | 34679.70 | 969860.71 | 12233.79 | 446.92 |
| OVAR 015 | 61198.88 | 5051.47 | 1873.63 | 27351.54 | 1217800.61 | 6196.62 | 184.79 |
| OVAR 016 | 36195.41 | 6095.86 | 1641.51 | 15.10 | 986695.19 | 15.50 | 262.70 |
| OVAR 017 | 23302.36 | 11.90 | 1547.76 | 25693.08 | 629372.86 | 4790.08 | 5.10 |
| OVAR 018 | 20982.68 | 4721.41 | 1486.77 | 19985.76 | 597182.17 | 5394.74 | 208.87 |
| OVAR 019 | 35545.84 | 5666.16 | 3696.11 | 26163.65 | 1130446.35 | 6647.24 | 1209.64 |
| OVAR 020 | 27965.73 | 5369.92 | 2014.45 | 15692.07 | 300166.76 | 8701.31 | 2216.42 |
| OVAR 021 | 26468.78 | 4279.67 | 1972.27 | 20383.15 | 452692.54 | 4903.79 | 536.78 |
| OVAR 022 | 44819.76 | 4668.27 | 2421.23 | 29410.07 | 700273.63 | 5562.70 | 665.43 |
| OVAR 023 | 47401.18 | 3647.83 | 17.90 | 23367.38 | 1393897.82 | 6463.44 | 273.88 |
| OVAR 024 | 54567.49 | 11.90 | 1573.61 | 14459.86 | 577797.90 | 5214.81 | 701.25 |
| OVAR 025 | 58635.88 | 6238.55 | 2086.44 | 30275.44 | 642532.16 | 8413.06 | 365.10 |
| OVAR 026 | 48805.87 | 5149.59 | 2723.47 | 31217.69 | 1027051.23 | 6767.19 | 927.85 |
| OVAR 027 | 30710.81 | 5232.13 | 2870.00 | 12204.27 | 481511.87 | 8323.81 | 1652.87 |
| OVAR 028 | 39276.79 | 4769.71 | 2852.18 | 13699.75 | 473980.63 | 5150.11 | 207.34 |
| OVAR 029 | 36753.32 | 6237.03 | 4586.46 | 34602.69 | 907098.78 | 8177.19 | 381.93 |
| OVAR 030 | 53527.14 | 6692.73 | 1835.98 | 29063.19 | 412617.68 | 7243.18 | 161.19 |
| OVAR 031 | 48352.79 | 6162.30 | 1598.84 | 19677.38 | 402572.35 | 5967.67 | 38.69 |
| OVAR 032 | 25543.25 | 4122.46 | 1244.83 | 40424.23 | 517317.82 | 4601.98 | 172.90 |
| OVAR 033 | 26461.57 | 5458.87 | 3404.22 | 16267.03 | 940059.37 | 7794.14 | 728.18 |
| OVAR 034 | 25079.65 | 3837.23 | 629.53 | 28663.04 | 723801.90 | 6652.30 | 762.27 |
| OVAR 035 | 73189.17 | 4748.75 | 3408.97 | 27023.58 | 1138977.48 | 8325.44 | 1177.64 |
| OVAR 036 | 27479.65 | 4987.96 | 2776.01 | 26708.75 | 776057.83 | 9515.62 | 167.04 |
| OVAR 037 | 14269.09 | 4091.54 | 1122.65 | 16239.63 | 445761.91 | 6389.86 | 659.81 |
| OVAR 038 | 36897.93 | 4914.35 | 679.83 | 9942.49 | 681200.54 | 5229.77 | 532.37 |
| OVAR 039 | 32676.58 | 4842.75 | 692.01 | 19007.29 | 740836.74 | 8580.41 | 557.05 |
| OVAR 040 | 51665.06 | 6438.40 | 398.96 | 25869.98 | 1060218.59 | 6992.89 | 412.43 |
| OVAR 041 | 25629.30 | 2953.06 | 740.05 | 27676.34 | 316876.93 | 6727.45 | 354.36 |
| OVAR 042 | 33812.75 | 5182.10 | 2253.25 | 21637.61 | 662379.36 | 6697.49 | 906.80 |
| OVAR 043 | 18078.38 | 11.90 | 927.55 | 11579.95 | 388142.50 | 13139.40 | 992.06 |
| OVAR 044 | 45603.87 | 6788.20 | 1906.71 | 23540.78 | 788058.16 | 11733.85 | 283.29 |
| BLDR 001 | 38389.16 | 4666.64 | 4096.54 | 26366.58 | 975589.58 | 7869.73 | 1755.31 |
| BLDR 002 | 21559.22 | 5477.26 | 2114.01 | 41551.01 | 641313.91 | 6285.94 | 138.00 |
| BLDR 003 | 42732.03 | 4776.15 | 2671.74 | 28194.41 | 510181.31 | 4471.79 | 314.68 |
| BLDR 004 | 64237.77 | 5019.41 | 3291.52 | 26721.39 | 926398.95 | 6028.82 | 414.65 |
| BLDR 005 | 39282.35 | 4948.68 | 2655.20 | 36534.41 | 1118746.28 | 6348.22 | 342.56 |
| BLDR 006 | 46070.46 | 6467.22 | 1210.96 | 24142.37 | 862670.89 | 7123.51 | 353.45 |
| BLDR 007 | 24958.92 | 5247.86 | 425.41 | 27896.12 | 874007.01 | 4123.41 | 375.82 |
| BLDR 008 | 45876.71 | 7772.91 | 662.77 | 30398.65 | 277263.45 | 12388.68 | 670.41 |
| BLDR 009 | 25662.28 | 3287.38 | 723.53 | 21876.03 | 544762.73 | 4210.32 | 235.37 |
| BLDR 010 | 39913.17 | 6870.40 | 2173.49 | 42839.34 | 672941.66 | 11961.86 | 296.21 |
| BLDR 011 | 41725.56 | 4862.35 | 614.03 | 26377.04 | 452590.61 | 6353.42 | 345.67 |
| BLDR 012 | 41427.18 | 5358.59 | 658.75 | 36742.62 | 491737.81 | 6561.59 | 345.78 |
| BLDR 013 | 53730.86 | 11.90 | 2306.75 | 25646.40 | 321989.37 | 7355.63 | 930.09 |
| BLDR 014 | 45412.48 | 5019.41 | 835.20 | 28370.45 | 407915.99 | 9237.47 | 468.63 |
| BLDR 015 | 34092.55 | 6684.66 | 1934.79 | 46948.33 | 983005.36 | 8856.20 | 931.65 |
| BLDR 016 | 25787.77 | 5436.96 | 3094.45 | 22262.26 | 983241.55 | 5871.44 | 316.08 |
| BLDR 017 | 53719.71 | 6082.22 | 3917.54 | 29932.59 | 410453.18 | 7319.80 | 793.02 |
| BLDR 018 | 59505.24 | 8644.53 | 1287.34 | 34518.81 | 669306.56 | 9159.49 | 497.43 |
| BLDR 019 | 63758.71 | 7552.60 | 3350.17 | 28296.48 | 795223.22 | 10032.52 | 1398.30 |
| BLDR 020 | 46366.40 | 11.90 | 3133.43 | 38382.62 | 1083176.68 | 10802.25 | 542.64 |
| BLDR 021 | 3791.99 | 8055.29 | 1886.59 | 28445.42 | 790007.80 | 9388.90 | 893.09 |
| BLDR 022 | 37909.62 | 5566.63 | 1540.13 | 23297.69 | 881365.51 | 6311.96 | 231.91 |
| BLDR 023 | 53840.81 | 7398.33 | 2338.84 | 26660.38 | 765281.11 | 8454.01 | 218.46 |
| BLDR 024 | 41025.00 | 8071.05 | 5276.98 | 28790.62 | 1009774.72 | 13571.80 | 802.18 |
| BLDR 025 | 44326.68 | 11.90 | 3158.47 | 26116.37 | 1124629.33 | 8640.47 | 274.27 |
| BLDR 026 | 34566.26 | 5211.71 | 63.97 | 23759.08 | 550106.51 | 6014.71 | 243.95 |
| BLDR 027 | 53927.89 | 7592.30 | 3895.47 | 20985.44 | 892418.94 | 8652.99 | 1586.26 |
| BLDR 028 | 39115.65 | 7000.97 | 2054.64 | 21084.28 | 1261080.50 | 6196.62 | 690.04 |
| BLDR 029 | 45631.78 | 5929.72 | 1630.41 | 34824.52 | 1133333.39 | 8398.92 | 846.77 |
| BLDR 030 | 57116.97 | 5147.43 | 2470.76 | 23969.38 | 589887.61 | 7610.42 | 817.97 |
| BLDR 031 | 49228.35 | 6900.45 | 4847.61 | 42949.71 | 1171874.48 | 7943.04 | 1097.34 |
| BLDR 032 | 31435.45 | 6238.55 | 2838.81 | 18554.85 | 728944.62 | 5319.88 | 527.98 |
| BLDR 033 | 35546.38 | 4674.99 | 1747.27 | 29408.37 | 451124.15 | 5627.77 | 468.37 |
| BLDR 034 | 48503.16 | 6033.51 | 1811.00 | 25804.44 | 970584.77 | 6170.13 | 153.43 |
| BLDR 035 | 38847.34 | 7017.11 | 3461.86 | 34401.60 | 1418768.58 | 8508.69 | 1834.61 |
| BLDR 036 | 64693.93 | 8465.74 | 3355.93 | 27880.04 | 1122638.15 | 11310.14 | 1139.10 |
| BLDR 037 | 52162.95 | 8457.84 | 1524.87 | 21993.73 | 707789.36 | 8798.92 | 1151.03 |
| BLDR 038 | 27697.90 | 6610.33 | 3048.99 | 32148.42 | 1680220.10 | 10088.57 | 772.33 |
| BLDR 039 | 42251.33 | 8347.23 | 980.21 | 27007.60 | 689509.13 | 14986.44 | 1780.85 |
| BLDR 040 | 26964.45 | 6779.13 | 1452.54 | 23914.66 | 1106708.75 | 7739.51 | 324.51 |
| BLDR 041 | 29630.58 | 4418.37 | 2265.93 | 13737.04 | 660708.89 | 5720.15 | 676.98 |
| BLDR 042 | 30753.95 | 5762.15 | 3076.38 | 16644.42 | 653808.75 | 9077.23 | 1268.24 |
| BLDR 043 | 31468.09 | 6113.87 | 1844.15 | 30780.04 | 1501358.89 | 8967.35 | 628.89 |
| BLDR 044 | 37101.20 | 8051.22 | 4621.91 | 32836.62 | 1088760.40 | 7821.46 | 998.17 |
| BLDR 045 | 37409.23 | 6128.58 | 1576.71 | 22172.06 | 422834.55 | 9667.79 | 827.92 |
| BLDR 046 | 32994.25 | 4033.99 | 1793.59 | 25020.24 | 998551.73 | 4996.07 | 239.60 |
| BLDR 047 | 51986.22 | 7331.60 | 2547.17 | 27425.79 | 1283199.28 | 11662.80 | 1286.47 |
| BLDR 048 | 19804.53 | 6607.09 | 2307.00 | 30142.42 | 470458.40 | 10580.26 | 1318.39 |
| PDAC 001 | 12287.00 | 4302.91 | 3071.87 | 23746.78 | 824302.67 | 4151.23 | 162.43 |
| PDAC 002 | 36738.44 | 6763.35 | 2103.43 | 24034.28 | 1007223.98 | 5491.50 | 786.83 |
| PDAC 003 | 18496.23 | 6043.25 | 2723.47 | 22071.88 | 434748.82 | 7131.90 | 1945.24 |
| PDAC 004 | 28859.40 | 5295.09 | 1024.48 | 28817.13 | 1175508.11 | 4385.17 | 530.18 |
| PDAC 005 | 44600.07 | 6896.16 | 3085.42 | 32284.54 | 645767.11 | 4338.07 | 130.30 |
| PDAC 006 | 59227.71 | 11.90 | 3395.13 | 27758.30 | 762692.74 | 7710.03 | 203.23 |
| PDAC 007 | 19075.05 | 4380.47 | 1934.42 | 17335.64 | 591714.42 | 6778.14 | 1834.61 |
| PDAC 008 | 76243.34 | 6298.45 | 3489.54 | 41104.24 | 1104230.67 | 8276.37 | 813.99 |
| PDAC 009 | 26191.02 | 4722.87 | 1423.01 | 26730.13 | 477349.16 | 3784.96 | 286.50 |
| PDAC 010 | 68959.93 | 7396.47 | 4303.76 | 28477.46 | 1216132.17 | 8657.16 | 582.39 |
| PDAC 011 | 59661.47 | 8913.07 | 2981.86 | 18323.24 | 634412.23 | 10915.36 | 1543.55 |
| PDAC 012 | 51496.07 | 6390.39 | 2464.68 | 23416.17 | 792898.19 | 5100.53 | 598.64 |
| PDAC 013 | 58189.15 | 6430.40 | 1786.06 | 24302.75 | 519279.06 | 6739.86 | 904.99 |
| PDAC 014 | 51425.68 | 4279.67 | 2295.90 | 20857.82 | 1065762.66 | 5160.05 | 829.87 |
| PDAC 015 | 73580.20 | 7698.10 | 2696.96 | 8432.32 | 873518.60 | 8187.73 | 902.71 |
| PDAC 016 | 51425.68 | 11.90 | 2050.40 | 15281.90 | 941838.20 | 5603.53 | 868.51 |
| PDAC 017 | 26860.88 | 4957.41 | 1942.82 | 21419.09 | 1316117.02 | 6452.69 | 1135.23 |
| PDAC 018 | 31124.57 | 5896.13 | 2630.89 | 17116.86 | 1056437.51 | 7481.20 | 764.24 |
| PDAC 019 | 57671.17 | 7231.27 | 735.39 | 30528.12 | 1122095.61 | 7131.90 | 207.34 |
| PDAC 020 | 48499.28 | 6502.47 | 3004.32 | 21836.90 | 219002.75 | 9780.67 | 2426.26 |
| PDAC 021 | 5920.40 | 5326.59 | 702.79 | 27753.86 | 791453.58 | 6463.44 | 1714.90 |
| PDAC 022 | 110769.32 | 5721.60 | 4823.37 | 35667.39 | 1121001.84 | 4894.02 | 316.08 |
| PDAC 023 | 50372.00 | 5674.07 | 1947.02 | 29208.60 | 1244156.97 | 6260.28 | 594.21 |
| PDAC 024 | 38726.69 | 6430.40 | 3121.58 | 18213.33 | 1243229.34 | 5840.31 | 918.70 |
| PDAC 025 | 22249.06 | 4039.98 | 2434.26 | 24429.23 | 586341.09 | 9067.36 | 1446.56 |
| PDAC 026 | 32318.85 | 7114.01 | 1182.41 | 18538.27 | 779653.15 | 5562.70 | 475.41 |
| PDAC 027 | 45672.83 | 5212.47 | 1769.44 | 36620.38 | 638741.93 | 3685.96 | 484.14 |
| PDAC 028 | 45287.23 | 8541.61 | 2186.53 | 29787.71 | 641070.19 | 8430.96 | 466.68 |
| PDAC 029 | 43900.80 | 4539.23 | 822.32 | 14701.98 | 611300.38 | 3128.10 | 259.87 |
| PDAC 030 | 72921.38 | 8065.48 | 3225.32 | 24984.28 | 1143176.67 | 10236.51 | 2243.53 |
| PDAC 031 | 27832.57 | 5862.18 | 1437.34 | 14963.57 | 1099830.14 | 6737.95 | 548.51 |
| PDAC 032 | 38694.24 | 11.90 | 710.32 | 30010.47 | 1056024.29 | 9619.13 | 782.44 |
| PDAC 033 | 71813.23 | 11.90 | 1948.84 | 27794.56 | 1210430.88 | 8263.25 | 613.20 |
| PDAC 034 | 52557.60 | 6056.89 | 2652.28 | 32590.99 | 1232333.80 | 7112.32 | 542.64 |
| PDAC 035 | 34595.35 | 5512.23 | 1259.54 | 23645.61 | 819047.15 | 10061.16 | 1674.49 |
| PDAC 036 | 38952.07 | 11.90 | 2563.30 | 29180.00 | 1079918.39 | 8159.84 | 1059.60 |
| PDAC 037 | 43217.84 | 8485.84 | 4242.71 | 23282.35 | 1179112.98 | 11026.57 | 2247.10 |
| PDAC 038 | 27869.34 | 5741.57 | 1697.20 | 28979.73 | 417667.32 | 6333.06 | 272.34 |
| PDAC 039 | 27795.82 | 3594.98 | 898.18 | 8871.82 | 429407.88 | 3905.04 | 132.51 |
| PDAC 040 | 29407.15 | 3849.03 | 650.85 | 23430.23 | 391893.76 | 3803.26 | 289.71 |
| PDAC 041 | 37756.20 | 4952.24 | 2294.48 | 21809.82 | 620446.75 | 8945.44 | 1906.08 |
| PDAC 042 | 57480.24 | 4901.33 | 3048.93 | 22031.65 | 771081.77 | 6617.17 | 756.04 |
| PDAC 043 | 44010.30 | 4806.53 | 1570.68 | 19442.76 | 783616.68 | 5480.54 | 98.44 |
| PDAC 044 | 25881.58 | 4269.30 | 1154.24 | 17329.21 | 813488.66 | 4129.22 | 124.92 |
| HC 045 | 20427.98 | 5691.01 | 1874.94 | 19387.18 | 289004.51 | 6269.83 | 285.85 |
| HC 046 | 33976.21 | 5248.26 | 762.36 | 17730.98 | 1005158.81 | 4956.66 | 178.24 |
| HC 047 | 51706.09 | 6660.60 | 1369.01 | 20843.50 | 1491433.43 | 7013.30 | 315.09 |
| HC 048 | 50785.62 | 6002.48 | 1353.78 | 20926.35 | 1431872.39 | 6880.13 | 379.82 |
| HC 049 | 33183.60 | 7101.68 | 5296.69 | 27534.17 | 504793.27 | 8466.52 | 641.03 |
| HC 050 | 24717.21 | 6687.49 | 743.30 | 21516.07 | 1273402.69 | 8012.99 | 126.15 |
| HC 051 | 22353.55 | 6490.69 | 789.78 | 21186.28 | 1546192.85 | 9955.05 | 367.17 |
| HC 052 | 36215.15 | 7592.45 | 6320.05 | 30588.31 | 611099.85 | 7864.43 | 249.98 |
| HC 053 | 47887.13 | 7231.27 | 1600.24 | 24766.70 | 858136.22 | 7606.72 | 576.49 |
| HC 054 | 64033.49 | 7683.86 | 3214.13 | 33649.93 | 1121566.60 | 8408.27 | 310.96 |
| HC 055 | 28130.81 | 7319.20 | 3468.97 | 12379.60 | 624021.42 | 6610.65 | 458.99 |
| HC 056 | 27727.36 | 7796.36 | 1674.43 | 16970.58 | 465825.84 | 5944.36 | 122.76 |
| HC 057 | 28475.89 | 5224.27 | 3344.67 | 29017.03 | 909945.36 | 7929.95 | 1786.66 |
| HC 058 | 30063.31 | 7074.56 | 6724.23 | 36571.68 | 1109799.59 | 7571.18 | 186.87 |
| HC 059 | 34258.87 | 7319.20 | 8007.33 | 36885.79 | 1579157.23 | 8642.43 | 315.09 |
| HC 060 | 37297.15 | 6158.77 | 1461.51 | 26178.70 | 538439.26 | 7311.37 | 440.59 |
| HC 061 | 62884.33 | 6885.13 | 3048.93 | 35006.45 | 752912.48 | 8612.99 | 348.30 |
| HC 062 | 47329.19 | 8625.31 | 6740.75 | 31829.93 | 1154663.18 | 6578.10 | 645.57 |
| HC 063 | 45714.60 | 9455.11 | 6962.90 | 32035.13 | 1173495.19 | 10810.96 | 809.90 |
| HC 064 | 22101.16 | 6286.46 | 1921.82 | 12724.97 | 518009.08 | 6302.85 | 798.14 |
| HC 065 | 23753.91 | 8420.87 | 3176.33 | 28296.28 | 1337237.84 | 7878.52 | 682.10 |
| HC 066 | 80434.98 | 9469.27 | 8501.46 | 33565.51 | 1499410.39 | 9750.67 | 723.55 |
| HC 067 | 35704.49 | 6550.56 | 535.15 | 18376.45 | 751411.20 | 4830.66 | 71.67 |
| HC 068 | 72032.06 | 6768.24 | 6408.38 | 30368.06 | 1128797.64 | 6333.03 | 365.07 |
| HC 069 | 57943.59 | 7885.52 | 3360.19 | 33113.76 | 673007.01 | 8554.26 | 506.97 |
| HC 070 | 54181.30 | 7002.30 | 5560.69 | 25104.03 | 1407769.68 | 6840.40 | 420.22 |
| HC 071 | 37236.05 | 6571.10 | 5994.81 | 35125.41 | 1123521.06 | 11093.36 | 1564.45 |
| HC 072 | 52235.68 | 5887.90 | 4881.54 | 24531.17 | 1115965.11 | 5848.54 | 286.38 |
| HC 073 | 33629.73 | 5747.30 | 5189.19 | 33056.22 | 1426989.91 | 9165.51 | 1311.66 |
| HC 074 | 32835.80 | 5791.19 | 4697.91 | 32416.63 | 1532146.95 | 10386.49 | 1345.02 |
| HC 075 | 34300.88 | 5967.19 | 5236.57 | 28530.72 | 1158245.74 | 6435.73 | 1561.80 |
| HC 076 | 40502.19 | 7083.60 | 4006.06 | 14999.30 | 1068571.23 | 7337.53 | 1569.75 |
| HC 077 | 97498.41 | 7720.46 | 5503.24 | 32977.85 | 387305.61 | 9287.20 | 2619.47 |
| HC 078 | 101360.68 | 7867.16 | 5439.54 | 32305.54 | 389083.86 | 10993.25 | 2885.97 |
| HC 079 | 41703.50 | 7310.12 | 4124.10 | 15123.81 | 1053558.83 | 10145.64 | 1598.99 |
| HC 080 | 85260.93 | 6678.52 | 4688.60 | 29915.12 | 377938.94 | 8205.91 | 2441.75 |
| HC 081 | 43407.83 | 7982.05 | 4484.71 | 15894.34 | 1258219.55 | 11354.23 | 1725.18 |
| HC 082 | 34749.62 | 7346.46 | 6036.92 | 26603.41 | 411875.83 | 10483.66 | 1162.27 |
| HC 083 | 53069.98 | 10205.59 | 1752.62 | 26280.87 | 821006.42 | 5512.25 | 65.13 |
| HC 084 | 36753.32 | 8737.13 | 5804.38 | 14386.67 | 1026223.01 | 10402.65 | 950.67 |
| HC 085 | 35833.63 | 7446.53 | 5862.36 | 14271.38 | 1067138.87 | 6695.59 | 1134.79 |
| HC 086 | 49598.31 | 6575.57 | 6767.21 | 27720.19 | 622117.01 | 9891.93 | 1258.10 |
| HC 087 | 38576.54 | 8774.46 | 6173.31 | 29987.06 | 451707.46 | 11160.37 | 695.88 |
| HC 088 | 32183.70 | 5646.55 | 4169.65 | 32543.48 | 768937.07 | 10305.88 | 1438.26 |
| HC 089 | 28428.76 | 6250.34 | 4206.16 | 21617.09 | 833421.91 | 9478.93 | 1612.32 |
| HC 090 | 29981.21 | 5233.12 | 4261.01 | 21083.87 | 917130.64 | 6578.10 | 1535.34 |
| HC 091 | 33239.31 | 7720.46 | 5535.15 | 28170.98 | 465418.74 | 10943.37 | 1240.34 |
| HC 092 | 30748.98 | 6090.84 | 4608.09 | 21778.67 | 900442.27 | 8716.23 | 1894.73 |
| HC 093 | 34048.92 | 6602.40 | 5484.12 | 32665.41 | 1267133.15 | 10893.62 | 2247.10 |
| HC 094 | 56448.08 | 6201.54 | 5516.00 | 24839.64 | 1191544.07 | 6800.77 | 695.88 |
| HC 095 | 53252.98 | 8629.11 | 3684.05 | 28671.09 | 624675.83 | 7834.57 | 415.67 |
| HC 096 | 58067.28 | 6786.20 | 5420.46 | 25811.92 | 1202440.56 | 8627.70 | 718.93 |
| HC 097 | 48341.81 | 5773.63 | 4414.02 | 22591.24 | 780094.12 | 4909.72 | 647.85 |
| HC 098 | 32219.97 | 5743.56 | 2994.58 | 29637.01 | 988063.59 | 9290.25 | 1300.79 |
| HC 099 | 22789.49 | 5814.92 | 2139.86 | 15512.49 | 518312.27 | 9911.81 | 1044.10 |
| HC 100 | 34098.67 | 4299.20 | 1169.46 | 30622.44 | 417092.83 | 7143.41 | 251.35 |
| HC 101 | 33838.26 | 3462.67 | 1400.47 | 30167.31 | 274546.73 | 4671.77 | 114.02 |
| HC 102 | 33708.17 | 3794.06 | 1364.98 | 32463.76 | 296915.54 | 5395.53 | 97.67 |
| HC 103 | 33682.16 | 4842.56 | 1719.53 | 31125.36 | 284800.59 | 7746.82 | 291.02 |
| HC 104 | 31380.27 | 5739.16 | 3649.51 | 34848.13 | 1133226.35 | 9332.93 | 1329.15 |
| HC 105 | 44416.04 | 6010.60 | 1948.14 | 29468.25 | 750846.01 | 5111.30 | 132.30 |
| HC 106 | 41100.39 | 5293.76 | 1616.33 | 26396.45 | 517281.53 | 4219.98 | 99.49 |
| HC 107 | 36772.94 | 5858.81 | 1515.56 | 26294.92 | 620656.89 | 4427.69 | 117.67 |
| HC 108 | 41006.33 | 5898.73 | 3329.33 | 29601.05 | 886748.44 | 9824.71 | 1222.78 |
| HC 109 | 40402.53 | 6018.60 | 1899.32 | 28022.56 | 900230.31 | 7883.29 | 987.61 |
| HC 110 | 41396.26 | 5675.40 | 3218.86 | 31274.29 | 1129417.12 | 9078.79 | 867.22 |
| HC 111 | 41382.80 | 6383.11 | 1806.13 | 29357.87 | 881844.20 | 7448.52 | 843.25 |
| HC 112 | 43219.88 | 6672.53 | 3378.06 | 32196.36 | 1277496.83 | 9713.17 | 995.67 |
| HC 113 | 53595.20 | 8997.71 | 1977.06 | 32024.31 | 504970.40 | 8179.41 | 292.51 |

| Subject | | | | | | | |
|---|---|---|---|---|---|---|---|
| Cohort | | sc- | | | | | |
| ID | sVEGFR3 | kit/SCFR | CA 125 | CA 15-3 | CA 19-9 | CEA | FGF2 |
| OVAR 001 | 1342.36 | 411.09 | 2.09 | 0.08 | 1.05 | 5.20 | 3.60 |
| OVAR 002 | 1025.29 | 333.61 | 0.20 | 0.04 | 4.44 | 5.20 | 3.60 |
| OVAR 003 | 1104.11 | 228.26 | 0.20 | 0.12 | 0.81 | 5.20 | 3.60 |
| OVAR 004 | 233.00 | 53.12 | 10.78 | 0.12 | 1.19 | 5.20 | 12.15 |
| OVAR 005 | 643.65 | 263.99 | 4.41 | 0.53 | 0.30 | 8.52 | 3.60 |
| OVAR 006 | 233.00 | 249.04 | 1.32 | 0.28 | 2.25 | 5.20 | 3.60 |
| OVAR 007 | 473.59 | 347.00 | 11.92 | 0.05 | 71.06 | 5.20 | 6.65 |
| OVAR 008 | 233.00 | 81.03 | 78.03 | 2.09 | 95.71 | 5.20 | 6.65 |
| OVAR 009 | 233.00 | 140.17 | 27.09 | 32.70 | 22.23 | 24.36 | 9.27 |
| OVAR 010 | 242.17 | 47.44 | 0.70 | 0.03 | 2.24 | 5.20 | 11.02 |
| OVAR 011 | 233.00 | 99.29 | 7.71 | 0.14 | 3.00 | 5.20 | 12.15 |
| OVAR 012 | 233.00 | 149.01 | 39.43 | 0.16 | 82.74 | 5.20 | 3.60 |
| OVAR 013 | 233.00 | 785.74 | 8.64 | 0.24 | 1.07 | 9.17 | 3.60 |
| OVAR 014 | 233.00 | 226.88 | 37.79 | 2.07 | 127.18 | 9.57 | 10.31 |
| OVAR 015 | 233.00 | 164.22 | 18.97 | 0.92 | 3182.93 | 11.43 | 9.25 |
| OVAR 016 | 1193.88 | 149.55 | 3.17 | 0.20 | 12.69 | 8.42 | 3.60 |
| OVAR 017 | 1521.92 | 175.17 | 10.62 | 0.66 | 8.72 | 5.20 | 3.60 |
| OVAR 018 | 1429.66 | 186.30 | 9.65 | 0.50 | 16.36 | 5.84 | 3.60 |
| OVAR 019 | 653.92 | 231.07 | 3.90 | 1.02 | 5.05 | 5.20 | 10.24 |
| OVAR 020 | 973.37 | 87.35 | 4.87 | 0.03 | 28.32 | 5.20 | 3.60 |
| OVAR 021 | 1740.51 | 30.50 | 1.06 | 0.03 | 0.89 | 5.20 | 10.24 |
| OVAR 022 | 1548.34 | 30.50 | 1.49 | 0.27 | 5.29 | 5.20 | 8.12 |
| OVAR 023 | 1800.37 | 351.60 | 10.45 | 0.12 | 13.20 | 8.85 | 8.12 |
| OVAR 024 | 1083.33 | 364.47 | 1.18 | 0.41 | 3.53 | 5.20 | 11.63 |
| OVAR 025 | 1807.03 | 325.20 | 16.89 | 0.37 | 27.48 | 13.95 | 8.12 |
| OVAR 026 | 921.84 | 971.04 | 28.19 | 0.13 | 24.89 | 9.41 | 14.36 |
| OVAR 027 | 410.05 | 567.59 | 51.08 | 2.20 | 17.50 | 12.41 | 10.98 |
| OVAR 028 | 284.17 | 74.03 | 13.26 | 0.57 | 6.63 | 10.53 | 10.81 |
| OVAR 029 | 473.84 | 69.37 | 24.50 | 0.76 | 205.88 | 5.20 | 9.65 |
| OVAR 030 | 323.70 | 317.49 | 18.21 | 0.62 | 14.50 | 5.20 | 7.90 |
| OVAR 031 | 233.00 | 761.88 | 2.12 | 0.19 | 1.80 | 5.20 | 9.26 |
| OVAR 032 | 793.67 | 86.00 | 0.56 | 0.03 | 0.48 | 5.20 | 3.60 |
| OVAR 033 | 1218.87 | 186.94 | 0.20 | 0.09 | 14.29 | 5.20 | 3.60 |
| OVAR 034 | 451.03 | 286.62 | 5.97 | 0.10 | 17.61 | 7.51 | 10.98 |
| OVAR 035 | 267.40 | 433.51 | 4.36 | 0.40 | 1880.47 | 85.72 | 4.60 |
| OVAR 036 | 301.05 | 178.93 | 98.12 | 1.17 | 5.82 | 10.61 | 4.60 |
| OVAR 037 | 233.00 | 143.37 | 15.96 | 0.10 | 3.12 | 5.20 | 9.25 |
| OVAR 038 | 782.87 | 52.95 | 25.96 | 0.24 | 28.66 | 62.84 | 4.60 |
| OVAR 039 | 233.00 | 51.69 | 36.37 | 2.14 | 74.21 | 5.20 | 12.15 |
| OVAR 040 | 321.66 | 160.97 | 0.63 | 0.24 | 0.69 | 5.20 | 3.60 |
| OVAR 041 | 835.21 | 133.99 | 2.40 | 0.05 | 9.65 | 5.20 | 15.74 |
| OVAR 042 | 812.39 | 81.92 | 7.06 | 1.53 | 38.22 | 23.89 | 46.17 |
| OVAR 043 | 464.43 | 38.14 | 34.68 | 0.09 | 49.28 | 6.20 | 15.74 |
| OVAR 044 | 934.28 | 202.96 | 1.11 | 1.37 | 2.66 | 128.81 | 41.44 |
| BLDR 001 | 233.00 | 839.21 | 0.70 | 0.50 | 11.46 | 9.93 | 6.00 |
| BLDR 002 | 1082.58 | 152.02 | 0.28 | 0.17 | 0.71 | 5.20 | 9.65 |
| BLDR 003 | 233.00 | 205.81 | 0.28 | 0.19 | 1.73 | 5.20 | 6.00 |
| BLDR 004 | 312.35 | 130.59 | 2.36 | 0.13 | 133.15 | 10.93 | 6.00 |
| BLDR 005 | 233.00 | 730.15 | 0.39 | 0.70 | 2.39 | 20.10 | 6.00 |
| BLDR 006 | 451.03 | 410.08 | 0.45 | 0.42 | 1.84 | 9.86 | 6.00 |
| BLDR 007 | 786.51 | 66.93 | 5.93 | 1.40 | 36.01 | 22037.96 | 12.10 |
| BLDR 008 | 233.00 | 257.65 | 0.20 | 0.27 | 2.90 | 5.20 | 13.72 |
| BLDR 009 | 382.18 | 30.50 | 1.49 | 0.21 | 0.30 | 5.20 | 11.68 |
| BLDR 010 | 233.00 | 116.38 | 3.43 | 0.06 | 0.30 | 5.20 | 16.27 |
| BLDR 011 | 520.80 | 230.36 | 0.23 | 0.05 | 0.39 | 5.20 | 9.65 |
| BLDR 012 | 261.09 | 1519.26 | 1.86 | 0.11 | 0.47 | 5.20 | 3.60 |
| BLDR 013 | 394.54 | 977.41 | 1.00 | 0.29 | 0.54 | 5.20 | 8.52 |
| BLDR 014 | 306.00 | 184.91 | 1.17 | 0.03 | 0.43 | 5.20 | 3.60 |
| BLDR 015 | 630.77 | 215.24 | 0.55 | 0.10 | 0.30 | 9.96 | 7.60 |
| BLDR 016 | 1003.10 | 137.25 | 0.90 | 0.05 | 0.39 | 5.20 | 6.56 |
| BLDR 017 | 572.52 | 437.49 | 0.76 | 0.22 | 0.85 | 5.20 | 13.72 |
| BLDR 018 | 1179.90 | 343.17 | 1.21 | 0.25 | 2.81 | 29.86 | 6.56 |
| BLDR 019 | 233.00 | 408.17 | 0.63 | 0.05 | 2.04 | 5.20 | 10.31 |
| BLDR 020 | 351.03 | 161.17 | 2.54 | 0.18 | 1.64 | 5.20 | 13.48 |
| BLDR 021 | 427.56 | 119.34 | 0.78 | 0.58 | 5.27 | 20.14 | 6.56 |
| BLDR 022 | 233.00 | 454.35 | 0.53 | 0.08 | 0.33 | 5.20 | 3.60 |
| BLDR 023 | 453.46 | 303.15 | 0.23 | 0.06 | 0.30 | 5.20 | 3.60 |
| BLDR 024 | 515.69 | 139.21 | 0.61 | 0.09 | 0.30 | 5.20 | 3.60 |
| BLDR 025 | 233.00 | 194.66 | 0.84 | 0.16 | 0.30 | 5.20 | 3.78 |
| BLDR 026 | 653.58 | 238.48 | 0.79 | 0.21 | 2.24 | 5.20 | 13.72 |
| BLDR 027 | 233.00 | 168.18 | 0.67 | 0.16 | 3.34 | 5.20 | 8.52 |
| BLDR 028 | 2762.42 | 167.38 | 2.46 | 0.04 | 0.30 | 5.20 | 8.52 |
| BLDR 029 | 637.34 | 30.50 | 1.43 | 0.07 | 0.47 | 5.20 | 13.72 |
| BLDR 030 | 1444.17 | 187.24 | 0.87 | 0.12 | 1.09 | 5.20 | 9.65 |
| BLDR 031 | 1068.24 | 259.22 | 0.83 | 0.19 | 13.76 | 49.86 | 9.65 |
| BLDR 032 | 727.97 | 472.63 | 0.20 | 0.14 | 0.30 | 5.20 | 10.07 |
| BLDR 033 | 251.06 | 129.00 | 1.13 | 1.56 | 5.56 | 40.07 | 13.63 |
| BLDR 034 | 233.00 | 116.38 | 0.20 | 0.03 | 9.38 | 5.20 | 13.63 |
| BLDR 035 | 336.26 | 30.50 | 0.77 | 0.20 | 3.25 | 5.20 | 9.65 |
| BLDR 036 | 2688.94 | 329.48 | 0.28 | 0.07 | 0.84 | 5.20 | 3.60 |
| BLDR 037 | 233.00 | 514.06 | 2.36 | 1.34 | 1156.00 | 5.20 | 9.65 |
| BLDR 038 | 1175.93 | 60.18 | 0.36 | 0.03 | 10.86 | 7.33 | 15.74 |
| BLDR 039 | 570.53 | 138.56 | 1.44 | 0.10 | 2.16 | 6.90 | 11.02 |
| BLDR 040 | 3091.45 | 135.77 | 0.43 | 0.14 | 0.48 | 7.30 | 3.60 |
| BLDR 041 | 1687.24 | 30.50 | 12.61 | 0.29 | 16.87 | 7.10 | 15.74 |
| BLDR 042 | 1996.94 | 127.50 | 2.17 | 0.16 | 1.59 | 7.10 | 15.74 |
| BLDR 043 | 1494.29 | 756.12 | 2.98 | 0.32 | 0.93 | 10.79 | 15.74 |
| BLDR 044 | 675.74 | 156.67 | 0.80 | 1.74 | 5.17 | 31.48 | 50.60 |
| BLDR 045 | 1048.86 | 123.17 | 0.89 | 0.47 | 0.93 | 59.43 | 20.20 |
| BLDR 046 | 369.49 | 570.42 | 2.01 | 0.21 | 3.37 | 5.20 | 3.60 |
| BLDR 047 | 407.06 | 170.98 | 0.24 | 0.35 | 1.22 | 5.20 | 9.65 |
| BLDR 048 | 233.00 | 30.50 | 0.78 | 0.11 | 3.61 | 5.20 | 9.65 |
| PDAC 001 | 618.60 | 114.36 | 0.54 | 0.46 | 2.08 | 5.20 | 3.60 |
| PDAC 002 | 233.00 | 681.33 | 0.75 | 0.08 | 20.62 | 5.20 | 3.60 |
| PDAC 003 | 546.11 | 123.08 | 1.37 | 4.78 | 154.64 | 46.23 | 18.60 |
| PDAC 004 | 233.00 | 87.07 | 2.98 | 0.08 | 12.26 | 15.14 | 6.65 |
| PDAC 005 | 522.17 | 262.65 | 1.18 | 0.06 | 5.16 | 5.20 | 4.64 |
| PDAC 006 | 992.73 | 164.76 | 1.27 | 0.13 | 12.49 | 22.79 | 3.60 |
| PDAC 007 | 269.65 | 151.62 | 0.68 | 0.32 | 1702.03 | 10.49 | 4.64 |
| PDAC 008 | 967.32 | 134.69 | 1.85 | 0.17 | 221.51 | 15.92 | 7.90 |
| PDAC 009 | 342.58 | 173.78 | 2.22 | 0.05 | 86.11 | 5.20 | 10.98 |
| PDAC 010 | 360.92 | 334.26 | 0.49 | 0.08 | 5.56 | 5.20 | 3.60 |
| PDAC 011 | 2459.74 | 109.42 | 1.19 | 0.13 | 16.98 | 7.58 | 10.98 |
| PDAC 012 | 492.81 | 236.23 | 3.63 | 0.32 | 2.30 | 5.20 | 16.65 |
| PDAC 013 | 261.84 | 1269.88 | 3.47 | 0.16 | 74.18 | 5.20 | 6.00 |
| PDAC 014 | 233.00 | 53.12 | 1.56 | 0.06 | 119.25 | 5.20 | 6.65 |
| PDAC 015 | 1176.21 | 477.66 | 2.56 | 0.03 | 3.27 | 5.20 | 7.90 |
| PDAC 016 | 783.86 | 170.77 | 0.84 | 0.19 | 385.41 | 50.70 | 11.02 |
| PDAC 017 | 687.91 | 87.22 | 0.20 | 0.21 | 3.55 | 5.20 | 9.44 |
| PDAC 018 | 785.97 | 104.34 | 0.98 | 0.12 | 2.55 | 10.42 | 9.44 |
| PDAC 019 | 233.00 | 294.63 | 4.56 | 0.80 | 84.05 | 5.20 | 9.44 |
| PDAC 020 | 942.11 | 269.29 | 0.62 | 0.18 | 107.15 | 13.02 | 6.00 |
| PDAC 021 | 641.28 | 67.20 | 1.03 | 0.14 | 13.41 | 5.20 | 9.20 |
| PDAC 022 | 1246.08 | 569.73 | 2.97 | 0.15 | 258.33 | 5.20 | 9.44 |
| PDAC 023 | 819.22 | 279.04 | 1.38 | 0.38 | 10.01 | 8.96 | 9.25 |
| PDAC 024 | 1742.13 | 90.66 | 2.12 | 0.29 | 25.16 | 5.20 | 9.25 |
| PDAC 025 | 233.00 | 343.17 | 2.06 | 0.08 | 72.02 | 7.04 | 9.25 |
| PDAC 026 | 1735.59 | 30.50 | 2.68 | 0.08 | 1.35 | 17.70 | 9.25 |
| PDAC 027 | 510.24 | 249.41 | 0.40 | 0.16 | 5.74 | 6.67 | 4.13 |
| PDAC 028 | 233.00 | 625.43 | 3.22 | 0.31 | 48.47 | 5.20 | 13.63 |
| PDAC 029 | 609.88 | 372.05 | 0.20 | 0.54 | 3.76 | 24.00 | 11.02 |
| PDAC 030 | 644.55 | 270.57 | 1.32 | 0.41 | 68.16 | 5.20 | 12.43 |
| PDAC 031 | 233.00 | 346.11 | 1.02 | 0.38 | 1.43 | 5.20 | 3.60 |
| PDAC 032 | 233.00 | 358.61 | 3.05 | 0.42 | 6.95 | 9.03 | 16.27 |
| PDAC 033 | 233.00 | 67.64 | 0.80 | 0.13 | 175.48 | 5.20 | 14.21 |
| PDAC 034 | 512.12 | 1015.07 | 0.77 | 0.45 | 211.04 | 29.87 | 13.48 |
| PDAC 035 | 233.00 | 111.69 | 0.72 | 0.06 | 40.52 | 5.20 | 3.60 |
| PDAC 036 | 351.03 | 263.66 | 0.64 | 0.05 | 13.77 | 5.20 | 3.60 |
| PDAC 037 | 381.17 | 170.90 | 3.78 | 0.08 | 134.39 | 5.20 | 9.65 |
| PDAC 038 | 314.23 | 684.92 | 0.59 | 0.17 | 0.93 | 5.20 | 3.60 |
| PDAC 039 | 233.00 | 142.57 | 3.52 | 0.30 | 18.57 | 5.20 | 3.60 |
| PDAC 040 | 233.00 | 50.27 | 1.03 | 0.22 | 4.41 | 5.20 | 3.60 |
| PDAC 041 | 1903.91 | 30.50 | 0.69 | 0.19 | 0.30 | 5.20 | 15.74 |
| PDAC 042 | 233.00 | 109.56 | 0.67 | 0.09 | 1.39 | 5.20 | 9.65 |
| PDAC 043 | 233.00 | 117.95 | 1.23 | 0.17 | 730.63 | 17.30 | 16.65 |
| PDAC 044 | 233.00 | 81.25 | 0.50 | 0.23 | 7.73 | 5.20 | 3.60 |
| HC 001 | 2003.15 | 30.50 | 2.15 | 0.13 | 0.98 | 5.20 | 11.02 |
| HC 002 | 622.76 | 159.04 | 0.45 | 0.07 | 0.30 | 5.20 | 3.60 |
| HC 003 | 1678.31 | 943.53 | 1.18 | 0.25 | 1.82 | 5.20 | 24.40 |
| HC 004 | 1442.83 | 387.40 | 0.20 | 0.54 | 2.65 | 5.33 | 24.40 |
| HC 005 | 2887.75 | 193.53 | 1.35 | 0.42 | 3.79 | 5.67 | 24.40 |
| HC 006 | 748.66 | 869.14 | 0.88 | 0.05 | 0.30 | 5.20 | 3.60 |
| HC 007 | 1914.25 | 124.85 | 1.93 | 0.25 | 2.51 | 5.20 | 3.60 |
| HC 008 | 1973.49 | 194.31 | 0.95 | 0.13 | 1.13 | 5.20 | 41.13 |
| HC 009 | 1069.96 | 211.32 | 1.64 | 0.10 | 0.30 | 5.20 | 3.60 |
| HC 010 | 4026.15 | 217.18 | 0.87 | 0.08 | 1.30 | 5.20 | 24.40 |
| HC 011 | 233.00 | 486.24 | 1.58 | 0.50 | 0.68 | 8.96 | 11.49 |
| HC 012 | 629.72 | 924.29 | 0.84 | 0.10 | 0.77 | 5.20 | 24.40 |
| HC 013 | 484.49 | 1048.41 | 0.33 | 0.23 | 0.77 | 6.98 | 24.40 |
| HC 014 | 1168.83 | 481.82 | 0.51 | 0.16 | 0.77 | 5.20 | 24.40 |
| HC 015 | 546.48 | 174.68 | 0.57 | 0.17 | 0.77 | 5.20 | 24.40 |
| HC 016 | 2766.81 | 214.02 | 0.33 | 0.11 | 0.40 | 8.26 | 24.40 |
| HC 017 | 233.00 | 1263.86 | 0.20 | 0.13 | 0.30 | 5.20 | 9.29 |
| HC 018 | 1899.46 | 263.99 | 0.39 | 0.10 | 0.30 | 5.82 | 24.40 |
| HC 019 | 484.49 | 66.73 | 0.21 | 0.08 | 0.30 | 5.20 | 24.40 |
| HC 020 | 567.23 | 1773.72 | 0.49 | 0.06 | 0.40 | 5.20 | 24.40 |
| HC 021 | 734.61 | 430.56 | 0.54 | 0.10 | 0.98 | 5.20 | 3.60 |
| HC 022 | 911.23 | 360.17 | 0.53 | 0.15 | 0.98 | 5.20 | 6.65 |
| HC 023 | 996.71 | 256.83 | 1.05 | 0.12 | 1.14 | 5.20 | 11.02 |
| HC 024 | 769.77 | 285.53 | 1.23 | 0.10 | 1.14 | 5.20 | 6.65 |
| HC 025 | 1128.03 | 490.42 | 4.03 | 0.64 | 0.92 | 21.39 | 6.00 |
| HC 026 | 1364.14 | 166.85 | 0.20 | 0.06 | 0.81 | 5.20 | 8.06 |
| HC 027 | 768.16 | 180.73 | 1.16 | 0.12 | 20.08 | 30.69 | 3.60 |
| HC 028 | 233.00 | 178.93 | 1.92 | 0.10 | 0.30 | 12.23 | 3.60 |
| HC 029 | 1272.23 | 439.20 | 1.44 | 0.09 | 0.30 | 5.20 | 3.60 |
| HC 030 | 233.00 | 30.50 | 1.23 | 0.20 | 1.37 | 8.47 | 24.02 |
| HC 031 | 233.00 | 30.50 | 2.83 | 0.22 | 0.30 | 19.92 | 24.02 |
| HC 032 | 494.51 | 136.15 | 2.52 | 0.14 | 0.30 | 8.47 | 15.74 |
| HC 033 | 1041.21 | 224.44 | 0.47 | 0.91 | 0.93 | 5.20 | 19.88 |
| HC 034 | 1248.11 | 288.71 | 0.36 | 0.09 | 0.30 | 8.01 | 15.74 |
| HC 035 | 842.82 | 459.26 | 0.97 | 3.08 | 3.08 | 12.20 | 30.60 |
| HC 036 | 873.36 | 418.71 | 1.07 | 0.91 | 2.43 | 11.94 | 37.31 |
| HC 037 | 2916.21 | 301.54 | 1.03 | 0.12 | 0.48 | 12.91 | 24.02 |
| HC 038 | 873.29 | 590.90 | 1.88 | 1.26 | 1.81 | 132.32 | 41.44 |
| HC 039 | 675.74 | 133.99 | 0.58 | 0.09 | 1.37 | 6.65 | 24.02 |
| HC 040 | 233.00 | 176.32 | 0.20 | 0.12 | 0.30 | 5.20 | 13.72 |
| HC 041 | 2276.58 | 288.71 | 0.36 | 0.03 | 0.30 | 5.20 | 15.74 |
| HC 042 | 233.00 | 30.50 | 0.20 | 0.03 | 0.47 | 5.20 | 13.72 |
| HC 043 | 233.00 | 143.19 | 0.89 | 0.07 | 1.23 | 5.20 | 8.85 |
| HC 044 | 636.86 | 487.63 | 0.57 | 0.14 | 0.57 | 5.20 | 10.24 |
| HC 045 | 233.00 | 378.55 | 0.54 | 0.11 | 0.30 | 5.20 | 4.60 |
| HC 046 | 847.39 | 367.64 | 1.92 | 0.20 | 3.08 | 6.77 | 3.60 |
| HC 047 | 691.97 | 232.27 | 1.79 | 0.25 | 3.49 | 15.36 | 3.60 |
| HC 048 | 1057.10 | 160.98 | 1.61 | 0.19 | 2.97 | 6.30 | 3.60 |
| HC 049 | 323.38 | 30.89 | 1.60 | 0.09 | 0.58 | 5.20 | 3.60 |
| HC 050 | 284.17 | 55.04 | 4.45 | 0.38 | 1.31 | 5.60 | 3.60 |
| HC 051 | 278.57 | 681.33 | 3.84 | 0.57 | 1.98 | 10.35 | 3.60 |
| HC 052 | 630.77 | 421.09 | 0.20 | 0.36 | 4.10 | 5.20 | 3.60 |
| HC 053 | 1259.56 | 175.01 | 3.38 | 0.17 | 1.44 | 7.77 | 13.60 |
| HC 054 | 392.61 | 704.27 | 2.52 | 0.20 | 0.97 | 9.09 | 13.72 |
| HC 055 | 415.88 | 531.00 | 2.52 | 1.07 | 3.40 | 20.96 | 12.35 |
| HC 056 | 355.95 | 805.08 | 5.07 | 0.29 | 1.22 | 5.25 | 13.72 |
| HC 057 | 1198.41 | 197.13 | 1.46 | 0.20 | 0.63 | 5.20 | 10.98 |
| HC 058 | 234.19 | 1084.45 | 2.08 | 0.05 | 2.03 | 5.20 | 9.44 |
| HC 059 | 233.00 | 171.22 | 3.97 | 0.49 | 4.96 | 8.74 | 3.60 |
| HC 060 | 335.98 | 736.60 | 1.25 | 0.10 | 1.29 | 9.68 | 7.90 |
| HC 061 | 1037.84 | 128.08 | 1.02 | 0.13 | 1.53 | 10.12 | 14.36 |
| HC 062 | 1199.41 | 160.98 | 1.15 | 0.29 | 1.54 | 6.64 | 4.13 |
| HC 063 | 381.03 | 480.67 | 1.00 | 0.31 | 1.66 | 6.72 | 12.57 |
| HC 064 | 600.80 | 667.66 | 2.81 | 0.82 | 3.34 | 18.93 | 19.17 |
| HC 065 | 233.00 | 326.93 | 0.20 | 0.03 | 0.30 | 5.20 | 3.60 |
| HC 066 | 267.40 | 158.43 | 0.20 | 0.03 | 0.30 | 5.20 | 3.60 |
| HC 067 | 936.84 | 291.05 | 1.10 | 0.11 | 5.57 | 22.51 | 14.36 |
| HC 068 | 278.57 | 359.47 | 9.70 | 0.21 | 1.07 | 8.77 | 9.25 |
| HC 069 | 1102.80 | 167.54 | 2.45 | 0.12 | 0.76 | 5.20 | 8.65 |
| HC 070 | 233.00 | 113.25 | 2.61 | 0.26 | 0.70 | 19.45 | 17.72 |
| HC 071 | 925.45 | 431.37 | 0.49 | 0.03 | 0.48 | 5.20 | 6.99 |
| HC 072 | 233.00 | 694.58 | 1.56 | 0.85 | 1.06 | 26.07 | 19.44 |
| HC 073 | 463.93 | 188.81 | 0.39 | 0.03 | 0.48 | 5.20 | 6.99 |
| HC 074 | 341.65 | 440.30 | 1.65 | 0.05 | 0.93 | 5.20 | 6.99 |
| HC 075 | 288.02 | 766.74 | 2.19 | 0.11 | 1.39 | 5.20 | 6.99 |
| HC 076 | 233.00 | 193.28 | 1.51 | 0.18 | 1.51 | 5.20 | 11.18 |
| HC 077 | 546.48 | 75.04 | 0.98 | 0.03 | 0.93 | 5.20 | 6.99 |
| HC 078 | 233.00 | 103.27 | 1.03 | 0.03 | 1.03 | 5.20 | 8.72 |
| HC 079 | 233.00 | 180.85 | 0.74 | 0.14 | 0.94 | 5.20 | 9.40 |
| HC 080 | 1700.35 | 134.14 | 2.07 | 0.05 | 0.95 | 5.20 | 12.15 |
| HC 081 | 233.00 | 30.50 | 1.56 | 0.17 | 1.29 | 5.20 | 8.65 |
| HC 082 | 1082.58 | 306.33 | 0.80 | 0.03 | 0.48 | 5.20 | 4.37 |
| HC 083 | 233.00 | 54.56 | 0.63 | 0.04 | 1.51 | 5.20 | 4.46 |
| HC 084 | 233.00 | 996.90 | 8.30 | 0.82 | 3.31 | 29.76 | 15.72 |
| HC 085 | 657.60 | 299.92 | 2.77 | 0.23 | 0.76 | 5.20 | 3.60 |
| HC 086 | 233.00 | 94.68 | 0.34 | 0.19 | 1.51 | 5.20 | 8.65 |
| HC 087 | 1469.17 | 76.58 | 1.06 | 0.06 | 0.57 | 5.20 | 6.99 |
| HC 088 | 546.48 | 154.36 | 0.58 | 0.03 | 0.57 | 5.20 | 6.99 |
| HC 089 | 795.54 | 122.67 | 2.59 | 0.13 | 1.04 | 5.20 | 8.65 |
| HC 090 | 233.00 | 491.73 | 2.95 | 0.19 | 0.58 | 5.20 | 8.65 |
| HC 091 | 2450.75 | 284.73 | 0.87 | 0.05 | 0.30 | 5.20 | 3.60 |
| HC 092 | 233.00 | 30.50 | 3.11 | 0.19 | 0.58 | 5.20 | 4.46 |
| HC 093 | 1648.93 | 127.17 | 0.76 | 0.10 | 0.30 | 5.20 | 8.28 |
| HC 094 | 233.00 | 175.91 | 2.26 | 0.40 | 0.70 | 21.11 | 11.18 |
| HC 095 | 427.72 | 328.13 | 3.03 | 0.18 | 0.47 | 16.19 | 13.72 |
| HC 096 | 233.00 | 1730.19 | 2.13 | 0.55 | 0.68 | 24.46 | 6.00 |
| HC 097 | 233.00 | 531.00 | 2.24 | 0.18 | 0.30 | 14.00 | 3.60 |
| HC 098 | 953.92 | 322.36 | 1.18 | 0.09 | 0.39 | 5.20 | 8.28 |
| HC 099 | 233.00 | 901.52 | 1.98 | 0.16 | 0.30 | 7.00 | 6.00 |
| HC 100 | 2271.13 | 202.98 | 0.69 | 0.27 | 0.76 | 7.03 | 8.28 |
| HC 101 | 1075.41 | 275.15 | 1.33 | 6.69 | 72.30 | 96.43 | 166.83 |
| HC 102 | 463.93 | 887.50 | 1.18 | 1.99 | 9.91 | 42.16 | 122.56 |
| HC 103 | 615.80 | 117.13 | 1.32 | 0.13 | 0.76 | 5.20 | 3.60 |
| HC 104 | 395.74 | 184.10 | 1.40 | 0.05 | 0.83 | 5.20 | 19.06 |
| HC 105 | 323.70 | 430.75 | 1.91 | 0.06 | 0.30 | 5.20 | 3.60 |
| HC 106 | 301.05 | 384.02 | 1.57 | 0.10 | 0.33 | 5.20 | 3.60 |
| HC 107 | 335.09 | 217.85 | 3.34 | 0.05 | 0.74 | 5.20 | 3.60 |
| HC 108 | 233.00 | 2479.31 | 0.57 | 0.08 | 1.20 | 13.53 | 3.60 |
| HC 109 | 1553.68 | 188.81 | 0.35 | 0.67 | 0.61 | 23.08 | 29.83 |
| HC 110 | 1140.04 | 340.04 | 0.78 | 0.24 | 1.93 | 51.63 | 8.28 |
| HC 111 | 1751.86 | 250.47 | 0.22 | 0.11 | 0.30 | 5.20 | 3.60 |
| HC 112 | 671.57 | 233.01 | 0.50 | 0.10 | 0.98 | 19.59 | 3.60 |
| HC 113 | 233.00 | 308.89 | 4.08 | 0.10 | 0.85 | 5.20 | 13.72 |
| OVAR 001 | 3491.05 | 26911.82 | 135.31 | 6.95 | 75.32 | 477.86 | 82.30 |
| OVAR 002 | 13284.49 | 23829.48 | 33.36 | 3.42 | 9.06 | 532.00 | 123.09 |
| OVAR 003 | 17065.04 | 37306.84 | 16.21 | 15.19 | 29.12 | 269.28 | 82.30 |
| OVAR 004 | 9830.14 | 13421.94 | 5.17 | 6.81 | 7.73 | 262.73 | 125.93 |
| OVAR 005 | 1399.94 | 19869.15 | 24.36 | 27.53 | 7.35 | 707.66 | 82.30 |
| OVAR 006 | 2890.65 | 27217.54 | 12.95 | 15.55 | 43.79 | 146.49 | 141.82 |
| OVAR 007 | 8961.62 | 43602.74 | 154.95 | 7.11 | 95.21 | 109.57 | 72.89 |
| OVAR 008 | 23414.78 | 26023.22 | 2235.84 | 54.80 | 2184.60 | 192.88 | 97.63 |
| OVAR 009 | 12234.80 | 15218.78 | 383.71 | 124.53 | 231.88 | 2819.27 | 182.10 |
| OVAR 010 | 11361.26 | 21799.09 | 3.70 | 2.28 | 41.70 | 195.28 | 141.82 |
| OVAR 011 | 4393.17 | 19370.71 | 99.17 | 9.52 | 23.86 | 286.27 | 162.64 |
| OVAR 012 | 4057.41 | 31185.73 | 76.68 | 11.19 | 69.52 | 340.89 | 67.09 |
| OVAR 013 | 14471.74 | 22664.41 | 50.60 | 6.76 | 16.50 | 336.24 | 116.32 |
| OVAR 014 | 14135.29 | 15492.26 | 513.62 | 30.93 | 2512.62 | 481.20 | 175.20 |
| OVAR 015 | 2474.98 | 15736.96 | 293.78 | 8.40 | 5237.94 | 1053.29 | 84.24 |
| OVAR 016 | 6762.13 | 21253.54 | 23.30 | 18.36 | 476.73 | 1048.47 | 213.60 |
| OVAR 017 | 5438.60 | 30.50 | 139.17 | 37.05 | 196.39 | 511.82 | 269.86 |
| OVAR 018 | 2260.34 | 28928.24 | 158.62 | 42.34 | 306.04 | 1104.21 | 269.86 |
| OVAR 019 | 12824.57 | 26251.88 | 46.37 | 43.91 | 58.86 | 111.98 | 24.67 |
| OVAR 020 | 19178.42 | 16234.06 | 46.62 | 4.96 | 1349.79 | 1125.24 | 332.58 |
| OVAR 021 | 6453.06 | 19156.01 | 3.33 | 2.80 | 13.35 | 127.84 | 94.71 |
| OVAR 022 | 6170.82 | 16212.24 | 17.16 | 20.94 | 210.70 | 456.15 | 57.13 |
| OVAR 023 | 5453.35 | 15863.35 | 57.34 | 8.50 | 70.45 | 1758.02 | 255.25 |
| OVAR 024 | 6515.89 | 22682.17 | 3.28 | 19.30 | 74.08 | 268.43 | 57.13 |
| OVAR 025 | 6390.27 | 25561.88 | 108.74 | 15.97 | 527.93 | 1582.38 | 68.09 |
| OVAR 026 | 12300.25 | 33225.34 | 404.02 | 9.43 | 117.58 | 2365.90 | 115.03 |
| OVAR 027 | 7780.22 | 7286.01 | 251.44 | 23.79 | 111.50 | 279.08 | 116.44 |
| OVAR 028 | 4772.67 | 20208.59 | 33.96 | 13.02 | 31.78 | 207.10 | 93.40 |
| OVAR 029 | 3192.32 | 34058.65 | 329.78 | 23.32 | 2338.40 | 207.24 | 82.30 |
| OVAR 030 | 1712.90 | 39273.38 | 645.60 | 16.83 | 107.98 | 101.87 | 56.04 |
| OVAR 031 | 237.57 | 36861.88 | 6.46 | 9.00 | 34.32 | 254.43 | 234.19 |
| OVAR 032 | 3586.35 | 19634.10 | 5.48 | 7.06 | 55.01 | 474.30 | 175.20 |
| OVAR 033 | 9946.89 | 21202.46 | 53.96 | 5.23 | 463.27 | 215.34 | 120.75 |
| OVAR 034 | 8802.25 | 35636.35 | 153.51 | 7.26 | 259.62 | 2196.81 | 112.37 |
| OVAR 035 | 14261.59 | 21022.70 | 79.54 | 6.88 | 23291.45 | 1815.05 | 68.09 |
| OVAR 036 | 2640.92 | 11740.98 | 926.89 | 39.14 | 145.39 | 390.91 | 68.09 |
| OVAR 037 | 9468.52 | 18038.11 | 174.46 | 6.03 | 53.95 | 324.85 | 145.47 |
| OVAR 038 | 3491.05 | 7646.22 | 65.09 | 4.29 | 129.19 | 1901.08 | 151.01 |
| OVAR 039 | 6557.49 | 23081.15 | 141.30 | 6.91 | 486.99 | 386.04 | 110.91 |
| OVAR 040 | 1773.14 | 15170.92 | 5.84 | 15.19 | 44.83 | 120.83 | 93.02 |
| OVAR 041 | 7086.28 | 20118.12 | 15.09 | 8.43 | 50.62 | 140.35 | 183.61 |
| OVAR 042 | 10372.70 | 27422.84 | 2.80 | 10.04 | 33.47 | 164.45 | 94.45 |
| OVAR 043 | 6432.56 | 6972.70 | 548.96 | 13.74 | 328.48 | 107.78 | 303.62 |
| OVAR 044 | 2171.98 | 16492.57 | 39.68 | 12.15 | 106.03 | 405.54 | 144.15 |
| BLDR 001 | 26475.67 | 36370.60 | 3.36 | 12.09 | 176.40 | 508.14 | 68.94 |
| BLDR 002 | 8138.57 | 46763.31 | 1.95 | 20.44 | 46.53 | 193.58 | 82.30 |
| BLDR 003 | 2267.29 | 33081.94 | 2.02 | 11.65 | 94.05 | 398.26 | 82.17 |
| BLDR 004 | 6688.55 | 32151.54 | 20.54 | 10.69 | 1147.53 | 1898.62 | 114.04 |
| BLDR 005 | 3968.36 | 16588.20 | 1.93 | 25.63 | 62.63 | 684.06 | 55.71 |
| BLDR 006 | 4802.37 | 12418.04 | 2.52 | 16.04 | 31.46 | 713.31 | 36.01 |
| BLDR 007 | 3733.14 | 10517.33 | 18.57 | 18.96 | 235.92 | 151833.12 | 196.40 |
| BLDR 008 | 10262.41 | 27507.92 | 4.55 | 13.12 | 51.49 | 260.75 | 122.45 |
| BLDR 009 | 577.77 | 16826.56 | 2.52 | 13.16 | 4.77 | 141.57 | 82.30 |
| BLDR 010 | 8669.30 | 42443.17 | 3.30 | 4.45 | 33.08 | 402.11 | 106.30 |
| BLDR 011 | 9001.87 | 19023.25 | 1.81 | 4.57 | 26.14 | 334.48 | 82.30 |
| BLDR 012 | 1344.76 | 15968.01 | 3.16 | 3.95 | 11.46 | 699.97 | 153.58 |
| BLDR 013 | 9771.11 | 36638.62 | 4.78 | 46.70 | 15.88 | 261.17 | 68.41 |
| BLDR 014 | 3396.11 | 19762.91 | 1.95 | 3.09 | 9.87 | 350.86 | 150.88 |
| BLDR 015 | 7543.62 | 23383.82 | 1.57 | 4.36 | 14.03 | 458.75 | 62.94 |
| BLDR 016 | 3189.94 | 61709.31 | 3.05 | 4.54 | 37.73 | 306.18 | 72.08 |
| BLDR 017 | 11280.50 | 22793.61 | 3.86 | 10.90 | 41.07 | 360.46 | 122.45 |
| BLDR 018 | 8403.72 | 28286.73 | 3.14 | 4.11 | 37.89 | 1016.09 | 51.13 |
| BLDR 019 | 12494.87 | 32987.40 | 3.34 | 4.64 | 59.87 | 511.82 | 57.89 |
| BLDR 020 | 9568.48 | 27134.48 | 5.73 | 24.01 | 113.98 | 347.91 | 70.10 |
| BLDR 021 | 6694.06 | 32581.24 | 2.57 | 9.95 | 138.34 | 775.53 | 129.42 |
| BLDR 022 | 1084.59 | 21194.33 | 3.18 | 14.06 | 54.32 | 397.19 | 114.37 |
| BLDR 023 | 3455.12 | 36965.02 | 1.40 | 14.30 | 9.87 | 217.89 | 3.60 |
| BLDR 024 | 9843.69 | 20094.41 | 2.41 | 9.23 | 21.63 | 596.87 | 3.60 |
| BLDR 025 | 4527.69 | 27806.98 | 4.15 | 8.12 | 14.74 | 133.57 | 51.13 |
| BLDR 026 | 2781.81 | 15424.78 | 4.55 | 14.59 | 88.49 | 284.56 | 122.45 |
| BLDR 027 | 17935.31 | 20853.05 | 3.00 | 14.24 | 156.03 | 640.41 | 67.96 |
| BLDR 028 | 6390.27 | 21742.12 | 23.24 | 6.86 | 24.64 | 868.18 | 114.40 |
| BLDR 029 | 19224.75 | 16988.87 | 14.84 | 8.61 | 37.54 | 2455.82 | 180.10 |
| BLDR 030 | 13752.84 | 22708.15 | 6.71 | 9.83 | 92.22 | 671.33 | 82.30 |
| BLDR 031 | 19027.11 | 38042.01 | 13.67 | 5.52 | 550.38 | 3828.25 | 113.41 |
| BLDR 032 | 15399.51 | 16290.79 | 71.26 | 3.69 | 8.99 | 297.69 | 114.95 |
| BLDR 033 | 6489.97 | 26111.20 | 3.33 | 11.80 | 51.32 | 987.88 | 141.82 |
| BLDR 034 | 1604.06 | 13507.39 | 5.79 | 4.28 | 114.35 | 238.57 | 79.52 |
| BLDR 035 | 9507.87 | 19384.77 | 2.66 | 15.50 | 172.01 | 778.73 | 123.09 |
| BLDR 036 | 15110.16 | 38435.72 | 1.22 | 6.45 | 23.35 | 157.97 | 3.60 |
| BLDR 037 | 15145.01 | 43559.11 | 3.51 | 90.83 | 17624.01 | 324.00 | 109.79 |
| BLDR 038 | 8388.18 | 21156.05 | 2.80 | 6.04 | 144.30 | 582.89 | 183.61 |
| BLDR 039 | 15179.86 | 21629.94 | 5.01 | 13.19 | 52.11 | 194.50 | 145.57 |
| BLDR 040 | 8469.99 | 35753.60 | 2.04 | 16.83 | 35.63 | 118.97 | 3.60 |
| BLDR 041 | 6740.02 | 26415.21 | 13.36 | 8.74 | 31.00 | 308.17 | 144.15 |
| BLDR 042 | 9790.85 | 15028.75 | 3.47 | 4.78 | 132.35 | 446.80 | 183.61 |
| BLDR 043 | 7741.31 | 24248.81 | 5.99 | 26.83 | 45.74 | 741.41 | 144.15 |
| BLDR 044 | 16578.18 | 24141.23 | 5.49 | 6.99 | 120.39 | 2936.03 | 94.45 |
| BLDR 045 | 10916.40 | 8719.53 | 2.47 | 5.42 | 23.53 | 491.77 | 183.61 |
| BLDR 046 | 7853.88 | 27438.99 | 5.73 | 14.14 | 52.58 | 254.01 | 181.43 |
| BLDR 047 | 17135.29 | 33260.61 | 3.16 | 37.29 | 53.66 | 322.13 | 66.09 |
| BLDR 048 | 10050.25 | 22568.64 | 2.88 | 5.42 | 99.00 | 331.77 | 125.79 |
| PDAC 001 | 13547.76 | 20702.87 | 1.20 | 17.67 | 65.76 | 176.17 | 68.09 |
| PDAC 002 | 11483.86 | 23357.66 | 3.62 | 6.42 | 190.74 | 805.41 | 46.18 |
| PDAC 003 | 8289.78 | 21206.65 | 3.62 | 16.08 | 655.20 | 269.13 | 57.13 |
| PDAC 004 | 5422.28 | 27087.13 | 14.42 | 9.58 | 66.75 | 234.65 | 85.26 |
| PDAC 005 | 1428.44 | 24132.48 | 5.97 | 4.04 | 101.46 | 170.68 | 54.40 |
| PDAC 006 | 5515.51 | 19213.19 | 15.42 | 8.83 | 186.07 | 2112.17 | 57.13 |
| PDAC 007 | 5577.71 | 16151.16 | 8.27 | 14.96 | 3313.02 | 815.08 | 140.81 |
| PDAC 008 | 12169.38 | 32327.66 | 6.46 | 12.36 | 1609.95 | 1755.03 | 76.54 |
| PDAC 009 | 3854.57 | 19178.00 | 13.36 | 9.71 | 1348.41 | 562.27 | 813.09 |
| PDAC 010 | 4884.79 | 41589.95 | 2.64 | 6.48 | 498.49 | 142.38 | 57.89 |
| PDAC 011 | 25055.20 | 9105.48 | 5.45 | 7.95 | 90.77 | 585.94 | 77.45 |
| PDAC 012 | 12693.36 | 32547.24 | 2.99 | 3.79 | 315.20 | 576.25 | 125.93 |
| PDAC 013 | 11255.92 | 51321.87 | 14.22 | 8.07 | 1007.76 | 373.22 | 55.71 |
| PDAC 014 | 12693.36 | 21675.70 | 8.29 | 5.38 | 1911.22 | 756.83 | 125.79 |
| PDAC 015 | 8737.31 | 25391.83 | 4.40 | 2.49 | 50.11 | 126.68 | 98.99 |
| PDAC 016 | 10282.45 | 38961.84 | 5.99 | 20.19 | 1973.74 | 2427.05 | 82.30 |
| PDAC 017 | 11321.01 | 23273.16 | 1.62 | 16.17 | 108.06 | 113.65 | 71.84 |
| PDAC 018 | 11549.03 | 25311.31 | 2.12 | 19.48 | 39.03 | 1575.06 | 118.09 |
| PDAC 019 | 5639.96 | 37115.88 | 9.14 | 35.98 | 566.06 | 503.80 | 61.89 |
| PDAC 020 | 31890.93 | 29963.88 | 3.53 | 6.86 | 1311.41 | 1080.92 | 123.83 |
| PDAC 021 | 21985.40 | 25857.42 | 3.62 | 10.73 | 531.97 | 853.36 | 97.63 |
| PDAC 022 | 4680.31 | 17492.90 | 28.56 | 4.08 | 1472.75 | 811.01 | 49.33 |
| PDAC 023 | 4834.30 | 23718.15 | 3.26 | 9.67 | 155.49 | 204.05 | 69.90 |
| PDAC 024 | 20291.50 | 18628.63 | 9.52 | 13.60 | 514.80 | 242.37 | 62.94 |
| PDAC 025 | 11288.46 | 19156.01 | 13.19 | 2.46 | 1790.55 | 352.54 | 129.42 |
| PDAC 026 | 4281.46 | 16526.53 | 10.45 | 5.85 | 12.14 | 591.17 | 72.36 |
| PDAC 027 | 2712.00 | 15867.71 | 2.43 | 11.27 | 160.80 | 245.12 | 72.62 |
| PDAC 028 | 8481.40 | 27789.27 | 18.22 | 24.20 | 385.18 | 566.03 | 117.32 |
| PDAC 029 | 2911.51 | 17432.28 | 2.15 | 12.66 | 127.39 | 1109.75 | 107.47 |
| PDAC 030 | 30582.80 | 33073.90 | 9.81 | 7.59 | 629.18 | 292.72 | 97.63 |
| PDAC 031 | 5337.15 | 16430.14 | 403.86 | 21.06 | 397.48 | 133.90 | 141.04 |
| PDAC 032 | 8127.54 | 18261.30 | 3.77 | 10.71 | 54.37 | 76.30 | 82.30 |
| PDAC 033 | 8984.42 | 25532.01 | 3.61 | 9.97 | 72.35 | 275.94 | 94.30 |
| PDAC 034 | 6830.22 | 43316.54 | 3.61 | 7.87 | 360.26 | 389.00 | 67.09 |
| PDAC 035 | 12503.39 | 20686.74 | 7.98 | 3.50 | 2003.00 | 311.56 | 106.30 |
| PDAC 036 | 7853.88 | 51787.63 | 4.97 | 2.77 | 111.65 | 1377.23 | 103.74 |
| PDAC 037 | 25440.11 | 26329.92 | 32.83 | 5.64 | 5143.00 | 356.54 | 82.30 |
| PDAC 038 | 2425.36 | 29457.37 | 2.24 | 15.36 | 59.24 | 111.63 | 113.41 |
| PDAC 039 | 2458.40 | 19413.04 | 7.89 | 19.12 | 182.15 | 365.16 | 123.09 |
| PDAC 040 | 1865.78 | 11628.44 | 3.24 | 29.76 | 145.42 | 264.48 | 165.77 |
| PDAC 041 | 16695.82 | 21167.03 | 4.14 | 18.03 | 10.83 | 257.32 | 94.45 |
| PDAC 042 | 6563.12 | 16683.60 | 3.20 | 7.24 | 50.20 | 487.36 | 3.60 |
| PDAC 043 | 4797.08 | 15538.97 | 7.04 | 3.29 | 11082.99 | 489.12 | 100.56 |
| PDAC 044 | 3655.56 | 25916.72 | 4.13 | 9.94 | 79.23 | 516.84 | 219.11 |
| HC 001 | 12572.71 | 27815.44 | 4.02 | 8.37 | 16.11 | 18.72 | 195.12 |
| HC 002 | 14901.10 | 30473.79 | 1.95 | 7.19 | 8.21 | 7.25 | 120.05 |
| HC 003 | 14274.44 | 25405.19 | 4.28 | 11.52 | 21.77 | 158.84 | 601.33 |
| HC 004 | 14344.03 | 26013.96 | 3.58 | 12.63 | 18.85 | 139.25 | 564.82 |
| HC 005 | 4931.94 | 37734.09 | 4.18 | 17.60 | 64.66 | 119.47 | 120.75 |
| HC 006 | 16226.55 | 21904.80 | 4.75 | 2.74 | 3.47 | 234.62 | 145.26 |
| HC 007 | 11050.34 | 20454.06 | 4.39 | 12.65 | 90.53 | 161.85 | 149.40 |
| HC 008 | 10739.67 | 25037.41 | 4.80 | 9.65 | 20.80 | 143.54 | 529.73 |
| HC 009 | 1604.06 | 13192.44 | 3.53 | 10.13 | 16.34 | 699.47 | 122.98 |
| HC 010 | 12122.33 | 25963.23 | 4.11 | 8.76 | 19.83 | 142.46 | 566.24 |
| HC 011 | 667.80 | 13558.45 | 3.70 | 10.43 | 16.99 | 700.33 | 104.25 |
| HC 012 | 2425.36 | 37990.68 | 4.11 | 13.44 | 30.35 | 137.57 | 246.79 |
| HC 013 | 5506.26 | 38992.56 | 4.11 | 14.60 | 32.24 | 130.56 | 246.79 |
| HC 014 | 3521.90 | 42723.78 | 3.93 | 13.60 | 31.30 | 127.31 | 196.59 |
| HC 015 | 4191.62 | 39301.24 | 3.93 | 12.06 | 26.56 | 117.10 | 246.79 |
| HC 016 | 5438.60 | 18820.33 | 3.58 | 7.30 | 48.33 | 730.66 | 529.73 |
| HC 017 | 3055.26 | 30431.42 | 0.69 | 3.33 | 1.76 | 442.58 | 68.94 |
| HC 018 | 5201.98 | 19717.78 | 2.35 | 4.83 | 11.75 | 554.27 | 281.50 |
| HC 019 | 10119.14 | 18659.42 | 2.70 | 5.98 | 24.19 | 683.54 | 454.90 |
| HC 020 | 5946.65 | 19150.58 | 3.06 | 7.14 | 18.86 | 777.75 | 491.42 |
| HC 021 | 2590.70 | 39241.20 | 3.48 | 14.72 | 43.92 | 128.58 | 102.70 |
| HC 022 | 2194.43 | 40623.96 | 3.55 | 16.27 | 48.45 | 131.25 | 111.73 |
| HC 023 | 5878.83 | 43065.47 | 3.62 | 14.34 | 43.90 | 121.03 | 92.50 |
| HC 024 | 3121.80 | 43619.78 | 3.90 | 16.05 | 48.11 | 139.30 | 111.73 |
| HC 025 | 6286.06 | 38666.95 | 4.71 | 15.52 | 23.37 | 589.08 | 93.21 |
| HC 026 | 17065.33 | 32352.06 | 3.06 | 8.79 | 11.40 | 7.10 | 120.75 |
| HC 027 | 6150.23 | 22429.11 | 2.97 | 10.48 | 11.18 | 24.63 | 3.60 |
| HC 028 | 5743.27 | 22966.04 | 2.97 | 12.14 | 9.54 | 32.81 | 3.60 |
| HC 029 | 11983.84 | 38401.47 | 2.92 | 9.49 | 12.83 | 396.22 | 162.32 |
| HC 030 | 10139.87 | 22675.91 | 1.97 | 7.32 | 8.23 | 13.82 | 144.15 |
| HC 031 | 10217.47 | 20068.32 | 5.15 | 14.92 | 23.53 | 84.59 | 183.61 |
| HC 032 | 3454.49 | 9642.78 | 3.47 | 9.26 | 18.50 | 744.90 | 183.61 |
| HC 033 | 9132.50 | 18198.33 | 3.64 | 8.34 | 22.24 | 415.64 | 210.59 |
| HC 034 | 5359.07 | 13756.35 | 2.47 | 7.09 | 13.41 | 829.13 | 217.80 |
| HC 035 | 2322.15 | 10195.54 | 3.13 | 9.05 | 21.02 | 143.34 | 277.03 |
| HC 036 | 7760.79 | 18170.37 | 3.30 | 9.18 | 21.00 | 449.86 | 243.62 |
| HC 037 | 686.53 | 2423.83 | 2.97 | 7.81 | 13.41 | 894.70 | 303.62 |
| HC 038 | 4404.52 | 10147.67 | 2.80 | 9.17 | 15.97 | 744.90 | 183.61 |
| HC 039 | 9713.32 | 17342.61 | 3.13 | 9.79 | 28.52 | 167.49 | 328.76 |
| HC 040 | 15555.92 | 18434.53 | 3.16 | 6.49 | 9.42 | 803.49 | 153.58 |
| HC 041 | 16382.14 | 19626.18 | 1.81 | 1.97 | 2.86 | 343.59 | 144.15 |
| HC 042 | 28918.91 | 27114.92 | 0.72 | 2.95 | 0.30 | 415.34 | 122.45 |
| HC 043 | 1931.38 | 22095.08 | 3.45 | 5.58 | 59.43 | 358.11 | 68.09 |
| HC 044 | 3288.34 | 12020.84 | 8.59 | 5.88 | 69.84 | 212.02 | 411.31 |
| HC 045 | 3455.12 | 12536.58 | 7.78 | 6.03 | 67.31 | 206.48 | 391.70 |
| HC 046 | 1020.11 | 18439.19 | 1.91 | 7.76 | 34.32 | 675.32 | 62.61 |
| HC 047 | 1855.14 | 18519.85 | 2.25 | 7.60 | 34.30 | 623.25 | 57.13 |
| HC 048 | 2161.78 | 19102.39 | 2.25 | 8.14 | 37.69 | 693.01 | 68.09 |
| HC 049 | 11948.10 | 33538.31 | 5.63 | 8.14 | 22.73 | 145.44 | 170.75 |
| HC 050 | 1357.70 | 25535.78 | 9.32 | 9.92 | 177.62 | 359.52 | 370.25 |
| HC 051 | 2421.37 | 24566.22 | 9.89 | 10.53 | 188.64 | 351.12 | 386.30 |
| HC 052 | 10567.54 | 41438.56 | 4.13 | 8.79 | 22.73 | 146.14 | 178.26 |
| HC 053 | 13218.72 | 39676.03 | 3.27 | 5.92 | 16.02 | 316.47 | 65.82 |
| HC 054 | 6976.61 | 47375.57 | 3.61 | 7.19 | 16.82 | 326.21 | 61.89 |
| HC 055 | 1480.30 | 30425.58 | 6.74 | 23.43 | 59.51 | 444.25 | 128.31 |
| HC 056 | 721.79 | 24082.57 | 7.69 | 24.14 | 51.49 | 449.11 | 153.58 |
| HC 057 | 23824.22 | 26763.56 | 1.73 | 4.74 | 7.52 | 11.74 | 94.16 |
| HC 058 | 14377.23 | 19449.41 | 4.37 | 9.21 | 37.25 | 225.45 | 126.47 |
| HC 059 | 12709.36 | 21784.49 | 5.80 | 9.25 | 57.83 | 255.13 | 170.75 |
| HC 060 | 4281.46 | 21149.17 | 3.26 | 2.52 | 25.71 | 269.09 | 103.54 |
| HC 061 | 2369.18 | 27100.24 | 3.47 | 2.61 | 26.39 | 278.66 | 103.54 |
| HC 062 | 7872.37 | 34615.82 | 4.02 | 12.16 | 25.03 | 532.74 | 112.37 |
| HC 063 | 8476.47 | 35223.50 | 3.82 | 10.25 | 19.58 | 458.92 | 94.71 |
| HC 064 | 5608.83 | 21534.04 | 4.98 | 17.60 | 39.79 | 402.27 | 94.71 |
| HC 065 | 5543.24 | 28330.43 | 3.47 | 9.92 | 24.36 | 190.33 | 120.34 |
| HC 066 | 5399.27 | 22694.98 | 3.05 | 2.59 | 98.78 | 254.85 | 120.34 |
| HC 067 | 866.35 | 9917.48 | 3.75 | 3.70 | 82.45 | 584.19 | 120.34 |
| HC 068 | 1357.70 | 23332.84 | 3.26 | 13.09 | 20.95 | 63.80 | 61.89 |
| HC 069 | 8597.92 | 43619.14 | 2.18 | 6.61 | 17.46 | 279.95 | 65.23 |
| HC 070 | 2007.77 | 18831.99 | 3.52 | 15.24 | 19.35 | 31.48 | 73.77 |
| HC 071 | 18378.87 | 32187.64 | 2.79 | 5.71 | 6.40 | 5.55 | 65.23 |
| HC 072 | 1260.55 | 17930.78 | 4.13 | 17.56 | 20.87 | 31.14 | 73.77 |
| HC 073 | 14635.96 | 27135.31 | 3.04 | 6.48 | 6.40 | 7.55 | 73.77 |
| HC 074 | 15937.62 | 25858.37 | 2.79 | 6.29 | 8.70 | 8.07 | 82.30 |
| HC 075 | 18545.29 | 25740.62 | 3.04 | 6.58 | 8.70 | 9.37 | 82.30 |
| HC 076 | 9792.44 | 17318.59 | 3.77 | 8.97 | 40.45 | 391.68 | 129.77 |
| HC 077 | 21807.54 | 16433.62 | 13.21 | 3.86 | 37.45 | 187.42 | 126.43 |
| HC 078 | 22351.53 | 16267.19 | 13.93 | 4.05 | 36.69 | 181.85 | 116.09 |
| HC 079 | 9699.91 | 18145.14 | 3.77 | 9.81 | 50.21 | 434.43 | 139.65 |
| HC 080 | 18511.99 | 14522.25 | 12.48 | 3.62 | 32.93 | 183.71 | 119.51 |
| HC 081 | 12011.32 | 18781.34 | 4.01 | 9.12 | 49.46 | 413.49 | 139.65 |
| HC 082 | 10598.69 | 32938.28 | 3.28 | 6.48 | 16.32 | 117.05 | 112.19 |
| HC 083 | 1212.31 | 10906.94 | 2.30 | 4.44 | 91.09 | 788.70 | 105.27 |
| HC 084 | 8902.43 | 40394.31 | 7.28 | 12.98 | 46.46 | 816.89 | 97.95 |
| HC 085 | 11380.89 | 40238.85 | 7.65 | 14.39 | 51.71 | 953.47 | 97.95 |
| HC 086 | 11632.58 | 13005.76 | 4.01 | 4.42 | 16.32 | 122.02 | 105.27 |
| HC 087 | 9269.48 | 35146.91 | 3.52 | 7.97 | 18.60 | 129.11 | 115.90 |
| HC 088 | 17318.13 | 32151.94 | 1.57 | 5.10 | 4.86 | 5.20 | 73.77 |
| HC 089 | 7842.31 | 14370.25 | 3.77 | 6.73 | 21.62 | 661.66 | 97.95 |
| HC 090 | 6769.49 | 14005.30 | 3.04 | 5.62 | 17.08 | 619.07 | 90.13 |
| HC 091 | 10132.62 | 31243.02 | 2.79 | 4.82 | 14.04 | 99.66 | 97.44 |
| HC 092 | 9269.48 | 14538.69 | 3.04 | 5.88 | 17.08 | 600.29 | 90.13 |
| HC 093 | 25301.85 | 36112.27 | 1.32 | 4.17 | 3.30 | 6.82 | 64.11 |
| HC 094 | 4601.70 | 21356.37 | 3.77 | 15.79 | 22.38 | 48.54 | 73.77 |
| HC 095 | 2419.24 | 24120.13 | 4.55 | 16.22 | 21.17 | 150.42 | 122.45 |
| HC 096 | 5112.69 | 22127.62 | 4.01 | 15.82 | 22.38 | 40.68 | 65.23 |
| HC 097 | 4320.66 | 21681.66 | 4.01 | 13.06 | 16.32 | 28.59 | 55.58 |
| HC 098 | 18575.63 | 31226.92 | 1.08 | 5.06 | 4.08 | 5.65 | 55.58 |
| HC 099 | 9293.00 | 13117.03 | 2.91 | 6.07 | 17.84 | 507.15 | 158.48 |
| HC 100 | 4104.43 | 27730.84 | 1.57 | 7.75 | 17.08 | 301.50 | 60.59 |
| HC 101 | 1932.96 | 34265.58 | 1.45 | 8.06 | 15.56 | 312.55 | 73.77 |
| HC 102 | 1645.15 | 35660.12 | 2.06 | 7.14 | 27.65 | 302.72 | 73.77 |
| HC 103 | 3959.12 | 35117.83 | 1.88 | 8.05 | 19.48 | 330.51 | 107.17 |
| HC 104 | 18439.63 | 32762.65 | 4.23 | 6.73 | 7.78 | 8.03 | 79.66 |
| HC 105 | 12271.13 | 40639.18 | 3.62 | 11.79 | 22.55 | 362.62 | 80.30 |
| HC 106 | 7899.81 | 36975.37 | 3.62 | 12.28 | 22.92 | 372.18 | 80.30 |
| HC 107 | 5487.66 | 36944.80 | 3.90 | 10.73 | 26.18 | 347.48 | 87.58 |
| HC 108 | 10541.78 | 49377.74 | 2.38 | 6.85 | 53.73 | 2101.20 | 93.02 |
| HC 109 | 12750.04 | 32844.25 | 2.66 | 10.93 | 18.47 | 474.43 | 203.32 |
| HC 110 | 7533.67 | 50632.72 | 2.24 | 5.88 | 44.83 | 1889.14 | 82.30 |
| HC 111 | 10468.27 | 35512.50 | 2.38 | 10.70 | 15.06 | 425.46 | 203.57 |
| HC 112 | 8412.76 | 51467.19 | 2.81 | 6.59 | 51.62 | 2061.85 | 93.02 |
| HC 113 | 13070.07 | 40022.86 | 3.16 | 8.36 | 15.43 | 327.54 | 122.45 |

| Subject | | | | | | | |
|---|---|---|---|---|---|---|---|
| Cohort | | | | | | | |
| ID | HE4 | HGF | IL-6 | IL-8 | Leptin | MIF | OPN |
| OVAR 001 | 280.35 | 6.80 | 0.20 | 0.30 | 301.20 | 7.60 | 285.30 |
| OVAR 002 | 208.23 | 6.80 | 0.20 | 0.30 | 74.59 | 7.60 | 285.30 |
| OVAR 003 | 193.50 | 6.80 | 0.20 | 0.30 | 137.85 | 7.60 | 285.30 |
| OVAR 004 | 237.09 | 6.80 | 0.84 | 0.30 | 61.98 | 7.60 | 818.88 |
| OVAR 005 | 1742.56 | 6.80 | 0.20 | 0.30 | 146.12 | 7.60 | 469.98 |
| OVAR 006 | 193.50 | 6.80 | 0.20 | 0.30 | 42.80 | 7.60 | 285.30 |
| OVAR 007 | 394.61 | 6.80 | 1.56 | 0.30 | 58.03 | 38.61 | 285.30 |
| OVAR 008 | 279.38 | 6.80 | 1.90 | 0.30 | 58.03 | 10.69 | 285.30 |
| OVAR 009 | 397.90 | 6.80 | 1.69 | 0.30 | 83.26 | 29.63 | 453.99 |
| OVAR 010 | 222.86 | 6.80 | 1.40 | 0.30 | 70.65 | 26.56 | 285.30 |
| OVAR 011 | 195.52 | 6.80 | 1.13 | 0.30 | 76.93 | 8.75 | 429.34 |
| OVAR 012 | 193.50 | 6.80 | 0.96 | 0.30 | 61.98 | 8.33 | 471.63 |
| OVAR 013 | 193.50 | 11.40 | 1.04 | 0.30 | 187.74 | 33.44 | 939.97 |
| OVAR 014 | 241.35 | 7.46 | 1.40 | 0.30 | 259.89 | 9.58 | 285.30 |
| OVAR 015 | 193.50 | 10.17 | 1.68 | 0.32 | 146.93 | 19.59 | 678.54 |
| OVAR 016 | 193.50 | 6.80 | 0.20 | 0.30 | 233.03 | 7.60 | 285.30 |
| OVAR 017 | 193.50 | 7.00 | 0.20 | 0.39 | 107.51 | 9.86 | 285.30 |
| OVAR 018 | 193.50 | 6.80 | 0.20 | 0.30 | 88.55 | 7.60 | 285.30 |
| OVAR 019 | 193.50 | 6.80 | 1.00 | 0.33 | 86.68 | 13.16 | 522.11 |
| OVAR 020 | 193.50 | 6.80 | 0.20 | 0.30 | 42.80 | 7.60 | 285.30 |
| OVAR 021 | 193.50 | 6.80 | 1.66 | 0.38 | 86.68 | 49.96 | 285.30 |
| OVAR 022 | 193.50 | 6.80 | 0.81 | 0.34 | 120.48 | 21.77 | 285.30 |
| OVAR 023 | 1355.46 | 14.44 | 0.68 | 1.69 | 54.44 | 19.12 | 2509.77 |
| OVAR 024 | 193.50 | 6.96 | 0.84 | 0.37 | 252.83 | 17.09 | 832.18 |
| OVAR 025 | 193.50 | 6.80 | 0.60 | 0.30 | 96.03 | 27.24 | 552.18 |
| OVAR 026 | 193.50 | 6.80 | 1.30 | 0.44 | 62.69 | 30.12 | 469.84 |
| OVAR 027 | 193.50 | 8.17 | 1.70 | 0.38 | 1568.37 | 41.77 | 319.06 |
| OVAR 028 | 193.50 | 10.64 | 2.12 | 0.43 | 467.68 | 45.13 | 309.23 |
| OVAR 029 | 193.50 | 6.80 | 1.27 | 0.30 | 356.42 | 20.62 | 405.23 |
| OVAR 030 | 193.50 | 6.80 | 1.20 | 0.30 | 219.35 | 62.55 | 285.30 |
| OVAR 031 | 193.50 | 6.80 | 1.21 | 0.37 | 174.52 | 13.46 | 378.41 |
| OVAR 032 | 193.50 | 6.80 | 0.20 | 0.30 | 285.11 | 7.60 | 285.30 |
| OVAR 033 | 389.85 | 6.80 | 1.25 | 0.30 | 73.12 | 9.25 | 808.50 |
| OVAR 034 | 193.50 | 6.80 | 1.02 | 0.78 | 63.56 | 7.60 | 285.30 |
| OVAR 035 | 193.50 | 12.16 | 1.05 | 0.92 | 360.50 | 23.31 | 684.10 |
| OVAR 036 | 193.50 | 9.01 | 1.12 | 1.58 | 103.33 | 15.69 | 898.21 |
| OVAR 037 | 193.50 | 6.80 | 0.86 | 0.44 | 62.69 | 38.45 | 380.55 |
| OVAR 038 | 193.50 | 7.49 | 0.81 | 0.37 | 223.42 | 78.18 | 343.81 |
| OVAR 039 | 551.16 | 6.80 | 1.03 | 0.30 | 61.98 | 15.74 | 876.39 |
| OVAR 040 | 492.73 | 6.80 | 0.20 | 0.30 | 74.59 | 13.72 | 409.76 |
| OVAR 041 | 193.50 | 6.80 | 1.95 | 0.53 | 84.92 | 14.52 | 349.64 |
| OVAR 042 | 1153.06 | 30.88 | 0.49 | 0.64 | 10644.81 | 15.60 | 2180.78 |
| OVAR 043 | 193.50 | 6.80 | 2.15 | 0.30 | 84.92 | 9.30 | 372.60 |
| OVAR 044 | 1934.48 | 30.88 | 0.54 | 1.17 | 3481.39 | 20.90 | 1442.19 |
| BLDR 001 | 431.62 | 8.57 | 1.38 | 0.30 | 103.43 | 27.80 | 2090.48 |
| BLDR 002 | 193.50 | 6.80 | 1.39 | 0.30 | 65.51 | 7.60 | 285.30 |
| BLDR 003 | 193.50 | 6.80 | 1.21 | 0.30 | 77.70 | 8.39 | 386.70 |
| BLDR 004 | 346.07 | 6.80 | 1.38 | 0.30 | 91.85 | 7.60 | 285.30 |
| BLDR 005 | 255.54 | 8.54 | 1.42 | 0.30 | 142.78 | 12.48 | 1056.83 |
| BLDR 006 | 431.62 | 10.65 | 1.25 | 0.30 | 113.56 | 7.60 | 347.38 |
| BLDR 007 | 193.50 | 10.13 | 0.20 | 0.58 | 214.97 | 27.30 | 2487.20 |
| BLDR 008 | 193.50 | 6.98 | 1.97 | 0.39 | 135.97 | 16.32 | 285.30 |
| BLDR 009 | 386.70 | 6.80 | 1.15 | 0.30 | 76.02 | 7.60 | 285.30 |
| BLDR 010 | 281.34 | 6.80 | 0.90 | 0.30 | 46.75 | 7.60 | 644.35 |
| BLDR 011 | 278.34 | 6.80 | 1.21 | 0.30 | 65.51 | 15.53 | 285.30 |
| BLDR 012 | 193.50 | 6.98 | 2.06 | 0.30 | 82.13 | 11.51 | 285.30 |
| BLDR 013 | 193.50 | 6.80 | 1.30 | 0.30 | 171.89 | 9.07 | 285.30 |
| BLDR 014 | 193.50 | 6.80 | 0.20 | 0.30 | 42.80 | 7.60 | 285.30 |
| BLDR 015 | 193.50 | 6.80 | 1.32 | 0.35 | 71.22 | 56.33 | 285.30 |
| BLDR 016 | 193.50 | 6.80 | 1.18 | 0.30 | 51.98 | 7.60 | 285.30 |
| BLDR 017 | 193.50 | 6.80 | 2.11 | 0.30 | 82.13 | 14.11 | 285.30 |
| BLDR 018 | 193.50 | 18.06 | 1.28 | 0.35 | 181.75 | 33.93 | 1380.63 |
| BLDR 019 | 193.50 | 6.80 | 1.23 | 0.30 | 42.80 | 10.56 | 294.20 |
| BLDR 020 | 454.32 | 6.80 | 1.47 | 0.30 | 42.80 | 13.71 | 376.90 |
| BLDR 021 | 193.50 | 8.19 | 1.09 | 0.31 | 71.22 | 50.88 | 285.30 |
| BLDR 022 | 193.50 | 6.80 | 0.20 | 0.30 | 42.80 | 7.60 | 285.30 |
| BLDR 023 | 193.50 | 6.80 | 0.20 | 0.30 | 42.80 | 7.60 | 285.30 |
| BLDR 024 | 193.50 | 6.80 | 0.20 | 0.30 | 42.80 | 7.60 | 285.30 |
| BLDR 025 | 193.50 | 6.80 | 0.78 | 0.30 | 117.94 | 15.95 | 437.60 |
| BLDR 026 | 193.50 | 6.80 | 2.08 | 0.30 | 82.13 | 15.22 | 285.30 |
| BLDR 027 | 193.50 | 6.80 | 1.30 | 0.30 | 71.22 | 45.16 | 285.30 |
| BLDR 028 | 193.50 | 6.80 | 1.19 | 0.30 | 51.98 | 14.22 | 285.30 |
| BLDR 029 | 193.50 | 6.80 | 1.92 | 0.30 | 82.13 | 7.60 | 285.30 |
| BLDR 030 | 304.83 | 6.80 | 1.45 | 0.30 | 57.17 | 7.60 | 285.30 |
| BLDR 031 | 356.49 | 6.80 | 1.10 | 0.30 | 62.98 | 13.96 | 786.33 |
| BLDR 032 | 193.50 | 6.80 | 1.11 | 0.38 | 150.76 | 19.02 | 475.92 |
| BLDR 033 | 193.50 | 8.15 | 1.42 | 0.30 | 70.65 | 22.21 | 1018.02 |
| BLDR 034 | 284.86 | 6.80 | 1.37 | 0.34 | 70.65 | 11.42 | 285.30 |
| BLDR 035 | 356.32 | 6.80 | 1.36 | 0.30 | 166.10 | 40.73 | 285.30 |
| BLDR 036 | 193.50 | 6.80 | 0.20 | 0.30 | 48.22 | 17.36 | 285.30 |
| BLDR 037 | 347.98 | 6.80 | 1.53 | 0.30 | 73.85 | 14.60 | 285.30 |
| BLDR 038 | 193.50 | 6.80 | 1.84 | 0.43 | 60.35 | 7.60 | 285.30 |
| BLDR 039 | 284.86 | 6.80 | 0.68 | 0.34 | 58.03 | 52.83 | 285.30 |
| BLDR 040 | 193.50 | 6.80 | 0.20 | 0.30 | 42.80 | 8.32 | 285.30 |
| BLDR 041 | 193.50 | 8.02 | 1.71 | 0.43 | 120.74 | 54.99 | 314.07 |
| BLDR 042 | 193.50 | 6.80 | 1.80 | 0.32 | 84.92 | 21.25 | 285.30 |
| BLDR 043 | 193.50 | 6.80 | 1.95 | 0.32 | 42.80 | 60.78 | 355.43 |
| BLDR 044 | 1221.80 | 34.72 | 0.54 | 0.85 | 11095.72 | 16.68 | 2258.22 |
| BLDR 045 | 1012.82 | 38.49 | 0.49 | 4.49 | 514.05 | 74.85 | 1318.67 |
| BLDR 046 | 255.54 | 6.80 | 1.04 | 0.30 | 77.70 | 7.60 | 285.30 |
| BLDR 047 | 267.64 | 6.80 | 1.33 | 0.30 | 91.50 | 7.60 | 363.70 |
| BLDR 048 | 250.30 | 6.80 | 0.77 | 0.30 | 105.85 | 7.60 | 324.65 |
| PDAC 001 | 193.50 | 6.80 | 0.58 | 0.30 | 117.01 | 9.99 | 570.18 |
| PDAC 002 | 193.50 | 7.67 | 0.60 | 0.30 | 59.21 | 9.39 | 340.15 |
| PDAC 003 | 193.50 | 34.49 | 0.96 | 1.26 | 1401.29 | 12.85 | 8187.94 |
| PDAC 004 | 254.79 | 6.80 | 1.66 | 0.30 | 42.80 | 8.32 | 285.30 |
| PDAC 005 | 193.50 | 6.80 | 0.20 | 0.30 | 96.67 | 9.32 | 1310.69 |
| PDAC 006 | 193.50 | 6.80 | 1.36 | 0.30 | 96.67 | 11.00 | 479.25 |
| PDAC 007 | 193.50 | 6.80 | 0.76 | 0.30 | 115.40 | 7.60 | 432.08 |
| PDAC 008 | 245.65 | 6.80 | 1.91 | 0.38 | 60.73 | 149.21 | 285.30 |
| PDAC 009 | 193.50 | 6.80 | 1.49 | 0.38 | 76.60 | 19.96 | 285.30 |
| PDAC 010 | 193.50 | 6.80 | 1.47 | 0.30 | 73.12 | 7.60 | 368.03 |
| PDAC 011 | 193.50 | 7.38 | 1.89 | 0.68 | 63.56 | 62.42 | 459.42 |
| PDAC 012 | 458.19 | 6.80 | 1.23 | 0.30 | 157.51 | 7.6 | 623.45 |
| PDAC 013 | 386.36 | 7.47 | 1.05 | 0.30 | 91.85 | 7.60 | 322.92 |
| PDAC 014 | 222.86 | 6.80 | 1.33 | 0.30 | 42.80 | 8.05 | 285.30 |
| PDAC 015 | 193.50 | 8.25 | 0.32 | 0.30 | 63.56 | 56.84 | 428.38 |
| PDAC 016 | 385.28 | 6.80 | 1.21 | 0.34 | 42.80 | 16.47 | 809.09 |
| PDAC 017 | 498.07 | 6.80 | 1.06 | 0.31 | 60.73 | 7.60 | 426.69 |
| PDAC 018 | 245.65 | 11.43 | 0.97 | 0.38 | 83.86 | 54.90 | 432.11 |
| PDAC 019 | 193.50 | 8.17 | 1.83 | 0.33 | 179.68 | 41.87 | 334.83 |
| PDAC 020 | 409.26 | 6.80 | 0.83 | 0.30 | 91.85 | 7.60 | 684.30 |
| PDAC 021 | 222.86 | 6.80 | 1.64 | 0.30 | 42.80 | 7.60 | 285.30 |
| PDAC 022 | 193.50 | 9.81 | 0.20 | 0.30 | 86.68 | 16.66 | 360.61 |
| PDAC 023 | 193.50 | 7.67 | 0.51 | 0.48 | 154.06 | 20.05 | 1306.18 |
| PDAC 024 | 193.50 | 9.23 | 1.29 | 0.32 | 110.97 | 7.60 | 389.97 |
| PDAC 025 | 193.50 | 6.80 | 1.56 | 0.30 | 110.97 | 7.60 | 606.51 |
| PDAC 026 | 313.34 | 18.80 | 1.07 | 0.87 | 91.33 | 7.60 | 1070.15 |
| PDAC 027 | 193.50 | 6.80 | 0.91 | 0.30 | 395.44 | 13.80 | 541.33 |
| PDAC 028 | 193.50 | 6.80 | 1.53 | 0.30 | 58.03 | 19.95 | 285.30 |
| PDAC 029 | 193.50 | 6.80 | 2.48 | 0.30 | 42.80 | 7.60 | 330.77 |
| PDAC 030 | 332.61 | 6.80 | 0.59 | 0.30 | 49.69 | 39.06 | 927.26 |
| PDAC 031 | 193.50 | 6.80 | 0.96 | 0.30 | 76.93 | 12.45 | 744.76 |
| PDAC 032 | 193.50 | 6.80 | 0.64 | 0.30 | 175.30 | 53.11 | 726.32 |
| PDAC 033 | 193.50 | 6.80 | 1.05 | 0.30 | 46.75 | 13.19 | 857.86 |
| PDAC 034 | 241.35 | 13.28 | 1.28 | 0.49 | 96.21 | 14.89 | 1401.40 |
| PDAC 035 | 193.50 | 6.80 | 0.68 | 0.30 | 61.98 | 36.87 | 285.30 |
| PDAC 036 | 375.95 | 6.80 | 0.20 | 0.30 | 57.90 | 7.60 | 285.30 |
| PDAC 037 | 193.50 | 6.80 | 0.83 | 0.30 | 73.12 | 17.02 | 499.93 |
| PDAC 038 | 208.23 | 6.80 | 0.20 | 0.30 | 221.70 | 27.82 | 285.30 |
| PDAC 039 | 548.29 | 6.80 | 2.85 | 0.30 | 57.90 | 9.15 | 285.30 |
| PDAC 040 | 308.27 | 6.80 | 0.20 | 0.30 | 86.84 | 9.94 | 285.30 |
| PDAC 041 | 193.50 | 6.80 | 1.58 | 0.30 | 469.83 | 11.20 | 314.07 |
| PDAC 042 | 193.50 | 6.80 | 0.87 | 0.30 | 125.83 | 11.98 | 289.94 |
| PDAC 043 | 193.50 | 88.43 | 1.36 | 0.30 | 174.57 | 13.46 | 601.05 |
| PDAC 044 | 193.50 | 6.80 | 0.51 | 0.30 | 42.80 | 9.21 | 519.76 |
| HC 001 | 504.36 | 6.80 | 1.82 | 0.51 | 279.32 | 21.72 | 285.30 |
| HC 002 | 193.50 | 6.80 | 0.25 | 0.30 | 203.92 | 7.60 | 285.30 |
| HC 003 | 388.45 | 12.93 | 0.20 | 0.38 | 859.65 | 12.98 | 285.30 |
| HC 004 | 791.64 | 23.04 | 0.20 | 0.57 | 2016.77 | 11.15 | 285.30 |
| HC 005 | 1449.25 | 10.54 | 0.20 | 0.45 | 510.15 | 26.20 | 285.30 |
| HC 006 | 193.50 | 7.91 | 1.20 | 0.46 | 330.72 | 16.28 | 285.30 |
| HC 007 | 193.50 | 19.76 | 0.20 | 0.42 | 850.09 | 87.87 | 285.30 |
| HC 008 | 193.50 | 6.80 | 0.20 | 0.30 | 920.02 | 11.20 | 285.30 |
| HC 009 | 300.80 | 6.80 | 1.38 | 0.30 | 91.85 | 7.60 | 285.30 |
| HC 010 | 193.50 | 6.89 | 0.20 | 0.30 | 276.72 | 11.31 | 285.30 |
| HC 011 | 411.43 | 6.80 | 1.07 | 0.30 | 125.14 | 22.53 | 285.30 |
| HC 012 | 388.45 | 6.80 | 0.20 | 0.32 | 42.80 | 18.66 | 285.30 |
| HC 013 | 193.50 | 6.80 | 0.20 | 0.30 | 207.87 | 11.31 | 285.30 |
| HC 014 | 388.45 | 6.80 | 0.20 | 0.30 | 111.14 | 13.72 | 285.30 |
| HC 015 | 193.50 | 6.80 | 0.20 | 0.45 | 138.25 | 14.50 | 285.30 |
| HC 016 | 388.45 | 6.80 | 0.20 | 0.30 | 165.37 | 7.60 | 285.30 |
| HC 017 | 230.42 | 6.80 | 1.32 | 0.30 | 91.85 | 16.90 | 294.86 |
| HC 018 | 388.45 | 6.80 | 0.20 | 0.30 | 138.25 | 7.60 | 285.30 |
| HC 019 | 193.50 | 6.80 | 0.20 | 0.30 | 111.14 | 7.60 | 285.30 |
| HC 020 | 193.50 | 6.80 | 0.20 | 0.30 | 111.14 | 7.60 | 285.30 |
| HC 021 | 341.38 | 6.80 | 1.56 | 0.30 | 49.69 | 20.60 | 285.30 |
| HC 022 | 341.38 | 6.80 | 1.44 | 0.30 | 80.81 | 19.79 | 285.30 |
| HC 023 | 502.05 | 6.80 | 1.52 | 0.30 | 83.26 | 40.52 | 285.30 |
| HC 024 | 397.90 | 6.80 | 1.88 | 0.38 | 70.65 | 35.98 | 285.30 |
| HC 025 | 193.50 | 6.80 | 1.75 | 0.30 | 152.91 | 52.81 | 285.30 |
| HC 026 | 478.08 | 6.80 | 1.62 | 0.51 | 203.33 | 30.36 | 285.30 |
| HC 027 | 195622.51 | 6.80 | 0.20 | 6.50 | 42.80 | 72.35 | 285.30 |
| HC 028 | 193.50 | 6.80 | 0.20 | 0.30 | 42.80 | 7.60 | 285.30 |
| HC 029 | 193.50 | 6.80 | 0.42 | 0.30 | 42.80 | 7.60 | 285.30 |
| HC 030 | 193.50 | 10.31 | 1.16 | 0.64 | 368.06 | 249.93 | 337.94 |
| HC 031 | 193.50 | 9.18 | 1.73 | 0.64 | 42.80 | 102.60 | 285.30 |
| HC 032 | 193.50 | 6.80 | 2.04 | 0.30 | 42.80 | 12.69 | 285.30 |
| HC 033 | 1683.04 | 25.51 | 1.19 | 1.07 | 4547.72 | 107.08 | 2011.31 |
| HC 034 | 193.50 | 6.80 | 1.95 | 0.32 | 42.80 | 27.25 | 285.30 |
| HC 035 | 193.50 | 28.94 | 0.20 | 2.15 | 2080.30 | 51.12 | 5356.92 |
| HC 036 | 941.13 | 24.40 | 2.36 | 1.01 | 6958.75 | 59.42 | 1094.64 |
| HC 037 | 193.50 | 16.83 | 2.10 | 0.74 | 104.23 | 226.20 | 285.30 |
| HC 038 | 1554.35 | 32.81 | 0.52 | 1.72 | 3497.02 | 67.82 | 1590.85 |
| HC 039 | 193.50 | 8.02 | 1.91 | 0.43 | 148.77 | 61.42 | 285.30 |
| HC 040 | 193.50 | 8.60 | 2.18 | 0.50 | 82.13 | 48.67 | 285.30 |
| HC 041 | 193.50 | 10.31 | 1.89 | 0.64 | 42.80 | 106.76 | 285.30 |
| HC 042 | 193.50 | 23.08 | 2.11 | 1.48 | 82.13 | 216.35 | 285.30 |
| HC 043 | 193.50 | 6.80 | 0.83 | 0.66 | 321.20 | 14.86 | 368.73 |
| HC 044 | 193.50 | 6.80 | 0.43 | 0.66 | 54.44 | 23.95 | 398.95 |
| HC 045 | 193.50 | 7.19 | 0.37 | 0.49 | 78.88 | 47.85 | 375.56 |
| HC 046 | 193.50 | 6.80 | 0.77 | 0.30 | 90.19 | 13.87 | 448.33 |
| HC 047 | 193.50 | 7.08 | 0.79 | 0.30 | 142.27 | 24.94 | 489.91 |
| HC 048 | 193.50 | 6.80 | 0.20 | 0.30 | 90.19 | 18.51 | 669.68 |
| HC 049 | 193.50 | 6.80 | 0.21 | 0.30 | 234.58 | 13.87 | 306.55 |
| HC 050 | 193.50 | 7.07 | 0.57 | 0.30 | 90.19 | 35.90 | 496.43 |
| HC 051 | 193.50 | 7.07 | 0.46 | 0.30 | 90.19 | 14.64 | 627.93 |
| HC 052 | 193.50 | 7.07 | 0.20 | 0.30 | 1516.35 | 12.20 | 365.84 |
| HC 053 | 193.50 | 7.42 | 1.58 | 0.41 | 109.81 | 37.10 | 429.09 |
| HC 054 | 193.50 | 7.38 | 1.79 | 0.33 | 109.81 | 26.60 | 613.01 |
| HC 055 | 193.50 | 9.81 | 1.72 | 0.38 | 139.04 | 7.60 | 676.04 |
| HC 056 | 193.50 | 6.80 | 1.73 | 0.30 | 82.13 | 7.60 | 285.30 |
| HC 057 | 193.50 | 11.39 | 1.85 | 0.68 | 536.24 | 256.10 | 401.30 |
| HC 058 | 193.50 | 6.80 | 1.33 | 0.38 | 63.56 | 51.15 | 285.30 |
| HC 059 | 193.50 | 10.12 | 0.20 | 0.86 | 88.50 | 77.44 | 680.05 |
| HC 060 | 193.50 | 7.38 | 2.12 | 0.30 | 63.56 | 17.68 | 469.49 |
| HC 061 | 193.50 | 7.65 | 1.73 | 0.32 | 136.51 | 11.66 | 883.69 |
| HC 062 | 193.50 | 6.80 | 1.24 | 0.36 | 62.69 | 26.14 | 327.61 |
| HC 063 | 193.50 | 6.80 | 1.57 | 0.44 | 42.80 | 17.75 | 331.34 |
| HC 064 | 193.50 | 10.18 | 0.92 | 0.40 | 142.96 | 79.45 | 866.11 |
| HC 065 | 193.50 | 6.80 | 0.20 | 0.30 | 42.80 | 7.60 | 285.30 |
| HC 066 | 193.50 | 6.80 | 0.20 | 0.30 | 42.80 | 7.60 | 285.30 |
| HC 067 | 193.50 | 19.33 | 1.34 | 1.58 | 135.75 | 9.50 | 631.47 |
| HC 068 | 193.50 | 10.79 | 1.48 | 0.36 | 167.85 | 38.85 | 992.32 |
| HC 069 | 337.32 | 6.80 | 1.05 | 0.30 | 85.64 | 12.04 | 341.45 |
| HC 070 | 350.95 | 6.80 | 1.10 | 0.30 | 47.66 | 10.87 | 418.02 |
| HC 071 | 193.50 | 6.80 | 0.83 | 0.30 | 42.80 | 7.60 | 285.30 |
| HC 072 | 250.80 | 7.79 | 1.24 | 0.30 | 153.39 | 7.60 | 1006.11 |
| HC 073 | 193.50 | 6.80 | 0.90 | 0.30 | 46.50 | 7.60 | 315.42 |
| HC 074 | 193.50 | 6.80 | 0.89 | 0.30 | 68.89 | 9.84 | 285.30 |
| HC 075 | 193.50 | 6.80 | 0.94 | 0.30 | 143.53 | 24.61 | 285.30 |
| HC 076 | 436.14 | 6.80 | 0.79 | 0.30 | 66.65 | 14.37 | 487.16 |
| HC 077 | 193.50 | 6.80 | 0.58 | 0.30 | 42.80 | 13.58 | 285.30 |
| HC 078 | 193.50 | 6.80 | 0.67 | 0.30 | 42.80 | 13.02 | 285.30 |
| HC 079 | 267.54 | 6.80 | 0.87 | 0.30 | 47.66 | 8.60 | 285.30 |
| HC 080 | 239.77 | 6.80 | 0.20 | 0.30 | 82.99 | 9.95 | 285.30 |
| HC 081 | 193.50 | 6.80 | 0.89 | 0.30 | 47.66 | 10.58 | 285.30 |
| HC 082 | 193.50 | 6.80 | 0.94 | 0.30 | 46.50 | 8.26 | 285.30 |
| HC 083 | 193.50 | 6.80 | 1.26 | 0.30 | 47.66 | 7.60 | 300.23 |
| HC 084 | 334.86 | 8.78 | 0.71 | 0.34 | 99.11 | 30.99 | 892.64 |
| HC 085 | 193.50 | 6.80 | 1.03 | 0.30 | 61.98 | 7.60 | 303.26 |
| HC 086 | 250.80 | 8.78 | 0.20 | 0.30 | 112.58 | 18.51 | 719.37 |
| HC 087 | 193.50 | 6.80 | 0.81 | 0.30 | 117.87 | 7.60 | 285.30 |
| HC 088 | 193.50 | 6.80 | 0.87 | 0.30 | 62.54 | 7.60 | 285.30 |
| HC 089 | 272.80 | 6.80 | 0.98 | 0.30 | 47.66 | 11.46 | 472.08 |
| HC 090 | 193.50 | 7.28 | 0.78 | 0.30 | 47.66 | 26.63 | 348.93 |
| HC 091 | 193.50 | 6.80 | 0.20 | 0.30 | 163.12 | 7.60 | 285.30 |
| HC 092 | 204.91 | 8.28 | 1.02 | 0.30 | 47.66 | 38.50 | 410.07 |
| HC 093 | 193.50 | 6.80 | 0.37 | 0.30 | 381.32 | 7.60 | 285.30 |
| HC 094 | 250.80 | 6.80 | 1.10 | 0.30 | 91.26 | 7.82 | 588.36 |
| HC 095 | 193.50 | 6.80 | 2.15 | 0.30 | 82.13 | 11.81 | 285.30 |
| HC 096 | 193.50 | 6.80 | 1.28 | 0.30 | 125.14 | 20.09 | 285.30 |
| HC 097 | 193.50 | 6.80 | 1.20 | 0.30 | 63.55 | 11.38 | 285.30 |
| HC 098 | 193.50 | 6.80 | 0.30 | 0.30 | 236.29 | 7.60 | 285.30 |
| HC 099 | 419.57 | 6.80 | 1.12 | 0.30 | 91.85 | 20.81 | 497.77 |
| HC 100 | 193.50 | 6.80 | 0.54 | 0.30 | 358.20 | 7.60 | 285.30 |
| HC 101 | 890.32 | 23.78 | 0.20 | 2.70 | 15473.75 | 11.65 | 3124.31 |
| HC 102 | 482.62 | 23.78 | 0.20 | 1.76 | 4538.78 | 7.60 | 1845.75 |
| HC 103 | 302.92 | 6.80 | 0.20 | 0.30 | 183.74 | 14.35 | 285.30 |
| HC 104 | 193.50 | 6.80 | 0.33 | 0.30 | 79.09 | 7.60 | 285.30 |
| HC 105 | 193.50 | 6.80 | 0.20 | 0.30 | 42.80 | 7.60 | 285.30 |
| HC 106 | 193.50 | 6.80 | 0.20 | 0.30 | 48.22 | 7.60 | 285.30 |
| HC 107 | 193.50 | 6.80 | 0.20 | 0.30 | 42.80 | 7.60 | 285.30 |
| HC 108 | 193.50 | 6.80 | 0.20 | 0.30 | 60.89 | 7.60 | 285.30 |
| HC 109 | 193.50 | 14.29 | 0.20 | 0.30 | 206.70 | 9.00 | 390.43 |
| HC 110 | 193.50 | 6.80 | 0.20 | 0.42 | 212.76 | 7.60 | 285.30 |
| HC 111 | 193.50 | 6.80 | 0.20 | 0.30 | 79.09 | 7.60 | 285.30 |
| HC 112 | 193.50 | 6.80 | 0.39 | 0.30 | 42.80 | 7.60 | 285.30 |
| HC 113 | 193.50 | 6.80 | 1.82 | 0.30 | 82.13 | 8.93 | 285.30 |
| OVAR 001 | 193.50 | 155.76 | 0.20 | 4.77 | 76237.44 | 78.05 | 6132.67 |
| OVAR 002 | 193.50 | 134.81 | 0.20 | 6.05 | 16753.57 | 31.88 | 23852.13 |
| OVAR 003 | 193.50 | 143.22 | 9.65 | 10.47 | 80016.99 | 37.70 | 21592.77 |
| OVAR 004 | 193.50 | 103.43 | 0.20 | 0.30 | 8419.01 | 30.79 | 34512.47 |
| OVAR 005 | 193.50 | 168.17 | 0.20 | 5.53 | 26233.08 | 75.43 | 41891.24 |
| OVAR 006 | 609.62 | 88.78 | 0.20 | 0.30 | 6950.70 | 43.12 | 21953.42 |
| OVAR 007 | 193.50 | 129.89 | 0.20 | 1.92 | 24238.25 | 57.84 | 18143.91 |
| OVAR 008 | 3322.37 | 286.56 | 0.20 | 9.04 | 18670.50 | 591.79 | 37375.72 |
| OVAR 009 | 5240.70 | 195.55 | 10.07 | 3.13 | 1500.63 | 395.93 | 70876.96 |
| OVAR 010 | 311.59 | 132.23 | 0.20 | 3.46 | 18914.12 | 132.81 | 32426.96 |
| OVAR 011 | 938.70 | 350.84 | 24.86 | 11.30 | 10261.75 | 56.50 | 12427.41 |
| OVAR 012 | 5369.04 | 151.12 | 1.66 | 12.37 | 28318.67 | 54.23 | 58992.28 |
| OVAR 013 | 193.50 | 251.68 | 2.14 | 5.47 | 16561.48 | 76.94 | 47899.40 |
| OVAR 014 | 4409.66 | 615.54 | 0.20 | 4.14 | 43221.86 | 52.20 | 31677.71 |
| OVAR 015 | 9963.20 | 255.39 | 4.13 | 17.54 | 19872.74 | 39.82 | 51484.30 |
| OVAR 016 | 4742.23 | 545.38 | 3.37 | 22.56 | 77981.73 | 80.78 | 76705.22 |
| OVAR 017 | 28140.49 | 777.93 | 22.74 | 33.84 | 9618.29 | 57.47 | 63821.59 |
| OVAR 018 | 6119.77 | 439.43 | 0.20 | 10.31 | 30230.46 | 67.25 | 41397.08 |
| OVAR 019 | 193.50 | 139.62 | 1.82 | 6.53 | 12514.75 | 30.84 | 18046.16 |
| OVAR 020 | 24968.04 | 459.37 | 0.20 | 19.27 | 41437.14 | 59.55 | 125847.33 |
| OVAR 021 | 2033.54 | 190.14 | 1.36 | 4.68 | 22291.92 | 55.03 | 10533.02 |
| OVAR 022 | 882.39 | 122.33 | 0.20 | 6.36 | 21715.14 | 92.84 | 14170.51 |
| OVAR 023 | 284029.96 | 821.75 | 66.56 | 100.61 | 2809.79 | 127.96 | 93632.78 |
| OVAR 024 | 2628.40 | 110.79 | 0.20 | 2.68 | 46212.59 | 64.78 | 19339.80 |
| OVAR 025 | 5717.16 | 98.89 | 0.20 | 3.97 | 2186.61 | 154.63 | 54812.22 |
| OVAR 026 | 24688.27 | 122.79 | 0.20 | 10.86 | 9258.60 | 60.85 | 50825.84 |
| OVAR 027 | 193.50 | 146.13 | 2.90 | 5.61 | 70794.13 | 61.58 | 10904.98 |
| OVAR 028 | 193.50 | 106.55 | 1.37 | 2.13 | 20852.77 | 52.05 | 3267.80 |
| OVAR 029 | 2886.32 | 213.74 | 2.18 | 10.43 | 71197.93 | 37.06 | 58668.63 |
| OVAR 030 | 193.50 | 78.78 | 0.20 | 10.67 | 51820.51 | 92.01 | 2836.08 |
| OVAR 031 | 8283.49 | 215.55 | 3.72 | 5.19 | 26409.23 | 99.55 | 17724.56 |
| OVAR 032 | 16428.04 | 548.57 | 48.73 | 32.20 | 370132.66 | 68.27 | 49903.68 |
| OVAR 033 | 1766.02 | 108.96 | 0.20 | 3.49 | 6572.20 | 32.96 | 47862.24 |
| OVAR 034 | 193.50 | 109.90 | 0.20 | 31.37 | 1534.98 | 24.63 | 13462.38 |
| OVAR 035 | 5326.94 | 288.92 | 16.60 | 44.62 | 20830.46 | 206.71 | 30290.76 |
| OVAR 036 | 19787.17 | 468.37 | 28.94 | 51.25 | 7750.10 | 76.93 | 40337.62 |
| OVAR 037 | 3819.68 | 208.87 | 1.63 | 13.20 | 7103.26 | 70.13 | 36054.38 |
| OVAR 038 | 8572.63 | 173.00 | 0.20 | 5.65 | 5570.83 | 76.40 | 12553.53 |
| OVAR 039 | 938.70 | 114.25 | 3.31 | 2.62 | 33166.71 | 38.82 | 3815.64 |
| OVAR 040 | 193.50 | 236.99 | 4.76 | 29.88 | 2093.46 | 37.06 | 47329.23 |
| OVAR 041 | 193.50 | 213.99 | 2.52 | 3.82 | 23958.72 | 77.24 | 13123.55 |
| OVAR 042 | 193.50 | 137.99 | 0.20 | 6.39 | 29173.32 | 89.29 | 22987.87 |
| OVAR 043 | 21317.49 | 566.03 | 38.93 | 27.63 | 8990.10 | 46.47 | 53781.07 |
| OVAR 044 | 193.50 | 196.84 | 0.20 | 4.46 | 49816.08 | 51.17 | 20985.37 |
| BLDR 001 | 193.50 | 230.65 | 6.24 | 4.61 | 4176.50 | 188.41 | 79933.27 |
| BLDR 002 | 193.50 | 232.72 | 0.20 | 5.28 | 9722.75 | 92.46 | 23759.83 |
| BLDR 003 | 193.50 | 121.94 | 0.20 | 0.30 | 6947.85 | 185.31 | 49110.19 |
| BLDR 004 | 193.50 | 142.67 | 0.20 | 2.79 | 9810.34 | 61.52 | 3358.92 |
| BLDR 005 | 193.50 | 422.71 | 0.20 | 2.07 | 15054.78 | 97.49 | 31057.36 |
| BLDR 006 | 193.50 | 214.97 | 0.20 | 0.68 | 9954.29 | 57.92 | 35086.51 |
| BLDR 007 | 34265.57 | 207.31 | 0.20 | 13.30 | 5990.50 | 39.25 | 67354.89 |
| BLDR 008 | 193.50 | 318.09 | 4.53 | 10.81 | 25042.98 | 129.65 | 19710.73 |
| BLDR 009 | 193.50 | 126.34 | 0.20 | 2.53 | 731.16 | 20.94 | 21071.94 |
| BLDR 010 | 938.70 | 86.95 | 0.20 | 2.05 | 3704.22 | 178.19 | 28456.23 |
| BLDR 011 | 193.50 | 295.96 | 4.25 | 3.27 | 6297.23 | 124.66 | 22497.69 |
| BLDR 012 | 193.50 | 201.17 | 8.51 | 3.69 | 7204.22 | 52.66 | 29569.52 |
| BLDR 013 | 212.53 | 218.06 | 1.17 | 2.83 | 36055.14 | 115.89 | 22619.14 |
| BLDR 014 | 193.50 | 88.84 | 0.20 | 1.46 | 1911.19 | 18.47 | 951.10 |
| BLDR 015 | 193.50 | 176.46 | 0.20 | 5.34 | 3831.07 | 229.42 | 30835.53 |
| BLDR 016 | 193.50 | 142.84 | 0.20 | 2.20 | 3456.32 | 25.77 | 29593.56 |
| BLDR 017 | 193.50 | 154.51 | 3.30 | 5.00 | 15177.11 | 220.42 | 3017.92 |
| BLDR 018 | 193.50 | 272.17 | 0.64 | 5.46 | 9601.99 | 108.11 | 31661.90 |
| BLDR 019 | 193.50 | 301.29 | 0.20 | 1.81 | 16310.59 | 111.72 | 52393.46 |
| BLDR 020 | 1466.16 | 267.45 | 0.20 | 1.81 | 16160.73 | 133.46 | 46967.26 |
| BLDR 021 | 193.50 | 149.76 | 0.20 | 3.94 | 1874.38 | 100.74 | 12019.08 |
| BLDR 022 | 193.50 | 158.09 | 0.98 | 1.76 | 23801.73 | 105.88 | 3222.94 |
| BLDR 023 | 193.50 | 143.02 | 0.20 | 2.37 | 3046.01 | 19.09 | 10968.21 |
| BLDR 024 | 193.50 | 143.02 | 0.20 | 2.30 | 1000.89 | 23.40 | 8122.80 |
| BLDR 025 | 193.50 | 139.50 | 0.20 | 2.31 | 28789.39 | 45.90 | 2939.42 |
| BLDR 026 | 193.50 | 138.45 | 0.20 | 8.89 | 3068.22 | 131.19 | 4169.73 |
| BLDR 027 | 193.50 | 209.08 | 0.20 | 5.34 | 20344.22 | 149.91 | 14415.93 |
| BLDR 028 | 193.50 | 247.32 | 7.77 | 9.10 | 22018.09 | 66.96 | 45722.13 |
| BLDR 029 | 1403.45 | 346.11 | 5.43 | 10.81 | 46776.69 | 135.03 | 48108.45 |
| BLDR 030 | 193.50 | 130.59 | 0.20 | 8.63 | 1240.44 | 36.41 | 44376.20 |
| BLDR 031 | 193.50 | 317.29 | 0.20 | 10.47 | 1240.44 | 140.83 | 48745.34 |
| BLDR 032 | 193.50 | 186.33 | 1.72 | 5.34 | 6288.45 | 105.13 | 15841.98 |
| BLDR 033 | 466.98 | 155.90 | 0.20 | 1.60 | 1974.15 | 21.65 | 26970.10 |
| BLDR 034 | 193.50 | 56.32 | 0.20 | 3.13 | 4289.49 | 99.86 | 30053.38 |
| BLDR 035 | 193.50 | 433.08 | 0.20 | 27.65 | 65490.07 | 96.38 | 30177.35 |
| BLDR 036 | 193.50 | 135.63 | 0.20 | 2.24 | 15785.24 | 96.63 | 11419.97 |
| BLDR 037 | 1709.19 | 202.20 | 0.20 | 4.33 | 2671.22 | 149.70 | 51234.80 |
| BLDR 038 | 193.50 | 340.22 | 0.20 | 15.57 | 2208.33 | 302.83 | 52849.42 |
| BLDR 039 | 193.50 | 217.49 | 0.20 | 1.64 | 13588.82 | 27.93 | 64298.20 |
| BLDR 040 | 193.50 | 66.66 | 0.20 | 4.49 | 18082.77 | 51.87 | 16358.88 |
| BLDR 041 | 193.50 | 137.99 | 0.20 | 4.46 | 13993.49 | 62.68 | 44882.59 |
| BLDR 042 | 193.50 | 275.36 | 0.20 | 14.91 | 7871.61 | 117.04 | 41380.16 |
| BLDR 043 | 193.50 | 230.94 | 2.23 | 5.10 | 1163.48 | 220.59 | 43675.03 |
| BLDR 044 | 193.50 | 219.66 | 0.20 | 5.10 | 6026.27 | 188.71 | 28642.70 |
| BLDR 045 | 193.50 | 185.29 | 0.20 | 5.10 | 1671.11 | 117.04 | 25505.70 |
| BLDR 046 | 2850.42 | 157.34 | 0.20 | 0.35 | 6537.00 | 67.91 | 13752.53 |
| BLDR 047 | 212.53 | 161.28 | 0.20 | 1.77 | 2142.33 | 66.51 | 45177.97 |
| BLDR 048 | 907.66 | 139.40 | 0.20 | 1.36 | 12526.85 | 24.73 | 24007.02 |
| PDAC 001 | 882.39 | 144.49 | 0.20 | 8.61 | 17116.95 | 67.03 | 13311.33 |
| PDAC 002 | 5326.94 | 239.21 | 0.20 | 8.61 | 7497.21 | 52.12 | 30086.62 |
| PDAC 003 | 1619.96 | 321.51 | 0.20 | 4.58 | 11411.48 | 41.22 | 27668.87 |
| PDAC 004 | 193.50 | 134.65 | 3.02 | 10.51 | 2186.34 | 19.42 | 84276.76 |
| PDAC 005 | 2691.70 | 110.79 | 0.20 | 5.77 | 6988.24 | 80.16 | 24755.19 |
| PDAC 006 | 4072.25 | 220.35 | 37.50 | 9.70 | 4265.84 | 60.25 | 37878.29 |
| PDAC 007 | 7537.41 | 248.28 | 17.82 | 16.12 | 2623.90 | 69.25 | 42550.33 |
| PDAC 008 | 193.50 | 126.98 | 1.03 | 6.54 | 2354.43 | 43.03 | 17453.90 |
| PDAC 009 | 9496.12 | 168.37 | 2.15 | 5.84 | 13280.20 | 49.66 | 12756.52 |
| PDAC 010 | 193.50 | 248.72 | 0.20 | 2.36 | 8690.57 | 66.51 | 285191.54 |
| PDAC 011 | 193.50 | 183.11 | 2.79 | 16.91 | 751.81 | 33.81 | 34672.20 |
| PDAC 012 | 938.70 | 119.63 | 0.20 | 2.05 | 12552.75 | 48.89 | 5571.91 |
| PDAC 013 | 193.50 | 361.41 | 0.20 | 3.51 | 6364.15 | 21.42 | 3577.03 |
| PDAC 014 | 875.10 | 113.10 | 0.20 | 7.15 | 3310.68 | 43.12 | 31263.39 |
| PDAC 015 | 193.50 | 200.09 | 0.20 | 5.70 | 11912.41 | 46.54 | 27659.35 |
| PDAC 016 | 193.50 | 172.28 | 0.20 | 7.72 | 4289.92 | 60.75 | 47750.03 |
| PDAC 017 | 656.44 | 129.52 | 0.20 | 4.81 | 1256.65 | 39.01 | 34287.11 |
| PDAC 018 | 347.77 | 236.17 | 2.63 | 6.02 | 1913.68 | 54.17 | 49543.72 |
| PDAC 019 | 193.50 | 256.17 | 1.91 | 8.84 | 34516.60 | 40.62 | 34262.86 |
| PDAC 020 | 193.50 | 176.23 | 0.20 | 7.79 | 1070.73 | 55.01 | 90671.47 |
| PDAC 021 | 1438.60 | 141.77 | 0.20 | 4.95 | 3589.65 | 45.22 | 50337.71 |
| PDAC 022 | 193.50 | 139.67 | 0.20 | 3.76 | 3270.68 | 38.80 | 2284.29 |
| PDAC 023 | 193.50 | 148.27 | 0.20 | 7.47 | 6738.79 | 43.33 | 44559.58 |
| PDAC 024 | 193.50 | 142.99 | 0.20 | 13.70 | 2640.47 | 36.56 | 44878.79 |
| PDAC 025 | 193.50 | 205.84 | 7.17 | 10.04 | 1289.84 | 29.44 | 31332.78 |
| PDAC 026 | 965.11 | 646.28 | 10.07 | 50.63 | 2544.81 | 99.55 | 58628.42 |
| PDAC 027 | 193.50 | 156.46 | 0.20 | 4.88 | 31045.96 | 54.52 | 2814.21 |
| PDAC 028 | 907.66 | 291.04 | 0.20 | 12.18 | 4460.52 | 38.70 | 64379.59 |
| PDAC 029 | 907.66 | 160.62 | 5.47 | 2.97 | 1562.48 | 30.38 | 55430.38 |
| PDAC 030 | 311.59 | 161.81 | 0.20 | 3.62 | 2871.31 | 64.99 | 81997.71 |
| PDAC 031 | 7440.49 | 155.19 | 0.20 | 1.64 | 1640.14 | 56.95 | 34430.14 |
| PDAC 032 | 193.50 | 122.30 | 0.20 | 0.77 | 7404.74 | 25.00 | 43136.08 |
| PDAC 033 | 2009.16 | 140.83 | 0.20 | 1.72 | 16875.85 | 73.52 | 30915.42 |
| PDAC 034 | 193.50 | 156.49 | 0.20 | 2.62 | 3000.81 | 35.56 | 33577.10 |
| PDAC 035 | 193.50 | 132.93 | 0.20 | 1.89 | 2181.39 | 37.43 | 25026.26 |
| PDAC 036 | 193.50 | 91.80 | 17.09 | 5.79 | 1999.75 | 39.00 | 49795.07 |
| PDAC 037 | 1153.98 | 184.41 | 0.20 | 6.54 | 2394.10 | 69.21 | 128617.60 |
| PDAC 038 | 193.50 | 151.59 | 0.20 | 4.39 | 44459.35 | 33.18 | 57863.83 |
| PDAC 039 | 193.50 | 206.92 | 0.20 | 9.42 | 3246.38 | 73.47 | 16731.81 |
| PDAC 040 | 193.50 | 151.59 | 0.20 | 4.02 | 7537.63 | 29.63 | 14187.08 |
| PDAC 041 | 193.50 | 219.66 | 0.20 | 12.93 | 4786.12 | 158.40 | 28853.77 |
| PDAC 042 | 193.50 | 137.16 | 0.20 | 1.26 | 26947.84 | 44.02 | 50631.29 |
| PDAC 043 | 193.50 | 416.40 | 0.20 | 1.56 | 9258.33 | 52.04 | 43026.56 |
| PDAC 044 | 8625.46 | 282.37 | 1.46 | 20.49 | 3509.84 | 99.04 | 32345.74 |
| HC 001 | 1075.83 | 380.36 | 0.20 | 12.90 | 49293.75 | 167.09 | 64492.81 |
| HC 002 | 193.50 | 268.32 | 0.20 | 10.22 | 34668.23 | 121.04 | 19350.51 |
| HC 003 | 6312.99 | 462.63 | 0.20 | 12.41 | 113624.89 | 136.06 | 6679.56 |
| HC 004 | 6814.59 | 433.77 | 0.20 | 10.38 | 107105.88 | 112.90 | 5793.65 |
| HC 005 | 2339.12 | 301.23 | 0.20 | 4.25 | 60030.73 | 185.06 | 38786.06 |
| HC 006 | 193.50 | 338.46 | 0.20 | 6.82 | 77141.70 | 23.51 | 24773.20 |
| HC 007 | 193.50 | 208.91 | 0.20 | 5.53 | 122618.86 | 146.44 | 4689.22 |
| HC 008 | 5280.62 | 202.14 | 0.20 | 5.19 | 148686.73 | 45.46 | 1326.47 |
| HC 009 | 957.02 | 117.47 | 0.20 | 1.90 | 4277.99 | 59.84 | 2955.77 |
| HC 010 | 4178.06 | 202.14 | 0.20 | 4.83 | 145792.10 | 43.72 | 1281.37 |
| HC 011 | 648.39 | 127.89 | 0.20 | 1.72 | 4277.35 | 57.44 | 3264.34 |
| HC 012 | 4178.06 | 176.81 | 0.20 | 4.83 | 31243.77 | 56.89 | 1183.90 |
| HC 013 | 4178.06 | 176.92 | 0.20 | 5.19 | 34220.16 | 48.87 | 1348.45 |
| HC 014 | 3540.26 | 170.56 | 0.20 | 4.12 | 32285.84 | 50.90 | 1235.66 |
| HC 015 | 2330.69 | 157.74 | 0.20 | 3.76 | 31280.62 | 49.56 | 1118.67 |
| HC 016 | 11503.18 | 308.32 | 0.20 | 10.71 | 46447.63 | 65.31 | 8366.25 |
| HC 017 | 193.50 | 163.17 | 9.09 | 0.30 | 1904.02 | 77.41 | 3068.32 |
| HC 018 | 8634.89 | 212.79 | 0.20 | 8.98 | 33859.83 | 66.25 | 6007.83 |
| HC 019 | 9800.19 | 316.78 | 0.20 | 13.73 | 42227.51 | 68.49 | 7582.62 |
| HC 020 | 11984.00 | 335.47 | 0.20 | 16.76 | 45837.48 | 84.54 | 8942.86 |
| HC 021 | 1153.98 | 267.45 | 0.20 | 3.49 | 29979.45 | 148.16 | 29588.04 |
| HC 022 | 1153.98 | 298.16 | 0.20 | 4.63 | 31832.24 | 216.45 | 34502.55 |
| HC 023 | 1153.98 | 273.26 | 0.20 | 4.25 | 30924.65 | 135.45 | 31111.18 |
| HC 024 | 1153.98 | 299.77 | 0.20 | 5.01 | 31317.10 | 206.42 | 34888.67 |
| HC 025 | 957.02 | 183.45 | 0.20 | 1.90 | 12511.66 | 100.15 | 2082.72 |
| HC 026 | 1153.98 | 468.50 | 0.20 | 12.40 | 88542.06 | 175.67 | 57650.90 |
| HC 027 | 193.50 | 150.40 | 0.20 | 7.51 | 12866.97 | 28.08 | 19686.83 |
| HC 028 | 193.50 | 113.47 | 0.20 | 6.33 | 13682.20 | 29.77 | 1761.42 |
| HC 029 | 193.50 | 125.78 | 0.20 | 4.09 | 11059.61 | 26.05 | 7880.84 |
| HC 030 | 193.50 | 318.80 | 0.20 | 8.99 | 66245.04 | 125.42 | 20553.70 |
| HC 031 | 193.50 | 242.15 | 0.20 | 11.61 | 16035.38 | 190.72 | 38300.51 |
| HC 032 | 193.50 | 150.01 | 0.20 | 7.04 | 4479.11 | 64.95 | 8867.73 |
| HC 033 | 4309.16 | 229.02 | 0.20 | 8.02 | 51663.50 | 89.77 | 10163.26 |
| HC 034 | 12345.68 | 230.94 | 4.08 | 7.04 | 23958.72 | 123.33 | 7013.31 |
| HC 035 | 193.50 | 208.29 | 2.38 | 5.10 | 74829.78 | 69.45 | 1661.24 |
| HC 036 | 193.50 | 189.56 | 0.20 | 6.07 | 42093.69 | 69.14 | 5734.95 |
| HC 037 | 13075.35 | 275.36 | 2.66 | 8.34 | 25388.29 | 142.00 | 8000.22 |
| HC 038 | 193.50 | 125.83 | 0.20 | 7.04 | 4290.00 | 67.20 | 1425.70 |
| HC 039 | 193.50 | 264.36 | 2.95 | 7.04 | 84965.21 | 89.29 | 11046.18 |
| HC 040 | 5265.37 | 216.31 | 3.61 | 8.24 | 26386.07 | 152.44 | 7149.00 |
| HC 041 | 193.50 | 350.85 | 0.20 | 31.69 | 3867.87 | 426.10 | 7956.16 |
| HC 042 | 193.50 | 231.27 | 0.20 | 27.84 | 4026.61 | 228.13 | 2125.74 |
| HC 043 | 2155.83 | 229.78 | 0.20 | 23.55 | 84174.39 | 252.09 | 30973.80 |
| HC 044 | 13820.27 | 369.39 | 12.08 | 24.92 | 5415.97 | 355.88 | 18550.86 |
| HC 045 | 12479.31 | 361.37 | 11.07 | 16.37 | 5419.36 | 351.08 | 18882.38 |
| HC 046 | 1619.96 | 98.89 | 0.20 | 3.65 | 24487.49 | 85.50 | 24891.51 |
| HC 047 | 882.39 | 92.83 | 0.20 | 3.32 | 24401.93 | 57.95 | 23288.80 |
| HC 048 | 2155.83 | 110.79 | 0.20 | 3.97 | 25434.07 | 86.54 | 26293.13 |
| HC 049 | 7912.17 | 344.66 | 1.70 | 28.58 | 102717.04 | 461.38 | 8670.96 |
| HC 050 | 12778.90 | 348.95 | 10.95 | 23.55 | 3698.41 | 119.14 | 26333.33 |
| HC 051 | 13365.85 | 327.63 | 11.04 | 17.83 | 3711.18 | 111.15 | 25948.64 |
| HC 052 | 7570.19 | 310.77 | 2.15 | 7.48 | 77638.92 | 117.17 | 9064.89 |
| HC 053 | 193.50 | 99.29 | 0.20 | 2.68 | 767.89 | 24.35 | 18280.16 |
| HC 054 | 193.50 | 97.96 | 0.20 | 2.83 | 983.93 | 30.22 | 6017.81 |
| HC 055 | 193.50 | 177.77 | 2.47 | 4.68 | 5353.93 | 41.23 | 24296.26 |
| HC 056 | 193.50 | 122.07 | 1.72 | 3.69 | 5645.50 | 60.09 | 3783.26 |
| HC 057 | 193.50 | 224.48 | 2.15 | 8.96 | 32788.98 | 98.83 | 18873.01 |
| HC 058 | 193.50 | 199.68 | 0.20 | 5.41 | 5037.95 | 49.07 | 20618.01 |
| HC 059 | 9458.00 | 284.55 | 2.47 | 11.29 | 6315.49 | 116.18 | 20079.85 |
| HC 060 | 193.50 | 109.94 | 0.80 | 3.76 | 4190.52 | 46.66 | 21064.72 |
| HC 061 | 193.50 | 109.94 | 0.80 | 3.53 | 4301.60 | 41.23 | 20883.72 |
| HC 062 | 193.50 | 199.38 | 0.20 | 10.90 | 3425.84 | 99.55 | 21982.31 |
| HC 063 | 193.50 | 171.52 | 0.20 | 11.36 | 3170.25 | 86.22 | 20577.80 |
| HC 064 | 193.50 | 113.29 | 0.20 | 3.76 | 4379.29 | 56.23 | 20135.41 |
| HC 065 | 193.50 | 232.58 | 0.20 | 9.53 | 2422.82 | 70.43 | 16598.82 |
| HC 066 | 193.50 | 146.13 | 2.79 | 6.07 | 1784.49 | 47.86 | 25716.31 |
| HC 067 | 193.50 | 460.51 | 1.93 | 49.44 | 2597.00 | 15.15 | 12299.79 |
| HC 068 | 193.50 | 196.32 | 0.20 | 16.41 | 7575.81 | 58.02 | 23646.86 |
| HC 069 | 193.50 | 221.51 | 0.20 | 3.40 | 1430.29 | 31.64 | 41153.71 |
| HC 070 | 193.50 | 213.56 | 0.20 | 8.73 | 8144.68 | 33.65 | 25503.08 |
| HC 071 | 193.50 | 283.79 | 0.20 | 4.86 | 23377.32 | 39.62 | 14689.23 |
| HC 072 | 193.50 | 193.29 | 0.20 | 9.33 | 8818.71 | 35.63 | 24956.86 |
| HC 073 | 193.50 | 321.54 | 0.20 | 6.04 | 31856.42 | 44.24 | 14864.20 |
| HC 074 | 193.50 | 347.99 | 0.20 | 8.43 | 28546.48 | 48.20 | 16150.96 |
| HC 075 | 193.50 | 314.24 | 0.20 | 7.83 | 25483.09 | 47.55 | 16394.19 |
| HC 076 | 193.50 | 173.08 | 0.20 | 16.33 | 2145.92 | 32.31 | 21902.24 |
| HC 077 | 193.50 | 468.26 | 0.20 | 20.95 | 24293.41 | 38.30 | 13544.66 |
| HC 078 | 193.50 | 453.93 | 0.20 | 22.81 | 22668.70 | 34.31 | 5750.89 |
| HC 079 | 193.50 | 177.24 | 0.20 | 10.54 | 2086.91 | 35.64 | 26013.43 |
| HC 080 | 193.50 | 457.51 | 0.20 | 26.54 | 22294.16 | 42.27 | 12587.87 |
| HC 081 | 193.50 | 164.90 | 0.20 | 10.24 | 2185.35 | 38.30 | 24957.09 |
| HC 082 | 193.50 | 335.75 | 0.20 | 17.57 | 101133.24 | 48.81 | 6934.27 |
| HC 083 | 193.50 | 737.94 | 0.20 | 61.73 | 3594.75 | 16.04 | 20582.76 |
| HC 084 | 193.50 | 217.46 | 0.20 | 15.11 | 3556.87 | 78.09 | 19362.12 |
| HC 085 | 193.50 | 245.13 | 0.20 | 17.87 | 3703.81 | 101.17 | 21702.08 |
| HC 086 | 193.50 | 252.93 | 0.20 | 8.88 | 2086.91 | 61.27 | 28971.64 |
| HC 087 | 193.50 | 241.22 | 0.20 | 3.39 | 81687.13 | 34.31 | 8655.32 |
| HC 088 | 193.50 | 247.08 | 0.20 | 5.60 | 65695.99 | 56.06 | 11367.08 |
| HC 089 | 193.50 | 295.16 | 0.20 | 13.27 | 2165.80 | 50.17 | 15333.57 |
| HC 090 | 193.50 | 247.06 | 0.20 | 10.84 | 2127.02 | 58.01 | 14136.89 |
| HC 091 | 193.50 | 366.42 | 0.20 | 12.05 | 102699.13 | 65.81 | 8195.40 |
| HC 092 | 193.50 | 268.44 | 0.20 | 15.41 | 2127.02 | 75.84 | 14184.44 |
| HC 093 | 193.50 | 259.52 | 0.20 | 9.65 | 32290.20 | 59.27 | 12523.11 |
| HC 094 | 193.50 | 189.40 | 0.20 | 7.53 | 12715.45 | 52.79 | 17335.84 |
| HC 095 | 193.50 | 162.44 | 4.53 | 8.57 | 11146.84 | 156.92 | 2089.91 |
| HC 096 | 193.50 | 205.57 | 0.20 | 7.53 | 11916.05 | 47.54 | 17467.22 |
| HC 097 | 193.50 | 185.17 | 0.20 | 8.43 | 13654.63 | 54.08 | 19644.40 |
| HC 098 | 193.50 | 254.42 | 0.20 | 9.34 | 41233.67 | 67.71 | 10500.00 |
| HC 099 | 193.50 | 213.56 | 0.20 | 20.18 | 2492.14 | 286.43 | 13976.74 |
| HC 100 | 193.50 | 109.74 | 0.20 | 3.10 | 33734.50 | 93.50 | 10985.22 |
| HC 101 | 193.50 | 87.37 | 0.20 | 2.53 | 29093.35 | 62.56 | 14036.41 |
| HC 102 | 193.50 | 78.25 | 0.20 | 2.53 | 33492.34 | 49.48 | 13060.64 |
| HC 103 | 193.50 | 109.05 | 0.20 | 3.67 | 31050.04 | 84.82 | 13907.26 |
| HC 104 | 193.50 | 132.96 | 0.20 | 4.63 | 41382.30 | 37.35 | 12717.11 |
| HC 105 | 193.50 | 169.36 | 0.20 | 1.99 | 1378.44 | 40.32 | 74421.56 |
| HC 106 | 193.50 | 187.59 | 0.20 | 2.18 | 1372.00 | 43.04 | 76024.43 |
| HC 107 | 193.50 | 144.03 | 0.20 | 1.62 | 1378.44 | 31.38 | 60080.59 |
| HC 108 | 193.50 | 204.91 | 0.20 | 21.55 | 11707.60 | 100.98 | 14164.21 |
| HC 109 | 193.50 | 217.00 | 0.20 | 8.37 | 7474.02 | 68.90 | 19828.17 |
| HC 110 | 193.50 | 176.36 | 0.20 | 13.67 | 11294.28 | 78.70 | 14201.67 |
| HC 111 | 193.50 | 200.86 | 0.20 | 6.56 | 6972.48 | 72.82 | 18895.22 |
| HC 112 | 193.50 | 200.86 | 0.20 | 17.04 | 11617.21 | 102.95 | 15274.37 |
| HC 113 | 193.50 | 130.30 | 0.20 | 5.00 | 1558.31 | 97.93 | 7192.64 |

| Subject | | | | | | | |
|---|---|---|---|---|---|---|---|
| Cohort | | | | | | | |
| ID | Prolactin | SCF | TNFa | TRAIL | Total PSA | VEGF | b-HCG |
| OVAR 001 | 36.93 | 2.00 | 0.30 | 0.50 | 2.00 | 6.40 | 0.03 |
| OVAR 002 | 30.20 | 2.00 | 0.30 | 0.50 | 2.00 | 6.40 | 0.03 |
| OVAR 003 | 30.20 | 2.00 | 0.30 | 0.50 | 2.00 | 6.40 | 0.03 |
| OVAR 004 | 30.20 | 2.00 | 0.51 | 0.61 | 2.51 | 8.95 | 0.04 |
| OVAR 005 | 39.50 | 2.00 | 0.30 | 0.50 | 2.21 | 6.40 | 0.03 |
| OVAR 006 | 39.14 | 2.00 | 0.30 | 0.50 | 2.00 | 6.40 | 0.03 |
| OVAR 007 | 30.20 | 2.00 | 0.58 | 0.50 | 2.00 | 22.67 | 0.03 |
| OVAR 008 | 30.20 | 3.33 | 0.47 | 0.50 | 2.21 | 14.97 | 0.03 |
| OVAR 009 | 82.53 | 2.00 | 0.30 | 0.50 | 2.00 | 18.43 | 0.09 |
| OVAR 010 | 39.39 | 2.00 | 0.35 | 1.92 | 2.00 | 14.51 | 0.03 |
| OVAR 011 | 49.79 | 2.00 | 0.30 | 0.61 | 2.01 | 9.58 | 0.04 |
| OVAR 012 | 32.38 | 2.00 | 0.80 | 0.61 | 2.31 | 10.12 | 0.04 |
| OVAR 013 | 61.15 | 3.24 | 0.83 | 1.45 | 2.00 | 6.40 | 0.07 |
| OVAR 014 | 179.45 | 2.03 | 0.94 | 1.54 | 2.00 | 13.20 | 0.03 |
| OVAR 015 | 54.69 | 2.10 | 0.77 | 0.50 | 2.00 | 10.34 | 0.06 |
| OVAR 016 | 44.50 | 2.00 | 0.77 | 0.65 | 2.35 | 6.40 | 0.03 |
| OVAR 017 | 96.72 | 2.00 | 0.30 | 0.77 | 2.00 | 6.40 | 0.03 |
| OVAR 018 | 46.56 | 4.02 | 0.30 | 0.89 | 2.00 | 6.40 | 0.03 |
| OVAR 019 | 160.56 | 2.01 | 0.58 | 1.18 | 2.00 | 14.09 | 0.03 |
| OVAR 020 | 30.20 | 2.00 | 0.30 | 0.50 | 2.00 | 6.40 | 0.03 |
| OVAR 021 | 30.20 | 2.00 | 0.42 | 0.50 | 2.00 | 11.80 | 0.05 |
| OVAR 022 | 38.08 | 2.00 | 0.30 | 1.73 | 2.00 | 26.18 | 0.03 |
| OVAR 023 | 129.90 | 2.75 | 1.22 | 1.37 | 2.02 | 8.51 | 0.04 |
| OVAR 024 | 85.71 | 2.00 | 0.82 | 1.66 | 2.00 | 21.14 | 0.03 |
| OVAR 025 | 78.01 | 2.00 | 1.04 | 1.01 | 2.00 | 14.49 | 0.03 |
| OVAR 026 | 30.20 | 2.10 | 0.81 | 0.82 | 2.00 | 9.58 | 0.03 |
| OVAR 027 | 116.32 | 5.50 | 0.53 | 4.99 | 2.00 | 10.86 | 0.05 |
| OVAR 028 | 240.25 | 5.50 | 0.53 | 2.51 | 2.00 | 15.83 | 0.07 |
| OVAR 029 | 57.26 | 2.00 | 0.41 | 1.51 | 2.37 | 10.20 | 0.06 |
| OVAR 030 | 30.20 | 2.01 | 0.30 | 1.56 | 2.00 | 13.06 | 0.03 |
| OVAR 031 | 104.65 | 2.75 | 0.50 | 2.08 | 2.00 | 14.90 | 0.06 |
| OVAR 032 | 43.75 | 2.00 | 0.30 | 0.50 | 2.00 | 6.40 | 0.03 |
| OVAR 033 | 43.96 | 2.18 | 2.14 | 0.63 | 5.91 | 6.40 | 0.03 |
| OVAR 034 | 30.20 | 2.00 | 0.30 | 0.50 | 2.00 | 6.40 | 0.03 |
| OVAR 035 | 129.77 | 2.78 | 0.98 | 2.43 | 2.06 | 27.40 | 0.04 |
| OVAR 036 | 112.38 | 2.00 | 1.48 | 1.19 | 2.00 | 15.92 | 0.03 |
| OVAR 037 | 83.06 | 2.10 | 0.38 | 0.66 | 2.00 | 6.40 | 0.03 |
| OVAR 038 | 306.63 | 2.22 | 0.82 | 1.84 | 2.00 | 12.72 | 0.03 |
| OVAR 039 | 48.72 | 2.00 | 1.67 | 0.61 | 2.14 | 6.40 | 0.04 |
| OVAR 040 | 89.65 | 2.00 | 0.30 | 0.50 | 2.00 | 6.40 | 0.03 |
| OVAR 041 | 30.20 | 2.47 | 0.91 | 0.50 | 3.08 | 19.47 | 0.03 |
| OVAR 042 | 1278.95 | 22.94 | 2.26 | 24.72 | 2.59 | 6.40 | 0.15 |
| OVAR 043 | 30.20 | 2.47 | 0.66 | 0.50 | 3.32 | 21.01 | 0.03 |
| OVAR 044 | 792.52 | 16.30 | 2.26 | 20.29 | 2.10 | 6.40 | 0.18 |
| BLDR 001 | 104.79 | 2.88 | 1.26 | 1.13 | 16.91 | 13.41 | 0.03 |
| BLDR 002 | 30.20 | 2.00 | 0.30 | 0.63 | 2.00 | 8.47 | 0.03 |
| BLDR 003 | 35.01 | 2.25 | 0.89 | 0.80 | 12.29 | 6.40 | 0.03 |
| BLDR 004 | 30.20 | 2.00 | 1.02 | 0.50 | 2.00 | 16.44 | 0.08 |
| BLDR 005 | 49.04 | 8.89 | 0.91 | 1.37 | 24.47 | 14.45 | 0.03 |
| BLDR 006 | 45.05 | 2.25 | 0.30 | 1.29 | 2.00 | 8.90 | 0.03 |
| BLDR 007 | 256.31 | 4.87 | 0.91 | 2.07 | 2.00 | 6.40 | 0.09 |
| BLDR 008 | 268.49 | 3.64 | 0.30 | 1.40 | 2.00 | 6.40 | 0.06 |
| BLDR 009 | 94.68 | 2.00 | 1.70 | 1.22 | 32.99 | 7.53 | 0.05 |
| BLDR 010 | 98.80 | 2.00 | 0.65 | 0.74 | 10.71 | 10.12 | 0.03 |
| BLDR 011 | 144.44 | 2.00 | 0.30 | 0.50 | 2.17 | 9.08 | 0.03 |
| BLDR 012 | 326.62 | 3.64 | 0.30 | 2.30 | 2.00 | 14.06 | 0.06 |
| BLDR 013 | 71.10 | 5.64 | 0.78 | 1.71 | 10.76 | 9.96 | 0.03 |
| BLDR 014 | 63.30 | 2.00 | 0.86 | 0.77 | 4.94 | 6.40 | 0.03 |
| BLDR 015 | 51.22 | 2.49 | 1.62 | 1.53 | 6.50 | 15.40 | 0.03 |
| BLDR 016 | 30.20 | 2.00 | 0.62 | 0.50 | 2.00 | 11.38 | 0.03 |
| BLDR 017 | 30.20 | 2.00 | 0.33 | 0.95 | 3.90 | 14.06 | 0.06 |
| BLDR 018 | 214.83 | 5.26 | 0.93 | 3.87 | 81.56 | 6.40 | 0.05 |
| BLDR 019 | 138.63 | 2.00 | 0.30 | 0.58 | 2.00 | 14.76 | 0.03 |
| BLDR 020 | 34.25 | 2.00 | 0.30 | 0.77 | 8.85 | 17.19 | 0.07 |
| BLDR 021 | 95.38 | 3.30 | 0.91 | 1.71 | 22.15 | 10.44 | 0.05 |
| BLDR 022 | 30.20 | 2.00 | 0.30 | 0.50 | 5.28 | 6.40 | 0.03 |
| BLDR 023 | 30.20 | 2.00 | 0.30 | 0.50 | 2.19 | 6.40 | 0.03 |
| BLDR 024 | 30.20 | 2.00 | 0.30 | 0.50 | 2.00 | 6.40 | 0.03 |
| BLDR 025 | 360.20 | 2.00 | 0.58 | 0.99 | 2.00 | 13.43 | 0.03 |
| BLDR 026 | 44.53 | 2.67 | 0.30 | 0.95 | 2.00 | 17.95 | 0.06 |
| BLDR 027 | 30.20 | 2.00 | 0.54 | 0.64 | 3.24 | 9.71 | 0.03 |
| BLDR 028 | 30.20 | 2.00 | 0.58 | 0.50 | 2.00 | 13.43 | 0.03 |
| BLDR 029 | 30.20 | 2.00 | 0.30 | 0.50 | 6.80 | 17.01 | 0.03 |
| BLDR 030 | 48.14 | 2.00 | 4.23 | 0.63 | 5.01 | 16.98 | 0.03 |
| BLDR 031 | 90.99 | 3.22 | 0.50 | 1.14 | 23.82 | 7.90 | 0.03 |
| BLDR 032 | 109.85 | 2.81 | 0.30 | 1.92 | 2.00 | 23.13 | 0.24 |
| BLDR 033 | 308.80 | 5.37 | 0.30 | 2.09 | 2.00 | 9.08 | 0.11 |
| BLDR 034 | 30.20 | 2.00 | 2.93 | 0.50 | 6.27 | 13.18 | 0.04 |
| BLDR 035 | 43.38 | 2.00 | 0.30 | 0.50 | 5.17 | 18.24 | 0.03 |
| BLDR 036 | 30.20 | 2.00 | 0.30 | 0.65 | 6.96 | 6.40 | 0.03 |
| BLDR 037 | 31.65 | 2.00 | 0.87 | 0.50 | 2.57 | 15.48 | 0.03 |
| BLDR 038 | 30.20 | 3.68 | 0.79 | 0.50 | 2.00 | 16.81 | 0.03 |
| BLDR 039 | 201.20 | 2.91 | 0.30 | 1.12 | 323.29 | 7.15 | 0.03 |
| BLDR 040 | 30.20 | 2.00 | 1.59 | 0.50 | 4.42 | 7.99 | 0.03 |
| BLDR 041 | 675.17 | 2.47 | 0.30 | 0.95 | 2.71 | 13.40 | 0.03 |
| BLDR 042 | 30.20 | 2.47 | 0.53 | 0.95 | 7.36 | 15.71 | 0.03 |
| BLDR 043 | 30.20 | 2.47 | 0.30 | 0.50 | 3.32 | 16.81 | 0.03 |
| BLDR 044 | 1289.08 | 22.94 | 2.38 | 24.50 | 3.82 | 6.40 | 0.15 |
| BLDR 045 | 1395.31 | 11.80 | 2.26 | 14.06 | 146.24 | 9.62 | 0.05 |
| BLDR 046 | 73.04 | 2.10 | 0.30 | 0.50 | 2.00 | 7.43 | 0.03 |
| BLDR 047 | 35.00 | 2.00 | 2.07 | 0.77 | 7.40 | 13.76 | 0.42 |
| BLDR 048 | 190.34 | 2.00 | 3.41 | 1.07 | 4.16 | 6.40 | 0.03 |
| PDAC 001 | 248.11 | 2.00 | 1.05 | 2.45 | 2.00 | 6.40 | 0.03 |
| PDAC 002 | 337.83 | 2.00 | 0.71 | 0.84 | 2.00 | 19.43 | 0.03 |
| PDAC 003 | 13410.77 | 12.89 | 2.42 | 16.35 | 2.00 | 17.86 | 0.30 |
| PDAC 004 | 30.58 | 5.57 | 0.30 | 0.50 | 2.00 | 13.74 | 0.03 |
| PDAC 005 | 791.59 | 2.00 | 1.42 | 1.54 | 2.00 | 6.40 | 0.03 |
| PDAC 006 | 728.67 | 2.00 | 1.67 | 1.54 | 2.00 | 21.20 | 0.03 |
| PDAC 007 | 175.88 | 2.00 | 0.51 | 1.42 | 2.00 | 20.88 | 0.03 |
| PDAC 008 | 523.10 | 5.03 | 0.58 | 2.33 | 2.00 | 13.80 | 0.03 |
| PDAC 009 | 143.78 | 2.47 | 0.58 | 0.50 | 2.00 | 7.07 | 0.05 |
| PDAC 010 | 492.44 | 2.00 | 0.41 | 0.50 | 2.00 | 12.80 | 0.03 |
| PDAC 011 | 237.77 | 2.01 | 0.97 | 0.50 | 16.49 | 14.56 | 0.03 |
| PDAC 012 | 725.60 | 3.00 | 6.64 | 1.54 | 2.11 | 17.05 | 0.03 |
| PDAC 013 | 1425.00 | 2.00 | 1.83 | 0.80 | 2.72 | 11.28 | 0.03 |
| PDAC 014 | 444.46 | 2.00 | 0.30 | 0.88 | 2.00 | 12.13 | 0.03 |
| PDAC 015 | 198.10 | 2.01 | 0.63 | 1.56 | 2.00 | 6.40 | 0.05 |
| PDAC 016 | 145.09 | 2.00 | 1.60 | 0.88 | 8.14 | 12.66 | 0.03 |
| PDAC 017 | 122.99 | 6.24 | 0.47 | 2.71 | 2.15 | 6.40 | 0.08 |
| PDAC 018 | 124.16 | 2.00 | 0.44 | 1.75 | 5.26 | 8.43 | 0.03 |
| PDAC 019 | 324.71 | 2.47 | 0.58 | 1.56 | 2.00 | 15.57 | 0.03 |
| PDAC 020 | 1369.68 | 2.25 | 0.30 | 0.96 | 4.17 | 6.40 | 0.03 |
| PDAC 021 | 332.82 | 2.00 | 0.80 | 0.50 | 2.00 | 9.18 | 0.03 |
| PDAC 022 | 342.98 | 2.92 | 0.33 | 3.65 | 2.00 | 22.19 | 0.05 |
| PDAC 023 | 356.68 | 4.35 | 0.46 | 3.57 | 2.00 | 12.45 | 0.04 |
| PDAC 024 | 282.23 | 4.33 | 0.62 | 1.94 | 2.00 | 16.13 | 0.03 |
| PDAC 025 | 1629.91 | 3.24 | 0.46 | 1.29 | 2.00 | 9.95 | 0.04 |
| PDAC 026 | 324.49 | 2.48 | 1.42 | 0.97 | 2.00 | 16.47 | 0.03 |
| PDAC 027 | 806.16 | 2.10 | 0.77 | 0.82 | 2.00 | 14.91 | 0.03 |
| PDAC 028 | 104.21 | 2.00 | 0.63 | 0.50 | 2.00 | 17.92 | 0.03 |
| PDAC 029 | 1190.83 | 4.92 | 0.35 | 1.20 | 2.00 | 7.15 | 0.03 |
| PDAC 030 | 30.20 | 2.00 | 0.30 | 0.88 | 8.80 | 8.88 | 0.03 |
| PDAC 031 | 2954.99 | 2.09 | 0.62 | 1.14 | 2.00 | 6.40 | 0.05 |
| PDAC 032 | 679.74 | 4.99 | 0.62 | 1.93 | 34.03 | 14.50 | 0.07 |
| PDAC 033 | 203.83 | 2.00 | 1.08 | 1.27 | 16.28 | 6.40 | 0.03 |
| PDAC 034 | 816.93 | 4.52 | 0.83 | 2.88 | 24.95 | 14.98 | 0.05 |
| PDAC 035 | 88.00 | 2.00 | 0.94 | 0.74 | 2.00 | 8.42 | 0.03 |
| PDAC 036 | 844.73 | 2.00 | 0.30 | 0.50 | 2.33 | 6.40 | 0.03 |
| PDAC 037 | 30.33 | 2.00 | 0.33 | 0.63 | 3.48 | 7.29 | 0.03 |
| PDAC 038 | 744.61 | 2.00 | 1.58 | 0.50 | 2.00 | 6.40 | 0.11 |
| PDAC 039 | 545.80 | 2.00 | 0.30 | 1.91 | 2.33 | 36.36 | 0.03 |
| PDAC 040 | 449.84 | 2.00 | 0.30 | 0.50 | 2.00 | 6.40 | 0.03 |
| PDAC 041 | 89.18 | 2.00 | 0.30 | 0.50 | 2.00 | 13.40 | 0.03 |
| PDAC 042 | 523.95 | 2.00 | 0.53 | 0.92 | 2.00 | 8.50 | 0.04 |
| PDAC 043 | 3642.03 | 3.20 | 0.94 | 2.50 | 2.00 | 18.30 | 0.03 |
| PDAC 044 | 108.00 | 2.00 | 0.50 | 0.50 | 2.00 | 6.40 | 0.03 |
| HC 001 | 77.82 | 2.00 | 0.58 | 1.04 | 2.00 | 17.24 | 0.04 |
| HC 002 | 30.20 | 2.00 | 0.30 | 0.50 | 2.00 | 6.40 | 0.03 |
| HC 003 | 137.02 | 5.15 | 0.64 | 1.03 | 3.66 | 6.40 | 0.05 |
| HC 004 | 275.73 | 9.80 | 0.61 | 2.09 | 2.00 | 6.40 | 0.05 |
| HC 005 | 87.12 | 2.64 | 0.85 | 1.50 | 2.00 | 6.40 | 0.05 |
| HC 006 | 91.35 | 2.00 | 0.30 | 0.50 | 2.00 | 10.26 | 0.03 |
| HC 007 | 38.63 | 2.00 | 1.50 | 1.15 | 2.00 | 6.40 | 0.03 |
| HC 008 | 95.84 | 3.59 | 0.91 | 1.74 | 2.00 | 6.40 | 0.03 |
| HC 009 | 30.20 | 2.00 | 1.19 | 0.80 | 2.04 | 9.76 | 0.03 |
| HC 010 | 41.95 | 2.64 | 2.02 | 0.79 | 2.00 | 6.40 | 0.05 |
| HC 011 | 368.93 | 3.80 | 0.44 | 3.26 | 10.15 | 6.40 | 0.05 |
| HC 012 | 50.11 | 2.00 | 0.30 | 0.50 | 2.00 | 6.40 | 0.03 |
| HC 013 | 180.28 | 2.64 | 0.30 | 0.55 | 2.00 | 6.40 | 0.03 |
| HC 014 | 94.43 | 2.00 | 0.30 | 0.55 | 2.00 | 6.40 | 0.05 |
| HC 015 | 109.88 | 2.00 | 0.30 | 0.50 | 2.00 | 6.40 | 0.05 |
| HC 016 | 42.91 | 2.00 | 0.55 | 0.55 | 2.00 | 6.40 | 0.05 |
| HC 017 | 416.99 | 3.03 | 0.44 | 2.11 | 7.12 | 8.45 | 0.03 |
| HC 018 | 43.63 | 2.00 | 0.85 | 0.50 | 2.00 | 6.40 | 0.03 |
| HC 019 | 40.24 | 2.00 | 0.30 | 0.50 | 2.00 | 6.40 | 0.03 |
| HC 020 | 30.20 | 2.00 | 0.67 | 0.50 | 2.00 | 6.40 | 0.03 |
| HC 021 | 57.58 | 2.00 | 0.30 | 0.50 | 2.00 | 11.55 | 0.03 |
| HC 022 | 88.10 | 2.00 | 0.30 | 0.50 | 2.00 | 10.31 | 0.03 |
| HC 023 | 100.64 | 2.09 | 0.69 | 0.50 | 2.00 | 16.26 | 0.06 |
| HC 024 | 78.70 | 2.09 | 0.30 | 0.50 | 2.00 | 18.43 | 0.04 |
| HC 025 | 1317.77 | 3.18 | 0.55 | 2.03 | 7.23 | 15.26 | 0.03 |
| HC 026 | 127.97 | 2.00 | 0.30 | 0.88 | 2.00 | 15.68 | 0.03 |
| HC 027 | 30.20 | 2.33 | 0.84 | 15.72 | 2.88 | 6.40 | 0.03 |
| HC 028 | 30.20 | 2.00 | 0.86 | 0.50 | 2.00 | 6.40 | 0.03 |
| HC 029 | 74.27 | 2.00 | 0.30 | 0.50 | 2.00 | 6.40 | 0.03 |
| HC 030 | 166.32 | 4.28 | 3.15 | 1.41 | 6.34 | 18.95 | 0.03 |
| HC 031 | 38.99 | 3.68 | 1.47 | 1.41 | 3.08 | 30.38 | 0.03 |
| HC 032 | 30.20 | 2.00 | 0.40 | 0.50 | 4.57 | 15.71 | 0.03 |
| HC 033 | 3184.41 | 6.00 | 1.70 | 9.51 | 2.65 | 12.86 | 0.06 |
| HC 034 | 30.20 | 2.00 | 0.79 | 0.50 | 2.00 | 19.99 | 0.03 |
| HC 035 | 1973.40 | 11.80 | 2.85 | 20.73 | 66.53 | 6.40 | 0.09 |
| HC 036 | 665.41 | 12.80 | 2.74 | 14.39 | 2.59 | 21.01 | 0.09 |
| HC 037 | 30.20 | 3.68 | 1.65 | 1.18 | 2.10 | 37.15 | 0.03 |
| HC 038 | 714.41 | 14.06 | 2.26 | 20.73 | 3.32 | 9.62 | 0.15 |
| HC 039 | 30.20 | 2.47 | 1.28 | 0.95 | 4.57 | 22.99 | 0.03 |
| HC 040 | 34.84 | 2.00 | 0.30 | 0.95 | 2.00 | 22.34 | 0.06 |
| HC 041 | 30.20 | 3.68 | 0.30 | 0.95 | 2.00 | 25.85 | 0.03 |
| HC 042 | 30.20 | 3.64 | 0.30 | 1.40 | 2.00 | 27.93 | 0.06 |
| HC 043 | 52.47 | 2.00 | 0.45 | 1.19 | 2.00 | 16.22 | 0.03 |
| HC 044 | 63.04 | 2.00 | 0.64 | 1.37 | 3.20 | 15.08 | 0.04 |
| HC 045 | 58.87 | 2.00 | 0.55 | 1.19 | 4.05 | 12.48 | 0.03 |
| HC 046 | 44.27 | 2.00 | 1.05 | 0.74 | 2.07 | 11.87 | 0.03 |
| HC 047 | 80.84 | 2.00 | 1.22 | 1.91 | 18.07 | 23.58 | 0.03 |
| HC 048 | 38.54 | 2.00 | 2.98 | 1.20 | 3.79 | 6.40 | 0.03 |
| HC 049 | 30.20 | 2.00 | 0.50 | 0.50 | 2.37 | 6.40 | 0.03 |
| HC 050 | 135.40 | 2.00 | 0.92 | 1.19 | 9.81 | 6.40 | 0.03 |
| HC 051 | 305.74 | 4.61 | 0.64 | 2.45 | 3.45 | 6.40 | 0.03 |
| HC 052 | 192.48 | 2.00 | 0.30 | 2.19 | 2.00 | 6.40 | 0.03 |
| HC 053 | 292.78 | 2.92 | 0.63 | 3.75 | 3.43 | 15.32 | 0.05 |
| HC 054 | 347.62 | 3.61 | 0.73 | 3.85 | 4.49 | 15.07 | 0.03 |
| HC 055 | 198.34 | 4.27 | 0.92 | 3.28 | 11.08 | 9.37 | 0.05 |
| HC 056 | 44.53 | 2.67 | 0.54 | 0.95 | 4.08 | 15.07 | 0.04 |
| HC 057 | 181.64 | 2.94 | 1.15 | 3.09 | 2.00 | 16.04 | 0.05 |
| HC 058 | 34.37 | 2.00 | 0.35 | 0.50 | 2.00 | 15.09 | 0.03 |
| HC 059 | 317.21 | 2.00 | 1.11 | 1.86 | 4.19 | 23.58 | 0.03 |
| HC 060 | 112.04 | 3.17 | 0.77 | 5.36 | 2.00 | 6.40 | 0.05 |
| HC 061 | 164.32 | 4.34 | 1.04 | 5.91 | 2.00 | 8.94 | 0.05 |
| HC 062 | 178.43 | 4.33 | 1.45 | 1.45 | 3.54 | 16.46 | 0.03 |
| HC 063 | 190.98 | 3.97 | 0.30 | 1.29 | 3.08 | 22.76 | 0.03 |
| HC 064 | 206.33 | 4.34 | 1.42 | 3.90 | 10.55 | 6.46 | 0.04 |
| HC 065 | 30.20 | 2.00 | 0.30 | 0.50 | 2.00 | 6.40 | 0.03 |
| HC 066 | 30.20 | 2.00 | 0.30 | 0.50 | 2.00 | 6.40 | 0.03 |
| HC 067 | 140.06 | 2.86 | 0.62 | 1.61 | 11.18 | 8.53 | 0.04 |
| HC 068 | 61.33 | 3.61 | 0.77 | 1.61 | 2.00 | 12.51 | 0.06 |
| HC 069 | 41.54 | 2.00 | 0.99 | 1.31 | 2.00 | 12.61 | 0.03 |
| HC 070 | 56.60 | 3.51 | 0.58 | 0.75 | 2.43 | 10.21 | 0.03 |
| HC 071 | 30.20 | 2.00 | 0.63 | 0.50 | 2.00 | 6.48 | 0.03 |
| HC 072 | 322.35 | 6.04 | 1.22 | 2.45 | 15.73 | 7.03 | 0.05 |
| HC 073 | 30.20 | 2.00 | 0.54 | 0.57 | 2.00 | 7.60 | 0.03 |
| HC 074 | 30.20 | 2.00 | 0.30 | 0.57 | 2.00 | 6.40 | 0.03 |
| HC 075 | 47.88 | 2.00 | 0.41 | 1.26 | 3.10 | 16.68 | 0.03 |
| HC 076 | 92.01 | 4.08 | 1.45 | 1.17 | 2.04 | 11.05 | 0.03 |
| HC 077 | 55.61 | 2.00 | 1.06 | 0.50 | 2.00 | 6.40 | 0.03 |
| HC 078 | 73.86 | 2.00 | 0.64 | 0.50 | 2.00 | 6.40 | 0.03 |
| HC 079 | 65.55 | 2.08 | 0.39 | 0.61 | 2.00 | 7.40 | 0.03 |
| HC 080 | 174.76 | 2.00 | 0.44 | 0.50 | 2.00 | 6.40 | 0.07 |
| HC 081 | 63.70 | 2.65 | 0.66 | 0.67 | 2.00 | 9.36 | 0.03 |
| HC 082 | 30.20 | 2.00 | 1.44 | 0.50 | 2.00 | 6.40 | 0.03 |
| HC 083 | 50.12 | 2.00 | 0.30 | 0.50 | 2.36 | 6.40 | 0.03 |
| HC 084 | 233.51 | 3.23 | 1.40 | 1.73 | 16.25 | 10.63 | 0.06 |
| HC 085 | 30.20 | 2.00 | 0.49 | 0.50 | 3.18 | 6.40 | 0.03 |
| HC 086 | 485.89 | 2.52 | 0.30 | 1.45 | 2.01 | 6.40 | 0.03 |
| HC 087 | 30.20 | 2.00 | 0.43 | 0.84 | 2.00 | 6.40 | 0.03 |
| HC 088 | 30.20 | 2.00 | 0.30 | 0.50 | 2.00 | 6.40 | 0.03 |
| HC 089 | 30.20 | 2.00 | 0.64 | 0.61 | 2.00 | 6.40 | 0.03 |
| HC 090 | 30.20 | 2.00 | 0.66 | 1.31 | 2.21 | 6.40 | 0.06 |
| HC 091 | 30.20 | 2.00 | 0.30 | 0.50 | 2.00 | 6.40 | 0.03 |
| HC 092 | 34.60 | 2.00 | 0.83 | 1.17 | 2.00 | 6.40 | 0.04 |
| HC 093 | 40.04 | 2.00 | 0.30 | 0.59 | 2.00 | 6.40 | 0.03 |
| HC 094 | 185.48 | 3.51 | 0.73 | 0.89 | 3.53 | 6.40 | 0.03 |
| HC 095 | 44.53 | 2.00 | 0.30 | 0.50 | 7.34 | 13.01 | 0.03 |
| HC 096 | 313.28 | 3.18 | 0.91 | 1.78 | 6.72 | 11.28 | 0.03 |
| HC 097 | 30.20 | 2.00 | 0.51 | 0.50 | 2.00 | 9.48 | 0.03 |
| HC 098 | 53.11 | 2.00 | 0.34 | 0.50 | 2.00 | 6.40 | 0.03 |
| HC 099 | 53.11 | 2.00 | 0.98 | 1.13 | 6.66 | 7.16 | 0.03 |
| HC 100 | 130.51 | 3.26 | 0.30 | 0.77 | 2.00 | 15.92 | 0.03 |
| HC 101 | 3501.68 | 20.53 | 4.61 | 61.43 | 4.66 | 10.26 | 0.20 |
| HC 102 | 3720.69 | 17.62 | 3.50 | 21.95 | 2.27 | 12.80 | 0.17 |
| HC 103 | 77.12 | 2.00 | 0.30 | 0.96 | 2.00 | 6.99 | 0.03 |
| HC 104 | 30.20 | 2.00 | 0.30 | 0.50 | 2.00 | 6.40 | 0.03 |
| HC 105 | 34.11 | 2.00 | 3.20 | 0.89 | 2.00 | 6.40 | 0.03 |
| HC 106 | 96.02 | 2.00 | 2.18 | 1.27 | 2.90 | 6.40 | 0.03 |
| HC 107 | 30.20 | 2.00 | 1.10 | 0.50 | 2.00 | 6.40 | 0.03 |
| HC 108 | 30.20 | 2.00 | 0.30 | 0.50 | 2.00 | 6.40 | 0.03 |
| HC 109 | 122.92 | 6.60 | 0.55 | 3.85 | 2.00 | 6.40 | 0.08 |
| HC 110 | 114.35 | 2.00 | 0.48 | 1.88 | 2.00 | 6.99 | 0.03 |
| HC 111 | 30.20 | 2.00 | 0.75 | 0.50 | 2.00 | 6.40 | 0.03 |
| HC 112 | 30.20 | 2.00 | 1.47 | 0.50 | 2.00 | 11.13 | 0.03 |
| HC 113 | 44.53 | 2.00 | 0.30 | 1.40 | 2.00 | 15.07 | 0.03 |
| OVAR 001 | 10890.43 | 66.67 | 9.84 | 131.03 | 4.22 | 6.40 | 0.58 |
| OVAR 002 | 6796.93 | 110.79 | 11.62 | 126.10 | 3.49 | 6.40 | 0.86 |
| OVAR 003 | 12048.40 | 90.11 | 10.43 | 163.07 | 2.19 | 28.53 | 0.62 |
| OVAR 004 | 17764.28 | 57.74 | 6.54 | 78.16 | 6.48 | 6.40 | 0.39 |
| OVAR 005 | 4202.78 | 94.00 | 9.24 | 127.95 | 3.49 | 6.40 | 0.42 |
| OVAR 006 | 24260.21 | 66.22 | 9.83 | 79.04 | 2.56 | 6.40 | 1.16 |
| OVAR 007 | 10967.70 | 48.57 | 6.26 | 91.81 | 14.34 | 6.40 | 0.46 |
| OVAR 008 | 4869.03 | 175.28 | 18.89 | 105.92 | 3.48 | 6.40 | 0.46 |
| OVAR 009 | 11200.48 | 97.37 | 16.23 | 84.56 | 9.35 | 6.40 | 1.66 |
| OVAR 010 | 10132.18 | 73.08 | 17.46 | 433.06 | 2.00 | 6.40 | 0.90 |
| OVAR 011 | 8829.51 | 61.02 | 11.81 | 36.35 | 12.39 | 6.40 | 0.77 |
| OVAR 012 | 18861.83 | 69.20 | 20.77 | 183.28 | 5.82 | 6.40 | 1.09 |
| OVAR 013 | 5128.12 | 66.58 | 9.02 | 77.76 | 2.80 | 6.40 | 0.97 |
| OVAR 014 | 22891.15 | 77.71 | 17.89 | 115.81 | 17.03 | 6.40 | 0.78 |
| OVAR 015 | 7023.04 | 61.61 | 17.53 | 7.86 | 89.04 | 18.13 | 0.86 |
| OVAR 016 | 22997.49 | 129.03 | 24.25 | 135.74 | 48.50 | 6.40 | 0.94 |
| OVAR 017 | 29480.78 | 77.70 | 15.24 | 134.61 | 16.03 | 6.40 | 1.11 |
| OVAR 018 | 16324.88 | 137.09 | 18.51 | 146.80 | 28.08 | 6.40 | 1.16 |
| OVAR 019 | 14831.95 | 51.10 | 11.77 | 95.36 | 2.35 | 6.40 | 0.40 |
| OVAR 020 | 18897.65 | 100.51 | 16.00 | 134.09 | 11.82 | 6.40 | 1.20 |
| OVAR 021 | 3273.09 | 81.69 | 8.38 | 53.16 | 6.01 | 24.89 | 0.77 |
| OVAR 022 | 7425.33 | 56.07 | 14.17 | 112.31 | 2.00 | 6.40 | 0.32 |
| OVAR 023 | 26392.14 | 276.39 | 89.04 | 68.79 | 30.36 | 143.51 | 3.48 |
| OVAR 024 | 9263.83 | 83.08 | 10.10 | 198.55 | 2.00 | 6.40 | 0.13 |
| OVAR 025 | 14569.06 | 75.56 | 12.84 | 74.81 | 2.00 | 6.40 | 0.61 |
| OVAR 026 | 14892.75 | 74.72 | 9.61 | 123.45 | 2.98 | 6.40 | 0.56 |
| OVAR 027 | 3915.27 | 75.68 | 11.33 | 96.32 | 8.50 | 6.40 | 0.87 |
| OVAR 028 | 7394.29 | 76.68 | 7.46 | 62.76 | 6.84 | 6.40 | 0.74 |
| OVAR 029 | 10988.61 | 65.36 | 18.07 | 173.25 | 2.00 | 6.40 | 0.46 |
| OVAR 030 | 3405.20 | 51.10 | 8.61 | 199.53 | 3.15 | 6.40 | 0.40 |
| OVAR 031 | 18720.17 | 165.81 | 15.90 | 146.76 | 22.29 | 62.61 | 2.61 |
| OVAR 032 | 60121.23 | 120.91 | 32.10 | 32.03 | 6.69 | 6.40 | 0.91 |
| OVAR 033 | 23330.12 | 73.16 | 9.31 | 113.61 | 7.15 | 6.40 | 0.91 |
| OVAR 034 | 14591.65 | 64.49 | 13.11 | 92.49 | 5.81 | 6.40 | 0.84 |
| OVAR 035 | 7139.39 | 90.47 | 20.69 | 113.38 | 2.00 | 471.72 | 0.98 |
| OVAR 036 | 12043.40 | 71.69 | 29.49 | 56.70 | 2.00 | 6.40 | 0.69 |
| OVAR 037 | 19356.83 | 70.60 | 10.50 | 101.68 | 7.82 | 13.63 | 0.60 |
| OVAR 038 | 19444.59 | 83.80 | 9.89 | 72.11 | 8.85 | 18.21 | 1.33 |
| OVAR 039 | 13252.87 | 83.87 | 9.28 | 81.26 | 8.98 | 6.40 | 0.88 |
| OVAR 040 | 13873.83 | 77.12 | 33.12 | 58.72 | 2.00 | 6.40 | 0.58 |
| OVAR 041 | 77518.97 | 104.48 | 16.41 | 81.71 | 6.93 | 6.40 | 1.99 |
| OVAR 042 | 75155.00 | 91.09 | 15.70 | 84.38 | 4.85 | 6.40 | 0.52 |
| OVAR 043 | 7628.07 | 124.42 | 17.13 | 58.88 | 16.99 | 87.42 | 0.92 |
| OVAR 044 | 7704.09 | 97.80 | 9.92 | 132.38 | 5.54 | 6.40 | 0.42 |
| BLDR 001 | 7834.62 | 74.58 | 15.17 | 72.85 | 689.67 | 13.67 | 0.30 |
| BLDR 002 | 4809.21 | 77.11 | 13.38 | 155.67 | 809.73 | 53.03 | 0.52 |
| BLDR 003 | 10084.77 | 93.77 | 10.76 | 139.07 | 1204.09 | 6.40 | 0.42 |
| BLDR 004 | 9776.97 | 71.07 | 9.28 | 83.72 | 2.44 | 6.49 | 4.9 |
| BLDR 005 | 3729.94 | 148.83 | 12.45 | 80.75 | 1216.95 | 6.40 | 0.25 |
| BLDR 006 | 4300.08 | 83.33 | 8.54 | 114.35 | 445.18 | 6.40 | 0.30 |
| BLDR 007 | 10262.12 | 126.87 | 21.21 | 71.31 | 5.92 | 6.40 | 1.08 |
| BLDR 008 | 36226.36 | 130.22 | 11.10 | 123.31 | 2774.16 | 50.19 | 0.54 |
| BLDR 009 | 18866.24 | 66.67 | 11.62 | 96.01 | 1822.00 | 6.40 | 0.62 |
| BLDR 010 | 14248.88 | 75.74 | 10.26 | 65.33 | 10539.02 | 6.40 | 0.55 |
| BLDR 011 | 50969.96 | 79.72 | 10.13 | 152.59 | 813.23 | 16.45 | 0.67 |
| BLDR 012 | 31438.40 | 77.26 | 11.69 | 115.33 | 455.71 | 6.40 | 0.82 |
| BLDR 013 | 10288.15 | 103.60 | 20.60 | 163.06 | 803.14 | 6.40 | 0.38 |
| BLDR 014 | 16137.98 | 44.41 | 5.30 | 90.11 | 254.76 | 6.40 | 0.86 |
| BLDR 015 | 5908.69 | 94.33 | 9.51 | 137.73 | 299.51 | 6.40 | 0.57 |
| BLDR 016 | 12040.46 | 55.84 | 9.67 | 100.40 | 2.31 | 22.75 | 0.40 |
| BLDR 017 | 4572.74 | 85.31 | 8.73 | 197.66 | 284.30 | 11.04 | 0.69 |
| BLDR 018 | 10913.03 | 105.22 | 9.98 | 103.80 | 5898.73 | 6.40 | 0.45 |
| BLDR 019 | 28875.18 | 67.32 | 11.30 | 118.72 | 1387.45 | 6.40 | 0.03 |
| BLDR 020 | 21082.49 | 234.18 | 21.72 | 193.84 | 3522.12 | 6.40 | 4.28 |
| BLDR 021 | 5586.89 | 80.06 | 9.75 | 75.40 | 863.15 | 6.40 | 0.85 |
| BLDR 022 | 17355.99 | 112.79 | 15.49 | 137.96 | 1342.06 | 6.40 | 0.76 |
| BLDR 023 | 3354.63 | 73.60 | 5.72 | 76.78 | 496.30 | 6.40 | 0.07 |
| BLDR 024 | 3422.32 | 71.06 | 5.58 | 41.50 | 1792.90 | 6.40 | 0.28 |
| BLDR 025 | 30645.35 | 85.56 | 8.56 | 113.65 | 3.57 | 29.32 | 0.41 |
| BLDR 026 | 7411.72 | 87.99 | 16.35 | 110.00 | 776.08 | 6.40 | 0.69 |
| BLDR 027 | 1773.13 | 81.17 | 9.63 | 142.12 | 959.70 | 88.89 | 0.37 |
| BLDR 028 | 6835.72 | 105.20 | 12.10 | 67.77 | 488.59 | 6.40 | 0.57 |
| BLDR 029 | 15989.33 | 145.84 | 20.09 | 131.28 | 1866.71 | 84.35 | 1.04 |
| BLDR 030 | 8503.89 | 90.11 | 17.49 | 127.33 | 624.65 | 6.40 | 0.42 |
| BLDR 031 | 6872.33 | 172.19 | 29.00 | 121.18 | 2426.09 | 6.40 | 0.86 |
| BLDR 032 | 5921.35 | 83.36 | 9.98 | 78.67 | 670.55 | 6.40 | 4.80 |
| BLDR 033 | 10983.76 | 82.22 | 8.99 | 90.10 | 5.70 | 6.40 | 1.06 |
| BLDR 034 | 8271.44 | 60.86 | 12.73 | 78.18 | 260.04 | 6.40 | 0.61 |
| BLDR 035 | 12189.72 | 108.22 | 18.65 | 111.35 | 721.09 | 252.91 | 0.83 |
| BLDR 036 | 2444.95 | 65.98 | 5.30 | 106.08 | 4171.61 | 26.14 | 0.11 |
| BLDR 037 | 9414.99 | 113.25 | 11.05 | 117.82 | 502.14 | 6.40 | 0.61 |
| BLDR 038 | 4848.52 | 196.32 | 20.67 | 111.09 | 1963.12 | 65.60 | 0.85 |
| BLDR 039 | 177859.59 | 96.02 | 13.27 | 132.05 | 33.55 | 6.40 | 0.55 |
| BLDR 040 | 3733.11 | 46.94 | 6.14 | 100.60 | 594.82 | 35.05 | 0.22 |
| BLDR 041 | 79503.06 | 91.09 | 11.38 | 103.09 | 4.17 | 6.40 | 0.42 |
| BLDR 042 | 3338.99 | 107.82 | 13.55 | 177.42 | 1026.58 | 119.94 | 0.30 |
| BLDR 043 | 2906.99 | 104.48 | 24.53 | 137.69 | 1051.02 | 6.40 | 0.52 |
| BLDR 044 | 3179.46 | 50.25 | 15.70 | 187.99 | 1029.51 | 20.06 | 0.15 |
| BLDR 045 | 15781.02 | 104.48 | 11.38 | 145.65 | 391.14 | 20.06 | 0.52 |
| BLDR 046 | 32356.35 | 93.77 | 10.55 | 75.32 | 20.03 | 6.40 | 1.02 |
| BLDR 047 | 6119.86 | 53.78 | 9.22 | 78.89 | 638.19 | 6.40 | 2.66 |
| BLDR 048 | 44661.25 | 68.51 | 13.97 | 116.65 | 315.82 | 6.40 | 0.75 |
| PDAC 001 | 43812.25 | 51.98 | 10.57 | 159.61 | 29.55 | 6.40 | 0.13 |
| PDAC 002 | 57963.61 | 39.42 | 12.84 | 85.18 | 2.00 | 6.40 | 0.13 |
| PDAC 003 | 70624.98 | 67.89 | 10.10 | 91.71 | 2.00 | 6.40 | 0.52 |
| PDAC 004 | 6397.98 | 213.83 | 10.66 | 55.37 | 3.48 | 6.40 | 0.54 |
| PDAC 005 | 64888.31 | 43.66 | 14.17 | 143.54 | 2.00 | 6.40 | 0.52 |
| PDAC 006 | 76575.31 | 51.98 | 13.73 | 126.87 | 2.00 | 6.40 | 0.13 |
| PDAC 007 | 15745.45 | 111.87 | 15.68 | 144.61 | 8.45 | 124.26 | 1.85 |
| PDAC 008 | 107780.51 | 118.06 | 10.31 | 93.56 | 4.04 | 51.39 | 0.56 |
| PDAC 009 | 47042.96 | 174.96 | 15.11 | 133.16 | 8.50 | 63.51 | 1.95 |
| PDAC 010 | 180588.58 | 43.67 | 12.84 | 63.68 | 2.00 | 6.40 | 0.23 |
| PDAC 011 | 46822.11 | 69.54 | 13.51 | 53.12 | 2526.05 | 23.64 | 0.65 |
| PDAC 012 | 19490.55 | 67.58 | 9.19 | 104.36 | 4.30 | 11.07 | 0.69 |
| PDAC 013 | 286827.18 | 83.33 | 10.97 | 89.65 | 22.96 | 6.40 | 0.83 |
| PDAC 014 | 189955.47 | 59.33 | 10.03 | 97.79 | 2.00 | 6.40 | 0.65 |
| PDAC 015 | 58349.89 | 84.62 | 12.69 | 119.52 | 4.37 | 6.40 | 0.80 |
| PDAC 016 | 61365.38 | 95.30 | 17.05 | 139.65 | 1323.20 | 25.04 | 0.62 |
| PDAC 017 | 14924.43 | 82.31 | 7.10 | 90.08 | 5.08 | 6.40 | 1.29 |
| PDAC 018 | 44094.83 | 72.99 | 13.28 | 80.93 | 1137.54 | 6.40 | 0.55 |
| PDAC 019 | 76502.04 | 69.60 | 11.10 | 78.11 | 11.02 | 24.89 | 0.67 |
| PDAC 020 | 311948.69 | 79.83 | 9.92 | 85.69 | 806.18 | 18.72 | 0.46 |
| PDAC 021 | 163921.87 | 56.26 | 6.68 | 84.14 | 2.00 | 6.40 | 0.37 |
| PDAC 022 | 136867.70 | 51.10 | 14.89 | 130.77 | 3.15 | 6.40 | 0.50 |
| PDAC 023 | 18508.21 | 86.99 | 15.53 | 191.49 | 2.00 | 6.40 | 0.49 |
| PDAC 024 | 15518.00 | 76.70 | 11.05 | 84.68 | 211.03 | 6.40 | 0.45 |
| PDAC 025 | 143649.42 | 83.36 | 12.10 | 92.87 | 8.27 | 50.60 | 0.96 |
| PDAC 026 | 38122.11 | 45.93 | 26.20 | 14.47 | 2.08 | 64.76 | 0.43 |
| PDAC 027 | 86274.81 | 52.15 | 13.98 | 90.68 | 2.82 | 78.92 | 0.49 |
| PDAC 028 | 21284.98 | 65.45 | 8.78 | 69.28 | 2.00 | 6.40 | 0.68 |
| PDAC 029 | 106564.55 | 124.49 | 10.86 | 58.74 | 2.00 | 6.40 | 0.65 |
| PDAC 030 | 2588.92 | 47.02 | 8.36 | 33.66 | 507.95 | 6.40 | 0.48 |
| PDAC 031 | 9858.71 | 75.74 | 11.81 | 77.27 | 8.20 | 6.40 | 0.85 |
| PDAC 032 | 383404.52 | 56.10 | 6.84 | 79.48 | 12.81 | 6.40 | 1.10 |
| PDAC 033 | 43746.65 | 80.62 | 11.91 | 113.28 | 1225.41 | 6.40 | 0.47 |
| PDAC 034 | 57528.92 | 59.38 | 7.52 | 93.68 | 1257.79 | 6.40 | 0.34 |
| PDAC 035 | 11149.22 | 51.15 | 9.38 | 92.80 | 1048.24 | 6.40 | 0.43 |
| PDAC 036 | 148085.40 | 86.22 | 8.94 | 100.30 | 2.44 | 6.40 | 0.58 |
| PDAC 037 | 13362.33 | 53.58 | 16.33 | 91.36 | 606.94 | 6.40 | 0.46 |
| PDAC 038 | 159727.57 | 74.51 | 29.27 | 159.37 | 3.94 | 21.59 | 1.36 |
| PDAC 039 | 88795.60 | 100.47 | 18.80 | 92.95 | 459.08 | 29.41 | 0.83 |
| PDAC 040 | 118333.39 | 95.30 | 11.92 | 126.10 | 7.91 | 23.31 | 1.00 |
| PDAC 041 | 42182.04 | 84.36 | 11.38 | 140.34 | 1086.34 | 6.40 | 0.42 |
| PDAC 042 | 232193.54 | 61.45 | 8.86 | 178.86 | 2.00 | 6.40 | 0.03 |
| PDAC 043 | 201351.55 | 42.88 | 7.33 | 85.69 | 2.19 | 6.40 | 0.51 |
| PDAC 044 | 37302.54 | 185.07 | 26.43 | 145.69 | 483.19 | 6.40 | 3.03 |
| HC 001 | 17976.54 | 96.39 | 25.48 | 245.91 | 13.07 | 6.40 | 0.91 |
| HC 002 | 6081.76 | 69.25 | 16.02 | 167.40 | 5.75 | 82.92 | 0.85 |
| HC 003 | 14402.61 | 135.84 | 10.81 | 73.42 | 8.64 | 6.40 | 1.32 |
| HC 004 | 13199.64 | 129.64 | 10.16 | 51.74 | 7.44 | 6.40 | 1.21 |
| HC 005 | 8867.36 | 73.16 | 12.40 | 98.31 | 6.32 | 6.40 | 0.63 |
| HC 006 | 24902.79 | 87.52 | 12.21 | 51.41 | 34.76 | 6.40 | 0.94 |
| HC 007 | 9295.74 | 83.62 | 13.98 | 101.53 | 5.75 | 6.40 | 0.83 |
| HC 008 | 14587.62 | 98.15 | 9.17 | 180.57 | 6.25 | 6.40 | 1.21 |
| HC 009 | 18106.60 | 46.27 | 6.73 | 133.14 | 284.57 | 6.40 | 0.52 |
| HC 010 | 13683.94 | 110.80 | 9.49 | 169.91 | 6.25 | 6.40 | 1.11 |
| HC 011 | 17451.49 | 48.08 | 6.95 | 135.11 | 305.73 | 6.40 | 0.53 |
| HC 012 | 13303.04 | 75.47 | 8.51 | 53.09 | 2.00 | 6.40 | 0.78 |
| HC 013 | 17918.96 | 78.73 | 8.18 | 65.31 | 3.37 | 6.40 | 0.82 |
| HC 014 | 17306.50 | 68.86 | 8.18 | 57.17 | 2.41 | 6.40 | 0.71 |
| HC 015 | 15503.50 | 62.17 | 6.84 | 51.74 | 3.85 | 6.40 | 0.62 |
| HC 016 | 13092.89 | 88.52 | 12.09 | 121.81 | 4.81 | 6.40 | 1.48 |
| HC 017 | 30967.56 | 62.27 | 10.97 | 114.35 | 813.61 | 6.40 | 0.20 |
| HC 018 | 9490.92 | 65.19 | 7.28 | 77.36 | 4.81 | 6.40 | 0.97 |
| HC 019 | 13603.26 | 85.26 | 10.15 | 106.38 | 4.33 | 6.40 | 1.27 |
| HC 020 | 13499.63 | 94.91 | 11.77 | 111.09 | 5.29 | 6.40 | 1.45 |
| HC 021 | 13724.15 | 65.36 | 10.31 | 37.10 | 9.23 | 6.40 | 0.51 |
| HC 022 | 15305.99 | 69.26 | 10.86 | 38.93 | 9.64 | 6.40 | 0.55 |
| HC 023 | 16258.84 | 61.42 | 9.96 | 36.65 | 8.81 | 6.40 | 0.41 |
| HC 024 | 17701.37 | 73.16 | 10.75 | 39.84 | 10.48 | 6.40 | 0.51 |
| HC 025 | 88985.80 | 65.79 | 8.65 | 75.32 | 341.26 | 6.40 | 0.66 |
| HC 026 | 54680.33 | 65.36 | 15.90 | 136.86 | 8.40 | 6.40 | 0.73 |
| HC 027 | 11444.53 | 36.80 | 8.82 | 92.62 | 229.63 | 6.40 | 0.16 |
| HC 028 | 12506.67 | 40.60 | 9.52 | 90.95 | 195.43 | 6.40 | 0.19 |
| HC 029 | 46475.40 | 48.21 | 5.16 | 68.08 | 243.99 | 6.40 | 0.49 |
| HC 030 | 21283.66 | 57.14 | 13.55 | 108.43 | 11.18 | 20.06 | 0.61 |
| HC 031 | 13539.08 | 84.36 | 18.19 | 135.03 | 397.38 | 30.97 | 0.42 |
| HC 032 | 6560.23 | 50.25 | 9.18 | 143.00 | 459.36 | 6.40 | 0.61 |
| HC 033 | 7864.69 | 87.33 | 12.07 | 156.88 | 209.58 | 6.40 | 0.77 |
| HC 034 | 3956.56 | 91.09 | 10.65 | 113.76 | 9.76 | 40.60 | 0.92 |
| HC 035 | 8750.45 | 131.02 | 12.10 | 135.03 | 11.90 | 6.40 | 0.85 |
| HC 036 | 7610.02 | 90.63 | 11.00 | 141.66 | 172.93 | 6.40 | 0.72 |
| HC 037 | 4536.79 | 117.79 | 14.98 | 129.72 | 13.35 | 73.13 | 1.18 |
| HC 038 | 6544.93 | 50.25 | 7.71 | 135.03 | 368.01 | 6.40 | 0.52 |
| HC 039 | 9309.80 | 150.75 | 16.41 | 180.07 | 15.53 | 6.40 | 0.99 |
| HC 040 | 5375.50 | 82.63 | 9.92 | 115.33 | 3.90 | 13.51 | 1.24 |
| HC 041 | 9234.27 | 77.60 | 12.82 | 87.06 | 922.69 | 30.97 | 0.42 |
| HC 042 | 11402.33 | 66.44 | 11.69 | 83.37 | 779.85 | 11.04 | 0.37 |
| HC 043 | 11867.01 | 118.93 | 16.33 | 99.85 | 13.36 | 6.40 | 0.69 |
| HC 044 | 9475.42 | 285.06 | 37.39 | 107.96 | 729.62 | 334.44 | 3.84 |
| HC 045 | 9309.13 | 261.63 | 37.23 | 117.71 | 736.72 | 323.59 | 3.74 |
| HC 046 | 16918.31 | 56.07 | 10.57 | 115.01 | 2.00 | 6.40 | 1.39 |
| HC 047 | 14815.00 | 51.98 | 10.09 | 113.38 | 2.00 | 6.40 | 1.30 |
| HC 048 | 16778.05 | 60.04 | 11.03 | 124.18 | 2.00 | 6.40 | 1.56 |
| HC 049 | 8445.58 | 136.01 | 16.31 | 107.98 | 21.48 | 6.40 | 2.23 |
| HC 050 | 34815.13 | 261.64 | 33.60 | 76.45 | 177.10 | 316.04 | 3.98 |
| HC 051 | 33748.15 | 270.36 | 33.96 | 77.54 | 172.69 | 298.16 | 4.05 |
| HC 052 | 13311.50 | 132.44 | 15.45 | 103.65 | 25.52 | 98.86 | 2.23 |
| HC 053 | 24478.11 | 70.75 | 11.36 | 149.67 | 241.00 | 6.40 | 0.47 |
| HC 054 | 22994.66 | 75.63 | 12.00 | 128.85 | 252.37 | 6.40 | 0.45 |
| HC 055 | 8923.87 | 65.53 | 8.38 | 94.40 | 442.78 | 16.10 | 0.87 |
| HC 056 | 10152.99 | 55.53 | 7.53 | 107.34 | 440.22 | 6.40 | 0.75 |
| HC 057 | 5905.31 | 58.06 | 11.59 | 97.20 | 6.93 | 27.11 | 0.69 |
| HC 058 | 20882.63 | 65.15 | 12.21 | 63.83 | 6.70 | 6.40 | 0.86 |
| HC 059 | 26809.55 | 139.21 | 23.69 | 103.65 | 21.48 | 98.86 | 2.65 |
| HC 060 | 2996.02 | 61.44 | 11.55 | 107.81 | 6.01 | 6.40 | 0.81 |
| HC 061 | 2962.11 | 63.48 | 12.44 | 116.42 | 5.60 | 6.40 | 0.77 |
| HC 062 | 28087.24 | 186.28 | 16.43 | 103.02 | 234.41 | 78.65 | 0.81 |
| HC 063 | 24631.34 | 153.07 | 14.22 | 90.57 | 203.51 | 15.88 | 0.74 |
| HC 064 | 6806.82 | 51.10 | 7.92 | 99.19 | 358.97 | 6.40 | 0.70 |
| HC 065 | 26643.93 | 232.98 | 13.23 | 73.32 | 167.92 | 32.30 | 0.81 |
| HC 066 | 51541.45 | 59.38 | 11.77 | 47.40 | 6.42 | 6.40 | 0.87 |
| HC 067 | 6846.81 | 46.93 | 6.99 | 41.63 | 440.64 | 6.40 | 0.77 |
| HC 068 | 5075.26 | 79.71 | 11.77 | 99.67 | 311.32 | 6.40 | 0.40 |
| HC 069 | 8650.53 | 63.15 | 16.72 | 185.83 | 318.77 | 6.40 | 0.20 |
| HC 070 | 14810.50 | 64.41 | 10.47 | 116.78 | 381.03 | 6.40 | 0.12 |
| HC 071 | 3830.39 | 47.93 | 10.16 | 100.45 | 2.00 | 6.40 | 0.21 |
| HC 072 | 15505.51 | 63.13 | 10.77 | 127.25 | 392.78 | 6.40 | 0.09 |
| HC 073 | 3729.51 | 50.47 | 10.77 | 106.29 | 2.41 | 6.40 | 0.21 |
| HC 074 | 4110.55 | 53.02 | 11.09 | 111.53 | 3.26 | 6.40 | 0.22 |
| HC 075 | 4336.27 | 55.57 | 11.39 | 112.70 | 3.69 | 6.40 | 0.38 |
| HC 076 | 22165.47 | 73.21 | 15.96 | 158.61 | 81.71 | 6.40 | 0.87 |
| HC 077 | 57302.24 | 55.57 | 9.54 | 79.42 | 17.13 | 6.40 | 1.63 |
| HC 078 | 60609.09 | 53.02 | 8.60 | 77.07 | 16.65 | 6.40 | 1.63 |
| HC 079 | 25464.37 | 83.20 | 18.06 | 171.36 | 97.43 | 6.40 | 0.95 |
| HC 080 | 59963.21 | 53.02 | 8.60 | 73.56 | 15.21 | 6.40 | 1.50 |
| HC 081 | 24653.67 | 75.71 | 16.57 | 166.72 | 88.18 | 6.40 | 0.87 |
| HC 082 | 9899.62 | 68.14 | 10.61 | 112.68 | 18.16 | 6.40 | 0.50 |
| HC 083 | 22459.62 | 45.36 | 10.62 | 59.47 | 569.36 | 6.40 | 0.51 |
| HC 084 | 11891.23 | 42.80 | 7.34 | 71.21 | 791.53 | 6.40 | 0.48 |
| HC 085 | 13843.17 | 50.48 | 7.34 | 71.21 | 891.56 | 6.40 | 0.62 |
| HC 086 | 38825.25 | 68.19 | 12.01 | 137.71 | 588.03 | 6.40 | 0.57 |
| HC 087 | 14694.93 | 71.95 | 10.93 | 189.88 | 20.53 | 6.40 | 0.62 |
| HC 088 | 3034.44 | 58.10 | 10.78 | 127.83 | 7.26 | 6.40 | 0.38 |
| HC 089 | 9510.68 | 45.36 | 10.78 | 137.71 | 430.36 | 6.40 | 0.51 |
| HC 090 | 8025.41 | 42.80 | 10.16 | 121.43 | 390.80 | 6.40 | 0.38 |
| HC 091 | 11320.83 | 59.35 | 10.16 | 74.72 | 12.83 | 6.40 | 0.50 |
| HC 092 | 8409.48 | 42.80 | 9.22 | 115.61 | 379.82 | 6.40 | 0.38 |
| HC 093 | 7530.72 | 45.32 | 9.21 | 86.99 | 4.15 | 6.40 | 0.21 |
| HC 094 | 23709.71 | 66.92 | 11.70 | 109.78 | 496.41 | 6.40 | 0.20 |
| HC 095 | 15911.41 | 82.63 | 21.52 | 86.04 | 330.12 | 42.16 | 0.54 |
| HC 096 | 23181.25 | 63.15 | 9.85 | 107.45 | 466.22 | 6.40 | 0.12 |
| HC 097 | 7992.52 | 60.62 | 8.27 | 100.45 | 414.91 | 6.40 | 0.12 |
| HC 098 | 17064.72 | 40.22 | 9.21 | 74.72 | 3.26 | 6.40 | 0.12 |
| HC 099 | 10246.94 | 45.37 | 7.97 | 111.53 | 603.29 | 6.40 | 0.51 |
| HC 100 | 14462.01 | 35.07 | 7.97 | 85.27 | 2.41 | 6.40 | 0.20 |
| HC 101 | 26773.08 | 50.47 | 9.53 | 124.34 | 12.20 | 6.40 | 0.21 |
| HC 102 | 25851.27 | 47.90 | 9.53 | 129.57 | 2.85 | 6.40 | 0.25 |
| HC 103 | 22019.29 | 63.77 | 10.81 | 125.75 | 4.55 | 28.53 | 0.52 |
| HC 104 | 7015.09 | 54.70 | 8.91 | 160.65 | 3.32 | 16.41 | 0.43 |
| HC 105 | 21244.78 | 75.10 | 16.33 | 207.90 | 252.80 | 6.40 | 0.35 |
| HC 106 | 22188.12 | 78.99 | 17.31 | 210.23 | 265.63 | 6.40 | 0.51 |
| HC 107 | 8270.01 | 69.27 | 17.74 | 175.12 | 185.51 | 6.40 | 0.41 |
| HC 108 | 10021.39 | 66.67 | 15.45 | 78.25 | 2.00 | 40.89 | 0.62 |
| HC 109 | 5908.66 | 131.35 | 14.57 | 159.37 | 8.52 | 6.40 | 1.65 |
| HC 110 | 9541.61 | 56.17 | 13.09 | 68.48 | 2.00 | 16.86 | 0.58 |
| HC 111 | 5452.07 | 121.09 | 13.83 | 154.44 | 8.52 | 6.40 | 1.50 |
| HC 112 | 10288.35 | 66.67 | 15.45 | 78.25 | 2.00 | 51.44 | 0.67 |
| HC 113 | 6013.74 | 82.63 | 16.93 | 173.78 | 149.53 | 6.40 | 0.54 |

| Subject | | | | | | | |
|---|---|---|---|---|---|---|---|
| Cohort | | Cathepsin | FAP | | | | |
| ID | sFAS | D | alpha | Ferritin | Galectin-3 | IGFBP3 | MIA |
| OVAR 001 | 8.40 | 0.40 | 0.17 | 0.71 | 0.02 | 6.78 | 0.20 |
| OVAR 002 | 8.40 | 0.73 | 0.79 | 2.44 | 0.03 | 9.80 | 0.34 |
| OVAR 003 | 8.40 | 0.56 | 0.53 | 0.37 | 0.01 | 9.03 | 0.34 |
| OVAR 004 | 8.40 | 1.44 | 2.49 | 4.82 | 0.11 | 16.99 | 0.52 |
| OVAR 005 | 8.40 | 0.51 | 0.32 | 0.48 | 0.01 | 3.95 | 0.30 |
| OVAR 006 | 8.40 | 1.23 | 0.15 | 0.19 | 0.01 | 2.60 | 0.14 |
| OVAR 007 | 14.30 | 0.86 | 0.17 | 1.72 | 0.01 | 1.11 | 0.14 |
| OVAR 008 | 28.78 | 0.40 | 0.17 | 0.65 | 0.01 | 2.79 | 0.27 |
| OVAR 009 | 24.57 | 1.14 | 0.14 | 0.24 | 0.01 | 2.94 | 0.23 |
| OVAR 010 | 19.60 | 2.20 | 0.46 | 0.15 | 0.01 | 3.27 | 0.37 |
| OVAR 011 | 74.89 | 22.19 | 5.83 | 2.75 | 0.86 | 9.96 | 8.94 |
| OVAR 012 | 101.31 | 20.16 | 5.63 | 2.52 | 0.73 | 9.95 | 8.60 |
| OVAR 013 | 31.64 | 0.54 | 0.16 | 2.35 | 0.01 | 5.70 | 0.13 |
| OVAR 014 | 33.03 | 3.62 | 1.40 | 3.37 | 0.01 | 6.47 | 0.42 |
| OVAR 015 | 26.01 | 0.40 | 0.09 | 0.67 | 0.01 | 5.31 | 0.19 |
| OVAR 016 | 8.40 | 0.59 | 0.05 | 0.10 | 0.01 | 2.63 | 0.12 |
| OVAR 017 | 28.95 | 2.93 | 0.05 | 3.42 | 0.01 | 2.96 | 0.12 |
| OVAR 018 | 33.21 | 0.76 | 0.32 | 0.38 | 0.01 | 5.81 | 0.22 |
| OVAR 019 | 22.66 | 1.50 | 0.84 | 14.13 | 0.01 | 8.18 | 0.54 |
| OVAR 020 | 8.40 | 0.84 | 0.05 | 0.73 | 0.01 | 2.50 | 0.12 |
| OVAR 021 | 16.19 | 1.66 | 1.38 | 0.91 | 0.01 | 4.76 | 0.37 |
| OVAR 022 | 8.40 | 0.40 | 1.03 | 0.57 | 0.01 | 2.97 | 0.12 |
| OVAR 023 | 54.91 | 1.18 | 0.86 | 6.20 | 0.01 | 3.94 | 0.36 |
| OVAR 024 | 61.83 | 1.78 | 0.48 | 14.83 | 0.01 | 2.40 | 0.35 |
| OVAR 025 | 45.36 | 0.40 | 0.05 | 0.45 | 0.01 | 1.98 | 0.12 |
| OVAR 026 | 22.03 | 0.40 | 0.38 | 0.04 | 0.01 | 2.09 | 0.12 |
| OVAR 027 | 22.67 | 0.40 | 0.63 | 0.46 | 0.01 | 3.03 | 0.31 |
| OVAR 028 | 20.54 | 0.70 | 0.36 | 0.55 | 0.01 | 8.97 | 0.48 |
| OVAR 029 | 71.82 | 1.22 | 0.25 | 0.86 | 0.06 | 2.57 | 0.31 |
| OVAR 030 | 15.15 | 0.76 | 0.33 | 1.58 | 0.01 | 9.50 | 0.33 |
| OVAR 031 | 34.29 | 5.22 | 3.09 | 1.77 | 0.14 | 15.84 | 0.83 |
| OVAR 032 | 26.61 | 0.52 | 0.10 | 8.50 | 0.01 | 5.27 | 0.13 |
| OVAR 033 | 61.40 | 0.40 | 0.05 | 0.23 | 0.01 | 1.58 | 0.12 |
| OVAR 034 | 12.95 | 0.43 | 1.31 | 1.52 | 0.01 | 11.37 | 0.38 |
| OVAR 035 | 38.22 | 0.40 | 0.05 | 7.86 | 0.01 | 0.20 | 0.12 |
| OVAR 036 | 44.74 | 4.20 | 0.57 | 52.19 | 0.01 | 8.56 | 0.17 |
| OVAR 037 | 23.65 | 12.78 | 1.56 | 7.13 | 0.39 | 14.94 | 0.77 |
| OVAR 038 | 37.09 | 3.16 | 0.46 | 0.44 | 0.04 | 4.38 | 0.33 |
| OVAR 039 | 60.06 | 1.68 | 0.48 | 0.17 | 0.01 | 0.45 | 0.61 |
| OVAR 040 | 8.40 | 1.01 | 0.43 | 0.40 | 0.01 | 6.08 | 0.19 |
| OVAR 041 | 48.90 | 0.40 | 0.09 | 0.22 | 0.01 | 0.76 | 0.12 |
| OVAR 042 | 104.13 | 0.77 | 0.19 | 1.46 | 0.03 | 2.50 | 0.12 |
| OVAR 043 | 36.55 | 0.40 | 0.05 | 2.35 | 0.01 | 0.74 | 0.12 |
| OVAR 044 | 180.31 | 0.43 | 0.25 | 0.44 | 0.03 | 3.68 | 0.12 |
| BLDR 001 | 54.43 | 2.31 | 1.44 | 8.32 | 0.01 | 9.60 | 0.40 |
| BLDR 002 | 8.40 | 0.40 | 0.08 | 0.69 | 0.01 | 1.65 | 0.12 |
| BLDR 003 | 46.07 | 0.40 | 0.22 | 1.16 | 0.01 | 5.51 | 0.22 |
| BLDR 004 | 17.55 | 0.40 | 0.10 | 0.11 | 0.02 | 3.85 | 0.12 |
| BLDR 005 | 8.40 | 2.61 | 0.40 | 7.88 | 0.01 | 4.97 | 0.25 |
| BLDR 006 | 25.77 | 0.73 | 1.58 | 5.48 | 0.12 | 8.09 | 0.14 |
| BLDR 007 | 74.23 | 0.97 | 0.16 | 0.56 | 0.01 | 4.96 | 0.12 |
| BLDR 008 | 8.40 | 1.47 | 0.47 | 0.50 | 0.01 | 5.65 | 0.39 |
| BLDR 009 | 21.39 | 0.40 | 0.12 | 0.61 | 0.01 | 5.25 | 0.18 |
| BLDR 010 | 8.40 | 1.49 | 0.50 | 0.65 | 0.01 | 3.18 | 0.19 |
| BLDR 011 | 8.89 | 4.71 | 1.48 | 10.19 | 0.23 | 17.04 | 0.57 |
| BLDR 012 | 8.40 | 2.23 | 2.41 | 4.95 | 0.01 | 9.99 | 0.43 |
| BLDR 013 | 39.36 | 0.70 | 0.26 | 0.52 | 0.01 | 2.78 | 0.19 |
| BLDR 014 | 34.02 | 0.46 | 0.33 | 0.58 | 0.01 | 5.58 | 0.42 |
| BLDR 015 | 52.27 | 2.20 | 0.54 | 0.40 | 0.01 | 6.92 | 0.63 |
| BLDR 016 | 13.84 | 1.66 | 2.08 | 1.45 | 0.01 | 13.62 | 0.64 |
| BLDR 017 | 19.27 | 0.40 | 0.28 | 0.78 | 0.01 | 1.67 | 0.12 |
| BLDR 018 | 49.45 | 0.40 | 0.18 | 0.12 | 0.01 | 2.79 | 0.28 |
| BLDR 019 | 8.40 | 1.55 | 0.43 | 3.64 | 0.21 | 8.15 | 0.48 |
| BLDR 020 | 21.66 | 0.97 | 0.32 | 0.29 | 0.10 | 8.67 | 0.38 |
| BLDR 021 | 29.17 | 1.50 | 0.10 | 1.10 | 0.01 | 4.15 | 0.29 |
| BLDR 022 | 24.96 | 0.59 | 0.35 | 0.10 | 0.01 | 5.94 | 0.45 |
| BLDR 023 | 14.41 | 0.40 | 0.22 | 0.34 | 0.01 | 2.92 | 0.22 |
| BLDR 024 | 21.26 | 1.07 | 1.09 | 1.27 | 0.01 | 5.86 | 0.48 |
| BLDR 025 | 32.85 | 0.59 | 0.27 | 0.77 | 0.01 | 8.29 | 0.48 |
| BLDR 026 | 8.40 | 0.69 | 0.33 | 1.39 | 0.01 | 3.43 | 0.27 |
| BLDR 027 | 21.35 | 2.65 | 2.72 | 6.18 | 0.01 | 52.52 | 0.79 |
| BLDR 028 | 15.94 | 2.56 | 1.25 | 1.96 | 0.01 | 13.00 | 0.46 |
| BLDR 029 | 8.40 | 0.40 | 0.05 | 0.08 | 0.01 | 0.83 | 0.14 |
| BLDR 030 | 88.60 | 0.7 | 0.22 | 0.96 | 0.01 | 5.67 | 0.28 |
| BLDR 031 | 30.94 | 4.83 | 0.79 | 1.66 | 0.02 | 6.50 | 0.50 |
| BLDR 032 | 8.40 | 2.91 | 0.42 | 5.10 | 0.01 | 10.12 | 0.31 |
| BLDR 033 | 8.40 | 0.93 | 0.14 | 5.03 | 0.01 | 4.67 | 0.26 |
| BLDR 034 | 125.43 | 0.40 | 0.20 | 0.16 | 0.01 | 0.98 | 0.15 |
| BLDR 035 | 14.53 | 7.19 | 2.62 | 57.44 | 0.01 | 10.65 | 0.52 |
| BLDR 036 | 14.94 | 3.40 | 0.05 | 1.72 | 0.01 | 0.20 | 0.12 |
| BLDR 037 | 8.40 | 2.40 | 0.59 | 0.34 | 0.06 | 0.71 | 0.85 |
| BLDR 038 | 59.68 | 0.40 | 0.17 | 2.28 | 0.01 | 0.60 | 0.12 |
| BLDR 039 | 8.40 | 1.16 | 1.05 | 2.63 | 0.01 | 4.13 | 0.36 |
| BLDR 040 | 28.95 | 0.65 | 0.31 | 2.84 | 0.01 | 3.62 | 0.36 |
| BLDR 041 | 31.91 | 2.28 | 0.09 | 0.18 | 0.04 | 1.22 | 0.12 |
| BLDR 042 | 22.60 | 0.40 | 0.07 | 0.06 | 0.01 | 0.52 | 0.12 |
| BLDR 043 | 39.64 | 1.58 | 0.46 | 2.18 | 0.01 | 2.39 | 0.19 |
| BLDR 044 | 114.82 | 0.40 | 0.13 | 0.98 | 0.01 | 1.65 | 0.12 |
| BLDR 045 | 133.13 | 0.43 | 0.07 | 0.06 | 0.01 | 0.69 | 0.12 |
| BLDR 046 | 17.55 | 0.40 | 0.90 | 0.50 | 0.17 | 11.30 | 0.51 |
| BLDR 047 | 63.65 | 2.47 | 0.61 | 0.30 | 0.06 | 0.73 | 0.92 |
| BLDR 048 | 82.73 | 21.64 | 7.74 | 3.69 | 0.49 | 11.02 | 12.47 |
| PDAC 001 | 19.56 | 0.40 | 0.19 | 3.05 | 0.02 | 7.73 | 0.31 |
| PDAC 002 | 105.69 | 0.92 | 0.84 | 13.05 | 0.01 | 9.08 | 0.36 |
| PDAC 003 | 8.40 | 1.33 | 0.49 | 2.10 | 0.01 | 14.53 | 0.60 |
| PDAC 004 | 21.68 | 6.56 | 1.66 | 0.86 | 0.18 | 2.95 | 2.61 |
| PDAC 005 | 56.42 | 0.40 | 0.07 | 0.28 | 0.05 | 5.93 | 0.17 |
| PDAC 006 | 53.29 | 1.12 | 0.28 | 1.83 | 0.01 | 9.05 | 0.41 |
| PDAC 007 | 51.71 | 0.40 | 0.24 | 2.63 | 0.01 | 3.65 | 0.12 |
| PDAC 008 | 20.54 | 3.01 | 0.30 | 0.94 | 0.01 | 7.01 | 0.41 |
| PDAC 009 | 12.95 | 0.84 | 0.29 | 2.54 | 0.03 | 5.37 | 0.27 |
| PDAC 010 | 22.80 | 1.67 | 0.40 | 1.79 | 0.10 | 3.51 | 0.39 |
| PDAC 011 | 28.42 | 2.17 | 0.98 | 7.14 | 0.01 | 11.75 | 0.30 |
| PDAC 012 | 36.25 | 2.29 | 1.26 | 5.53 | 0.05 | 5.48 | 0.25 |
| PDAC 013 | 29.85 | 1.51 | 0.75 | 3.55 | 0.18 | 14.53 | 1.78 |
| PDAC 014 | 8.40 | 6.61 | 1.69 | 0.83 | 0.17 | 2.99 | 2.70 |
| PDAC 015 | 21.59 | 0.63 | 0.90 | 0.64 | 0.01 | 8.82 | 0.27 |
| PDAC 016 | 53.39 | 0.42 | 0.15 | 0.34 | 0.01 | 1.18 | 0.22 |
| PDAC 017 | 17.30 | 1.03 | 1.21 | 0.99 | 0.05 | 16.18 | 0.58 |
| PDAC 018 | 28.95 | 0.40 | 0.19 | 0.40 | 0.01 | 3.54 | 0.21 |
| PDAC 019 | 21.61 | 1.19 | 0.58 | 9.71 | 0.01 | 6.64 | 0.34 |
| PDAC 020 | 20.72 | 0.40 | 0.27 | 7.99 | 0.01 | 9.02 | 0.41 |
| PDAC 021 | 13.22 | 0.40 | 0.13 | 0.64 | 0.01 | 4.53 | 0.21 |
| PDAC 022 | 16.24 | 2.00 | 0.93 | 31.12 | 0.01 | 13.27 | 0.43 |
| PDAC 023 | 28.06 | 1.86 | 0.69 | 1.10 | 0.01 | 9.62 | 0.52 |
| PDAC 024 | 15.53 | 0.71 | 0.25 | 10.53 | 0.02 | 5.64 | 0.33 |
| PDAC 025 | 19.61 | 1.02 | 0.41 | 7.28 | 0.04 | 12.43 | 0.60 |
| PDAC 026 | 39.26 | 3.06 | 0.42 | 16.05 | 0.02 | 7.73 | 0.25 |
| PDAC 027 | 20.42 | 1.32 | 0.34 | 7.09 | 0.04 | 11.77 | 0.22 |
| PDAC 028 | 39.60 | 1.83 | 1.25 | 5.70 | 0.08 | 9.48 | 0.50 |
| PDAC 029 | 36.35 | 1.51 | 0.43 | 0.17 | 0.01 | 0.40 | 0.55 |
| PDAC 030 | 23.00 | 0.71 | 0.30 | 0.89 | 0.01 | 4.59 | 0.31 |
| PDAC 031 | 49.98 | 0.86 | 0.32 | 1.20 | 0.01 | 4.20 | 0.18 |
| PDAC 032 | 59.67 | 0.69 | 0.13 | 1.97 | 0.01 | 4.77 | 0.22 |
| PDAC 033 | 67.48 | 0.40 | 0.06 | 0.32 | 0.01 | 0.54 | 0.12 |
| PDAC 034 | 48.14 | 0.40 | 0.05 | 0.17 | 0.01 | 0.20 | 0.12 |
| PDAC 035 | 60.35 | 1.29 | 0.41 | 1.56 | 0.01 | 5.16 | 0.32 |
| PDAC 036 | 8.40 | 0.63 | 0.67 | 1.44 | 0.01 | 5.81 | 0.47 |
| PDAC 037 | 36.31 | 0.40 | 0.13 | 0.57 | 0.02 | 0.87 | 0.15 |
| PDAC 038 | 8.40 | 0.46 | 0.22 | 1.82 | 0.01 | 6.71 | 0.27 |
| PDAC 039 | 8.40 | 1.99 | 1.19 | 4.50 | 0.01 | 6.82 | 0.38 |
| PDAC 040 | 8.40 | 2.35 | 0.64 | 0.15 | 0.01 | 4.22 | 0.47 |
| PDAC 041 | 35.00 | 0.40 | 0.54 | 11.33 | 0.05 | 2.23 | 0.12 |
| PDAC 042 | 28.24 | 0.84 | 0.37 | 0.62 | 0.03 | 3.12 | 0.36 |
| PDAC 043 | 53.56 | 73.27 | 19.89 | 16.83 | 3.23 | 16.26 | 35.76 |
| PDAC 044 | 28.64 | 0.55 | 0.37 | 13.72 | 0.03 | 6.16 | 0.14 |
| HC 001 | 25.77 | 1.00 | 0.54 | 0.32 | 0.01 | 7.49 | 0.27 |
| HC 002 | 14.63 | 1.50 | 0.20 | 0.14 | 0.01 | 3.46 | 0.12 |
| HC 003 | 44.02 | 4.39 | 0.05 | 0.28 | 0.01 | 5.01 | 0.14 |
| HC 004 | 8.40 | 1.67 | 0.05 | 0.20 | 0.01 | 5.87 | 0.12 |
| HC 005 | 43.36 | 3.31 | 0.25 | 0.50 | 0.01 | 5.40 | 0.24 |
| HC 006 | 20.28 | 10.14 | 0.51 | 0.54 | 0.05 | 8.84 | 0.34 |
| HC 007 | 52.82 | 15.37 | 0.34 | 0.91 | 0.12 | 7.03 | 0.49 |
| HC 008 | 24.57 | 2.85 | 1.05 | 0.14 | 0.01 | 11.62 | 0.33 |
| HC 009 | 25.44 | 5.79 | 1.88 | 0.87 | 0.06 | 10.42 | 0.79 |
| HC 010 | 25.86 | 1.63 | 0.55 | 0.12 | 0.01 | 4.96 | 0.27 |
| HC 011 | 42.03 | 7.52 | 1.42 | 0.93 | 0.05 | 9.37 | 0.88 |
| HC 012 | 20.58 | 3.14 | 0.72 | 1.60 | 0.06 | 18.53 | 0.46 |
| HC 013 | 38.57 | 1.03 | 0.57 | 1.89 | 0.02 | 9.01 | 0.23 |
| HC 014 | 27.17 | 3.24 | 0.53 | 1.07 | 0.06 | 20.49 | 0.37 |
| HC 015 | 29.75 | 1.36 | 0.48 | 2.24 | 0.03 | 20.77 | 0.23 |
| HC 016 | 68.59 | 0.57 | 0.31 | 0.56 | 0.03 | 3.90 | 0.17 |
| HC 017 | 81.47 | 0.94 | 0.38 | 1.92 | 0.02 | 5.17 | 0.22 |
| HC 018 | 37.26 | 0.70 | 0.48 | 0.71 | 0.02 | 7.53 | 0.20 |
| HC 019 | 19.25 | 0.71 | 0.31 | 0.73 | 0.01 | 6.63 | 0.18 |
| HC 020 | 15.11 | 6.11 | 3.13 | 5.15 | 0.12 | 10.03 | 0.54 |
| HC 021 | 25.77 | 1.69 | 0.41 | 0.39 | 0.04 | 6.81 | 0.29 |
| HC 022 | 22.15 | 2.01 | 0.67 | 0.64 | 0.05 | 9.21 | 0.32 |
| HC 023 | 24.76 | 3.63 | 2.35 | 2.81 | 0.05 | 12.21 | 0.61 |
| HC 024 | 21.69 | 3.82 | 0.57 | 0.73 | 0.01 | 6.35 | 0.33 |
| HC 025 | 26.40 | 4.99 | 0.69 | 1.57 | 0.07 | 8.14 | 0.22 |
| HC 026 | 23.75 | 1.49 | 0.57 | 0.24 | 0.01 | 9.91 | 0.31 |
| HC 027 | 44.45 | 4.66 | 0.05 | 0.28 | 0.01 | 5.46 | 0.12 |
| HC 028 | 17.57 | 0.82 | 0.10 | 0.18 | 0.03 | 5.85 | 0.12 |
| HC 029 | 10.76 | 0.59 | 0.15 | 0.30 | 0.01 | 4.05 | 0.22 |
| HC 030 | 93.42 | 1.40 | 0.35 | 0.19 | 0.01 | 3.16 | 0.12 |
| HC 031 | 21.82 | 0.40 | 0.25 | 0.33 | 0.01 | 0.20 | 0.12 |
| HC 032 | 10.12 | 0.40 | 0.28 | 0.12 | 0.01 | 0.70 | 0.12 |
| HC 033 | 66.59 | 1.00 | 0.17 | 0.04 | 0.01 | 1.56 | 0.12 |
| HC 034 | 58.14 | 0.88 | 0.22 | 0.89 | 0.06 | 1.52 | 0.16 |
| HC 035 | 90.36 | 2.99 | 0.38 | 0.29 | 0.01 | 1.21 | 0.15 |
| HC 036 | 70.43 | 1.81 | 0.86 | 0.10 | 0.01 | 6.73 | 0.12 |
| HC 037 | 64.29 | 4.08 | 0.05 | 0.87 | 0.01 | 2.22 | 0.12 |
| HC 038 | 108.71 | 309.01 | 0.86 | 0.48 | 0.04 | 3.47 | 0.50 |
| HC 039 | 73.50 | 2.60 | 0.05 | 0.08 | 0.01 | 3.32 | 0.12 |
| HC 040 | 8.40 | 2.22 | 0.24 | 0.71 | 0.01 | 1.92 | 0.19 |
| HC 041 | 66.59 | 8.01 | 0.53 | 0.90 | 0.01 | 4.04 | 0.26 |
| HC 042 | 8.40 | 3.49 | 0.49 | 0.59 | 0.03 | 3.76 | 0.32 |
| HC 043 | 24.23 | 0.97 | 0.96 | 1.44 | 0.05 | 6.78 | 0.22 |
| HC 044 | 29.91 | 0.40 | 0.12 | 0.18 | 0.02 | 4.17 | 0.12 |
| HC 045 | 18.93 | 0.40 | 0.60 | 0.59 | 0.05 | 8.67 | 0.31 |
| HC 046 | 23.94 | 1.52 | 1.40 | 1.47 | 0.04 | 15.62 | 0.45 |
| HC 047 | 83.37 | 0.40 | 0.05 | 0.31 | 0.02 | 5.57 | 0.27 |
| HC 048 | 53.59 | 0.78 | 0.60 | 0.97 | 0.02 | 7.94 | 0.23 |
| HC 049 | 36.53 | 0.40 | 0.41 | 0.04 | 0.01 | 3.11 | 0.12 |
| HC 050 | 28.58 | 0.40 | 0.26 | 0.29 | 0.02 | 7.62 | 0.25 |
| HC 051 | 41.49 | 0.44 | 0.42 | 0.46 | 0.02 | 6.24 | 0.12 |
| HC 052 | 26.92 | 0.40 | 0.05 | 0.29 | 0.02 | 5.96 | 0.32 |
| HC 053 | 45.71 | 0.40 | 0.30 | 0.12 | 0.01 | 4.46 | 0.19 |
| HC 054 | 37.62 | 0.49 | 0.80 | 0.39 | 0.02 | 8.98 | 0.24 |
| HC 055 | 42.18 | 0.66 | 0.62 | 0.33 | 0.01 | 9.85 | 0.41 |
| HC 056 | 18.09 | 0.96 | 1.01 | 0.38 | 0.01 | 7.80 | 0.39 |
| HC 057 | 43.22 | 0.40 | 0.13 | 0.09 | 0.01 | 3.30 | 0.12 |
| HC 058 | 17.32 | 14.15 | 0.05 | 0.35 | 0.01 | 14.23 | 0.12 |
| HC 059 | 31.74 | 25.27 | 0.46 | 0.36 | 0.01 | 15.14 | 0.60 |
| HC 060 | 46.22 | 0.59 | 0.55 | 1.17 | 0.01 | 7.05 | 0.42 |
| HC 061 | 52.01 | 0.40 | 0.42 | 1.49 | 0.02 | 8.88 | 0.55 |
| HC 062 | 55.10 | 0.40 | 0.11 | 2.40 | 0.02 | 2.36 | 0.17 |
| HC 063 | 8.40 | 1.28 | 0.43 | 7.10 | 0.01 | 6.99 | 0.22 |
| HC 064 | 55.81 | 1.92 | 0.26 | 0.17 | 0.01 | 4.97 | 0.40 |
| HC 065 | 8.40 | 1.05 | 0.28 | 2.89 | 0.02 | 6.41 | 0.12 |
| HC 066 | 8.40 | 0.40 | 0.16 | 3.04 | 0.01 | 5.92 | 0.13 |
| HC 067 | 105.23 | 2.33 | 1.05 | 0.19 | 0.01 | 2.96 | 0.12 |
| HC 068 | 33.16 | 0.40 | 0.12 | 0.05 | 0.01 | 6.00 | 0.24 |
| HC 069 | 22.73 | 0.47 | 0.26 | 0.04 | 0.01 | 2.16 | 0.18 |
| HC 070 | 21.34 | 0.66 | 0.22 | 0.33 | 0.01 | 1.09 | 0.12 |
| HC 071 | 47.26 | 153.60 | 14.61 | 22.11 | 6.23 | 67.42 | 8.65 |
| HC 072 | 49.19 | 0.96 | 0.93 | 9.15 | 0.01 | 2.23 | 0.23 |
| HC 073 | 122.61 | 1.42 | 0.17 | 1.01 | 0.01 | 12.87 | 0.12 |
| HC 074 | 23.54 | 2.04 | 1.27 | 1.08 | 0.01 | 25.85 | 0.48 |
| HC 075 | 23.89 | 2.48 | 1.19 | 0.98 | 0.07 | 12.70 | 0.44 |
| HC 076 | 35.99 | 3.09 | 1.53 | 0.32 | 0.09 | 20.56 | 0.40 |
| HC 077 | 39.44 | 2.15 | 0.44 | 0.57 | 0.01 | 14.05 | 0.25 |
| HC 078 | 35.90 | 1.22 | 0.31 | 0.43 | 0.02 | 16.21 | 0.19 |
| HC 079 | 14.44 | 78.40 | 8.71 | 5.97 | 3.33 | 60.56 | 13.18 |
| HC 080 | 70.42 | 1.16 | 0.24 | 0.28 | 0.01 | 13.98 | 0.23 |
| HC 081 | 18.26 | 60.57 | 15.55 | 8.12 | 2.75 | 16.83 | 25.16 |
| HC 082 | 20.40 | 0.77 | 0.48 | 0.16 | 0.01 | 6.22 | 0.25 |
| HC 083 | 45.66 | 30.43 | 9.39 | 3.60 | 0.38 | 8.96 | 13.49 |
| HC 084 | 47.81 | 0.69 | 0.25 | 0.15 | 0.01 | 2.31 | 0.29 |
| HC 085 | 59.28 | 0.78 | 0.22 | 0.18 | 0.05 | 3.24 | 0.13 |
| HC 086 | 22.33 | 0.62 | 0.22 | 0.05 | 0.01 | 2.56 | 0.12 |
| HC 087 | 9.99 | 1.27 | 0.34 | 0.04 | 0.01 | 4.22 | 0.34 |
| HC 088 | 11.38 | 0.91 | 1.77 | 0.37 | 0.04 | 11.12 | 0.34 |
| HC 089 | 26.17 | 3.10 | 0.26 | 0.34 | 0.01 | 3.98 | 0.38 |
| HC 090 | 17.57 | 0.44 | 0.25 | 0.16 | 0.01 | 2.01 | 0.17 |
| HC 091 | 10.68 | 1.00 | 0.54 | 0.16 | 0.04 | 9.92 | 0.28 |
| HC 092 | 21.34 | 0.57 | 0.40 | 0.08 | 0.01 | 3.03 | 0.14 |
| HC 093 | 8.40 | 0.71 | 0.30 | 0.14 | 0.01 | 4.81 | 0.18 |
| HC 094 | 30.90 | 0.44 | 0.22 | 0.04 | 0.03 | 1.69 | 0.21 |
| HC 095 | 8.40 | 0.81 | 0.50 | 0.23 | 0.02 | 6.09 | 0.26 |
| HC 096 | 37.05 | 3.38 | 1.52 | 0.76 | 0.11 | 13.84 | 0.48 |
| HC 097 | 13.09 | 2.65 | 0.66 | 0.31 | 0.15 | 11.62 | 0.30 |
| HC 098 | 13.81 | 1.46 | 2.02 | 0.68 | 0.01 | 17.36 | 0.31 |
| HC 099 | 34.86 | 26.08 | 1.62 | 0.48 | 0.25 | 12.58 | 1.06 |
| HC 100 | 52.82 | 0.40 | 0.22 | 0.10 | 0.01 | 6.01 | 0.20 |
| HC 101 | 664.30 | 0.40 | 0.13 | 0.05 | 0.03 | 3.50 | 0.21 |
| HC 102 | 271.21 | 0.87 | 0.53 | 0.10 | 0.01 | 7.01 | 0.24 |
| HC 103 | 8.40 | 0.40 | 0.08 | 0.05 | 0.01 | 2.36 | 0.16 |
| HC 104 | 13.39 | 0.40 | 0.10 | 0.04 | 0.01 | 1.86 | 0.12 |
| HC 105 | 17.57 | 0.47 | 0.52 | 0.21 | 0.13 | 10.59 | 0.32 |
| HC 106 | 38.81 | 0.40 | 0.49 | 0.20 | 0.09 | 8.03 | 0.19 |
| HC 107 | 27.41 | 0.40 | 0.05 | 0.04 | 0.01 | 1.17 | 0.12 |
| HC 108 | 21.90 | 0.98 | 0.35 | 0.52 | 0.01 | 5.04 | 0.23 |
| HC 109 | 63.32 | 0.40 | 0.27 | 0.63 | 0.01 | 3.52 | 0.16 |
| HC 110 | 43.60 | 0.40 | 0.21 | 0.19 | 0.01 | 1.81 | 0.25 |
| HC 111 | 16.09 | 0.40 | 0.17 | 0.59 | 0.01 | 2.64 | 0.12 |
| HC 112 | 25.86 | 0.73 | 0.31 | 0.43 | 0.01 | 4.75 | 0.23 |
| HC 113 | 8.40 | 0.93 | 0.28 | 0.15 | 0.01 | 3.73 | 0.19 |
| OVAR 001 | 2504.13 | 77.84 | 74.59 | 209.92 | 6.24 | 671.70 | 16.98 |
| OVAR 002 | 1628.63 | 59.97 | 66.79 | 200.05 | 2.52 | 628.54 | 24.70 |
| OVAR 003 | 1718.65 | 60.57 | 113.68 | 66.57 | 3.93 | 592.19 | 22.43 |
| OVAR 004 | 680.02 | 79.41 | 36.14 | 84.36 | 4.32 | 312.71 | 15.81 |
| OVAR 005 | 1495.40 | 88.42 | 97.53 | 138.51 | 4.07 | 479.43 | 33.91 |
| OVAR 006 | 968.95 | 29.62 | 28.74 | 28.01 | 2.49 | 387.89 | 12.77 |
| OVAR 007 | 1126.08 | 69.71 | 59.42 | 48.17 | 2.55 | 503.14 | 27.32 |
| OVAR 008 | 2322.27 | 83.72 | 41.36 | 1410.22 | 4.87 | 810.33 | 25.45 |
| OVAR 009 | 1055.14 | 76.25 | 29.58 | 404.73 | 5.11 | 665.76 | 20.51 |
| OVAR 010 | 1134.66 | 4443.82 | 1033.46 | 549.17 | 97.80 | 909.42 | 1794.03 |
| OVAR 011 | 500.65 | 81.64 | 59.86 | 467.37 | 10.13 | 1017.60 | 33.18 |
| OVAR 012 | 615.19 | 44.62 | 78.89 | 268.57 | 4.66 | 482.11 | 29.90 |
| OVAR 013 | 933.58 | 85.89 | 77.17 | 4924.21 | 6.16 | 4332.95 | 20.86 |
| OVAR 014 | 2637.61 | 169.60 | 82.30 | 176.15 | 6.26 | 706.27 | 20.51 |
| OVAR 015 | 1969.95 | 207.47 | 126.15 | 720.99 | 17.45 | 2719.83 | 51.09 |
| OVAR 016 | 2671.19 | 68.94 | 76.33 | 16.84 | 5.42 | 825.95 | 18.57 |
| OVAR 017 | 1847.18 | 87.76 | 43.94 | 1331.93 | 5.40 | 441.01 | 15.40 |
| OVAR 018 | 1403.73 | 90.38 | 83.88 | 52.07 | 5.11 | 551.68 | 21.66 |
| OVAR 019 | 1059.58 | 73.74 | 54.57 | 958.40 | 4.22 | 688.26 | 26.38 |
| OVAR 020 | 1994.75 | 84.85 | 91.95 | 352.90 | 3.10 | 643.83 | 22.43 |
| OVAR 021 | 1043.52 | 53.86 | 102.59 | 68.45 | 7.57 | 459.28 | 19.74 |
| OVAR 022 | 8.40 | 54.89 | 124.44 | 70.32 | 2.54 | 529.39 | 20.70 |
| OVAR 023 | 3322.14 | 76.72 | 61.45 | 543.04 | 24.98 | 941.92 | 22.05 |
| OVAR 024 | 1024.28 | 77.19 | 78.26 | 2747.93 | 6.96 | 689.43 | 26.57 |
| OVAR 025 | 1617.05 | 52.38 | 75.92 | 93.81 | 5.51 | 490.17 | 26.20 |
| OVAR 026 | 1017.03 | 96.01 | 47.66 | 308.27 | 4.79 | 443.78 | 22.43 |
| OVAR 027 | 906.84 | 85.82 | 59.61 | 49.73 | 4.08 | 447.47 | 20.90 |
| OVAR 028 | 1175.47 | 69.71 | 101.08 | 74.79 | 3.95 | 647.51 | 31.00 |
| OVAR 029 | 2113.13 | 1329.65 | 452.27 | 188.97 | 18.20 | 501.78 | 659.92 |
| OVAR 030 | 1823.07 | 165.06 | 134.89 | 427.00 | 10.15 | 568.45 | 28.54 |
| OVAR 031 | 1241.08 | 140.02 | 206.00 | 76.89 | 8.05 | 2473.63 | 51.63 |
| OVAR 032 | 2414.58 | 254.85 | 144.51 | 4617.90 | 25.56 | 2744.97 | 38.76 |
| OVAR 033 | 1514.46 | 12.93 | 15.44 | 9.37 | 0.01 | 89.06 | 7.50 |
| OVAR 034 | 1212.74 | 128.70 | 181.11 | 141.91 | 7.37 | 1538.69 | 49.22 |
| OVAR 035 | 1186.19 | 252.08 | 103.97 | 2785.57 | 15.37 | 625.41 | 58.19 |
| OVAR 036 | 1360.87 | 360.78 | 128.83 | 8329.85 | 12.90 | 628.55 | 30.15 |
| OVAR 037 | 1604.94 | 137.62 | 104.44 | 439.10 | 9.61 | 428.39 | 30.82 |
| OVAR 038 | 824.26 | 169.32 | 132.85 | 63.26 | 5.90 | 409.17 | 21.52 |
| OVAR 039 | 522.45 | 102.33 | 141.18 | 328.19 | 10.33 | 350.10 | 49.22 |
| OVAR 040 | 2356.12 | 172.85 | 108.49 | 95.96 | 8.21 | 888.30 | 30.63 |
| OVAR 041 | 588.07 | 60.66 | 156.29 | 306.10 | 15.96 | 7631.35 | 40.83 |
| OVAR 042 | 935.86 | 251.00 | 228.86 | 245.95 | 27.98 | 7683.40 | 71.80 |
| OVAR 043 | 661.44 | 718.33 | 59.72 | 7493.45 | 20.19 | 4348.96 | 37.31 |
| OVAR 044 | 578.89 | 753.94 | 378.18 | 2440.63 | 17.04 | 8679.66 | 92.11 |
| BLDR 001 | 872.26 | 102.91 | 108.44 | 487.71 | 11.22 | 908.98 | 27.90 |
| BLDR 002 | 1572.21 | 54.97 | 61.78 | 210.00 | 5.74 | 330.10 | 14.97 |
| BLDR 003 | 1299.59 | 56.83 | 70.38 | 115.36 | 8.05 | 590.06 | 23.83 |
| BLDR 004 | 1049.92 | 65.68 | 71.46 | 21.93 | 4.05 | 638.18 | 23.83 |
| BLDR 005 | 455.67 | 100.97 | 75.37 | 1101.37 | 7.94 | 526.45 | 22.53 |
| BLDR 006 | 426.22 | 56.70 | 129.39 | 452.69 | 5.86 | 484.75 | 9.43 |
| BLDR 007 | 1758.59 | 51.31 | 55.82 | 178.04 | 16.55 | 863.65 | 17.52 |
| BLDR 008 | 2291.85 | 64.35 | 71.70 | 48.59 | 7.03 | 669.38 | 22.02 |
| BLDR 009 | 1388.85 | 29.86 | 44.81 | 159.61 | 3.29 | 432.69 | 12.18 |
| BLDR 010 | 496.69 | 59.45 | 77.19 | 40.98 | 4.14 | 396.31 | 21.23 |
| BLDR 011 | 1341.11 | 55.71 | 66.30 | 259.77 | 5.71 | 647.25 | 12.74 |
| BLDR 012 | 1406.28 | 76.10 | 117.28 | 178.96 | 5.75 | 709.64 | 22.43 |
| BLDR 013 | 1353.37 | 55.71 | 77.48 | 139.33 | 5.71 | 418.74 | 20.94 |
| BLDR 014 | 1193.24 | 37.91 | 75.17 | 123.04 | 3.37 | 510.79 | 20.66 |
| BLDR 015 | 1342.57 | 62.73 | 55.32 | 26.84 | 4.72 | 536.64 | 18.23 |
| BLDR 016 | 1424.81 | 147.86 | 146.83 | 156.65 | 10.85 | 796.05 | 25.17 |
| BLDR 017 | 1837.37 | 57.53 | 94.52 | 257.74 | 5.03 | 516.32 | 17.03 |
| BLDR 018 | 1794.71 | 66.72 | 146.74 | 51.44 | 8.06 | 837.73 | 33.89 |
| BLDR 019 | 1394.43 | 106.01 | 109.32 | 541.32 | 7.01 | 716.01 | 38.21 |
| BLDR 020 | 2608.40 | 30.57 | 30.67 | 21.93 | 5.25 | 374.93 | 13.40 |
| BLDR 021 | 880.45 | 109.11 | 63.11 | 772.75 | 7.28 | 590.59 | 23.39 |
| BLDR 022 | 2356.64 | 43.34 | 71.63 | 248.71 | 14.03 | 794.40 | 17.23 |
| BLDR 023 | 873.97 | 61.59 | 81.75 | 90.77 | 5.74 | 458.94 | 18.37 |
| BLDR 024 | 1150.12 | 79.61 | 80.37 | 77.56 | 9.23 | 601.02 | 22.96 |
| BLDR 025 | 2580.70 | 257.93 | 60.20 | 140.56 | 6.67 | 1014.84 | 18.37 |
| BLDR 026 | 2326.73 | 130.05 | 85.68 | 419.62 | 5.00 | 674.10 | 34.15 |
| BLDR 027 | 1831.16 | 80.24 | 134.71 | 149.71 | 4.97 | 707.10 | 40.17 |
| BLDR 028 | 1093.88 | 82.42 | 80.63 | 127.04 | 6.32 | 614.89 | 27.95 |
| BLDR 029 | 2740.91 | 104.59 | 103.31 | 46.87 | 9.46 | 644.41 | 28.14 |
| BLDR 030 | 2038.64 | 62.22 | 83.93 | 263.76 | 8.18 | 664.19 | 27.61 |
| BLDR 031 | 2141.01 | 61.21 | 49.68 | 36.07 | 5.42 | 494.33 | 23.83 |
| BLDR 032 | 945.35 | 117.95 | 89.34 | 735.13 | 4.46 | 1007.75 | 26.94 |
| BLDR 033 | 1365.67 | 39.06 | 47.52 | 28.69 | 8.93 | 411.66 | 10.80 |
| BLDR 034 | 899.80 | 58.57 | 49.52 | 221.06 | 10.26 | 507.22 | 19.22 |
| BLDR 035 | 2273.99 | 170.21 | 210.30 | 4455.98 | 6.11 | 655.84 | 41.35 |
| BLDR 036 | 1636.10 | 112.97 | 113.69 | 429.96 | 6.63 | 421.33 | 18.37 |
| BLDR 037 | 1392.70 | 46.99 | 53.98 | 22.85 | 5.54 | 333.51 | 18.94 |
| BLDR 038 | 871.92 | 91.02 | 239.47 | 2347.72 | 66.15 | 4868.86 | 47.15 |
| BLDR 039 | 2146.16 | 78.52 | 47.46 | 47.75 | 10.03 | 576.30 | 26.01 |
| BLDR 040 | 2219.16 | 69.58 | 99.80 | 1281.95 | 11.19 | 672.72 | 22.09 |
| BLDR 041 | 707.25 | 49.33 | 93.61 | 173.51 | 3.03 | 444.54 | 31.02 |
| BLDR 042 | 917.60 | 92.84 | 138.69 | 141.86 | 7.46 | 2301.06 | 50.43 |
| BLDR 043 | 780.48 | 156.97 | 250.21 | 974.85 | 31.40 | 9459.05 | 54.51 |
| BLDR 044 | 1072.72 | 129.92 | 205.10 | 1717.42 | 11.44 | 4372.99 | 64.75 |
| BLDR 045 | 734.72 | 172.75 | 157.19 | 185.59 | 4.06 | 2607.34 | 114.10 |
| BLDR 046 | 1051.74 | 33.18 | 76.59 | 18.24 | 2.57 | 489.39 | 14.13 |
| BLDR 047 | 1765.76 | 41.13 | 69.25 | 24.38 | 8.04 | 380.71 | 13.29 |
| BLDR 048 | 1406.21 | 55.84 | 68.51 | 29.92 | 7.61 | 587.87 | 17.80 |
| PDAC 001 | 1721.93 | 49.73 | 57.60 | 419.52 | 4.53 | 491.47 | 25.82 |
| PDAC 002 | 1803.08 | 160.31 | 105.54 | 1636.46 | 8.44 | 687.99 | 26.20 |
| PDAC 003 | 1617.05 | 129.41 | 55.78 | 164.74 | 6.99 | 1152.11 | 31.73 |
| PDAC 004 | 1372.12 | 83.88 | 59.23 | 2785.82 | 6.67 | 724.32 | 25.82 |
| PDAC 005 | 779.35 | 142.27 | 58.49 | 87.28 | 7.27 | 675.78 | 20.90 |
| PDAC 006 | 1844.61 | 115.98 | 57.25 | 406.85 | 5.52 | 572.13 | 20.51 |
| PDAC 007 | 1464.07 | 78.14 | 43.62 | 711.73 | 6.61 | 598.60 | 22.81 |
| PDAC 008 | 1796.28 | 85.74 | 138.08 | 266.29 | 6.58 | 1782.75 | 26.76 |
| PDAC 009 | 1552.14 | 77.51 | 26.26 | 355.36 | 3.47 | 453.73 | 16.60 |
| PDAC 010 | 1215.01 | 282.43 | 31.81 | 496.94 | 2.32 | 501.38 | 19.23 |
| PDAC 011 | 1818.07 | 189.45 | 92.76 | 450.52 | 4.06 | 825.99 | 18.94 |
| PDAC 012 | 1092.59 | 53.27 | 74.88 | 95.61 | 3.39 | 448.62 | 19.74 |
| PDAC 013 | 704.31 | 58.77 | 63.43 | 226.19 | 3.50 | 570.96 | 29.53 |
| PDAC 014 | 871.64 | 84.85 | 58.27 | 354.75 | 5.34 | 767.27 | 18.96 |
| PDAC 015 | 2423.91 | 103.76 | 205.82 | 112.13 | 5.84 | 2251.36 | 26.36 |
| PDAC 016 | 1980.64 | 98.02 | 93.94 | 729.22 | 4.22 | 656.71 | 30.63 |
| PDAC 017 | 1467.58 | 47.80 | 83.41 | 75.78 | 4.71 | 2089.75 | 25.56 |
| PDAC 018 | 3309.17 | 131.78 | 150.01 | 248.03 | 8.37 | 2011.94 | 61.59 |
| PDAC 019 | 1567.79 | 189.43 | 93.15 | 1509.15 | 11.15 | 870.33 | 29.53 |
| PDAC 020 | 1230.34 | 55.79 | 31.60 | 698.73 | 2.89 | 413.27 | 22.24 |
| PDAC 021 | 586.06 | 80.04 | 74.27 | 405.94 | 4.93 | 814.36 | 20.51 |
| PDAC 022 | 1964.39 | 121.72 | 57.63 | 1439.82 | 10.52 | 1032.01 | 23.19 |
| PDAC 023 | 1987.18 | 100.72 | 89.18 | 140.92 | 5.45 | 856.63 | 32.82 |
| PDAC 024 | 2715.39 | 163.79 | 53.61 | 2486.00 | 6.64 | 719.21 | 33.18 |
| PDAC 025 | 1040.66 | 80.68 | 42.52 | 547.71 | 7.59 | 785.99 | 31.00 |
| PDAC 026 | 1587.57 | 336.39 | 63.34 | 2080.80 | 5.43 | 625.98 | 18.74 |
| PDAC 027 | 1066.13 | 132.96 | 55.30 | 734.29 | 6.48 | 923.97 | 18.96 |
| PDAC 028 | 2418.46 | 456.16 | 78.96 | 1141.14 | 6.63 | 1008.89 | 28.43 |
| PDAC 029 | 1367.74 | 83.87 | 91.02 | 149.77 | 3.41 | 892.36 | 36.76 |
| PDAC 030 | 1246.04 | 156.87 | 98.38 | 1252.15 | 5.71 | 650.56 | 19.08 |
| PDAC 031 | 900.75 | 71.29 | 51.97 | 161.84 | 4.83 | 1328.78 | 19.96 |
| PDAC 032 | 832.59 | 74.74 | 52.81 | 30.53 | 7.58 | 374.56 | 17.80 |
| PDAC 033 | 707.47 | 46.28 | 70.53 | 1453.92 | 10.15 | 550.45 | 17.80 |
| PDAC 034 | 1580.96 | 55.96 | 67.04 | 648.97 | 8.53 | 470.61 | 14.41 |
| PDAC 035 | 479.84 | 64.78 | 73.68 | 376.51 | 8.50 | 659.36 | 24.55 |
| PDAC 036 | 1418.29 | 71.55 | 63.76 | 492.32 | 7.75 | 1543.64 | 18.65 |
| PDAC 037 | 3154.28 | 34.93 | 0.05 | 19.64 | 0.01 | 74.71 | 9.96 |
| PDAC 038 | 2080.96 | 72.74 | 85.14 | 187.26 | 7.75 | 986.66 | 18.65 |
| PDAC 039 | 2092.07 | 85.77 | 99.73 | 377.35 | 5.87 | 697.68 | 29.53 |
| PDAC 040 | 1263.64 | 70.13 | 93.12 | 306.78 | 5.86 | 685.37 | 30.39 |
| PDAC 041 | 1264.02 | 63.74 | 171.59 | 4287.31 | 20.15 | 2731.46 | 36.40 |
| PDAC 042 | 2589.35 | 67.20 | 47.16 | 1120.97 | 0.01 | 246.35 | 22.28 |
| PDAC 043 | 1289.46 | 71.55 | 79.40 | 315.07 | 7.16 | 778.22 | 15.68 |
| PDAC 044 | 1659.62 | 80.15 | 61.89 | 1692.21 | 6.89 | 535.32 | 12.46 |
| HC 001 | 2185.49 | 83.74 | 88.10 | 37.30 | 8.98 | 624.17 | 18.08 |
| HC 002 | 1621.58 | 45.81 | 71.92 | 21.93 | 8.56 | 442.68 | 10.52 |
| HC 003 | 1269.58 | 50.10 | 83.58 | 9.63 | 8.45 | 544.53 | 11.07 |
| HC 004 | 1114.13 | 62.35 | 113.22 | 12.09 | 12.59 | 621.55 | 13.85 |
| HC 005 | 1975.14 | 86.25 | 62.80 | 74.18 | 13.75 | 609.57 | 23.83 |
| HC 006 | 1700.66 | 96.26 | 54.31 | 47.75 | 1.86 | 758.68 | 18.65 |
| HC 007 | 1581.18 | 95.38 | 80.71 | 104.30 | 7.29 | 896.36 | 18.37 |
| HC 008 | 1404.68 | 70.50 | 106.65 | 9.01 | 4.67 | 829.77 | 14.83 |
| HC 009 | 496.16 | 121.02 | 90.38 | 21.93 | 5.06 | 365.27 | 31.13 |
| HC 010 | 1481.97 | 68.66 | 105.90 | 9.01 | 4.90 | 810.13 | 13.85 |
| HC 011 | 762.48 | 127.50 | 100.59 | 21.31 | 4.83 | 440.79 | 30.84 |
| HC 012 | 2543.45 | 65.55 | 60.26 | 101.84 | 13.06 | 715.54 | 14.69 |
| HC 013 | 2790.78 | 79.33 | 74.48 | 127.96 | 15.04 | 706.38 | 17.09 |
| HC 014 | 2644.85 | 74.87 | 64.77 | 98.76 | 14.58 | 842.47 | 15.25 |
| HC 015 | 2520.38 | 89.35 | 79.68 | 164.52 | 17.50 | 776.50 | 19.80 |
| HC 016 | 2097.70 | 103.35 | 84.36 | 81.25 | 13.76 | 615.48 | 12.46 |
| HC 017 | 806.00 | 62.61 | 61.67 | 99.99 | 3.13 | 421.38 | 19.22 |
| HC 018 | 1352.65 | 123.31 | 93.76 | 119.64 | 10.44 | 649.11 | 18.11 |
| HC 019 | 1794.62 | 128.49 | 99.28 | 96.31 | 17.16 | 683.29 | 15.82 |
| HC 020 | 2164.44 | 125.05 | 94.46 | 93.54 | 18.62 | 667.35 | 14.97 |
| HC 021 | 1576.95 | 95.67 | 79.34 | 69.88 | 22.03 | 505.33 | 16.95 |
| HC 022 | 1683.27 | 97.43 | 76.48 | 62.50 | 22.06 | 526.07 | 15.54 |
| HC 023 | 1524.43 | 94.66 | 84.99 | 66.19 | 21.84 | 528.91 | 15.54 |
| HC 024 | 1661.30 | 102.76 | 82.69 | 69.26 | 24.37 | 547.65 | 16.10 |
| HC 025 | 733.43 | 62.22 | 78.14 | 62.19 | 6.42 | 432.09 | 13.29 |
| HC 026 | 1411.25 | 96.55 | 126.03 | 40.23 | 7.52 | 840.05 | 24.67 |
| HC 027 | 1262.97 | 163.49 | 80.60 | 36.99 | 7.35 | 714.74 | 17.52 |
| HC 028 | 1211.49 | 153.61 | 68.51 | 31.15 | 6.79 | 826.74 | 16.10 |
| HC 029 | 683.42 | 56.70 | 61.16 | 63.73 | 6.16 | 411.61 | 13.85 |
| HC 030 | 615.59 | 85.74 | 165.21 | 97.89 | 6.60 | 2694.76 | 31.27 |
| HC 031 | 871.92 | 168.87 | 94.21 | 111.87 | 9.21 | 2698.09 | 27.25 |
| HC 032 | 569.71 | 214.21 | 250.99 | 115.90 | 3.82 | 279.73 | 71.80 |
| HC 033 | 599.44 | 79.79 | 165.98 | 26.08 | 8.57 | 2150.78 | 26.12 |
| HC 034 | 624.77 | 15277.71 | 499.43 | 1191.29 | 160.23 | 10113.00 | 80.69 |
| HC 035 | 862.78 | 119.86 | 161.61 | 82.14 | 5.07 | 292.33 | 42.68 |
| HC 036 | 679.76 | 262.43 | 281.89 | 34.56 | 9.26 | 5756.94 | 38.00 |
| HC 037 | 1163.86 | 605.36 | 275.47 | 641.38 | 19.28 | 8203.22 | 30.01 |
| HC 038 | 647.69 | 12026.14 | 603.31 | 187.17 | 22.04 | 5056.29 | 146.83 |
| HC 039 | 578.89 | 128.65 | 214.05 | 28.08 | 12.57 | 5825.01 | 28.25 |
| HC 040 | 1416.83 | 184.01 | 119.99 | 201.39 | 4.56 | 698.25 | 18.91 |
| HC 041 | 739.30 | 74.01 | 146.85 | 208.64 | 26.23 | 1486.97 | 73.94 |
| HC 042 | 1672.72 | 68.51 | 90.00 | 49.45 | 9.99 | 532.93 | 30.15 |
| HC 043 | 1775.09 | 102.16 | 98.73 | 61.27 | 29.35 | 617.82 | 22.67 |
| HC 044 | 1443.46 | 58.70 | 83.70 | 29.92 | 31.78 | 420.52 | 19.80 |
| HC 045 | 1431.15 | 41.13 | 59.75 | 21.31 | 14.38 | 314.77 | 13.57 |
| HC 046 | 1234.92 | 56.45 | 64.63 | 36.68 | 6.95 | 674.56 | 13.57 |
| HC 047 | 1173.61 | 92.19 | 101.83 | 71.30 | 5.17 | 659.65 | 18.74 |
| HC 048 | 1259.22 | 75.49 | 99.43 | 68.61 | 5.39 | 650.71 | 17.93 |
| HC 049 | 979.57 | 82.11 | 148.77 | 13.31 | 13.31 | 699.33 | 21.96 |
| HC 050 | 2090.21 | 209.99 | 186.57 | 70.40 | 4.64 | 1794.22 | 27.55 |
| HC 051 | 2040.10 | 212.27 | 168.08 | 60.50 | 5.41 | 949.11 | 25.56 |
| HC 052 | 816.24 | 62.11 | 111.22 | 19.04 | 11.23 | 586.79 | 15.08 |
| HC 053 | 2138.63 | 62.96 | 126.49 | 23.76 | 3.36 | 910.01 | 25.17 |
| HC 054 | 1898.69 | 65.55 | 126.84 | 21.88 | 3.41 | 716.96 | 23.57 |
| HC 055 | 1498.03 | 65.26 | 31.72 | 19.99 | 0.01 | 434.89 | 13.44 |
| HC 056 | 1348.07 | 65.04 | 74.61 | 17.86 | 1.84 | 389.41 | 19.12 |
| HC 057 | 1156.02 | 114.30 | 165.06 | 48.71 | 9.45 | 864.49 | 27.55 |
| HC 058 | 2088.89 | 233.77 | 87.00 | 22.73 | 6.21 | 406.32 | 25.05 |
| HC 059 | 2079.28 | 147.65 | 111.99 | 22.82 | 4.83 | 2374.06 | 23.57 |
| HC 060 | 1352.36 | 61.82 | 174.50 | 294.20 | 2.40 | 2239.89 | 32.31 |
| HC 061 | 1292.36 | 59.13 | 171.56 | 283.64 | 2.53 | 1961.07 | 28.75 |
| HC 062 | 2416.92 | 214.06 | 100.20 | 1003.30 | 11.30 | 791.92 | 21.56 |
| HC 063 | 2228.09 | 225.79 | 107.25 | 1004.91 | 12.82 | 1827.89 | 23.17 |
| HC 064 | 1576.63 | 67.88 | 65.08 | 21.88 | 2.22 | 443.14 | 20.36 |
| HC 065 | 1313.59 | 244.06 | 88.67 | 383.57 | 5.68 | 882.30 | 16.71 |
| HC 066 | 1826.46 | 114.66 | 192.23 | 1633.33 | 7.53 | 2150.86 | 26.76 |
| HC 067 | 12967.33 | 787.78 | 252.85 | 48.71 | 3.43 | 439.28 | 9.71 |
| HC 068 | 1457.73 | 74.73 | 123.27 | 25.64 | 3.56 | 683.41 | 23.17 |
| HC 069 | 2347.00 | 95.32 | 106.43 | 30.31 | 2.63 | 1912.19 | 21.16 |
| HC 070 | 1519.98 | 103.76 | 95.24 | 262.90 | 2.73 | 810.56 | 18.74 |
| HC 071 | 782.56 | 57.87 | 106.95 | 63.21 | 4.10 | 659.65 | 19.55 |
| HC 072 | 1579.60 | 127.20 | 108.89 | 306.85 | 3.63 | 915.63 | 22.37 |
| HC 073 | 928.97 | 60.12 | 118.23 | 69.51 | 4.93 | 743.26 | 19.95 |
| HC 074 | 959.98 | 57.46 | 112.86 | 60.50 | 4.46 | 585.27 | 21.96 |
| HC 075 | 1028.89 | 59.56 | 110.18 | 57.79 | 3.83 | 665.54 | 18.34 |
| HC 076 | 1861.78 | 161.70 | 109.49 | 106.85 | 5.89 | 2056.34 | 33.10 |
| HC 077 | 1684.06 | 165.82 | 112.34 | 53.26 | 7.08 | 2538.66 | 33.10 |
| HC 078 | 1646.86 | 171.10 | 110.87 | 54.17 | 5.70 | 2244.88 | 34.67 |
| HC 079 | 2105.31 | 69.06 | 86.17 | 73.99 | 5.09 | 753.54 | 28.35 |
| HC 080 | 1564.83 | 177.39 | 113.98 | 55.07 | 6.72 | 2937.69 | 27.95 |
| HC 081 | 1972.83 | 141.99 | 106.64 | 100.67 | 5.91 | 2124.42 | 27.16 |
| HC 082 | 882.10 | 92.03 | 177.62 | 19.04 | 12.40 | 756.99 | 26.76 |
| HC 083 | 36143.53 | 327.53 | 243.43 | 28.44 | 2.18 | 575.09 | 15.29 |
| HC 084 | 1328.48 | 91.70 | 95.49 | 33.09 | 7.45 | 491.53 | 17.93 |
| HC 085 | 1519.99 | 81.49 | 79.02 | 32.16 | 4.30 | 334.43 | 19.15 |
| HC 086 | 1643.10 | 76.86 | 163.15 | 72.65 | 19.41 | 568.78 | 22.77 |
| HC 087 | 1112.78 | 84.63 | 119.40 | 17.14 | 3.70 | 583.91 | 18.34 |
| HC 088 | 1127.99 | 55.38 | 151.82 | 22.82 | 4.79 | 682.55 | 21.96 |
| HC 089 | 1245.53 | 94.33 | 93.74 | 8.44 | 13.85 | 485.08 | 34.28 |
| HC 090 | 1203.90 | 105.66 | 93.27 | 6.47 | 7.91 | 426.76 | 35.26 |
| HC 091 | 1021.13 | 92.19 | 150.27 | 15.23 | 12.36 | 953.30 | 25.96 |
| HC 092 | 1143.16 | 92.35 | 94.21 | 6.47 | 7.38 | 401.99 | 38.41 |
| HC 093 | 1134.92 | 53.73 | 143.97 | 28.44 | 4.95 | 587.12 | 25.56 |
| HC 094 | 1887.94 | 86.53 | 116.50 | 28.44 | 3.91 | 649.93 | 23.57 |
| HC 095 | 1527.11 | 111.67 | 96.38 | 27.69 | 3.82 | 664.22 | 23.25 |
| HC 096 | 1854.64 | 86.85 | 121.70 | 28.44 | 3.94 | 706.64 | 23.17 |
| HC 097 | 1369.64 | 115.38 | 100.03 | 19.04 | 5.70 | 603.83 | 20.76 |
| HC 098 | 1004.95 | 70.24 | 171.07 | 31.24 | 6.61 | 892.85 | 19.95 |
| HC 099 | 883.09 | 181.74 | 136.28 | 14.27 | 17.41 | 521.60 | 38.60 |
| HC 100 | 1377.48 | 56.76 | 102.56 | 9.42 | 11.07 | 2304.23 | 18.74 |
| HC 101 | 1972.81 | 135.25 | 133.72 | 10.17 | 11.80 | 621.21 | 41.98 |
| HC 102 | 1727.82 | 110.11 | 114.76 | 9.25 | 8.72 | 503.63 | 33.65 |
| HC 103 | 1780.67 | 46.74 | 55.53 | 5.47 | 1.04 | 587.60 | 15.90 |
| HC 104 | 790.08 | 36.56 | 68.50 | 10.40 | 1.59 | 368.96 | 11.79 |
| HC 105 | 2217.91 | 9.47 | 0.05 | 0.04 | 0.11 | 0.59 | 0.12 |
| HC 106 | 2342.70 | 152.23 | 146.05 | 20.49 | 11.34 | 414.29 | 27.73 |
| HC 107 | 1683.24 | 119.52 | 121.50 | 14.75 | 6.64 | 303.75 | 26.11 |
| HC 108 | 2207.64 | 22.23 | 29.91 | 18.09 | 7.02 | 255.09 | 0.12 |
| HC 109 | 1438.66 | 35.43 | 72.72 | 148.41 | 2.64 | 432.64 | 24.57 |
| HC 110 | 2011.89 | 58.15 | 63.13 | 52.35 | 10.90 | 415.53 | 28.55 |
| HC 111 | 1350.19 | 84.65 | 124.79 | 165.15 | 6.14 | 271.50 | 28.27 |
| HC 112 | 2123.15 | 208.70 | 135.87 | 85.56 | 29.62 | 337.88 | 47.34 |
| HC 113 | 1913.95 | 92.75 | 82.63 | 27.69 | 3.21 | 681.74 | 20.78 |

| Subject | | | | |
|---|---|---|---|---|
| Cohort | | | | |
| ID | MPO | SHBG | TIMP1 | TIMP2 |
| OVAR 001 | 0.09 | 0.05 | 0.52 | 0.20 |
| OVAR 002 | 0.16 | 0.48 | 1.26 | 0.55 |
| OVAR 003 | 0.12 | 0.07 | 1.01 | 0.32 |
| OVAR 004 | 0.26 | 0.44 | 1.38 | 1.04 |
| OVAR 005 | 0.10 | 0.07 | 0.59 | 0.24 |
| OVAR 006 | 0.37 | 0.05 | 0.95 | 0.13 |
| OVAR 007 | 0.16 | 0.05 | 1.30 | 0.15 |
| OVAR 008 | 0.25 | 0.05 | 0.79 | 0.10 |
| OVAR 009 | 0.26 | 0.05 | 1.36 | 0.19 |
| OVAR 010 | 0.34 | 0.10 | 1.40 | 0.35 |
| OVAR 011 | 1.74 | 4.14 | 2.25 | 5.84 |
| OVAR 012 | 1.65 | 4.07 | 2.05 | 5.37 |
| OVAR 013 | 0.26 | 0.11 | 1.19 | 0.19 |
| OVAR 014 | 0.50 | 0.17 | 1.91 | 0.60 |
| OVAR 015 | 0.43 | 0.05 | 0.69 | 0.10 |
| OVAR 016 | 0.57 | 0.05 | 1.30 | 0.19 |
| OVAR 017 | 0.73 | 0.05 | 2.76 | 0.19 |
| OVAR 018 | 0.47 | 0.15 | 0.59 | 0.36 |
| OVAR 019 | 0.50 | 1.07 | 1.74 | 0.67 |
| OVAR 020 | 0.18 | 0.05 | 0.99 | 0.16 |
| OVAR 021 | 0.34 | 0.34 | 1.67 | 0.65 |
| OVAR 022 | 0.03 | 0.05 | 1.17 | 0.04 |
| OVAR 023 | 0.96 | 0.05 | 2.75 | 0.40 |
| OVAR 024 | 0.14 | 0.05 | 1.33 | 0.42 |
| OVAR 025 | 0.07 | 0.46 | 0.01 | 0.47 |
| OVAR 026 | 0.09 | 0.05 | 0.01 | 0.04 |
| OVAR 027 | 0.19 | 0.05 | 1.34 | 0.48 |
| OVAR 028 | 0.08 | 0.12 | 0.48 | 0.15 |
| OVAR 029 | 0.32 | 0.13 | 1.20 | 0.20 |
| OVAR 030 | 0.33 | 0.08 | 0.34 | 0.12 |
| OVAR 031 | 0.33 | 2.24 | 1.13 | 0.53 |
| OVAR 032 | 0.29 | 0.05 | 2.00 | 0.72 |
| OVAR 033 | 0.03 | 0.05 | 0.01 | 0.04 |
| OVAR 034 | 0.22 | 1.07 | 1.37 | 0.38 |
| OVAR 035 | 0.27 | 0.05 | 2.45 | 0.43 |
| OVAR 036 | 1.64 | 0.75 | 2.55 | 0.53 |
| OVAR 037 | 1.17 | 0.42 | 1.87 | 0.34 |
| OVAR 038 | 0.76 | 0.31 | 1.85 | 0.38 |
| OVAR 039 | 0.18 | 0.23 | 0.32 | 0.81 |
| OVAR 040 | 0.28 | 0.32 | 2.07 | 0.24 |
| OVAR 041 | 0.13 | 0.05 | 0.44 | 0.09 |
| OVAR 042 | 0.62 | 0.06 | 0.97 | 0.14 |
| OVAR 043 | 0.65 | 0.05 | 1.21 | 0.30 |
| OVAR 044 | 0.32 | 0.05 | 0.56 | 0.23 |
| BLDR 001 | 0.11 | 0.71 | 2.07 | 0.59 |
| BLDR 002 | 0.08 | 0.05 | 0.52 | 0.11 |
| BLDR 003 | 0.19 | 0.14 | 0.50 | 0.22 |
| BLDR 004 | 0.12 | 0.09 | 0.45 | 0.20 |
| BLDR 005 | 1.84 | 0.08 | 0.92 | 0.34 |
| BLDR 006 | 0.30 | 0.23 | 0.80 | 0.41 |
| BLDR 007 | 0.23 | 0.30 | 1.68 | 0.21 |
| BLDR 008 | 1.74 | 0.07 | 1.49 | 0.60 |
| BLDR 009 | 0.13 | 0.05 | 0.37 | 0.14 |
| BLDR 010 | 0.20 | 0.08 | 1.20 | 0.29 |
| BLDR 011 | 0.33 | 2.12 | 1.63 | 0.74 |
| BLDR 012 | 0.42 | 1.09 | 1.78 | 1.12 |
| BLDR 013 | 0.31 | 0.05 | 1.21 | 0.17 |
| BLDR 014 | 0.11 | 0.11 | 0.58 | 0.22 |
| BLDR 015 | 0.14 | 0.80 | 1.95 | 0.45 |
| BLDR 016 | 0.41 | 1.28 | 2.04 | 1.16 |
| BLDR 017 | 0.13 | 0.12 | 0.32 | 0.15 |
| BLDR 018 | 0.13 | 0.05 | 0.61 | 0.21 |
| BLDR 019 | 1.00 | 0.32 | 0.95 | 0.18 |
| BLDR 020 | 0.32 | 0.20 | 1.02 | 0.18 |
| BLDR 021 | 0.16 | 0.05 | 1.52 | 0.16 |
| BLDR 022 | 0.17 | 0.05 | 0.93 | 0.37 |
| BLDR 023 | 0.10 | 0.06 | 0.33 | 0.13 |
| BLDR 024 | 0.42 | 0.58 | 1.65 | 0.65 |
| BLDR 025 | 0.15 | 0.29 | 1.73 | 0.23 |
| BLDR 026 | 0.21 | 0.10 | 0.58 | 0.25 |
| BLDR 027 | 0.24 | 0.41 | 2.24 | 1.06 |
| BLDR 028 | 0.43 | 0.40 | 2.78 | 0.91 |
| BLDR 029 | 0.20 | 0.05 | 0.23 | 0.06 |
| BLDR 030 | 0.21 | 0.10 | 1.26 | 0.27 |
| BLDR 031 | 2.72 | 1.62 | 3.23 | 1.18 |
| BLDR 032 | 0.40 | 0.05 | 1.96 | 0.35 |
| BLDR 033 | 0.13 | 0.08 | 1.19 | 0.25 |
| BLDR 034 | 0.32 | 0.07 | 0.45 | 0.11 |
| BLDR 035 | 0.82 | 0.52 | 4.03 | 1.20 |
| BLDR 036 | 0.24 | 0.05 | 2.38 | 0.52 |
| BLDR 037 | 0.18 | 0.41 | 0.25 | 0.61 |
| BLDR 038 | 0.04 | 0.05 | 0.19 | 0.08 |
| BLDR 039 | 0.21 | 0.05 | 1.62 | 0.50 |
| BLDR 040 | 0.10 | 0.05 | 0.74 | 0.22 |
| BLDR 041 | 0.31 | 0.08 | 11.12 | 0.61 |
| BLDR 042 | 0.42 | 0.05 | 0.52 | 0.19 |
| BLDR 043 | 0.25 | 0.96 | 0.96 | 0.19 |
| BLDR 044 | 0.11 | 0.08 | 0.73 | 0.22 |
| BLDR 045 | 0.05 | 0.05 | 0.76 | 0.21 |
| BLDR 046 | 0.38 | 1.11 | 0.36 | 0.22 |
| BLDR 047 | 0.19 | 0.42 | 0.26 | 0.59 |
| BLDR 048 | 2.72 | 5.79 | 1.78 | 3.36 |
| PDAC 001 | 0.11 | 0.05 | 0.75 | 0.23 |
| PDAC 002 | 0.32 | 0.57 | 0.98 | 0.33 |
| PDAC 003 | 0.25 | 0.36 | 0.84 | 0.21 |
| PDAC 004 | 0.54 | 1.17 | 0.83 | 1.83 |
| PDAC 005 | 0.06 | 0.05 | 0.22 | 0.07 |
| PDAC 006 | 0.43 | 0.20 | 1.87 | 0.93 |
| PDAC 007 | 0.33 | 0.10 | 0.52 | 0.31 |
| PDAC 008 | 0.66 | 0.14 | 2.20 | 0.25 |
| PDAC 009 | 0.14 | 0.12 | 0.87 | 0.27 |
| PDAC 010 | 0.18 | 0.49 | 0.89 | 0.25 |
| PDAC 011 | 0.86 | 0.42 | 2.47 | 0.62 |
| PDAC 012 | 0.19 | 1.11 | 1.48 | 0.66 |
| PDAC 013 | 1.81 | 0.96 | 0.51 | 0.16 |
| PDAC 014 | 0.55 | 1.20 | 0.84 | 1.84 |
| PDAC 015 | 0.38 | 0.28 | 1.08 | 0.29 |
| PDAC 016 | 0.08 | 0.09 | 0.48 | 0.09 |
| PDAC 017 | 2.00 | 1.16 | 1.81 | 0.73 |
| PDAC 018 | 0.11 | 0.06 | 0.69 | 0.14 |
| PDAC 019 | 0.26 | 0.12 | 1.12 | 0.32 |
| PDAC 020 | 0.21 | 0.69 | 0.62 | 0.17 |
| PDAC 021 | 0.07 | 0.06 | 0.46 | 0.14 |
| PDAC 022 | 0.33 | 0.52 | 1.93 | 0.80 |
| PDAC 023 | 0.21 | 0.58 | 2.09 | 0.41 |
| PDAC 024 | 0.14 | 0.16 | 1.09 | 0.23 |
| PDAC 025 | 0.38 | 1.21 | 0.85 | 0.17 |
| PDAC 026 | 0.91 | 1.39 | 2.59 | 0.39 |
| PDAC 027 | 0.28 | 0.08 | 0.85 | 0.21 |
| PDAC 028 | 0.40 | 1.90 | 2.24 | 0.89 |
| PDAC 029 | 0.16 | 0.20 | 0.31 | 0.78 |
| PDAC 030 | 0.26 | 0.05 | 1.12 | 0.22 |
| PDAC 031 | 0.27 | 0.05 | 0.98 | 0.20 |
| PDAC 032 | 0.10 | 0.09 | 1.82 | 0.21 |
| PDAC 033 | 0.97 | 0.05 | 0.19 | 0.05 |
| PDAC 034 | 0.03 | 0.05 | 0.01 | 0.04 |
| PDAC 035 | 0.18 | 0.05 | 1.09 | 0.32 |
| PDAC 036 | 0.12 | 0.25 | 0.68 | 0.89 |
| PDAC 037 | 0.13 | 0.11 | 0.43 | 0.12 |
| PDAC 038 | 0.29 | 0.14 | 0.84 | 0.20 |
| PDAC 039 | 0.42 | 0.89 | 1.25 | 0.73 |
| PDAC 040 | 0.27 | 0.18 | 1.68 | 0.45 |
| PDAC 041 | 4.14 | 0.05 | 0.95 | 0.52 |
| PDAC 042 | 0.16 | 0.05 | 0.53 | 0.36 |
| PDAC 043 | 10.02 | 31.23 | 10.34 | 23.72 |
| PDAC 044 | 0.31 | 0.29 | 2.08 | 0.41 |
| HC 001 | 0.37 | 0.06 | 1.30 | 0.39 |
| HC 002 | 0.22 | 0.05 | 1.19 | 0.24 |
| HC 003 | 1.29 | 0.05 | 2.32 | 0.39 |
| HC 004 | 0.76 | 0.05 | 1.58 | 0.62 |
| HC 005 | 0.82 | 0.19 | 2.85 | 0.37 |
| HC 006 | 1.43 | 0.06 | 3.88 | 0.61 |
| HC 007 | 0.69 | 0.11 | 11.14 | 0.50 |
| HC 008 | 0.28 | 0.17 | 1.82 | 0.54 |
| HC 009 | 0.16 | 0.23 | 1.83 | 0.70 |
| HC 010 | 0.22 | 0.08 | 1.47 | 0.35 |
| HC 011 | 0.17 | 0.33 | 2.83 | 1.00 |
| HC 012 | 0.34 | 0.97 | 2.21 | 0.64 |
| HC 013 | 0.16 | 0.87 | 1.00 | 0.49 |
| HC 014 | 0.45 | 0.73 | 2.17 | 0.54 |
| HC 015 | 0.15 | 0.71 | 1.48 | 0.62 |
| HC 016 | 0.33 | 0.20 | 0.59 | 0.26 |
| HC 017 | 0.27 | 0.35 | 1.21 | 0.37 |
| HC 018 | 0.20 | 0.34 | 1.02 | 0.45 |
| HC 019 | 0.18 | 0.41 | 0.61 | 0.29 |
| HC 020 | 0.80 | 2.50 | 2.16 | 1.56 |
| HC 021 | 0.50 | 0.48 | 1.41 | 0.31 |
| HC 022 | 0.57 | 0.74 | 1.43 | 0.37 |
| HC 023 | 0.37 | 2.60 | 1.64 | 1.03 |
| HC 024 | 1.39 | 0.62 | 2.11 | 0.34 |
| HC 025 | 0.52 | 0.30 | 3.18 | 0.62 |
| HC 026 | 0.47 | 0.11 | 1.88 | 0.40 |
| HC 027 | 0.51 | 0.05 | 2.80 | 0.31 |
| HC 028 | 0.27 | 0.10 | 0.90 | 0.20 |
| HC 029 | 0.12 | 0.05 | 0.86 | 0.20 |
| HC 030 | 0.41 | 0.05 | 2.37 | 0.51 |
| HC 031 | 0.42 | 0.27 | 0.01 | 0.61 |
| HC 032 | 0.04 | 0.05 | 0.78 | 0.28 |
| HC 033 | 0.40 | 0.05 | 2.63 | 0.36 |
| HC 034 | 0.30 | 0.19 | 0.98 | 0.16 |
| HC 035 | 0.20 | 0.05 | 11.50 | 0.58 |
| HC 036 | 0.59 | 0.09 | 2.35 | 0.36 |
| HC 037 | 0.41 | 0.05 | 2.65 | 0.29 |
| HC 038 | 0.54 | 0.12 | 8.87 | 0.31 |
| HC 039 | 0.55 | 0.05 | 2.34 | 0.34 |
| HC 040 | 0.38 | 0.05 | 2.29 | 0.31 |
| HC 041 | 1.24 | 0.05 | 3.47 | 0.73 |
| HC 042 | 0.62 | 0.15 | 2.11 | 0.36 |
| HC 043 | 0.78 | 0.27 | 0.93 | 0.57 |
| HC 044 | 0.37 | 0.16 | 0.40 | 0.18 |
| HC 045 | 0.63 | 0.68 | 0.59 | 0.27 |
| HC 046 | 0.50 | 1.50 | 0.51 | 0.40 |
| HC 047 | 0.41 | 0.06 | 0.32 | 0.11 |
| HC 048 | 0.39 | 0.59 | 0.42 | 0.29 |
| HC 049 | 0.03 | 0.05 | 0.01 | 0.04 |
| HC 050 | 0.20 | 0.05 | 0.41 | 0.20 |
| HC 051 | 0.22 | 0.06 | 0.48 | 0.33 |
| HC 052 | 0.55 | 0.05 | 0.79 | 0.44 |
| HC 053 | 0.17 | 0.55 | 0.95 | 0.38 |
| HC 054 | 0.45 | 1.20 | 1.14 | 0.50 |
| HC 055 | 0.21 | 0.33 | 0.45 | 0.34 |
| HC 056 | 0.14 | 0.24 | 0.67 | 0.65 |
| HC 057 | 0.15 | 0.05 | 0.47 | 0.14 |
| HC 058 | 0.80 | 0.05 | 1.58 | 0.28 |
| HC 059 | 0.89 | 0.40 | 2.14 | 0.18 |
| HC 060 | 0.50 | 0.47 | 1.13 | 0.27 |
| HC 061 | 0.41 | 0.34 | 0.36 | 0.15 |
| HC 062 | 0.50 | 0.07 | 0.38 | 0.11 |
| HC 063 | 0.49 | 0.36 | 0.96 | 0.28 |
| HC 064 | 0.10 | 0.17 | 1.86 | 0.37 |
| HC 065 | 0.53 | 0.18 | 0.64 | 0.28 |
| HC 066 | 0.38 | 0.09 | 0.83 | 0.18 |
| HC 067 | 0.20 | 0.18 | 1.26 | 0.34 |
| HC 068 | 0.30 | 0.05 | 0.25 | 0.14 |
| HC 069 | 0.20 | 0.10 | 0.91 | 0.22 |
| HC 070 | 0.13 | 0.05 | 0.46 | 0.10 |
| HC 071 | 2.24 | 10.78 | 3.42 | 8.55 |
| HC 072 | 0.44 | 0.29 | 0.80 | 0.41 |
| HC 073 | 0.37 | 0.09 | 1.37 | 0.76 |
| HC 074 | 0.63 | 0.22 | 1.82 | 0.71 |
| HC 075 | 0.79 | 0.19 | 2.19 | 0.62 |
| HC 076 | 0.63 | 1.08 | 1.19 | 0.82 |
| HC 077 | 0.71 | 0.05 | 0.93 | 0.34 |
| HC 078 | 0.75 | 0.87 | 0.55 | 0.33 |
| HC 079 | 3.00 | 6.14 | 9.16 | 20.66 |
| HC 080 | 0.71 | 0.05 | 0.47 | 0.28 |
| HC 081 | 4.95 | 11.33 | 18.01 | 41.41 |
| HC 082 | 0.31 | 0.05 | 0.70 | 0.24 |
| HC 083 | 3.22 | 4.92 | 1.73 | 3.72 |
| HC 084 | 0.12 | 0.05 | 1.05 | 0.15 |
| HC 085 | 0.07 | 0.05 | 0.58 | 0.22 |
| HC 086 | 0.10 | 0.05 | 0.68 | 0.07 |
| HC 087 | 0.19 | 0.05 | 1.04 | 0.04 |
| HC 088 | 0.36 | 0.22 | 1.08 | 0.65 |
| HC 089 | 0.24 | 0.10 | 2.40 | 0.27 |
| HC 090 | 0.14 | 0.05 | 0.77 | 0.14 |
| HC 091 | 0.39 | 0.08 | 1.00 | 0.35 |
| HC 092 | 0.11 | 0.10 | 1.04 | 0.23 |
| HC 093 | 0.30 | 0.05 | 0.91 | 0.26 |
| HC 094 | 0.21 | 0.12 | 0.29 | 0.17 |
| HC 095 | 0.35 | 0.17 | 0.54 | 0.35 |
| HC 096 | 0.95 | 1.49 | 1.14 | 0.55 |
| HC 097 | 0.92 | 0.47 | 0.45 | 0.22 |
| HC 098 | 0.57 | 0.50 | 1.44 | 0.72 |
| HC 099 | 0.66 | 0.16 | 2.07 | 0.31 |
| HC 100 | 0.48 | 0.16 | 0.49 | 0.21 |
| HC 101 | 0.13 | 0.10 | 0.43 | 0.19 |
| HC 102 | 0.18 | 0.56 | 1.42 | 0.55 |
| HC 103 | 0.08 | 0.05 | 0.30 | 0.11 |
| HC 104 | 0.04 | 0.05 | 0.28 | 0.09 |
| HC 105 | 0.20 | 0.45 | 0.46 | 0.20 |
| HC 106 | 0.22 | 0.43 | 0.55 | 0.31 |
| HC 107 | 0.03 | 0.11 | 0.23 | 0.13 |
| HC 108 | 0.67 | 0.38 | 1.17 | 0.39 |
| HC 109 | 0.27 | 0.19 | 0.46 | 0.21 |
| HC 110 | 0.22 | 0.15 | 0.63 | 0.26 |
| HC 111 | 0.15 | 0.08 | 0.27 | 0.14 |
| HC 112 | 0.46 | 0.30 | 0.70 | 0.29 |
| HC 113 | 0.13 | 0.23 | 1.39 | 0.27 |
| OVAR 001 | 9.96 | 35.25 | 56.85 | 42.67 |
| OVAR 002 | 10.43 | 42.99 | 78.87 | 48.01 |
| OVAR 003 | 9.73 | 20.34 | 63.44 | 46.01 |
| OVAR 004 | 31.00 | 72.36 | 155.50 | 53.71 |
| OVAR 005 | 11.38 | 34.93 | 69.72 | 57.78 |
| OVAR 006 | 3.12 | 66.95 | 25.71 | 20.79 |
| OVAR 007 | 15.20 | 45.65 | 63.19 | 44.25 |
| OVAR 008 | 14.07 | 38.64 | 121.75 | 58.17 |
| OVAR 009 | 11.30 | 39.38 | 129.41 | 50.94 |
| OVAR 010 | 460.22 | 1124.51 | 527.60 | 1269.84 |
| OVAR 011 | 42.58 | 62.02 | 118.66 | 62.03 |
| OVAR 012 | 10.78 | 40.70 | 64.26 | 53.08 |
| OVAR 013 | 34.59 | 97.52 | 127.12 | 96.96 |
| OVAR 014 | 14.77 | 13.35 | 71.31 | 41.18 |
| OVAR 015 | 75.56 | 38.47 | 146.60 | 67.57 |
| OVAR 016 | 29.08 | 22.91 | 95.82 | 64.01 |
| OVAR 017 | 30.47 | 13.58 | 120.18 | 38.41 |
| OVAR 018 | 36.82 | 46.12 | 70.25 | 57.39 |
| OVAR 019 | 27.16 | 76.19 | 87.67 | 49.99 |
| OVAR 020 | 16.11 | 30.14 | 89.60 | 48.09 |
| OVAR 021 | 17.28 | 31.09 | 64.50 | 52.88 |
| OVAR 022 | 13.08 | 62.07 | 93.98 | 59.62 |
| OVAR 023 | 42.58 | 23.66 | 625.44 | 54.02 |
| OVAR 024 | 10.82 | 47.38 | 92.38 | 61.45 |
| OVAR 025 | 5.61 | 133.44 | 92.08 | 83.49 |
| OVAR 026 | 9.03 | 72.03 | 120.51 | 61.84 |
| OVAR 027 | 13.03 | 46.65 | 98.23 | 50.31 |
| OVAR 028 | 5.53 | 52.88 | 66.02 | 59.93 |
| OVAR 029 | 166.87 | 248.29 | 82.52 | 214.31 |
| OVAR 030 | 74.98 | 24.70 | 78.93 | 89.69 |
| OVAR 031 | 22.13 | 106.40 | 76.46 | 95.73 |
| OVAR 032 | 46.99 | 29.91 | 558.79 | 98.78 |
| OVAR 033 | 6.31 | 5.44 | 20.78 | 8.70 |
| OVAR 034 | 31.28 | 122.74 | 176.75 | 84.49 |
| OVAR 035 | 72.61 | 82.33 | 185.23 | 89.54 |
| OVAR 036 | 125.72 | 72.50 | 169.21 | 69.31 |
| OVAR 037 | 49.91 | 22.34 | 137.60 | 78.48 |
| OVAR 038 | 46.18 | 86.69 | 71.46 | 66.89 |
| OVAR 039 | 18.64 | 106.00 | 101.40 | 80.77 |
| OVAR 040 | 33.39 | 104.36 | 360.91 | 63.53 |
| OVAR 041 | 27.37 | 49.72 | 80.66 | 46.40 |
| OVAR 042 | 110.12 | 223.62 | 99.38 | 60.87 |
| OVAR 043 | 257.34 | 77.28 | 559.93 | 111.68 |
| OVAR 044 | 75.80 | 258.52 | 105.06 | 74.35 |
| BLDR 001 | 5.04 | 55.31 | 108.84 | 46.08 |
| BLDR 002 | 6.74 | 33.17 | 53.36 | 31.26 |
| BLDR 003 | 11.64 | 41.62 | 59.28 | 40.80 |
| BLDR 004 | 29.39 | 59.83 | 61.85 | 37.51 |
| BLDR 005 | 32.16 | 17.61 | 64.62 | 38.68 |
| BLDR 006 | 16.70 | 21.16 | 64.19 | 52.89 |
| BLDR 007 | 40.00 | 86.51 | 75.56 | 45.71 |
| BLDR 008 | 77.36 | 9.37 | 82.73 | 50.98 |
| BLDR 009 | 9.19 | 25.43 | 39.12 | 30.78 |
| BLDR 010 | 44.13 | 94.59 | 59.14 | 51.12 |
| BLDR 011 | 29.83 | 27.77 | 68.21 | 37.09 |
| BLDR 012 | 11.44 | 60.97 | 69.62 | 53.70 |
| BLDR 013 | 13.98 | 14.23 | 52.62 | 38.93 |
| BLDR 014 | 12.52 | 38.02 | 41.40 | 35.33 |
| BLDR 015 | 6.29 | 85.44 | 55.64 | 29.81 |
| BLDR 016 | 21.72 | 98.83 | 78.94 | 61.89 |
| BLDR 017 | 18.46 | 65.70 | 56.13 | 46.94 |
| BLDR 018 | 14.66 | 43.34 | 73.22 | 71.63 |
| BLDR 019 | 30.31 | 60.60 | 88.29 | 54.48 |
| BLDR 020 | 11.00 | 28.10 | 29.22 | 19.39 |
| BLDR 021 | 20.83 | 46.55 | 77.55 | 39.53 |
| BLDR 022 | 25.85 | 33.88 | 49.75 | 44.75 |
| BLDR 023 | 9.42 | 29.78 | 54.79 | 39.98 |
| BLDR 024 | 19.51 | 46.70 | 67.66 | 45.24 |
| BLDR 025 | 15.29 | 83.76 | 53.89 | 37.93 |
| BLDR 026 | 35.02 | 35.88 | 93.92 | 57.94 |
| BLDR 027 | 10.62 | 37.71 | 91.79 | 56.88 |
| BLDR 028 | 12.85 | 28.07 | 83.48 | 49.18 |
| BLDR 029 | 23.11 | 17.03 | 88.49 | 56.37 |
| BLDR 030 | 23.24 | 47.73 | 62.40 | 40.40 |
| BLDR 031 | 115.25 | 58.64 | 81.98 | 38.26 |
| BLDR 032 | 24.21 | 28.57 | 92.06 | 48.36 |
| BLDR 033 | 11.88 | 38.64 | 36.46 | 25.51 |
| BLDR 034 | 10.60 | 27.68 | 50.93 | 37.49 |
| BLDR 035 | 27.30 | 47.48 | 116.29 | 64.31 |
| BLDR 036 | 9.66 | 51.15 | 69.63 | 48.70 |
| BLDR 037 | 13.57 | 35.87 | 62.23 | 30.35 |
| BLDR 038 | 36.01 | 120.14 | 122.64 | 56.97 |
| BLDR 039 | 13.90 | 29.72 | 67.51 | 36.23 |
| BLDR 040 | 6.82 | 64.62 | 67.66 | 50.28 |
| BLDR 041 | 10.00 | 141.59 | 103.07 | 92.10 |
| BLDR 042 | 282.38 | 71.35 | 596.49 | 118.71 |
| BLDR 043 | 22.19 | 903.92 | 88.19 | 40.90 |
| BLDR 044 | 49.00 | 267.51 | 123.31 | 91.68 |
| BLDR 045 | 23.15 | 146.25 | 95.40 | 113.09 |
| BLDR 046 | 9.03 | 80.67 | 35.83 | 37.79 |
| BLDR 047 | 8.49 | 45.79 | 48.04 | 39.28 |
| BLDR 048 | 12.84 | 32.84 | 50.89 | 35.63 |
| PDAC 001 | 12.43 | 9.56 | 74.68 | 49.04 |
| PDAC 002 | 32.83 | 121.93 | 123.30 | 64.47 |
| PDAC 003 | 14.72 | 51.44 | 96.60 | 50.55 |
| PDAC 004 | 8.03 | 108.78 | 94.89 | 65.71 |
| PDAC 005 | 8.86 | 26.77 | 91.48 | 54.61 |
| PDAC 006 | 21.09 | 51.10 | 74.98 | 56.10 |
| PDAC 007 | 59.82 | 27.50 | 107.19 | 58.53 |
| PDAC 008 | 11.62 | 109.86 | 79.82 | 56.31 |
| PDAC 009 | 7.1 | 25.35 | 41.95 | 39.31 |
| PDAC 010 | 27.48 | 25.72 | 570.87 | 28.22 |
| PDAC 011 | 91.77 | 43.81 | 146.64 | 45.11 |
| PDAC 012 | 15.37 | 77.17 | 61.42 | 48.48 |
| PDAC 013 | 11.04 | 83.98 | 56.32 | 38.08 |
| PDAC 014 | 26.38 | 31.40 | 72.31 | 34.21 |
| PDAC 015 | 28.62 | 88.89 | 83.59 | 47.77 |
| PDAC 016 | 15.98 | 61.69 | 99.29 | 52.29 |
| PDAC 017 | 118.75 | 78.89 | 80.57 | 44.73 |
| PDAC 018 | 14.55 | 73.54 | 114.18 | 69.63 |
| PDAC 019 | 24.91 | 38.39 | 106.43 | 50.75 |
| PDAC 020 | 11.99 | 106.21 | 61.31 | 40.04 |
| PDAC 021 | 9.82 | 56.57 | 67.38 | 41.99 |
| PDAC 022 | 21.26 | 35.99 | 105.38 | 55.04 |
| PDAC 023 | 14.94 | 99.84 | 90.59 | 58.21 |
| PDAC 024 | 14.94 | 41.95 | 124.34 | 46.53 |
| PDAC 025 | 15.72 | 105.51 | 98.79 | 36.19 |
| PDAC 026 | 107.66 | 135.17 | 190.30 | 49.30 |
| PDAC 027 | 18.23 | 21.54 | 93.61 | 38.08 |
| PDAC 028 | 20.22 | 73.46 | 166.32 | 66.95 |
| PDAC 029 | 8.84 | 62.90 | 54.75 | 74.68 |
| PDAC 030 | 19.56 | 74.61 | 92.75 | 42.71 |
| PDAC 031 | 9.26 | 36.03 | 63.27 | 39.00 |
| PDAC 032 | 17.37 | 8.49 | 59.45 | 36.54 |
| PDAC 033 | 13.53 | 60.53 | 49.82 | 44.40 |
| PDAC 034 | 18.12 | 32.05 | 46.47 | 39.38 |
| PDAC 035 | 10.68 | 115.72 | 71.45 | 46.78 |
| PDAC 036 | 30.90 | 89.23 | 71.95 | 44.20 |
| PDAC 037 | 16.89 | 0.05 | 25.23 | 12.87 |
| PDAC 038 | 28.14 | 28.72 | 50.46 | 41.99 |
| PDAC 039 | 47.07 | 51.74 | 94.53 | 66.48 |
| PDAC 040 | 15.32 | 22.18 | 63.60 | 50.21 |
| PDAC 041 | 216.54 | 92.44 | 174.49 | 104.04 |
| PDAC 042 | 23.45 | 0.05 | 94.76 | 36.92 |
| PDAC 043 | 6.07 | 84.54 | 46.37 | 45.69 |
| PDAC 044 | 21.30 | 51.27 | 109.23 | 44.55 |
| HC 001 | 21.86 | 15.65 | 83.18 | 56.14 |
| HC 002 | 13.25 | 9.90 | 53.97 | 36.71 |
| HC 003 | 13.41 | 11.92 | 40.77 | 38.41 |
| HC 004 | 20.15 | 16.94 | 48.49 | 49.81 |
| HC 005 | 10.92 | 95.04 | 63.99 | 45.19 |
| HC 006 | 41.48 | 12.81 | 75.41 | 62.28 |
| HC 007 | 12.72 | 31.34 | 69.29 | 45.99 |
| HC 008 | 9.58 | 19.80 | 46.41 | 47.91 |
| HC 009 | 5.77 | 29.51 | 54.78 | 55.31 |
| HC 010 | 9.03 | 19.82 | 47.66 | 50.43 |
| HC 011 | 6.07 | 29.47 | 59.39 | 60.61 |
| HC 012 | 5.11 | 109.57 | 43.60 | 47.74 |
| HC 013 | 5.92 | 127.25 | 51.50 | 51.56 |
| HC 014 | 5.04 | 118.28 | 51.83 | 55.21 |
| HC 015 | 6.26 | 138.72 | 55.62 | 51.96 |
| HC 016 | 13.90 | 79.93 | 54.33 | 48.31 |
| HC 017 | 14.06 | 62.01 | 51.72 | 35.88 |
| HC 018 | 18.52 | 83.02 | 78.83 | 59.40 |
| HC 019 | 18.04 | 92.32 | 67.95 | 58.35 |
| HC 020 | 22.90 | 87.71 | 58.10 | 56.34 |
| HC 021 | 10.13 | 109.43 | 51.77 | 40.33 |
| HC 022 | 11.16 | 101.15 | 54.69 | 38.03 |
| HC 023 | 9.50 | 107.73 | 49.51 | 39.46 |
| HC 024 | 11.60 | 108.22 | 50.62 | 36.94 |
| HC 025 | 11.08 | 35.37 | 47.61 | 44.50 |
| HC 026 | 26.31 | 36.42 | 105.73 | 62.09 |
| HC 027 | 40.00 | 78.33 | 50.96 | 51.12 |
| HC 028 | 33.24 | 69.12 | 52.16 | 46.75 |
| HC 029 | 11.56 | 28.35 | 43.02 | 41.17 |
| HC 030 | 44.53 | 52.37 | 121.38 | 110.60 |
| HC 031 | 51.12 | 131.32 | 93.26 | 101.97 |
| HC 032 | 13.75 | 54.57 | 108.99 | 102.46 |
| HC 033 | 20.35 | 36.07 | 98.76 | 107.23 |
| HC 034 | 84.91 | 1028.00 | 109.15 | 64.43 |
| HC 035 | 11.31 | 39.58 | 114.46 | 115.22 |
| HC 036 | 34.47 | 46.74 | 91.44 | 85.14 |
| HC 037 | 41.17 | 409.84 | 90.91 | 61.53 |
| HC 038 | 30.25 | 106.71 | 99.50 | 66.43 |
| HC 039 | 25.02 | 35.57 | 83.72 | 70.03 |
| HC 040 | 26.21 | 122.26 | 74.18 | 60.82 |
| HC 041 | 58.90 | 127.80 | 126.88 | 98.54 |
| HC 042 | 24.41 | 59.36 | 70.16 | 44.69 |
| HC 043 | 54.03 | 32.13 | 56.78 | 48.16 |
| HC 044 | 25.55 | 93.43 | 58.00 | 39.48 |
| HC 045 | 16.70 | 66.53 | 46.40 | 30.09 |
| HC 046 | 13.81 | 78.35 | 37.16 | 53.44 |
| HC 047 | 18.45 | 94.45 | 58.96 | 72.02 |
| HC 048 | 16.60 | 92.69 | 53.77 | 69.63 |
| HC 049 | 18.68 | 23.85 | 62.96 | 58.65 |
| HC 050 | 34.20 | 31.10 | 90.65 | 75.75 |
| HC 051 | 24.55 | 27.20 | 81.09 | 68.28 |
| HC 052 | 31.21 | 17.39 | 45.18 | 46.45 |
| HC 053 | 16.32 | 124.50 | 84.71 | 54.45 |
| HC 054 | 14.09 | 125.30 | 77.50 | 53.89 |
| HC 055 | 11.51 | 27.49 | 57.87 | 47.64 |
| HC 056 | 8.17 | 33.62 | 36.14 | 41.80 |
| HC 057 | 39.12 | 25.70 | 122.23 | 68.15 |
| HC 058 | 35.35 | 37.05 | 90.47 | 50.66 |
| HC 059 | 23.72 | 78.56 | 80.38 | 53.86 |
| HC 060 | 61.93 | 127.41 | 72.10 | 48.46 |
| HC 061 | 50.65 | 112.27 | 64.75 | 45.95 |
| HC 062 | 27.78 | 74.61 | 92.77 | 43.20 |
| HC 063 | 30.55 | 76.73 | 98.26 | 46.58 |
| HC 064 | 5.53 | 40.79 | 46.68 | 41.21 |
| HC 065 | 58.57 | 51.14 | 72.92 | 32.09 |
| HC 066 | 46.73 | 126.37 | 106.65 | 58.49 |
| HC 067 | 12.74 | 78.62 | 125.64 | 85.12 |
| HC 068 | 43.47 | 63.20 | 68.70 | 59.98 |
| HC 069 | 30.91 | 80.59 | 103.01 | 56.28 |
| HC 070 | 31.09 | 69.18 | 73.74 | 53.41 |
| HC 071 | 25.38 | 18.17 | 69.64 | 40.27 |
| HC 072 | 30.43 | 78.04 | 82.22 | 60.94 |
| HC 073 | 47.05 | 20.07 | 73.48 | 47.64 |
| HC 074 | 33.59 | 19.65 | 73.79 | 44.58 |
| HC 075 | 38.00 | 19.33 | 69.86 | 42.64 |
| HC 076 | 32.25 | 23.41 | 117.43 | 56.28 |
| HC 077 | 112.29 | 441.19 | 71.67 | 62.65 |
| HC 078 | 136.62 | 470.09 | 71.74 | 64.37 |
| HC 079 | 18.16 | 20.49 | 111.10 | 54.55 |
| HC 080 | 132.79 | 467.50 | 70.69 | 61.13 |
| HC 081 | 17.76 | 23.09 | 116.33 | 55.87 |
| HC 082 | 21.37 | 20.41 | 71.68 | 59.10 |
| HC 083 | 21.48 | 84.35 | 132.96 | 61.38 |
| HC 084 | 3.86 | 27.42 | 70.06 | 59.13 |
| HC 085 | 4.58 | 25.04 | 66.17 | 53.01 |
| HC 086 | 86.79 | 81.10 | 89.19 | 47.05 |
| HC 087 | 16.03 | 18.57 | 54.42 | 40.65 |
| HC 088 | 22.78 | 21.68 | 76.51 | 45.39 |
| HC 089 | 19.15 | 27.56 | 62.56 | 50.90 |
| HC 090 | 16.66 | 26.84 | 66.07 | 52.35 |
| HC 091 | 29.94 | 26.68 | 71.99 | 59.70 |
| HC 092 | 19.96 | 25.90 | 74.65 | 53.60 |
| HC 093 | 34.32 | 25.79 | 89.22 | 51.65 |
| HC 094 | 34.75 | 93.23 | 67.36 | 58.43 |
| HC 095 | 29.12 | 87.75 | 58.50 | 56.80 |
| HC 096 | 39.61 | 93.13 | 62.06 | 53.92 |
| HC 097 | 47.23 | 64.26 | 55.68 | 52.75 |
| HC 098 | 31.52 | 39.49 | 86.29 | 54.48 |
| HC 099 | 15.34 | 23.47 | 87.79 | 63.42 |
| HC 100 | 16.60 | 100.91 | 58.86 | 45.39 |
| HC 101 | 26.43 | 102.72 | 92.10 | 83.91 |
| HC 102 | 16.48 | 88.66 | 80.83 | 68.50 |
| HC 103 | 9.73 | 81.29 | 68.99 | 41.27 |
| HC 104 | 5.63 | 17.40 | 34.17 | 19.60 |
| HC 105 | 0.03 | 0.05 | 0.16 | 0.04 |
| HC 106 | 18.31 | 85.64 | 87.52 | 77.84 |
| HC 107 | 13.67 | 169.88 | 75.93 | 58.07 |
| HC 108 | 10.28 | 0.05 | 32.08 | 0.04 |
| HC 109 | 30.91 | 91.26 | 65.18 | 48.02 |
| HC 110 | 26.82 | 100.10 | 91.12 | 63.67 |
| HC 111 | 43.88 | 87.38 | 64.52 | 56.43 |
| HC 112 | 50.62 | 148.18 | 109.01 | 90.03 |
| HC 113 | 14.65 | 109.42 | 95.72 | 52.43 |

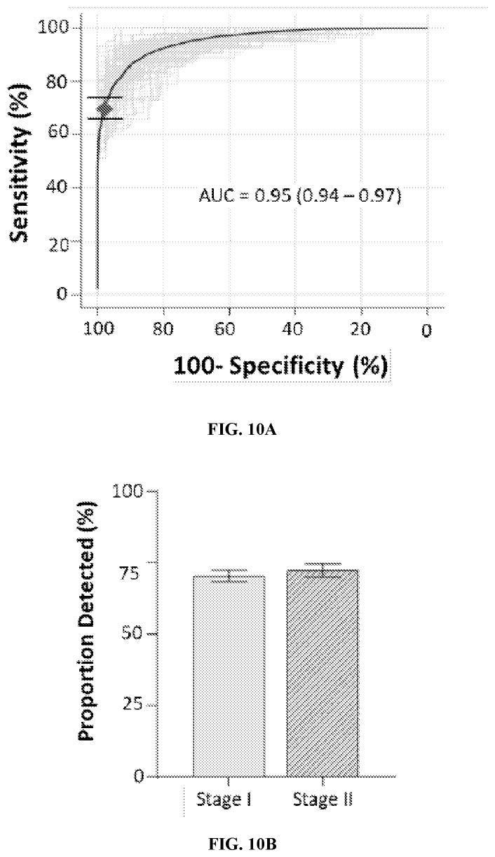

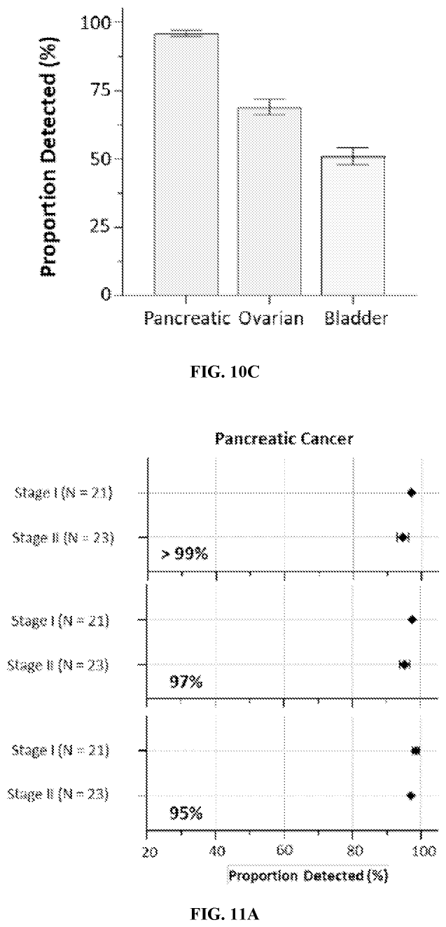

To calculate the overall average ROC (FIG. 9B, Methods), 100 computational iterations were conducted. For each iteration, the total dataset from Table 5 was randomly split into ⅔ Training and ⅓ Test sets. Training sets were used for generation of regression coefficients for each of the biomarkers; Test sets were used to generate Receiver-Operator-Characteristic (ROC) curves and AUC statistics. This rigorous statistical analysis identified 13 proteins that, when combined with patient age, could effectively identify early-stage cancer (FIG. 9C, Tables 2, 6). The resulting average ROC curve is shown in FIG. 10A. When the overall cancer cohort was compared with the healthy individuals using the EXPLORE test, the average AUC was found to be 0.95 (95% CI=0.94-0.97), with a mean sensitivity of 71.2% at specificity >99%.

TABLE 6
Logistic Regression Model Coefficients

| | Logistic | |
|---|---|---|
| | Regression | Standard |
| Feature | Coefficient | Error |
| CA 19-9 | 1.87 | 0.04 |
| Cathepsin D | −2.07 | 0.05 |
| Ferritin | 1.52 | 0.03 |
| IGFBP3 | −2.26 | 0.06 |
| MIA | 2.62 | 0.07 |
| MPO | −1.15 | 0.04 |
| sc-Kit/SCFR | −1.03 | 0.05 |
| sE-selectin | −1.83 | 0.06 |
| sFAS | −1.41 | 0.04 |
| sHER2 | 0.3 | 0.01 |
| sNeuropilin-1 | 0.97 | 0.03 |
| sVEGFR1 | −0.62 | 0.06 |
| TIMP1 | 1.15 | 0.04 |
| Donor Age | 0.17 | 0 |

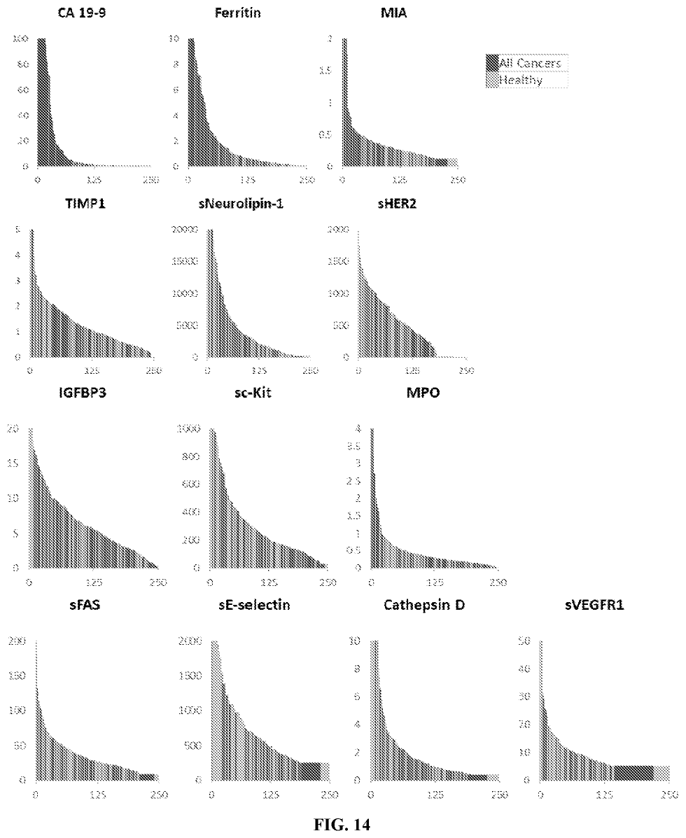

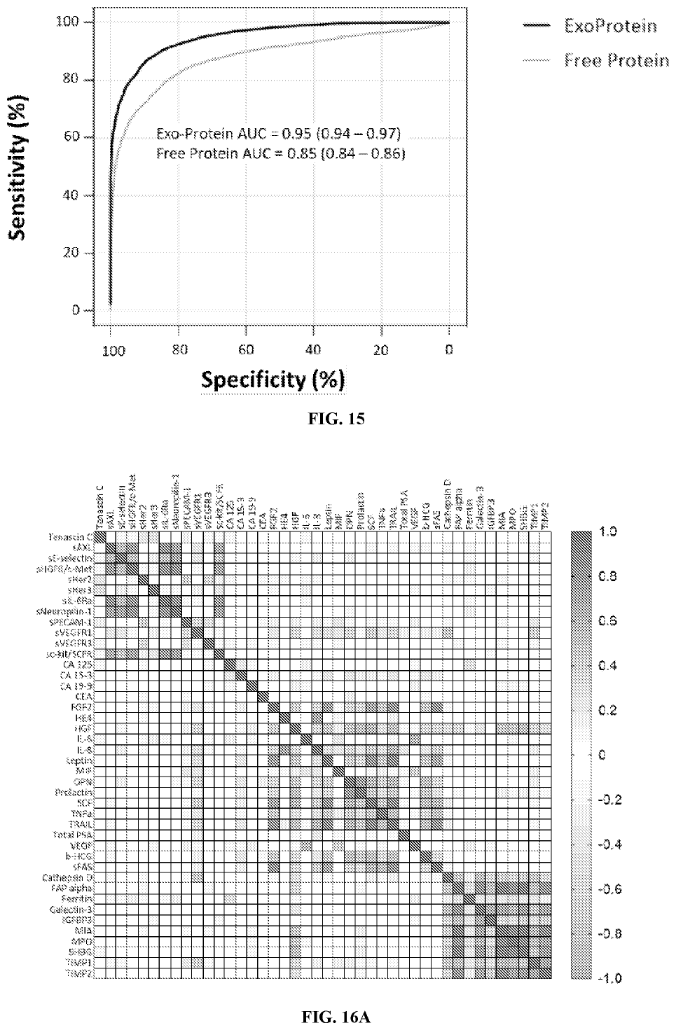

The 13 exo-protein biomarkers used in the EXPLORE test span a wide range of biological functions that may represent pivotal points in cancer development. Neuropilin-1 and HER2 are thought to mediate aberrant growth factor signaling in early malignancies (Niland, S. & Eble, J.A. Neuropilins in the Context of Tumor Vasculature. International Journal of Molecular Sciences 20, 639 (2019); Moasser, M. M. The oncogene HER2: its signaling and transforming functions and its role in human cancer pathogenesis. Oncogene 26, 6469-6487 (2007)). CA 19-9, MPO and TIMP-1 were previously identified in another multi-cancer assay (Liu, M. C., et al. Sensitive and specific multi-cancer detection and localization using methylation signatures in cell-free DNA. Annals of Oncology 31, 745-759 (2020)). VEGFR1, sc-kit/SCFR and sE-selectin may affect angiogenesis (Dvorak, H.F. Vascular Permeability Factor/Vascular Endothelial Growth Factor: A Critical Cytokine in Tumor Angiogenesis and a Potential Target for Diagnosis and Therapy. Journal of Clinical Oncology 20, 4368-4380 (2002); Lennartsson, J. & Römstrand, L. Stem cell factor receptor/c-Kit: from basic science to clinical implications. Physiol Rev 92, 1619-1649 (2012); Kjaergaard, A.G., Dige, A., Nielsen, J.S., Tonnesen, E. & Krog, J. The use of the soluble adhesion molecules sE-selectin, sICAM-1, sVCAM-1, sPECAM-1 and their ligands CD11 a and CD49d as diagnostic and prognostic biomarkers in septic and critically ill non-septic ICU patients. Apmis 124, 846-855 (2016)) while exosomal Cathepsin-D, MIA, IGFBP3, sFas and ferritin are known to impact tumor progression (Hoshino, A., et al. Extracellular Vesicle and Particle Biomarkers Define Multiple Human Cancers. Cell 182, 1044-1061.e1018 (2020); Hoshino, A., et al. Tumour exosome integrins determine organotropic metastasis. Nature 527, 329-335 (2015)) (Tables 6, 7). Waterfall plots for each of the exo-proteins mentioned are shown in FIG. 14 and FIG. 15 shows that the EXPLORE exo-proteins more accurately predict the presence of cancer than their equivalent free-proteins (0.95 vs. 0.85 AUC).

A key feature of a viable screening test is the ability to accurately detect early-stage cancer. At >99% specificity (where only 1 out of 110 healthy falsely identified as positive—Table 2), the EXPLORE test demonstrated sensitivities of 70.4% and 72.3% for stage I and II patients across all cancers, respectively (FIG. 10B). When analyzed by cancer type at >99% specificity, the EXPLORE test demonstrated 69% and 51% sensitivities in ovarian and bladder cancers, respectively, and a notable 96% sensitivity for detecting early-stage pancreatic cancer (FIG. 10C). Unlike previously described multi-cancer assays, EXPLORE demonstrated remarkable sensitivity in detecting early-stage disease in these challenging cancers.

To further understand the potential clinical significance of the EXPLORE test, performance was evaluated at stage and histological breakdowns for each cancer and compared mean sensitivities at three specificity levels used in various screening assays (99%, 97%, 95%).

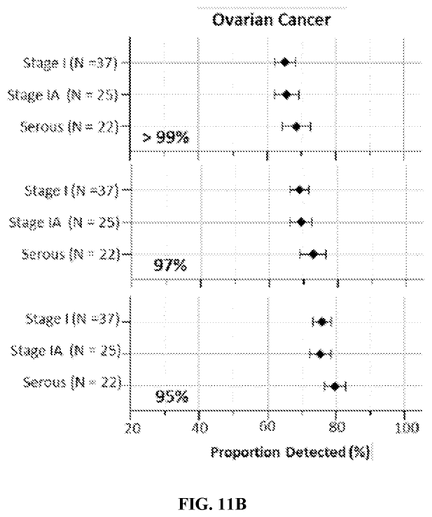

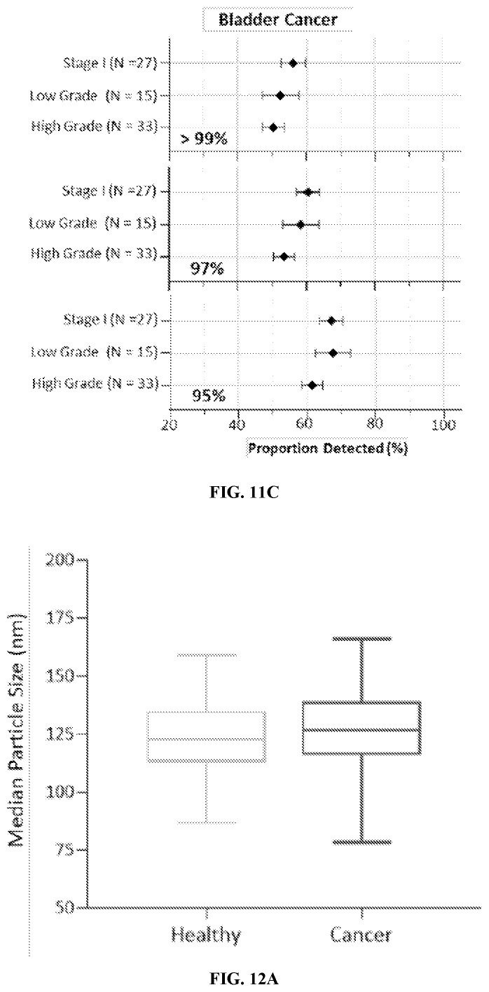

The test demonstrated near-perfect sensitivities in detecting both the 21 Stage I (97%, 98%, 99%) and 23 Stage II (95%, 96%, 97%) in PDAC patients at the highest levels of specificity (FIG. 11A), indicating a potentially dramatic clinical impact. Detection of stage I ovarian cancer (N=37) was also at potentially clinically impactful levels (65%, 69%, 76%), with significant gains in sensitivity observed for lower specificities. Crucially, the high sensitivity of detection for both stage IA (n=25, 66%, 69%, 75%), and the lethally aggressive serous adenocarcinoma histologies (stage I/II, n=22, 69%, 73%, 80%) clearly demonstrates the potential value of the EXPLORE test in ovarian cancer (FIG. 11B). Early detection of either subtype could drastically impact survival rates as surgery would be curative. In bladder cancer, the test was able to detect equally the 27 stage I patients (56%, 61%, 67%), 15 low-grade (52%, 58%, 68%), and 33 high-grade cancers (50%, 54%, 62%), within the 95% CI across all three specificities (FIG. 11C). Interestingly, given a −4% reduction in specificity from >99% to 95%, all three subtypes showed a dramatic increase in mean sensitivity, 11%-16%. Taken as a whole, these results suggest that the EXPLORE test is not biased toward any sub-cohort within each cancer.

While pancreatic and ovarian cancer detection requires −99% specificity to be viable for population-level screening, an argument could be made that bladder cancer may benefit from a lower specificity threshold. Late-stage bladder cancer has a significant impact on quality of life and is among the most expensive to treat in the US. A test with a higher sensitivity may help reduce burden on both patients and the healthcare system by detecting more positives at an early stage where treatment is simpler. The additional false positives (due to lower specificity) could be mitigated by use of non-invasive urine-based confirmatory tests.

In summary, a non-invasive test has been developed combining 13 exosomal proteins with age, a known cofactor in cancer, to detect stage I and II pancreatic, ovarian, and bladder cancers.

The three cancer types studied herein (pancreatic, ovarian and bladder) are estimated to account for roughly 88,000 deaths in the US in 2021, representing approximately 14% of all cancer-related deaths.

Methods

Sample Collection and Processing

All specimens for this study were obtained from a commercial biorepository (ProteoGenex, Culver City, CA, USA). Peripheral blood was collected under appropriate Institutional Review Board/Independent Ethical Committee approval, and all subjects filed informed consent. All subjects with confirmed diagnosis of cancer were treatment naïve (prior to surgery, local, and/or systemic anti-cancer therapy) at the time of blood collection. Demographics, surgical, and pathology information, and AJCC stage (7th edition) were provided by the biorepository and reviewed for accuracy by study authors. Since ovarian cancer patients did not uniformly undergo comprehensive surgical staging, an occult disease higher than the indicated stage cannot be ruled out. A total of 249 subjects were included in the study, including 136 subjects (‘Cancer cohort patients’) who were diagnosed with one of the three cancers between January 2014 and September 2020. In the cancer cohort, whole venous blood specimens were collected shortly after cancer diagnosis (median 1 day, mean 2.7 days), and prior to surgical intervention, radiation therapy, or cancer-related systemic therapy. Median age was 59 years [IQR 54-67] in subjects with known cancer diagnosis (n=136, 56 males, 82 females) and 53 years [IQR 45-61] in subjects without known cancer history (n=113, 49 males and 64 females, Table Si). Whole blood samples were collected in K2EDTA plasma vacutainer tubes and processed into plasma within 4 hours of collection. The whole blood was double spun at 1,500×g for 10 minutes at 4° C. with no brake used. After the first spin, plasma was transferred into fresh tubes and subjected to a second spin at 1,500×g for 10 minutes. After the second spin, plasma was aliquoted into lmL tubes and frozen within 1 hour at −80° C. All specimens used in this study were processed under identical conditions.

Exosome Isolation and Particle Characterization

Exosomes were extracted from 240 μL of plasma as previously described using an AC Electrokinetic-based isolation method (Biological Dynamics, CA, USA). Briefly, undiluted plasma was introduced to a Verita™ chip, where exosomes were captured on microelectrodes. With the AC Electrokinetic field still activated to maintain capture, the remaining plasma was washed away. The AC Electrokinetic field was then deactivated, releasing the exosomes from the microelectrodes, and the solution containing the isolated exosomes was eluted for proteomic analysis. This method has also been used previously for the isolation of cell-free DNA, exosomal RNA and for detection of both solid-tumors and hematological malignancies. Following extraction, EVs were characterized using nanoparticle tracking analysis (NTA) via ZetaView instrument (Particle Metrix, Inning am Ammersee, Germany). Table 3 shows the particle size and concentration values for the exosomes isolated.

Proteomic Analysis

Bead-based immunoassay kits (Human Circulating Biomarker Magnetic Bead Panel 1 (Cat #HCCBP1MAG-58K), Human Angiogenesis Magnetic Bead Panel 2 (Cat #HANG2MAG-12K), and Human Circulating Cancer Biomarker Panel 3 (Cat #HCCBP3MAG-58K)) were procured from a commercial source (Millipore Sigma, Burlington, MA). Extracted exosomes samples and free proteins were tested for concentration of target proteins on a MAGPIX system (Luminex Corp, Austin, TX). Belysa software v. 3.0 (Luminex) was used to determine final protein concentrations.

EXPLORE Test Development

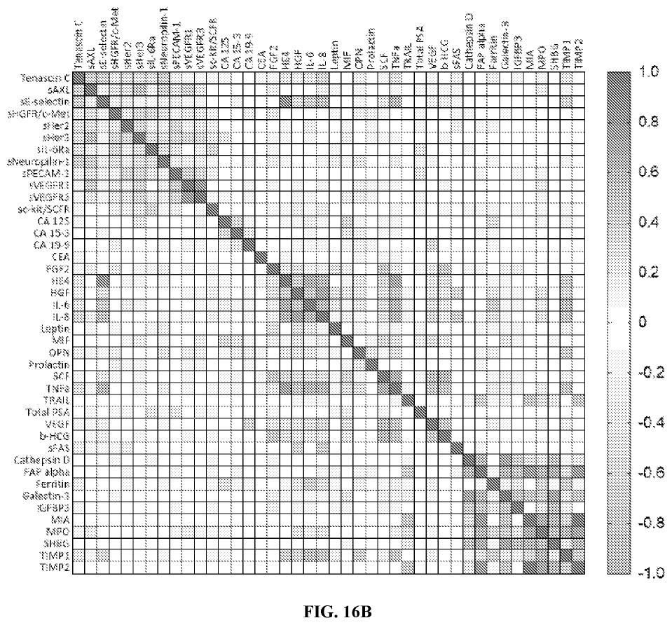

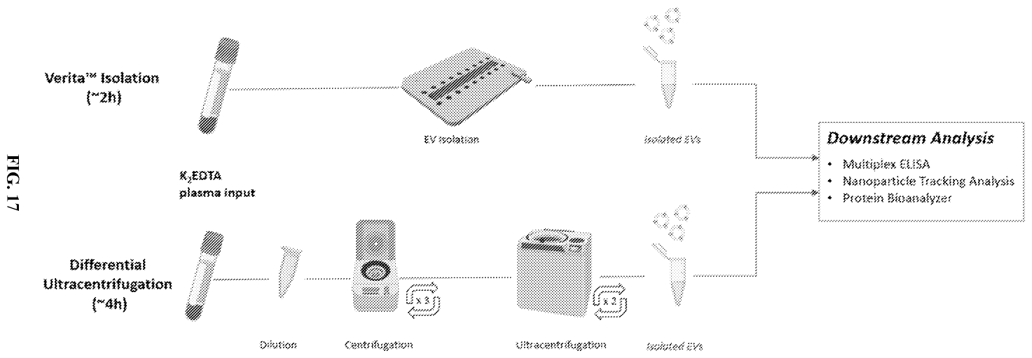

Following an initial evaluation of 54 proteins, 42 different biomarkers were selected for further analysis (Tables 4 & 5). In cases with missing values or results below the limit of detection (LOD), values for that protein were set (imputed) to the LOD. Distributions for all biomarkers were evaluated. Given the wide range of relevant concentrations and the imputed LOD values among the biomarkers, distributions were found to be highly skewed. Thus, a log 2 transformation of all exosomal protein biomarker values was used in subsequent analyses. The R modules ‘outlier’ and ‘GmAMisc’ were used for assessments of outlying values based on standard Grubbs and related tests and found evidence for some extreme values, but none reaching statistical significance, given the number of tests pursued and a conservative Bonferroni correction of relevant p-values. An analysis of outlying individuals based on their biomarker profiles relative to other individuals in the sample was also pursued. Euclidean distance matrices were built across the individuals using the ‘hclust’ module in R. One individual was identified whose profile was extreme relative to the others and this individual was removed from further analyses. The correlations among the biomarkers were explored using the R module ‘Corrplo’ to determine the potential for multicollinearity in building classification models (correlation plots from all the biomarkers measures are shown in FIG. 16). Both standard student t-tests, Wilcoxon non-parametric t-tests, and ANOVA for each biomarker were pursued to explore its association with cancer diagnosis using the R module ‘stats’ (Table 8). The results of these analyses, an assessment of missing and imputed values, and a qualitative literature-based assessment of the biological relevance of each biomarker were used to guide the choice of biomarkers to be evaluated in logistic regression analyses.

Logistic Regression and Receiver-Operator Characteristic Curve (ROC) Analyses.

A logistic regression-based classification models was developed using biomarkers with the ‘caret’ package in R, which is referred to as ‘EXPLORE’. To pursue a fair assessment of the models, given the relatively small sample size, and to avoid over—fitting, 100 random partitions of the data were generated with 66% devoted to a training set and 33% devoted to a test set to evaluate the performance of the EXPLORE classification model (FIG. 9B). Receiver-Operator Characteristic ROC Curves, Area Under the Curve (AUC), and related metrics were computed. The ROC curve and AUC analyses of the training sets resulted in, as expected, better prediction values than those obtained from the test set analyses, but clearly reflected the potential for overfitting. Therefore, the performance of the models is reported based on the training data sets and focus on the performance from the test sets. The resulting models were also used to assess EXPLORE's ability to correctly detect individuals with different cancers and stages of cancer, as well as compare models based on free as opposed to exosome protein levels. During the evaluation of the logistic regression modules, the influence of each individual's profile on the resulting module was assessed using, e.g., leverage statistics in the R module ‘car.’ Based on these analyses, 4 individuals had consistently large influence on the models and, although improvements in the model performance were achieved when these individuals were removed from analyses, this improved performance was not statistically significant from the performance of a model that retained these individuals (data not shown). To ensure robustness of the model and its performance evaluations in subsets of individuals with different cancers and stages, these individuals were excluded from all further analyses.

Automated Classifier Analyses.

As a complement to the choice of biomarkers for use in the classifier, the use of stepwise logistic regression and LASSO-based logistic regression was considered for automated choice of biomarkers in classification models using the R modules ‘caret’ and ‘glmnet’. The performance of the classifiers resulting from the application of these methods did not significantly improve the results, likely due to the small sample size and the multicollinearity among the biomarkers.

Additional Analysis and Plotting

Additional analysis and plotting in both the main text and the supplemental information was done in GraphPad Prism (Version 9.0.2) and IMP (Version 14.1.0).

### Example 5: Preparation of ACE-Purified Exosome Samples for Mass Spectrometry Analysis

Existing standard methods for the preparation of protein samples for mass spectrometry analysis are not sufficient to extract proteins from exosomes, due to the very low buoyant density and tough lipid exterior of exosomes. Furthermore, the components of some elution buffers used to collect exosomes from the ACE chip sometimes presents challenges to standard sample preparation methods for mass spectrometry. Therefore, the following methods were employed to ensure efficient extraction of the full range of proteins to be analyzed.

Using the elution protocol described above, exosomes were purified from human plasma using three separate chips, collected in elution buffer, and then, pooled. To lyse the exosomes, 100 of sample was added to 900 μL of lysis buffer containing the following: (1) detergents such as 2% octylglucoside; (2) protease inhibitors such as phenylmethylsulfonyl fluoride (PMSF), leupeptin, and/or ethylenediaminetetraacetic acid (EDTA); (3) phosphatase inhibitors such as sodium orthovanadate; and (4) denaturing agents such as 4-8 M urea. The mixture was vortexed for 5 minutes followed by probe sonication comprised of 3 separate pulses of 5 seconds each, with the probe set at 20% of the full power. To remove insoluble debris, protein samples were subjected to centrifugation for 10 minutes at 12,000 rpm, and supernatants containing the extracted proteins were collected. Protein disulfide bonds were reduced by the addition of 100 mM dithiothreitol (DTT), followed by alkylation using iodoacetamide. All proteins were precipitated from the sample mixture by addition of trichloroacetic acid (TCA).

To remove any residual TCA, the precipitated sample was washed twice with ice-cold acetone. If the sample pH remained too low, it was adjusted towards neutral by addition of NH₄HCO₃. Then, the sample was subjected to two separate enzymatic digestions, first using Lys C enzyme overnight at 37° C. followed by trypsin for 6 hours at 37° C. To desalt the resulting mixture of peptides, samples were run through a Waters C18 HPLC column, washed with aqueous solution, and eluted using acetonitrile. Peptides were quantified using a Pierce Pepquant kit, and 50 μg of each sample was subjected to mass spectrometry analysis.

Biomarker proteins identified via mass spectrometry analysis of ACE-purified exosomes (Table 7), using the sample preparation method outlined above:

TABLE 7
Exoverita - Proteins of Interest elevated in
pancreatic cancer vs. healthy plasma exosomes

| | |
|---|---|
| Tetraspanin | Ras-related protein Rab-5C |
| Sorbin and SH3 domain containing protein | GTP-binding nuclear protein Ran (Fragment) |
| Leucine-rich alpha-2-glycoprotein | Versican core protein |
| (Fibulin-3) | Ras GTPase-activating protein 3 |
| Vascular cell adhesion protein 1 | Phosphatidylinositide phosphatase SAC1 |
| Periostin | Annexin A3 |
| Thrombospondin-4 | Tenascin-X |

### Example 6: Early Stage Multi-Cancer Detection Using Extracellular Vesicle Protein-Based Blood Test

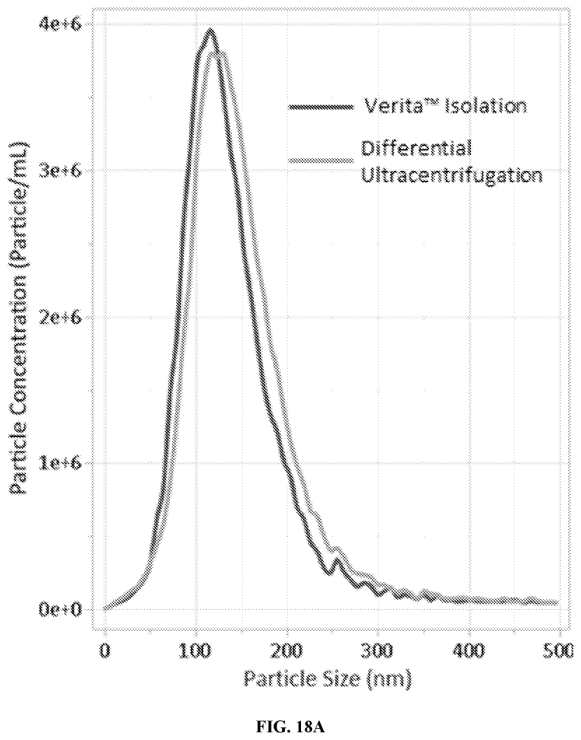

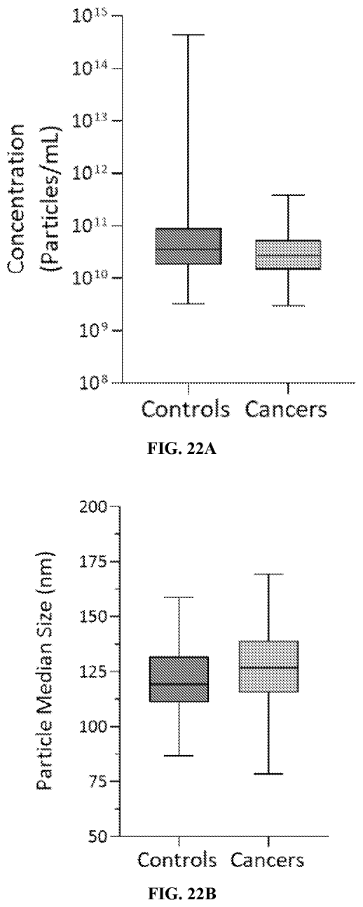

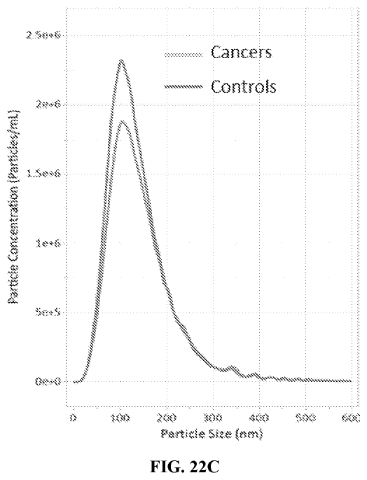

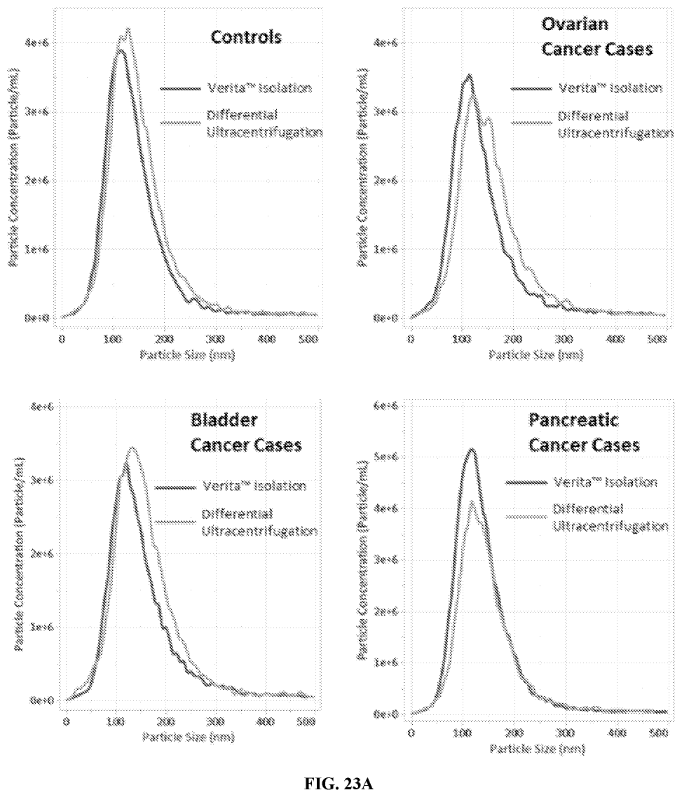

Extracellular vesicles (EV) were isolated from both control plasma and plasma from stage I and II pancreatic, ovarian, and bladder cancer cases (FIG. 17). EV populations isolated using the alternating current electrokinetic (ACE) technology are consistent with the presence of exosomes, in accordance with the ISEV 2018 guidelines24 (mean particle sizing ˜120 nm; CD63-positive; TSG101 can be detected only following membrane permeabilization; scanning electron microscope (SEM) images display rounded, cup-shaped morphology; contain functional RNA). After EV isolation, the particle size distribution and concentration were measured and equivalent isolation for both cohorts was confirmed (FIGS. 22A-22C). To simulate a real-world screening scenario, all cancer cases were treatment-naïve; to ensure that these were early-stage patients, histopathologic staging was confirmed using the American Joint Commission on Cancer (AJCC) guidelines. The median age of the cancer cases was 60 years (59.7% female, 40.3% male). Notably, 63.3% of the overall cancer cases were stage I, with the remaining 36.7% at stage II. Furthermore, the stage I ovarian cohort was comprised predominantly (60%) of stage IA samples. The control group had no known history of cancer or autoimmune disease, with a median age of 57 years (50.0% female, 50.0% male).

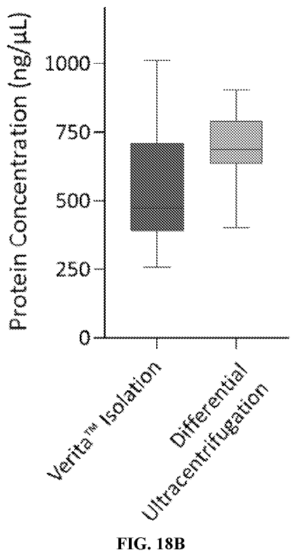

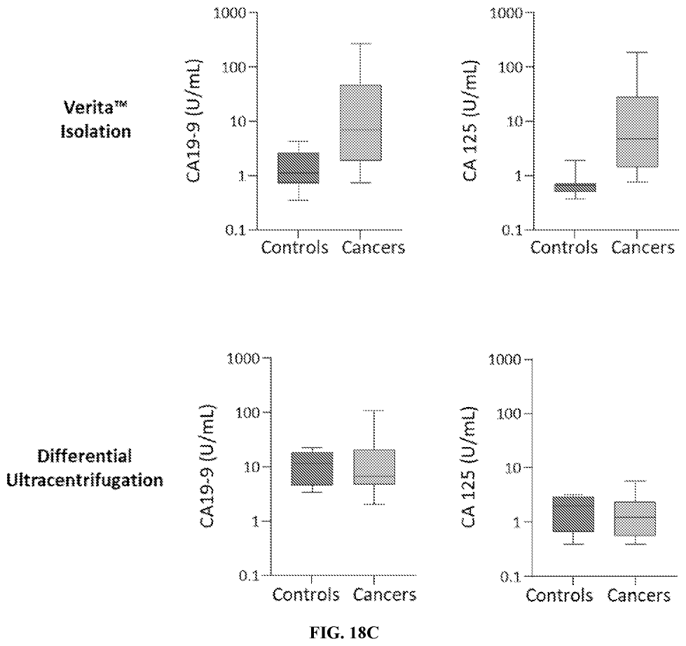

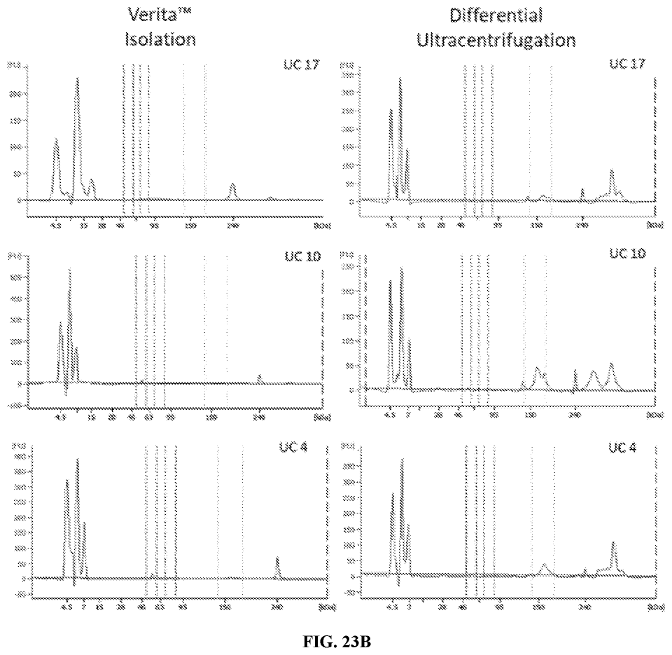

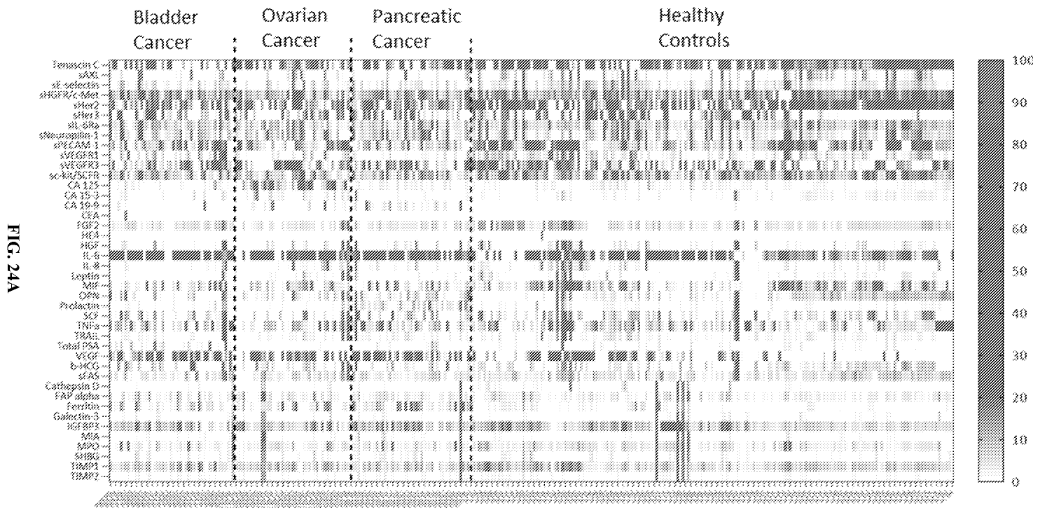

To evaluate the advantages of using ACE-isolated EVs for proteomic analysis, EVs were isolated from a subset of case and control patient samples using either ACE or a differential ultracentrifugation method (FIG. 17). Following isolation, the only physical difference observed between the two methods was a slight decrease in average particle size for EVs isolated on using ACE (138 nm for UC versus 120 nm for ACE EVs; FIG. 18A). Further breakdown of the particle size distributions is shown in FIGS. 23A-23B. When EVs prepared by the two methods were assessed for total plasma protein content, the UC EV preparations were found to contain much higher levels than the ACE EVs (FIG. 18B). For example, contamination with the plasma protein IgG was much higher in the UC isolated material (FIGS. 23A-23B). This is consistent with previous reports that UC-prepared EVs co-purify with protein and nucleic acid aggregates. When EVs purified by the two different methods were compared for their protein biomarker signals we found a strong differentiation between cases and controls for two key biomarkers (CA19-9 and CA 125) from the ACE-isolated EVs, but not for the UC-isolated EVs (FIG. 18C). A summary of the measurements for the EVs from both isolation techniques is shown in Table 8. These results suggests that the ACE EV isolation can be a suitable tool for the purification of EVs directly from plasma and may thus provide a relevant avenue for proteomic analysis. Furthermore, EV isolation using ACE is more efficient, the entire process takes about 2 hours since no added pre- or post-processing steps are required, it does not rely on immunoaffinities, and it involves less of the sample handling which can damage the EVs. Most importantly, unlike UC, ACE isolation of EVs has the potential to be integrated into high-throughput, automated systems.

TABLE 8
Donor histopathology and results for comparison to ultracentrifugation

| | | | | | AJCC | | | | | |
|---|---|---|---|---|---|---|---|---|---|---|
| ID | Method | Cohort | Sex | Age | Stage | A/B/C | T | N | M | Histopathology |
| UC1 | Verita ™ | Ovarian | F | 53 | I | A | T1a | N0 | M0 | serous papillary |
| | | | | | | | | | | cystadenocarcinoma |
| UC2 | Verita ™ | Ovarian | F | 37 | II | B | T2b | N0 | M0 | serous |
| | | | | | | | | | | cystadenocarcinoma |
| UC3 | Verita ™ | Ovarian | F | 43 | I | A | T1a | N0 | M0 | clear cell |
| | | | | | | | | | | adenocarcinoma |
| UC4 | Verita ™ | Ovarian | F | 55 | II | B | T2b | N0 | M0 | serous |
| | | | | | | | | | | adenocarcinoma |
| UC5 | Verita ™ | Bladder | M | 68 | II | | T2a | N0 | M0 | urothelial |
| | | | | | | | | | | carcinoma |
| UC6 | Verita ™ | Bladder | F | 45 | II | | T2b | N0 | M0 | urothelial |
| | | | | | | | | | | carcinoma |
| UC7 | Verita ™ | Bladder | M | 69 | I | | T1 | N0 | M0 | urothelial |
| | | | | | | | | | | carcinoma |
| UC8 | Verita ™ | Bladder | M | 68 | II | | T2a | N0 | M0 | urothelial |
| | | | | | | | | | | carcinoma |
| UC9 | Verita ™ | Pancreatic | M | 60 | II | A | T3 | N0 | M0 | adenocarcinoma |
| UC10 | Verita ™ | Pancreatic | F | 59 | II | A | T3 | N0 | M0 | adenocarcinoma |
| UC11 | Verita ™ | Pancreatic | M | 66 | II | B | T3 | N1 | M0 | adenocarcinoma |
| UC12 | Verita ™ | Pancreatic | F | 58 | II | B | T3 | N1 | M0 | mucinous |
| | | | | | | | | | | adenocarcinoma |
| UC13 | Verita ™ | Pancreatic | M | 67 | II | B | T3 | N1 | M0 | adenocarcinoma |
| UC14 | Verita ™ | Pancreatic | F | 70 | II | B | T2 | N1 | M0 | adenocarcinoma |
| UC15 | Verita ™ | Control | F | 62 | | | | | | |
| UC16 | Verita ™ | Control | F | 60 | | | | | | |
| UC17 | Verita ™ | Control | F | 58 | | | | | | |
| UC18 | Verita ™ | Control | F | 61 | | | | | | |
| UC19 | Verita ™ | Control | F | 55 | | | | | | |
| UC20 | Verita ™ | Control | F | 60 | | | | | | |
| UC21 | Verita ™ | Control | F | 58 | | | | | | |
| UC22 | Verita ™ | Control | M | 60 | | | | | | |
| UC23 | Verita ™ | Control | M | 58 | | | | | | |
| UC24 | Verita ™ | Control | F | 62 | | | | | | |
| UC25 | Verita ™ | Control | F | 58 | | | | | | |
| UC1 | DUC | Ovarian | F | 53 | I | A | T1a | N0 | M0 | serous papillary |
| | | | | | | | | | | cystadenocarcinoma |
| UC2 | DUC | Ovarian | F | 37 | II | B | T2b | N0 | M0 | serous |
| | | | | | | | | | | cystadenocarcinoma |
| UC3 | DUC | Ovarian | F | 43 | I | A | T1a | N0 | M0 | clear cell |
| | | | | | | | | | | adenocarcinoma |
| UC4 | DUC | Ovarian | F | 55 | II | B | T2b | N0 | M0 | serous |
| | | | | | | | | | | adenocarcinoma |
| UC5 | DUC | Bladder | M | 68 | II | | T2a | N0 | M0 | urothelial |
| | | | | | | | | | | carcinoma |
| UC6 | DUC | Bladder | F | 45 | II | | T2b | N0 | M0 | urothelial |
| | | | | | | | | | | carcinoma |
| UC7 | DUC | Bladder | M | 69 | I | | T1 | N0 | M0 | urothelial |
| | | | | | | | | | | carcinoma |
| UC8 | DUC | Bladder | M | 68 | II | | T2a | N0 | M0 | urothelial |
| | | | | | | | | | | carcinoma |
| UC9 | DUC | Pancreatic | M | 60 | II | A | T3 | N0 | M0 | adenocarcinoma |
| UC10 | DUC | Pancreatic | F | 59 | II | A | T3 | N0 | M0 | adenocarcinoma |
| UC11 | DUC | Parcreatic | M | 66 | II | B | T3 | N1 | M0 | adenocarcinoma |
| UC12 | DUC | Pancreatic | F | 58 | II | B | T3 | N1 | M0 | mucinous |
| | | | | | | | | | | adenocarcinoma |
| UC13 | DUC | Pancreatic | M | 67 | II | B | T3 | N1 | M0 | adenocarcinoma |
| UC14 | DUC | Pancreatic | F | 70 | II | B | T2 | N1 | M0 | adenocarcinoma |
| UC15 | DUC | Control | F | 62 | | | | | | |
| UC16 | DUC | Control | F | 60 | | | | | | |
| UC17 | DUC | Control | F | 58 | | | | | | |
| UC18 | DUC | Control | F | 61 | | | | | | |
| UC19 | DUC | Control | F | 55 | | | | | | |
| UC20 | DUC | Control | F | 60 | | | | | | |
| UC21 | DUC | Control | F | 58 | | | | | | |
| UC22 | DUC | Control | M | 60 | | | | | | |
| UC23 | DUC | Control | M | 58 | | | | | | |
| UC24 | DUC | Control | F | 62 | | | | | | |
| UC25 | DUC | Control | F | 58 | | | | | | |

| | Protein | | Median | Peak | | |
|---|---|---|---|---|---|---|
| | Concentration | Particle | Particle | Particle | CA | CA- |
| | Qubit ™ Assay | Concentration | Size | Size | 19-9 | 125 |
| ID | (ng/μL) | (Particles/mL) | (nm) | (nm) | (U/mL) | (U/mL) |
| UC1 | 661.5 | 4.8E+10 | 120.1 | 119.27 | 3.96 | 1.58 |
| UC2 | 1010 | 4.73E+11 | 110.7 | 112.73 | 25.99 | 9.2 |
| UC3 | 418 | 7.2E+10 | 121.9 | 124.9 | 35.01 | 40.69 |
| UC4 | 563 | 3.25E+10 | 112.05 | 110 | 0.73 | 186.81 |
| UC5 | 398.33 | 1.34E+11 | 133.03 | 131.53 | 1.57 | 1.44 |
| UC6 | 380 | 1.15E+11 | 135.93 | 137.67 | 1.95 | 4.09 |
| UC7 | 381 | 3.93E+10 | 119.6 | 125.77 | 7.58 | 1.22 |
| UC8 | 445.67 | 1.44E+11 | 111.8 | 115.63 | 6.22 | 5.59 |
| UC9 | 371 | 1.18E+11 | 111.3 | 116.27 | 273.64 | 63.96 |
| UC10 | 400.5 | 2.93E+11 | 115.43 | 116.57 | 19.44 | 1.38 |
| UC11 | 474 | 4.3E+11 | 117.6 | 117.3 | 1.7 | 0.76 |
| UC12 | 502 | 2.2E+11 | 118.4 | 121.5 | 5.47 | 2.2 |
| UC13 | 462 | 2.21E+11 | 121.9 | 123.2 | 100.72 | 24.17 |
| UC14 | 343 | 4E+11 | 127.03 | 128.03 | 85.83 | 21.88 |
| UC15 | 372.67 | 1.83E+11 | 123.4 | 124.13 | 0.83 | 0.38 |
| UC16 | 825.5 | 7.4E+11 | 117.23 | 114.47 | 1.1 | 0.51 |
| UC17 | 806.5 | 3.75E+11 | 130.5 | 133.65 | 3.27 | 0.61 |
| UC18 | 673 | 4.57E+11 | 127.97 | 127.43 | 1.94 | 0.46 |
| UC19 | 756.5 | 3.55E+11 | 128.55 | 129.05 | 4.37 | 0.68 |
| UC20 | 561 | 1.3E+11 | 117.35 | 117.4 | 1.3 | 0.71 |
| UC21 | 257 | 3.3E+10 | 121.4 | 112.8 | 2.66 | 1.9 |
| UC22 | 931.5 | 3.09E+11 | 125.17 | 127.47 | 0.35 | 0.6 |
| UC23 | 429.5 | 3.18E+11 | 110.8 | 113 | 0.45 | 1.25 |
| UC24 | 583.33 | 2.02E+11 | 111.23 | 114.6 | 0.82 | 0.69 |
| UC25 | 744.67 | 1.62E+11 | 114.13 | 118.2 | 0.73 | 0.69 |
| UC1 | 617.67 | 9.83E+11 | 138.8 | 146.8 | 5.12 | 1.26 |
| UC2 | 560 | 1.02E+12 | 141.17 | 147.57 | 13.74 | 1.67 |
| UC3 | 706.67 | 1.12E+12 | 143.07 | 154.03 | 19.15 | 2.56 |
| UC4 | 778.33 | 7.13E+11 | 135.97 | 139.43 | 5.25 | 5.67 |
| UC5 | 671.67 | 1.27E+12 | 127.4 | 134.33 | 2.02 | 0.39 |
| UC6 | 740.33 | 1.87E+12 | 157.1 | 170.47 | 3.78 | 1.03 |
| UC7 | 684.33 | 1.27E+12 | 140.77 | 148.53 | 7.08 | 0.59 |
| UC8 | 603.67 | 8.93E+11 | 136.67 | 143 | 2.02 | 2.38 |
| UC9 | 640 | 3.84E+11 | 137.1 | 140.87 | 24.81 | 1.52 |
| UC10 | 789 | 7E+11 | 133.3 | 130.25 | 109.06 | 4.04 |
| UC11 | 633.5 | 8.4E+11 | 127.9 | 131.45 | 6.01 | 1.22 |
| UC12 | 791.33 | 6.25E+11 | 140.05 | 146.05 | 6.5 | 0.45 |
| UC13 | 636.33 | 8.9E+11 | 137.93 | 145.8 | 24.35 | 1.16 |
| UC14 | 697 | 5.8E+10 | 139.77 | 142.37 | 8.49 | 0.49 |
| UC15 | 828.67 | 1.28E+12 | 132.9 | 139.1 | 18.53 | 3.16 |
| UC16 | 672.67 | 4.03E+11 | 129.33 | 132.03 | 9.14 | 0.56 |
| UC17 | 400.5 | 4.53E+11 | 132.63 | 139.2 | 4.59 | 0.67 |
| UC18 | 599 | 7.79E+11 | 128.57 | 131.67 | 3.44 | 0.39 |
| UC19 | 688.33 | 3.17E+11 | 135.53 | 140.1 | 4.44 | 1.33 |
| UC20 | 823.33 | 5.73E+11 | 146 | 156.4 | 23.03 | 2.02 |
| UC21 | 639 | 5.33E+11 | 148.77 | 157.63 | 6.25 | 1.42 |
| UC22 | 800.33 | 6.03E+11 | 139 | 150.63 | 12.28 | 2.54 |
| UC23 | 787.67 | 9.57E+11 | 134.47 | 141.87 | 11.65 | 2.31 |
| UC24 | 903.67 | 8.73E+11 | 137.1 | 139.23 | 17.5 | 2.94 |
| UC25 | 827 | 1.17E+12 | 139.1 | 145 | 19.11 | 3.18 |

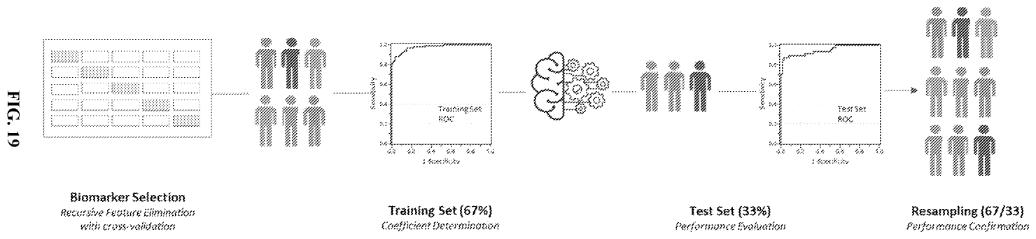

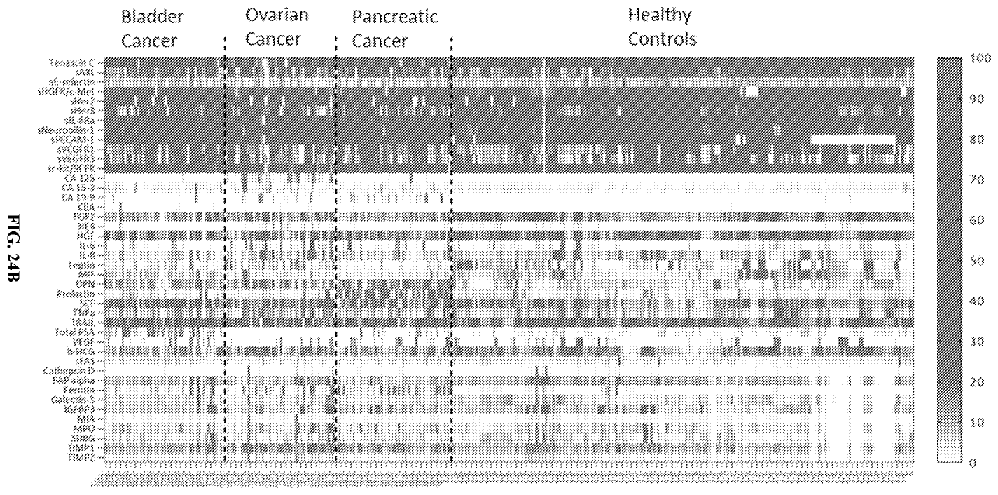

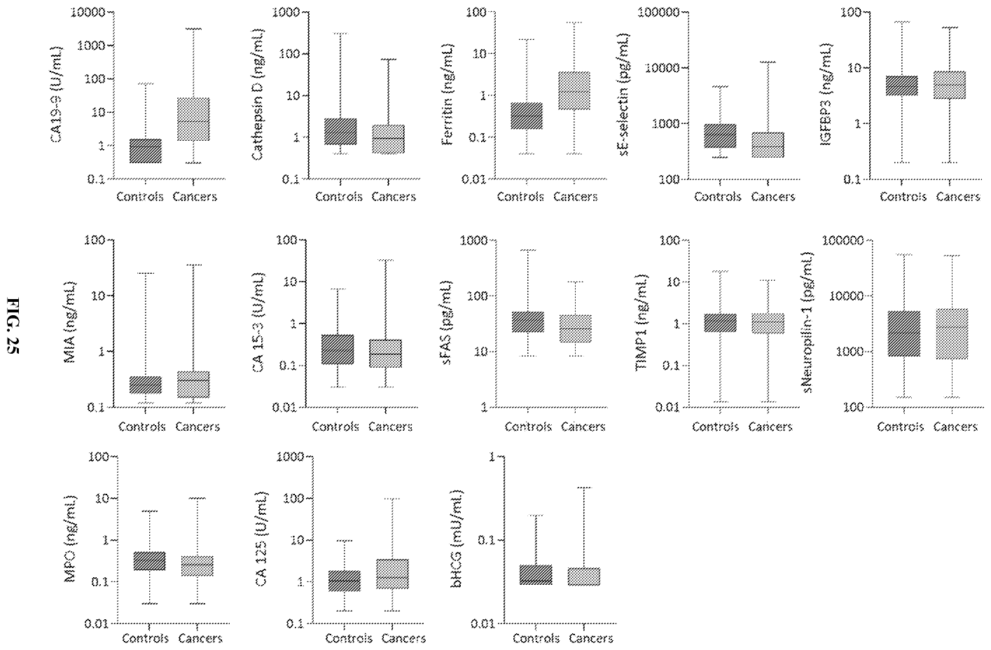

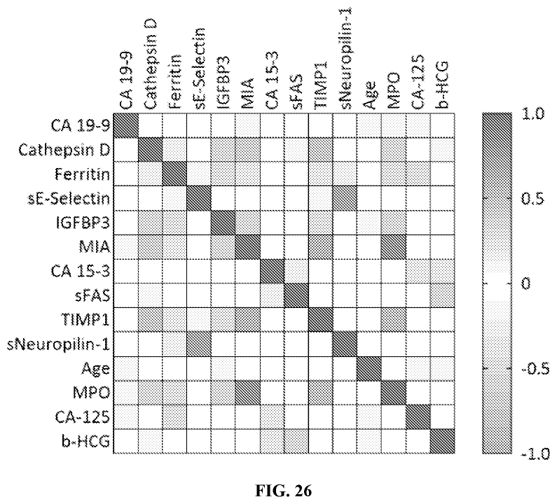

A case-control study involved measurements of the levels of 42 EV-associated protein biomarkers for both the study cohort cancer cases (47 pancreatic, 44 ovarian, 48 bladder) and the controls (184 controls) via a multiplex immunoassay, and an individual assessment of each protein level was performed (heatmaps of the normalized protein levels are shown in FIGS. 24A-24B). Additionally, levels of the unpurified, total circulating plasma proteins (“free proteins”) were measured from the same study cohorts (FIGS. 24A-24B). To identify the EV-associated protein biomarkers with the largest differentiation potential, a selection process was employed to select the most relevant biomarkers based on very high specificities (>99%) using Recursive Feature Elimination (RFE) with cross-validation. The use of repeated cross-validation worked best within the limitations of the sample size for this pilot study (N=323). One hundred repetitions of 5-fold cross validation were performed (FIG. 19), and across all repetitions, the RFE algorithm used stepwise backwards selection to arrive at the optimal number of biomarkers that maximized the partial AUC (pAUC).31 By optimizing the p(AUC) between specificities of 0.75 to 1.00 the biomarker selection was tailored towards the reduction of false positive occurrences (a control mistakenly called as cancer), since this is critical for MCED-type approaches in order to reduce the costs associated with false positive testing. This strategy resulted in the selection of 13 EV protein markers. After the biomarkers were selected, the cohort was separated at random into a training set (67% of the samples) and a “hold-out” set (33% of the samples) stratified by cancer type (pancreatic, ovarian, and bladder) to estimate the respective coefficients for each biomarker in the logistic regression model exploring the potential for detection of cancer at early stages (FIG. 19). The individual logistic regression coefficients were estimated using the training set, while the performance was evaluated in the hold-out test set. Box plots comparing cases and controls for the 13 selected biomarkers are shown in FIG. 25, their coefficient and importance score is shown in Table 9, and their Pearson correlation coefficients in Table 10 and FIG. 26.

TABLE 9
Logistic regression model coefficients

| Feature | Logistic Regression Coefficient | Importance Score |
|---|---|---|
| CA 19-9 | 1.43 | 3.84 |
| Cathepsin D | −1.85 | 2.92 |
| Ferritin | 1.17 | 2.48 |
| sE-selectin | −1.53 | 1.54 |
| IGFBP3 | −1.23 | 1.54 |
| MIA | 1.24 | 1.50 |
| CA 15-3 | −0.90 | 1.43 |
| sFAS | −1.04 | 1.12 |
| TIMP 1 | 0.83 | 1.11 |
| sNeuropilin-1 | 0.46 | 0.99 |
| Age | 0.09 | 0.86 |
| MPO | −0.52 | 0.68 |
| CA 125 | 0.41 | 0.67 |
| b-HCG | 0.85 | 0.54 |

TABLE 10
Pearson correlation coefficients

| | CA | Cathepsin- | | sE- | | | CA | | |
|---|---|---|---|---|---|---|---|---|---|
| Feature | 19-9 | D | Ferritin | Selectin | IGFBP3 | MIA | 15-3 | sFAS | TIMP1 |
| CA 19-9 | 1.00 | 0.01 | 0.06 | −0.03 | −0.04 | 0.10 | 0.02 | 0.00 | 0.01 |
| Cathepsin | 0.01 | 1.00 | 0.14 | 0.06 | 0.34 | 0.38 | 0.00 | 0.07 | 0.47 |
| D | | | | | | | | | |
| Ferritin | 0.06 | 0.14 | 1.00 | 0.07 | 0.27 | 0.20 | −0.02 | −0.06 | 0.21 |
| sE-selectin | −0.03 | 0.06 | 0.07 | 1.00 | −0.01 | −0.06 | −0.02 | 0.04 | 0.10 |
| IGFBP3 | −0.04 | 0.34 | 0.27 | −0.01 | 1.00 | 0.34 | −0.06 | −0.10 | 0.28 |
| MIA | 0.10 | 0.38 | 0.20 | −0.06 | 0.34 | 1.00 | −0.03 | 0.02 | 0.56 |
| CA 15-3 | 0.02 | 0.00 | −0.02 | −0.02 | −0.06 | −0.03 | 1.00 | 0.17 | 0.02 |
| sFAS | 0.00 | 0.07 | −0.06 | 0.04 | −0.10 | 0.02 | 0.17 | 1.00 | 0.01 |
| TIMP1 | 0.01 | 0.47 | 0.21 | 0.10 | 0.28 | 0.56 | 0.02 | 0.01 | 1.00 |
| sNeuropilin- | 0.04 | 0.02 | 0.13 | 0.72 | 0.03 | −0.05 | 0.00 | −0.02 | 0.04 |
| 1 | | | | | | | | | |
| Age | 0.10 | 0.03 | 0.02 | −0.06 | −0.11 | 0.02 | −0.07 | −0.04 | 0.02 |
| MPO | 0.09 | 0.33 | 0.27 | 0.06 | 0.32 | 0.83 | −0.04 | −0.02 | 0.53 |
| CA 125 | 0.10 | −0.01 | 0.29 | 0.03 | −0.04 | 0.01 | 0.21 | −0.01 | 0.01 |
| b-HCG | 0.01 | 0.11 | −0.07 | 0.02 | −0.06 | −0.04 | 0.23 | 0.35 | 0.01 |

| Feature | sNeuropilin | Age | MPO | CA-125 | bHCG |
|---|---|---|---|---|---|
| CA 19-9 | 0.04 | 0.10 | 0.09 | 0.10 | 0.01 |
| Cathepsin | 0.02 | 0.03 | 0.33 | −0.01 | 0.11 |
| D | | | | | |
| Ferritin | 0.13 | 0.02 | 0.27 | 0.29 | −0.07 |
| sE-selectin | 0.72 | −0.06 | 0.06 | 0.03 | 0.02 |
| IGFBP3 | 0.03 | −0.11 | 0.32 | −0.04 | −0.06 |
| MIA | −0.05 | 0.02 | 0.83 | 0.01 | −0.04 |
| CA 15-3 | 0.00 | −0.07 | −0.04 | 0.21 | 0.23 |
| sFAS | −0.02 | −0.04 | −0.02 | −0.01 | 0.35 |
| TIMP1 | 0.04 | 0.02 | 0.53 | 0.01 | 0.01 |
| sNeuropilin- | 1.00 | −0.05 | 0.04 | 0.01 | 0.00 |
| 1 | | | | | |
| Age | −0.05 | 1.00 | 0.05 | −0.17 | 0.08 |
| MPO | 0.04 | 0.05 | 1.00 | 0.04 | −0.06 |
| CA 125 | 0.01 | −0.17 | 0.04 | 1.00 | −0.03 |
| b-HCG | 0.00 | 0.08 | −0.06 | −0.03 | 1.00 |

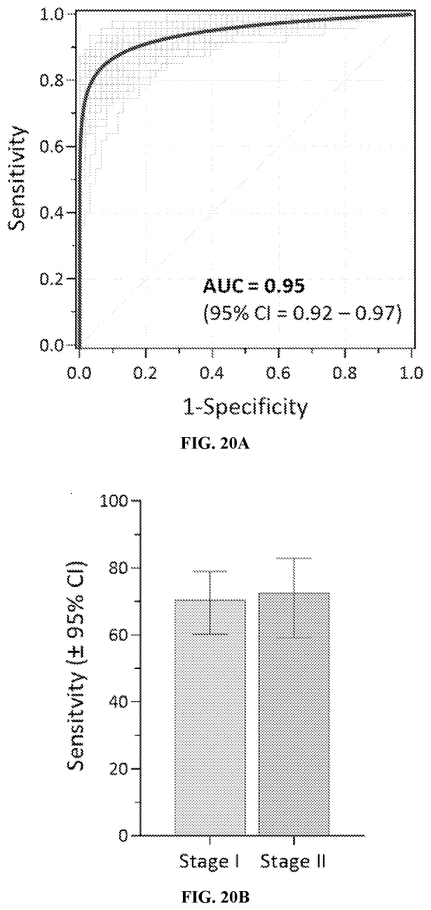

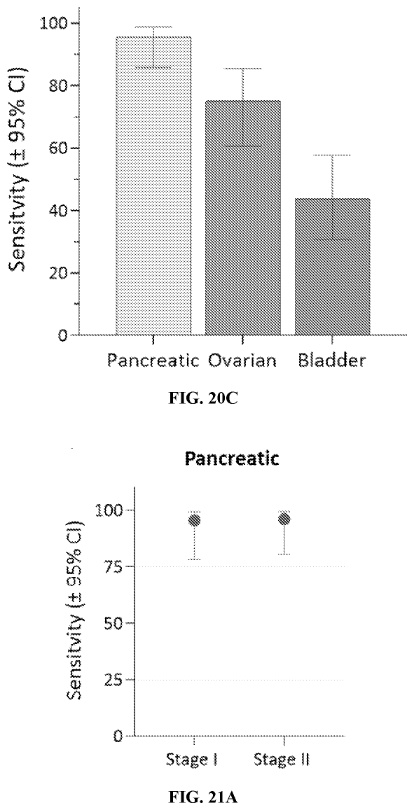

This performance evaluation was strengthened by employing the widely-used statistical process of resampling which better represents how a larger dataset will perform. By resampling, it was evaluated whether the initial random partition created an unrealistic model due to a rare distribution of subjects in that initial partition. One hundred training and test sets were randomly resampled (⅔ and ⅓ of the subjects, respectively) from the overall data and generated 100 individual logistic fits for the training portion; from these fits individual ROC curves were generated for the test sets (FIG. 20A). Likewise, each time a subject was featured in the hold-out test set, a fit for their logistic model was produced and subsequently averaged among all the times that specific subject was used in a test set and from these average fits the overall performance of the model was assessed. The performance for each of the 100 randomly partitioned test sets was assessed individually which, when an average threshold for the target specificity of >99% is computed, permits determination of the overall average sensitivity and confidence intervals.

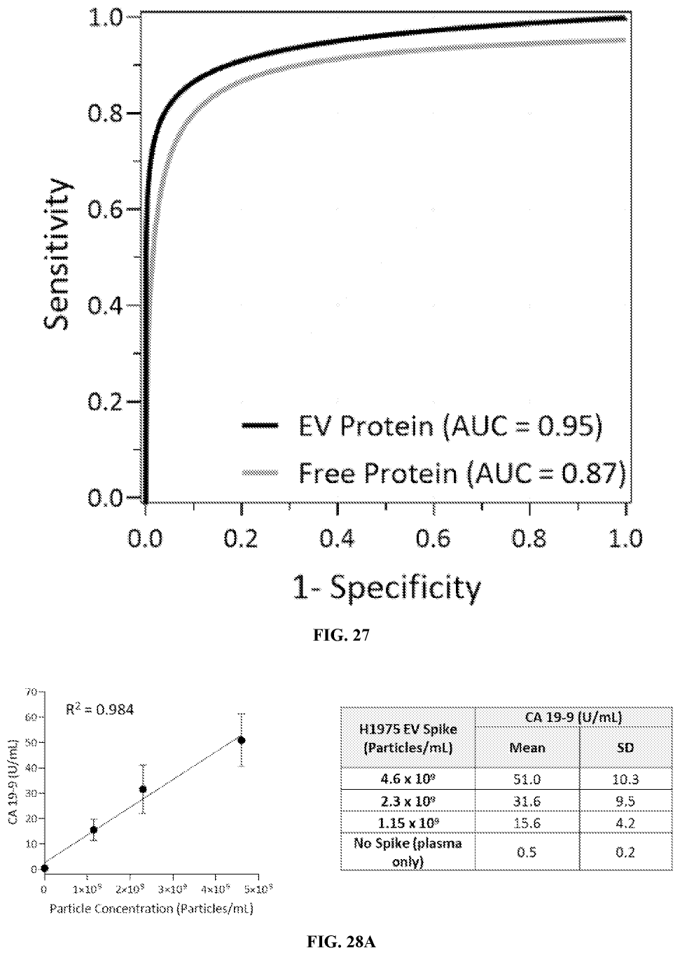

When the overall cancer case cohort was compared with the control individuals using the EV protein biomarker test, the average AUC was found to be 0.95 (95% CI=0.92 to 0.97) as shown in FIG. 20A, with an average sensitivity of 71.2% (95% CI: 63.2 to 78.1) at a specificity of 99.5% (95% CI: 97.0 to 99.9), as shown in Table 11. For the average of the 100 test sets, the AUC for the exo-proteins was found to be larger than that of the equivalent plasma free-proteins (FIG. 27), at 0.95 vs. 0.87, respectively. When considered across all the three cancers studied, our EV protein test demonstrated sensitivities of 70.5% (95% CI: 60.2 to 79.0) and 72.5% (95% CI: 59.1 to 82.9) for stage I and II patients, respectively (FIG. 20B, Table 11). Furthermore, we analyzed the sensitivity at >99% specificity for each individual cancer, finding values of 43.8% (95% CI: 30.7 to 57.7) for bladder cancer, 75.0% (95% CI: 60.6 to 85.4) for ovarian cancer and 95.7% (95% CI: 85.8 to 98.8) for pancreatic cancer (FIG. 20C). These results suggest that EV proteins have the potential for detecting early-stage cancers at screening-relevant sensitivities.

TABLE 11
Performance of Logistic Classifier using EV Proteins

| Category | # Subjects | Specificity (%, 95% CI) | Sensitivity (%, 95% CI) |
|---|---|---|---|
| Control | 184 | 99.5 (97.0-99.9) | |
| All Cancer Cases | 139 | | 71.2 (63.2-78.1) |
| Stage I | 88 | | 70.5 (60.2-79.0) |
| Stage II | 51 | | 72.5 (59.1-82.9) |
| Pancreatic Cancer | 47 | | 95.7 (58.8-98.8) |
| Ovarian Cancer | 44 | | 75.0 (58.9-85.4) |
| Bladder Cancer | 48 | | 43.8 (30.7-57.7) |

Two-sided 95% Wilson confidence intervals

The 13 EV protein biomarkers identified here span a wide range of biological functions that may represent pivotal points in cancer development. Neuropilin-1 and CA15-3 mediate aberrant growth factor signaling in early malignancies. CA 19-9, MPO and TIMP-1, known cancer drivers, were previously utilized in another multi-cancer test. Neuropilin-1 and sE-selectin are known drivers of angiogenesis processes37,38 while exosomal Cathepsin-D, MIA, IGFBP3, sFas and Ferritin have been shown to impact tumor progression. sFAS has been shown to promote cancer stem cell survival, and bHCG may regulate epithelial to mesenchymal transition events in ovarian cancer cell progression. Several of the proteins have previously been shown to be present in EVs. Total serum CA-125 is approved for use in monitoring treatment response and recurrence for ovarian cancer, but it is not recommended to be used as a screening marker. Similarly, total serum CA19-9 is FDA-approved for pancreatic cancer treatment and recurrence monitoring, but importantly, not for screening since on its own CA19-9 may be elevated in several benign conditions.

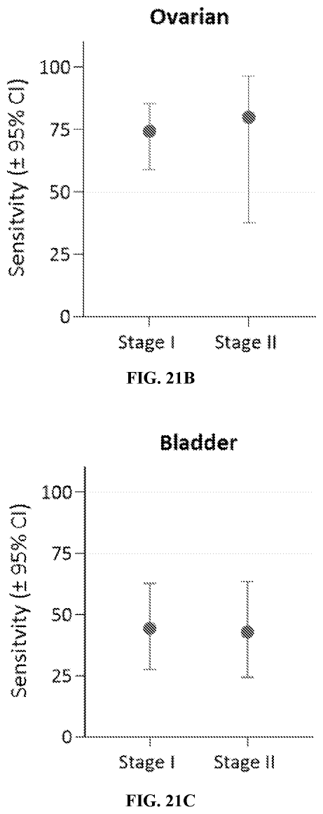

To further understand the potential utility of the EV protein-based test, performance was evaluated at stage for each cancer and compared sensitivities at the 99.5% specificity determined from the overall analysis. With the caveat that sample size for each cancer type was relatively small, the test demonstrated very high sensitivities in detecting both the 22 stage I (95.5%; CI: 78.2 to 99.2) and 25 stage II PDAC patients (96.0%, CI: 80.5 to 99.3) (FIG. 21A), indicating a potential breakthrough for the early detection of this malignancy. Detection of stage I ovarian cancer (N=39) was also at levels with potential clinical impact (74.4%, CI: 58.9 to 85.4) as shown in FIG. 21B. The ovarian cancer cohort was further broken down into both the lethally aggressive serous adenocarcinoma histology (stage I/II, N=22) and stage IA (N=26), showing sensitivities ranging from 68.2% (CI: 47.3 to 83.6) to 73.1% (CI: 53.9 to 86.3 CI) at >99% specificity, respectively. Early detection of either subtype could impact survival rates, as surgery would likely be curative. In bladder cancer, the test was able to detect the 27 stage I patients at 44.4% (CI: 27.6 to 62.7), and the 21 stage II patients at 42.9% (CI: 24.5 to 63.5) as shown in FIG. 21C. The lower sensitivities for bladder cancer, compared to the high sensitivities for pancreatic and ovarian cancer, may reflect the limited availability of suitable biomarkers for detecting early-stage bladder cancer in the panels that were evaluated. In addition, bladder cancer is known to have high molecular and histologic heterogeneity.

Taken as a whole, these results suggest that the EV-based protein biomarker test is not biased toward any sub-cohort within each cancer. While pancreatic ductal adenocarcinoma (PDAC) and ovarian cancer detection require −99% specificity to be viable for population-level screening, an argument could be made that bladder cancer may benefit from a lower specificity threshold. In the emerging field of multi-cancer early detection (MCED) testing, this test is unique because while other tests have the potential to improve the prognosis for later-stage cancer, this test can provide higher sensitivity for detection of early-stage cancer, as exemplified by our 96% sensitivity for stage I and II PDAC cases.

As with any pilot study, there are limitations to acknowledge. First, while informative for biomarker discovery purposes, our relatively small sample cohort, and the inclusion of 100% early-stage tumors does not reflect realistic cancer population characteristics, and sensitivities may be lower when screening large, asymptomatic populations.5,8 However, since survival is directly linked to detecting cancer early, we decided to exclusively focus our cohort on stages I and II. Second, both cohorts are ethnically homogenous, with sex ratios that may be skewed in comparison to the general frequency observed in cancer between males and females.5 Third, our control population consisted of individuals without history of cancer or known confounding comorbidities (e.g., chronic pancreatitis) that in a true screening setting may yield additional false-positive results. Finally, this pilot study will require independent external validation using larger cohorts of blinded samples to verify the potential utility of this MCED approach.

In summary, we have developed a blood-based EV protein detection test and demonstrated its potential role in MCED. The EV protein biomarker test requires less than 500 of plasma and permits integration into an automated workflow. Using a non-invasive blood-based approach, we selected a panel of 13 EV proteins that along with age, a known cofactor in cancer,54 allowed detection of stage I and II pancreatic, ovarian, and bladder cancers with high diagnostic potential (AUC=0.95). Most importantly, we obtained a sensitivity of 71.2% at high specificity (99.5%), a key factor for future screening efforts. This test is the first to effectively utilize EVs in early cancer detection via an AC electrokinetic, lab-on-a-chip, scalable platform. Because the Verita™ platform has multi-omic detection capabilities, addition of other exo-proteins, exosomal mRNA, and/or circulating DNA biomarkers is possible.

Materials & Methods

Sample Collection and Processing

All specimens for this retrospective study were collected over a period of several years by a commercial biorepository (ProteoGenex, Inglewood, CA, USA). Stage I and II samples were selectively obtained from available inventory. Samples had been collected from patients in hospital settings and following collection were maintained by the commercial biorepository. In the hospital settings, potential cancer patients were identified by any suspicious findings arising during imaging that was conducted either in response to patient symptoms or as part of routine, annual examinations. Information on which patients were symptomatic and which were asymptomatic was not available. Cancers were confirmed via subsequent tissue biopsy and staged by pathologists in the hospital using pathology and surgical reports, according to AJCC (7th edition) guidelines, along with imaging to assess any spread to distant sites. All subjects with confirmed diagnosis of cancer were treatment naïve (prior to surgery, local, and/or systemic anti-cancer therapy) at the time of blood collection. The biorepository provided the patient samples along with demographics, surgical, and pathology information. Through the analysis of these data, staging for patients was reviewed a second time for accuracy. Since ovarian cancer patients did not uniformly undergo comprehensive surgical staging, an occult disease higher than the indicated stage cannot be ruled out. The control group has no known cancer history, no known autoimmune diseases, or neurodegenerative diseases as well as no presence of diabetes mellitus (types 1 and 2). A total of 323 subjects were included in the study, including 139 subjects (Cancer case patient cohort′) who were diagnosed with one of the three cancers between January 2014 and September 2020. In the cancer case cohort, whole venous blood specimens were collected shortly before biopsy (median −1 day, mean −2.7 days), and prior to surgical intervention, radiation therapy, or cancer-related systemic therapy. Median age was 60 years [Min-Max 21-76] in the cancer case cohort (N=139, 56 males, 83 females) and 57 years [Min-Max 40-71] in the control cohort (N=184, 82 males and 82 females). Whole blood samples were collected in K2EDTA plasma vacutainer tubes and processed into plasma within 4 hours of collection. The whole blood was first spun at 1,500×g for 10 minutes at 4° C. with no brake used. After the first spin, plasma was transferred into fresh tubes and subjected to a second spin at 1,500×g for 10 minutes. After the second spin, plasma was aliquoted into 1 mL tubes and frozen within 1 hour at −80° C. All specimens used in this study were processed under identical conditions.

EV/Exosome Isolation and Particle Characterization

Isolation of EVs using AC Electrokinetics

EVs, including exosomes, were extracted from plasma as previously described using an AC Electrokinetic (ACE)-based isolation method (Biological Dynamics, CA, USA). Briefly, 240 of each undiluted plasma was introduced into a Verita™ chip, and an electrical signal of 7 Vpp and 14 KHz was applied while flowing the plasma across the chip at 3 μL/min for 120 min. EVs were captured onto the energized microelectrode array, and unbound materials were washed off the chip with Elution Buffer I (Biological Dynamics) for 30 min at 3 μL/min. The electrical signal was turned off, releasing EVs into the solution remaining on the chip (35 μL), which was then collected, and the solution containing purified, concentrated/eluted EVs was used directly for further analysis. This method has also been used previously for the isolation of cell-free DNA, exosomal RNA and exosomal protein markers in both solid-tumors and hematological malignancies. 25,26,55-58 The Verita-purified EVs were characterized using nanoparticle tracking analysis (NTA) via ZetaView instrument (Particle Metrix, Inning am Ammersee, Germany). FIGS. 22A-22C show the particle size and concentration values for the exosomes compared between the case and control cohorts.

Isolation of EVs via Differential Ultracentrifugation

A subset of case and control samples were subjected to differential ultracentrifugation as a conventional means of EV isolation. In brief, 760 μL of 1× PBS was added to 240 μL of each plasma, then spun successively at 500×g for 10 min, 3000×g for 20 min, and 12,000×g for 20 min, collecting the supernatants after each step. Subsequently, the resulting supernatant was subjected to ultracentrifugation at 100,000×g for 70 min, pellets were washed in 1× PBS and then ultracentrifuged again at 100,000×g for 70 minutes. The supernatant was discarded, and the resulting pellet was resuspended in 120 μL of 1× PBS for further analysis.

Protein Contamination Analysis

To determine the presence of contaminating total protein in the EV preparations from both the Verita™ platform and the differential ultracentrifugation process, samples were analyzed using the Qubit 4 fluorometer (ThermoFisher Scientific, Waltham, MA) with the Qubit™ Protein quantitation assay (Cat No. Q33212, ThermoFisher Scientific, Waltham, MA), run according to manufacturer specifications. To further understand the composition of the contaminating proteins on the isolation products, the 2100 Bioanalyzer (Agilent, Santa Clara, CA) with the Protein 230 kit for protein analysis (Cat No. 5067-1517) was used following manufacturer's directions.

Protein Biomarker Analysis

Verita-isolated EV samples, as well as original, unpurified plasma samples from the same patients, were used directly in commercial multiplex immunoassays to quantify the presence of marker proteins. In brief, 2×35 μL of each purified EV sample was used for analysis by each of three different bead-based immunoassay kits, according to the manufacturer's directions for each kit (Human Circulating Biomarker Magnetic Bead Panel 1 (Cat #HCCBP1MAG-58K), Human Angiogenesis Magnetic Bead Panel 2 (Cat #HANG2MAG-12K), and Human Circulating Cancer Biomarker Panel 3 (Cat #HCCBP3MAG-58K); Millipore Sigma, Burlington, MA). Protein biomarker concentration was assessed using the MAGPIX system (Luminex Corp, Austin, TX) according to manufacturer's protocols. Belysa software v. 3.0 (EMD Millipore) was used to determine final protein concentrations from the calibration curves. Limit of Detection (LOD) and units of measure for each of the biomarkers are listed in Table 12.

TABLE 12
Biomarker Limits of Detection

| Biomarker | | Limit of | |
|---|---|---|---|
| Abbreviation | Protein Name | Detection | Units |
| Tenascin C | Tenascin C | 20.2 | pg/mL |
| sAXL | Soluble Axl receptor tyrosine kinase | 5.6 | pg/mL |
| sE-selectin | Soluble E-selectin | 247.8 | pg/mL |
| sHGFR/c-Met | Soluble human growth factor receptor | 24.2 | pg/mL |
| sHer2 | Soluble human epidermal growth factor receptor 2 | 11.9 | pg/mL |
| sHer3 | Soluble human epidermal growth factor receptor 3 | 17.9 | pg/mL |
| sIL-6Ra | Soluble interleukin 6 receptor a | 15.1 | pg/mL |
| sNeuropilin-1 | Soluble Neuropilin-1 | 151 | pg/mL |
| sPECAM-1 | Soluble platelet-endothelial cell adhesion molecule-1 | 15.5 | pg/mL |
| sVEGFR1 | Soluble vascular endothelial growth factor receptor 1 | 5.1 | pg/mL |
| sVEGFR3 | Soluble vascular endothelial growth factor receptor 3 | 233 | pg/mL |
| sc-kit/SCFR | Stem cell factor receptor | 30.5 | pg/mL |
| CA 125 | Cancer Antigen 125 | 0.2 | U/mL |
| CA 15-3 | Cancer Antigen 15-3 | 0.03 | U/mL |
| CA 19-9 | Cancer Antigen 19-9 | 0.3 | U/mL |
| CEA | Carcinoembryonic antigen | 5.2 | pg/mL |
| FGF2 | Basic fibroblast growth factor | 3.6 | pg/mL |
| HE4 | Human epididymis protein 4 | 193.5 | pg/mL |
| HGF | Hepatocyte growth factor | 6.8 | pg/mL |
| IL-6 | Interleukin 6 | 0.2 | pg/mL |
| IL-8 | Interleukin 8 | 0.3 | pg/mL |
| Leptin | Leptin | 42.8 | pg/mL |
| MIF | Macrophage migration inhbitory factor | 7.6 | pg/mL |
| OPN | Osteopontin | 285.3 | pg/mL |
| Prolactin | Prolactin | 30.2 | pg/mL |
| SCF | Stem cell factor | 2 | pg/mL |
| TNFa | Tumor necrosis factor alpha | 0.3 | pg/mL |
| TRAIL | TNF-related apoptosis-inducing ligand | 0.5 | pg/mL |
| Total PSA | Total PSA | 2 | pg/mL |
| VEGF | Vascular endothelial growth factor | 6.4 | pg/mL |
| b-HCG | b-human chorionic gonadotropin | 0.029 | mU/mL |
| sFAS | Soluble tumor necrosis factor receptor superfamily member 6 | 8.4 | pg/mL |
| Cathepsin D | Cathepsin D | 0.4 | ng/mL |
| FAP alpha | Fibroblast activation protein alpha | 0.05 | ng/mL |
| Ferritin | Ferritin | 0.04 | ng/mL |
| Galectin-3 | Galectin-3 | 0.005 | ng/mL |
| IGFBP3 | Insulin-like growth factor binding protein 3 | 0.2 | ng/mL |
| MIA | Melanoma Inhibitory Activity | 0.12 | ng/mL |
| MPO | Myeloperoxidase | 0.03 | ng/mL |
| SHBG | Sex hormone-binding globulin | 0.05 | nM |
| TIMP1 | Tissue inhibitor of metallopeptidase 1 | 0.0136 | ng/mL |
| TIMP2 | Tissue inhibitor of metallopeptidase 2 | 0.0374 | ng/mL |

Spike EV Isolation Models for EV Biomarker Signal

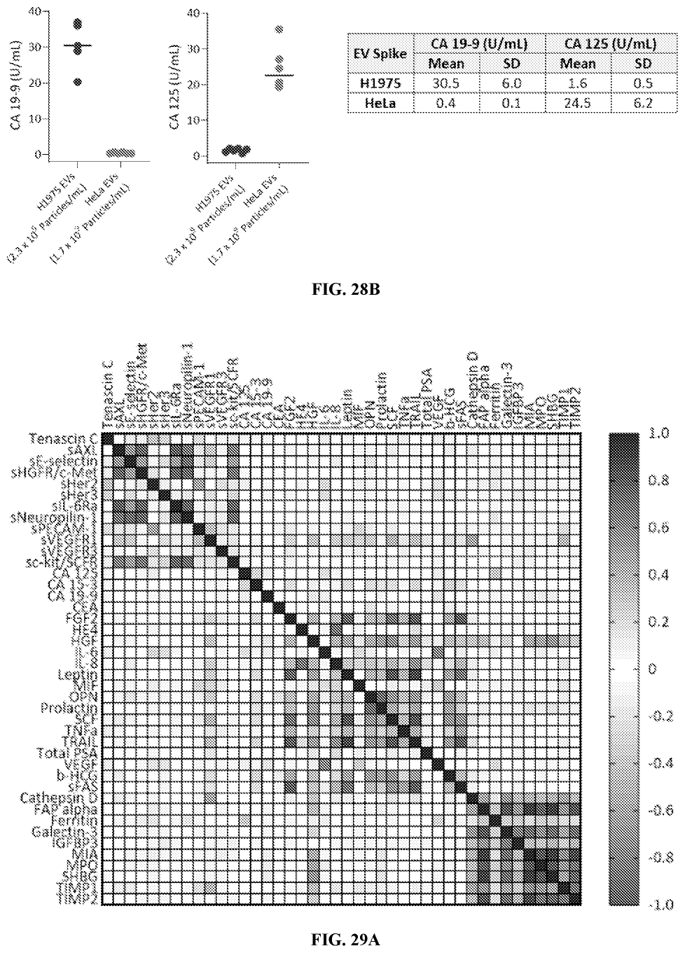

To further understand the presence of relevant protein biomarkers on the EVs, EVs purified from cell culture supernatants representing two different cell lines were employed as positive controls. The cell line H1975 (ATCC CRL-5908™) is known to express the CA19-9 marker while the cell line HeLa (ATCC CRM-CCL-2™) is known to express the CA 125 marker. Briefly, the H1975 EVs were spiked at three different dilution ratios (1:200, 1:400 and 1:800 from the original UC prep) into K2EDTA plasma, the EVs were isolated using the Verita™ platform and subsequently analyzed on the Luminex platform for the presence of the CA 19-9 biomarker (FIGS. 28A-28B). In another experiment, the H1975 EVs and the HeLa EVs were spiked into K2EDTA plasma and isolated using the Verita™ platform. The biomarker reading results confirm the positive detection of the respective expected signals with CA19-9 being elevated for the H1975 EVs and CA 125 being elevated for the HeLa EVs (FIGS. 28A-28B).

EV/exo-protein biomarker test development

Biomarker Selection

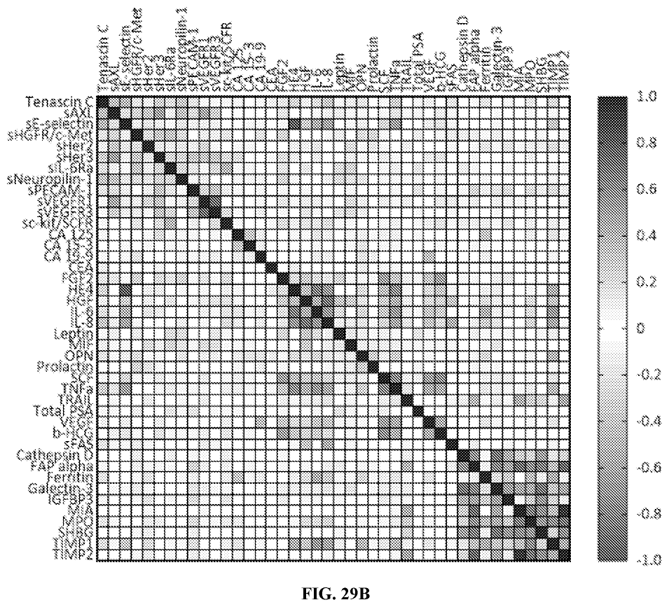

From an initial evaluation of 42 EV proteins, 34 different biomarkers with less than 50% of samples missing or below the limit of detection (LOD) were considered (Table 12). In cases with missing values or results below the LOD, values were set (imputed) to the LOD. Distributions for all biomarkers were evaluated and distributions were found to be wide; thus, a Log2 transformation was used on all EV protein biomarker values in subsequent analyses. The correlations among the biomarkers was explored using the R module ‘Corrplo’ to determine the potential for multicollinearity in building classification models (correlation heatmap from all the biomarkers measures are shown in FIGS. 29A-29B). Subsequently, recursive feature elimination with cross-validation was employed to determine the most informative biomarkers. In this methodology, 4 of the 5 folds are used for selecting a subset of biomarkers using stepwise backwards selection. This process was repeated 5 times, using each fold once as a held-out test set. As the folds of cross-validation were chosen at random, this was repeated 100 times and the subset of biomarkers that maximized the partial AUC (pAUC) over the range of specificities from 75% to 100% across all test sets was selected.

Coefficient Determination and Performance Evaluation

Once the biomarkers were selected, an initial partition of the data into training (67%) and test (33%) sets, stratified by cancer types, allowed determination of the performance of the biomarkers selected by estimating the regression coefficients for the model using the training set and evaluating the classification performance in the hold-out test set (FIG. 19). To pursue a fair assessment of the model, given our relatively small sample size and to avoid overfitting, 100 independent training and test sets (made up of ⅔ and ⅓ of the 323 individuals stratified by cancer type) were resampled from the overall data set. The subjects in the training set, for each resample, were used to estimate biomarker regression coefficients in the model whereas the diagnostic performance was assessed independently in subjects in the hold-out test set. Receiver-Operator Characteristic (ROC) Curves, Area Under the Curve (AUC), sensitivity, specificity and related metrics were computed for the test sets based on the individual fits for each of the subjects in each respective partition. For each of the test sets, a threshold determination of >99% specificity was computed (because there were 61 control subjects in each test set, this effectively means calling 61 out of 61 correctly) and subsequently the average threshold was computed. Using the average threshold and the average fit in the test set for each subject, the performance was evaluated for the overall cohort as well as for subcohorts (e.g., pancreatic cancer). The 95% confidence intervals for AUC were calculated using a bias-corrected bootstrapping method (N=2000) while the confidence intervals for performance metrics, i.e. sensitivity and specificity, were calculated based on the Wilson two-sided method. During the evaluation of the logistic regression model, the importance of each biomarker selected was assessed using the average standardized coefficients (Table 9). Here “importance” can be understood as a quantitative comparison between predictors. One predictor is more important than another if it contributes more to the prediction of the response variable across all the models considered in the regression.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for identifying a biomarker as associated with a disease state, the method comprising:
(a) isolating a first plurality of analytes in a first biological sample of an individual known to have the disease state using an electrode array configured to generate an AC dielectrophoretic field;
(b) isolating a second plurality of analytes in a second biological sample of healthy individual using an electrode array configured to generate an AC dielectrophoretic field; and
(c) identifying a subset of the first plurality of analytes, wherein the subset is quantitatively different in the first biological sample compared with the second biological sample, wherein the subset is identified as associated with the disease state.

2. The method of claim 1, wherein isolating comprises using electrodes configured to generate a dielectrophoretic low field region and a dielectrophoretic high field region.

3. The method of claim 1, wherein isolating comprises capturing the first plurality of analytes or the second plurality of analytes on one or more electrode.

4. The method of claim 1, wherein identifying the subset comprises mass spectrometry analysis of the first plurality of analytes and the second plurality of analytes.

5. The method of claim 1, wherein identifying the subset comprises quantifying each of the first plurality of analytes and the second plurality of analytes.

6. The method of claim 1, wherein the analyte comprises a protein or a polypeptide.

7. The method of claim 1, wherein the analyte comprises a nucleic acid.

8. The method of claim 1, wherein the analyte comprises an exosome.

9. The method of claim 1, wherein (c) comprises subjecting the first plurality of analytes and the second plurality of analytes to mass spectroscopy.

10. The method of claim 1, wherein the disease state is a cancer, a neurological disease, an infection, or an inflammatory disease.

11. The method of claim 9, wherein the cancer is a pancreatic cancer, an ovarian cancer, a bladder cancer, a colorectal cancer, a lung cancer, a brain cancer, a prostate cancer, a breast cancer, a skin cancer, a lymphoma, a tongue cancer, a mouth cancer, a pharynx cancer, an oral cavity cancer, an esophagus cancer, a stomach cancer, a small intestine cancer, a colon cancer, a rectum cancer, an anal cancer, an anorectum cancer, a liver cancer, an intrahepatic bile duct cancer, a gallbladder cancer, a biliary cancer, a digestive organ cancer, a larynx cancer, a bronchus cancer, a respiratory organ cancer, a bone cancer, a joint cancer, a soft tissue cancer, a heart cancer, a melanoma, a nonepithelial skin cancer, a uterine cancer, a cervical cancer, a vulva cancer, a vagina cancer, a penis cancer, a genital cancer, a testis cancer, a kidney cancer, a renal pelvis cancer, a ureter cancer, a urinary organ cancer, an eye cancer, an orbit cancer, a nervous system cancer, an endocrine cancer, a thyroid cancer, a Hodgkin lymphoma, a non-Hodgkin lymphoma, a myeloma, an acute lymphocytic leukemia, a chronic lymphocytic leukemia, an acute myeloid leukemia, a chronic myeloid leukemia, or a leukemia.

12. A method of analysis comprising:
(a) measuring an amount of an analyte in a biological sample from an individual; and
(b) identifying the individual as being at risk of developing a disease when the amount of the analyte is greater than or less than the amount observed in a control sample.

13. The method of claim 12, wherein the analyte comprises one or more biomarker identified in any of the method of claims 1 to 11.

14. The method of claim 12, wherein the analyte comprises one or more proteins provided in Table 5.

15. The method of any one of claims 12 to 14, wherein measuring comprises isolating the analytes in the biological sample using an electrode array configured to generate an AC dielectrophoretic field.

16. The method of claim 15, wherein isolating comprises using electrodes configured to generate a dielectrophoretic low field region and a dielectrophoretic high field region.

17. The method of claim 15, wherein isolating comprises capturing the first plurality of analytes or the second plurality of analytes on one or more electrode.

18. The method of any one of claims 12 to 14, wherein measuring comprises mass spectrometry analysis of the analyte.

19. The method of any one of claims 12 to 14, wherein the analyte comprises a protein or a polypeptide.

20. The method of any one of claims 12 to 14, wherein the analyte comprises a nucleic acid.

21. The method of any one of claims 12 to 14, wherein the analyte comprises an exosome.

22. The method of any one of claims 12 to 14, wherein the disease is a cancer, a neurological disease, an infection, or an inflammatory disease.

23. The method of claim 22, wherein the cancer is a pancreatic cancer, an ovarian cancer, a bladder cancer, a colorectal cancer, a lung cancer, a brain cancer, a prostate cancer, a breast cancer, a skin cancer, a lymphoma, a tongue cancer, a mouth cancer, a pharynx cancer, an oral cavity cancer, an esophagus cancer, a stomach cancer, a small intestine cancer, a colon cancer, a rectum cancer, an anal cancer, an anorectum cancer, a liver cancer, an intrahepatic bile duct cancer, a gallbladder cancer, a biliary cancer, a digestive organ cancer, a larynx cancer, a bronchus cancer, a respiratory organ cancer, a bone cancer, a joint cancer, a soft tissue cancer, a heart cancer, a melanoma, a nonepithelial skin cancer, a uterine cancer, a cervical cancer, a vulva cancer, a vagina cancer, a penis cancer, a genital cancer, a testis cancer, a kidney cancer, a renal pelvis cancer, a ureter cancer, a urinary organ cancer, an eye cancer, an orbit cancer, a nervous system cancer, an endocrine cancer, a thyroid cancer, a Hodgkin lymphoma, a non-Hodgkin lymphoma, a myeloma, an acute lymphocytic leukemia, a chronic lymphocytic leukemia, an acute myeloid leukemia, a chronic myeloid leukemia, or a leukemia.

24. A method of identifying a therapeutic target, the method comprising:
(a) isolating a first plurality of analytes in a first biological sample of an individual known to have the disease state using an electrode array configured to generate an AC dielectrophoretic field;
(b) isolating a second plurality of analytes in a second biological sample of healthy individual using an electrode configured to generate an AC dielectrophoretic field; and
(c) identifying a subset of the first plurality of analytes, wherein the subset is quantitatively different in the first biological sample compared with the second biological sample, wherein the subset is identified as the therapeutic target.

25. The method of claim 24, wherein (c) comprises mass spectroscopy of the first and second plurality of analytes.
